(19) United States
(12) Patent Application Publication
(10) Pub. No.: US 2022/0106547 A1
(43) Pub. Date: Apr. 7, 2022

(54) COMPOSITIONS AND METHODS OF USING PARTIAL GEL LAYERS IN A MICROFLUIDIC DEVICE

(71) Applicant: Emulate, Inc., Boston, MA (US)

(72) Inventors: Debora Barreiros Petropolis, Cambridge, MA (US); Remi Villenave, Boston, MA (US); Janna Nawroth, Boston, MA (US); Tanvi Shroff, Cambridge, MA (US); S. Jordan Kerns, Reading, MA (US); Antonio Varone, West Roxbury, MA (US)

(21) Appl. No.: 17/514,659

(22) Filed: Oct. 29, 2021

Related U.S. Application Data
(63) Continuation of application No. PCT/US2020/032145, filed on May 8, 2020.
(60) Provisional application No. 62/902,618, filed on Sep. 19, 2019.
(60) Provisional application No. 62/869,306, filed on Jul. 1, 2019.
(60) Provisional application No. 62/845,627, filed on May 9, 2019.

(51) Int. Cl. C12M 3/06; C12M 1/12; C12N 5/071

(52) U.S. Cl. CPC C12M 23/16; C12M 25/02; C12N 2535/00; C12N 2533/54; C12N 5/0688

(57) ABSTRACT

The present invention relates to the use of gels for cell cultures, including but not limited to microfluidic devices and transwell devices, for culturing cells, such as organ cells, e.g. airway cells, intestinal cells, etc., and co-culturing cells, (e.g. parenchymal cells and endothelial cells, etc). As one example, the use of gels results in improved lung cell cultures, such as when using transwells and microfluidic devices, (e.g. for culturing healthy airway epithelial cells, culturing diseased airway epithelial cells, e.g., CF epithelial cells that are ciliated). The present invention relates to fluidic devices, methods and systems for use with gel layers within a microfluidic device. In particular, a partial gel layer is disposed within a microchannel of a microfluidic device. For example, a partial gel layer has a thickness ranging between approximately 20-100 μm. A dilute partial gel layer of less than 100 μm may be formed from a polymer solution of 0.5 mg/ml. A cell-permeable partial gel layer having a thickness ranging between approximately 20-50 μm may be formed from a polymer solution of 1-3 mg/ml. A partial gel layer may be formed by a hydrodynamic shearing technique. Such thin gel layers can support a variety of cell cultures, including but not limited to single cells, cell populations, cell layers, differentiated cell layers, and/or primary tissues. The present invention is related to the field of imaging and image processing. In particular, the invention is related to imaging that supports the determination of cell membrane cilia beating frequency. For example, methods described herein encompass cilia beat frequency in the context of membrane region and/or distances between regions. Alternatively, the methods described here encompass cilia beat synchrony and correlation of beat frequency between cell membrane regions.

The present application claims priority to U.S. Provisional Application Ser. No. 62/845,627, filed 9 May 2019; U.S. Provisional Application Ser. No. 62/869,306, filed 1 Jul. 2019 and U.S. Provisional Application Ser. No. 62/902,618, filed 19 Sep. 2019.

### STATEMENT OF GOVERNMENT SUPPORT

This invention was made with government support under grant numbers 1R43AA026473-01 (SBIR grant) awarded by the National Institute of on Alcohol Abuse and Alcoholism and Award Number R43AA026473 awarded by the National Institute on Alcohol Abuse And Alcoholism of the National Institutes of Health. The government has certain rights in the invention.

### FIELD OF THE INVENTION

The present invention relates to the use of gels for cell cultures, including but not limited to microfluidic devices and transwell devices, for culturing cells, such as organ cells, e.g. airway cells, intestinal cells, etc., and co-culturing cells, (e.g. parenchymal cells and endothelial cells, etc). As one example, the use of gels results in improved lung cell cultures, such as when using transwells and microfluidic devices, (e.g. for culturing healthy airway epithelial cells, culturing diseased airway epithelial cells, e.g., CF epithelial cells that are ciliated). The present invention relates to fluidic devices, methods and systems for use with gel layers within a microfluidic device. In particular, in one embodiment, a partial gel layer is disposed within a microchannel of a microfluidic device. For example, a partial gel layer has a thickness ranging between 20 and 500 μm, and more typically approximately 20-100 μm. A dilute partial gel layer of less than 100 μm may be formed from a polymer solution of 0.5 mg/ml. A cell-permeable partial gel layer having a thickness ranging between approximately 20-50 μm may be formed from a polymer solution of 1-3 mg/ml. A partial gel layer may be formed by a hydrodynamic shearing technique. Such thin gel layers can support a variety of cell cultures, including but not limited to single cells, cell populations, cell layers, differentiated cell layers, and/or primary tissues. The present invention is also related to the field of imaging and image processing. In particular, the invention is related to imaging that supports the determination of cell membrane cilia beating frequency. For example, methods described herein encompass cilia beat frequency in the context of membrane region and/or distances between regions. Alternatively, the methods described here encompass cilia beat synchrony and correlation of beat frequency between cell membrane regions.

### BACKGROUND

One of the main functions of epithelia in vivo is to act as physical barriers that protect the underlying tissues from external insults. Epithelial barriers are usually mimicked in vitro by oversimplified models based on cell lines grown as monolayers on flat surfaces. While useful to answer certain questions, these models cannot fully capture the in vivo organ physiology and often yield poor predictions.

In order to engineer more realistic models of human tissues and organs, there has been an effort to combine microfluidic platforms with advances in tissue engineering. For example, to better mimic native tissues and layers between tissues, researchers have attempted to introduce gels and gel layers.

However, introducing gels and gel layers into closed microfluidic devices is surprisingly difficult. Indeed, some researchers have opted for so-called “open-top” microfluidic devices when working with gels. These “open-top” devices typically have an active area (often a region on a central membrane) where the desired conditions or environments for cell culturing introduced, including gel environments. Gels can be pipetted through the open-top and even patterned with a stamp. See PCT Application No.: PCT/US16/64798. However, the use of an open-top device limits the researcher in many respects, and creates a challenge to maintain sterility.

One approach to making gels and gel lumens in a closed microfluidic device is so-called “viscous fingering.” This process hydrostatically drives a less viscous fluid, such as culture medium, through an inlet into a device holding a more viscous fluid, such as a collagen solution in order to form a lumen. See PCT/US16/29164. After formation of a collagen gel, the lumen is seeded with cells. While a useful technique, viscous fingering suffers from non-uniformity. For example, it is difficult to maintain a lumen of uniform size along the length of a microfluidic channel. Indeed, such lumens can have narrow points and even occlusions.

What is needed are better options for forming gels in microfluidic platforms.

Respiratory diseases are on the rise globally. The yearly world market for prescription drugs treating asthma and chronic obstructive pulmonary disease (COPD) alone are projected to reach $47 billion in 2017. Dysfunction of mucociliary clearance is of the most exacerbating factors in many chronic and acute airway diseases. Affected patients suffer from dyspnea, frequent coughs and an increased risk of developing recurrent airways infection, resulting in lower health related quality of life. Athanazio, R. “Airway disease: similarities and differences between asthma, COPD and bronchiectasis” Clinics 67:1335-1343 (2012). Despite the clinical need, very few drugs apart from generic mucus thinning agents specifically target mucociliary clearance. Indeed, not only is the cumulative probability of respiratory drugs reaching the market exceptionally low (3%, versus 6-14% for other disease areas), but most of these “new” drugs are improvements on already approved compounds with bronchodilatory, anti-inflammatory, or antibiotic mechanisms. With few exceptions, there are no clinically approved drugs that control mucociliary clearance. Barnes et al., “Barriers to new drug development in respiratory disease” Eur. Respir. J. 45:1197-1207 (2015); and Ledford, H., “Cystic fibrosis drug Vertex's latest triumph” Nat. Biotechnol. 30:201-202 (2012).

The low rates of innovation for respiratory drugs have several contributory causes, including limited understanding of disease mechanisms and poor preclinical models of the biomechanics of mucociliary transport. Often, biomechanical biomarkers are limited to cell-level measurements of ciliary beat frequency and ultrastructural features, which, by themselves, are incomplete predictors of mucus clearance at the tissue level. These facts indicate the need to develop better preclinical models and a larger panel of biomechanical biomarkers that enable the profiling of human mucociliary transport in disease modeling, drug testing, and diagnostics.

To address this need, what is need in the art is a microphysiological model of the human small airway. Benam et al., “Small airway-on-a-chip enables analysis of human lung inflammation and drug responses in vitro” Nat. Methods (advance online publication) (2015). Such a model would be a powerful tool for studying human ciliated airway physiology in vitro, including real-time measurements of mucus-cilia mechanics at multiple scales, i.e., at the level of cilia, ciliated cells, ciliated tissue, and mucus flow.

### SUMMARY OF THE INVENTION

The present invention relates to the use of gels for cell cultures, including but not limited to microfluidic devices and transwell devices, for culturing cells, such as organ cells, e.g. airway cells, intestinal cells, etc., and co-culturing cells, (e.g. parenchymal cells and endothelial cells, etc). As one example, the use of gels results in improved lung cell cultures, such as when using transwells and microfluidic devices, (e.g. for culturing healthy airway epithelial cells, culturing diseased airway epithelial cells, e.g., CF epithelial cells that are ciliated). The present invention relates to fluidic devices, methods and systems for use with gel layers within a microfluidic device. In particular, a partial gel layer is disposed within a microchannel of a microfluidic device. For example, in one embodiment, a partial gel layer has a thickness ranging between 20-500 μm, and more typically between approximately 20-100 μm. A dilute partial gel layer of less than 100 μm may be formed from a polymer solution of 0.5 mg/ml. A cell-permeable partial gel layer having a thickness ranging between approximately 20-50 μm may be formed from a polymer solution of 1-3 mg/ml. A partial gel layer may be formed by a hydrodynamic shearing technique. Such thin gel layers can support a variety of cell cultures, including but not limited to single cells, cell populations, cell layers, differentiated cell layers, and/or primary tissues. In one embodiment, the gel is contained within the microchannel, i.e. the microchannel is closed (other than having inlet and outlet ports). The present invention is related to the field of imaging and image processing. In particular, the invention is related to imaging that supports the determination of cell membrane cilia beating frequency. For example, methods described herein encompass cilia beat frequency in the context of membrane region and/or distances between regions. Alternatively, the methods described here encompass cilia beat synchrony and correlation of beat frequency between cell membrane regions.

In one embodiment, the present invention contemplates a microfluidic device comprising first and second microchannels in fluidic communication, said first channel comprising a partial gel layer, said partial gel layer comprises a surface that is adjacent to at least one wall of said first microchannel, wherein said surface does not contact said at least one wall of said first microchannel and a fluid path between said partial gel layer and said at least one wall of said first microchannel. In one embodiment, said surface is flat. In one embodiment, said surface is concave. In one embodiment, said partial gel layer is of a uniform composition. In one embodiment, the microfluidic device further comprises a membrane having a first surface and a second surface. In one embodiment, said partial gel layer is disposed on said first membrane surface. In one embodiment, the gel is contained within the microchannel, i.e. the microchannel is closed (other than having inlet and outlet ports). In one embodiment, said partial gel layer is disposed on said second membrane surface. In on embodiment, the microfluidic device, further comprises a first living cell layer on said first membrane surface. In one embodiment, the microfluidic device further comprises a second living cell layer on said second membrane surface. In one embodiment, said partial gel layer is disposed on said first living cell layer. In one embodiment, said partial gel layer is disposed on said second living cell layer. In one embodiment, said fluid path is unoccluded.

In one embodiment, the present invention contemplates, a method, comprising: a) providing: i) immune cells, and ii) a microfluidic device comprising first and second microchannels in fluidic communication, said first channel comprising a partial gel layer, said partial gel layer comprises a surface that is adjacent to at least one wall of said first microchannel, wherein said partial gel layer does not contact said at least one wall of said first microchannel; and iii) a fluid path between said partial gel layer surface and said at least one wall of said first microchannel; and b) introducing said immune cells into said second channel under conditions such that one or more immune cells migrate into said first channel through said partial gel layer. In one embodiment, the method further comprises flowing a fluid through said fluid path. In one embodiment, the gel is contained within the microchannel, i.e. the microchannel is closed (other than having inlet and outlet ports). In one embodiment, the method further comprises detecting activation of at least a portion of said immune cells. In one embodiment, the method further comprises introducing an agent into said first or second microchannels, or both. In one embodiment, said fluid path passes through said first microchannel channel, above said partial gel layer. In one embodiment, said conditions comprise the presence of said agent. In one embodiment, said agent is selected from the group consisting of a virus, an antigen and a cytokine. In one embodiment, said agent creates a recruited immune cell. In one embodiment, said immune cells are T-cells or peripheral blood mononuclear cells.

In one embodiment, the present invention contemplates a method, comprising: a) providing: i) a solution comprising gel monomers; and ii) a microfluidic device comprising at least one microfluidic channel; b) introducing said solution into said at least one microfluidic channel; c) polymerizing said gel monomers to create at least a partially polymerized gel layer; and d) removing a portion of said partially polymerized gel layer to create a partial gel layer comprising a surface that is adjacent to at least one wall of said at least one microfluidic channel, wherein said surface does not contact said at least one wall of said at least one microfluidic channel. In one embodiment, the gel is contained within the microchannel, i.e. the microchannel is closed (other than having inlet and outlet ports). In one embodiment, said solution comprises gel monomers at a concentration of less than 0.5 mg/ml. In one embodiment, said solution comprises gel monomers at a concentration of between approximately 1-3 mg/mi. In one embodiment, said partially polymerized gel layer is a semi-solid gel layer. In one embodiment, said partially polymerized gel layer is a solid gel layer. In one embodiment, said removing further comprises shearing said partially polymerized gel layer with a hydrodynamic fluid device. In one embodiment, said hydrodynamic fluid device comprises a pipette or a syringe. In one embodiment, said surface is flat. In one embodiment, said surface is concave.

In one embodiment, the present invention contemplates, a method, comprising: a) providing; i) a microfluidic device comprising at least one microchannel; ii) a membrane disposed within said at least one microchannel, said membrane having a first surface and a second surface; and iii) a partial gel layer contacting said first surface; b) seeding a plurality of living cells on said partial gel layer; c) culturing said plurality of living cells, wherein said plurality of living cells exhibit at least one differentiation biomarker; and d) scoring said at least one differentiation biomarker to determine a quality control score value for said differentiated plurality of living cells. In one embodiment, the gel is contained within the microchannel, i.e. the microchannel is closed (other than having inlet and outlet ports). In one embodiment, the quality control score is less than one (1) and said cultured plurality of living cells are healthy and well differentiated. In one embodiment, the quality control score is between one (1) and two (2) and said cultured plurality of living cells are healthy and partially differentiated. In one embodiment, the quality control score is between two (2) and three (3) and said cultured plurality of living cells are healthy and not differentiated. In one embodiment, the quality control score is greater than three (3) and said cultured plurality of living cells are not healthy and not differentiated. In one embodiment, the at least one differentiation biomarker is selected from the group consisting of cell death, ciliation, cell attachment, cell movement, cell shape, cell overgrowth and cell invasion. In one embodiment, the cell death biomarker score value increases in proportion to increased cell death. In one embodiment, the ciliation biomarker score value increases in proportion to deceased ciliation. In one embodiment, the cell attachment biomarker score value increases in proportion to decreased cell attachment. In one embodiment, the cell movement biomarker score value increases with decreased cell movement. In one embodiment, the cell shape biomarker score value increases with an irregular shape. In one embodiment, the cell overgrowth biomarker score value increases with increased cell overgrowth. In one embodiment, the cell invasion biomarker score value increases with increased cell invasion. In one embodiment, the partial gel layer comprises bovine serum albumin. In one embodiment, the partial gel layer comprises collagen IV. In one embodiment, the partial gel layer comprises collagen I. In one embodiment, the partial gel layer comprises collagen I and collagen IV. In one embodiment, the microchannel further comprises a cell culture media. In one embodiment, the cell culture media comprises a retinoic acid-related compound, EC-23. In one embodiment, the quality control score further comprises a differentiation endpoint score. In one embodiment, the differentiation endpoint score comprises a cilia density score. In one embodiment, the differentiation endpoint score comprises a cilia beat frequency score. In one embodiment, the differentiation endpoint score comprises a cytokine score. In one embodiment, the differentiation endpoint score comprises a mucus production score. In one embodiment, the differentiation endpoint score comprises a morphology score. In one embodiment, the differentiation endpoint score comprises a cell integrity score. In one embodiment, the differentiation endpoint score comprises a cell function score.

In one embodiment, the present invention contemplates a method, comprising: a) providing; i) a microfluidic device comprising at least one microchannel; ii) a membrane disposed within said at least one microchannel, said membrane having a first surface and a second surface; and iii) a partial gel layer contacting said first surface; b) seeding a plurality of living diseased cells on said partial gel layer; c) culturing said plurality of living diseased cells, wherein said plurality of living diseased cells exhibit at least one differentiation biomarker; and d) scoring said at least one differentiation biomarker to determine a differentiated plurality of living diseased cells. In one embodiment, the gel is contained within the microchannel, i.e. the microchannel is closed (other than having inlet and outlet ports). In one embodiment, the plurality of living diseased cells are sensitive disease cells. In one embodiment, the sensitive disease cells are cystic fibrosis lung cells. In one embodiment, the method further comprises seeding said membrane with fibroblast cells to form an interstitial tissue layer beneath said plurality of living diseased cells. In one embodiment, the partial gel layer comprises a collagen I/collagen IV matrix. In one embodiment, the microchannel further comprises a cell culture media. In one embodiment, the cell culture media comprises a retinoic acid-related compound, EC-23. In one embodiment, the method further comprises flowing said cell culture media approximately six (6) hours after said diseased cell seeding. In one embodiment, the at least one differentiation biomarker is a ciliary biomarker and has a quality control score of less than two (2). In one embodiment, the at least one differentiation biomarker is a mucociliary biomarker and has a quality control score of less than two (2).

In one embodiment, the present invention contemplates a method, comprising: a) providing; i) a gelling fluid comprising at least one polymer monomer, said at least one polymer monomer having a concentration of less than 0.5 mg/ml; ii) a microchannel having an inlet port and a outlet port; b) injecting said gelling fluid at a slow flow rate into said inlet port to fill said microchannel; c) polymerizing said gelling fluid for approximately twelve to sixteen (12-16) hours to form a semi-solid partial gel layer. In one embodiment, the partial gel layer does not contact at least one microchannel wall. In one embodiment, the partial gel layer is approximately 100 μm thick. In one embodiment, the partial gel layer has a smooth surface. In one embodiment, the gel is contained within the microchannel, i.e. the microchannel is closed (other than having inlet and outlet ports).

In one embodiment, the present invention contemplates a method, comprising: a) providing; i) a gelling fluid comprising at least two polymer monomers, said at least two polymer monomers having a concentration of approximately 0.8 mg/ml; ii) a microchannel having an inlet port and a outlet port; and iii) an automatic pipettor comprising a fluid; b) injecting said gelling fluid into said inlet port to fill said microchannel; c) polymerizing said gelling fluid for approximately twelve-sixteen (12-16) hours to form a solid gel; and d) shearing said solid gel with said fluid at a high flow rate to create a partial gel layer. In one embodiment, the partial gel layer does not contact at least one microchannel wall. In one embodiment, the partial gel layer is approximately between 20-50 μm thick. In one embodiment, the partial gel layer has a smooth surface. In one embodiment, the gel is contained within the microchannel, i.e. the microchannel is closed (other than having inlet and outlet ports).

In one embodiment, the present invention contemplates a method, comprising: a) providing; i) a gelling fluid comprising at least one polymer monomer, said at least one polymer monomer having a concentration of approximately between 1-3 mg/ml; ii) a microchannel having an inlet port and a outlet port; and iii) a manual pipettor comprising a fluid; b) injecting said gelling fluid through said inlet port without bubble formation; c) polymerizing said gelling fluid for approximately forty-five (45) minutes; d) flushing said fluid into said inlet port at a slow flow rate with said manual pipettor until said gelling fluid ejects into said outlet port; and e) gravity flushing said fluid to create a partial gel layer. In one embodiment, the partial gel layer does not contact at least one microchannel wall. In one embodiment, the partial gel layer is approximately between 20-50 μm thick. In one embodiment, the partial gel layer has a smooth surface. In one embodiment, the gel is contained within the microchannel, i.e. the microchannel is closed (other than having inlet and outlet ports).

In one embodiment, the present invention contemplates a microchannel comprising at least two partial gel layers. In one embodiment, the microchannel comprises three partial gel layers. In one embodiment, a first thin partial gel layer is deposited on the membrane surface. Although it is not necessary to understand the mechanism of an invention, it is believed that the first thin partial gel layer prevents undesirable cell migration through the membrane. In one embodiment, a second partial gel layer comprises cells. In one embodiment, the cells are stromal cells. In one embodiment, the third partial gel layer is cell-free. In one embodiment, the third partial gel layer comprises epithelial cells. In one embodiment, the third partial gel layer comprises parenchymal cells. In one embodiment, the at least two partial gel layers comprise different compositions. In one embodiment, the at least two partial gel layers comprise different thicknesses. In one embodiment, the at least two partial gel layers comprise the same composition. In one embodiment, the at least two partial gel layers comprise the same thicknesses. In one embodiment, each of the at least two partial gel layers have a different cell type. In one embodiment, the different cell type are different neuronal cell types.

In one embodiment, the present invention contemplates a method, comprising: a) providing; i) an in vitro cell culture comprising a plurality of cells, wherein each of said plurality of cells express at least one viability/differentiation biomarker; and ii) a cell imaging system configured to detect said at least one viability/differentiation biomarker; b) detecting said at least one viability/different ion biomarker with said cell imaging system; c) scoring said detected at least one viability/differentiation biomarker; and d) determining the viability and differentiation status of said each of said plurality of cells based upon said score. In one embodiment, the at least one viability/differentiation biomarker is selected from the group consisting of cell death, ciliation, cell attachment, cell movement and shape, cell overgrowth, cell invasion, cilia density, ciliary beat frequency, cytokine production, mucus production, cell integrity and cell function.

In one embodiment, the scoring creates a score value that increases as the viability and differentiation status of said each of said plurality of cells decrease. In one embodiment, the score value is between 0-2 and predicts cell viability and differentiation. In one embodiment, the score value is between 3-5 and does not predict cell viability and differentiation. In one embodiment, the score value is a cell death biomarker score value that increases in proportion to increased cell death. In one embodiment, the score value is a ciliation biomarker score value that increases in proportion to deceased ciliation. In one embodiment, the score value is a cell attachment biomarker score value that increases in proportion to decreased cell attachment. In one embodiment, the score value is a cell movement and shape biomarker score value that increases with decreased cell movement/shape. In one embodiment, the score value is a cell overgrowth biomarker score value that increases with increased cell overgrowth. In one embodiment, the score value is a cell invasion biomarker score value that increases with increased cell invasion accompanied by fibroblast morphology development. In one embodiment, the in vitro cell culture further comprises a gel coating. In one embodiment, the scoring determines said viability and differentiation status for said each of said plurality of cells with said gel coating. In one embodiment, the in vitro cell culture further comprises a cell culture media. In one embodiment, the scoring determines said viability and differentiation status for said each of said plurality of cells with said cell culture media.

In one embodiment, the present invention contemplates a method, comprising: a) providing a microfluidic device having a microchannel with a partial gel overlay and a hepatocyte cell layer; b) differentiating said hepatocyte cell layer to create bile canaliculi; c) scoring said bile canaliculi to determine a differentiation stage. In one embodiment, the partial gel overlay is approximately between 25-100 μm. In one embodiment, the partial gel overlay comprises a polymer selected from the group consisting of collagen I, collagen IV, fibronectin and fibricol. In one embodiment, the scoring further comprises a geometrical matrix. In one embodiment, the geometrical metric comprises a branching density defined by the equation ΣLₙ/Area of RO_{I}. In one embodiment, the geometrical metric comprises a porosity defined by the equation Σ Aₙ/Area of RO_{I}. In one embodiment, the differentiation stage comprises an increased BC network quality as determined by a decrease in average BC radius. In one embodiment, the differentiation stage comprises an increased bile canaliculi network quality as determined by an increase in branching density. In one embodiment, the differentiation stage comprises an increased bile canaliculi network quality as determined by an increase in porosity. In one embodiment, the scoring determines said differentiation stage with said partial gel overlay. In one embodiment, the scoring further comprises a shape analysis parameter. In one embodiment, the shape analysis parameter is selected from the group consisting of bile canaliculi circularity, bile canaliculi length and bile canaliculi solidity. In one embodiment, the shape analysis parameter determines a maturity stage of said bile canaliculi. In one embodiment, the microchannel further comprises a gel underlay. In one embodiment, the gel underlay comprises a hepatic stellate cell. In one embodiment, the gel is contained within the microchannel, i.e. the microchannel is closed (other than having inlet and outlet ports)

In one embodiment, the present invention contemplates a method, comprising: a) providing a microfluidic device having a microchannel with a partial thick gel overlay and a hepatocyte cell layer; b) differentiating said hepatocyte cell layer to create bile canaliculi; c) scoring said bile canaliculi to determine a differentiation stage. In one embodiment, the partial thick gel overlay is approximately between 25-100 μm. In one embodiment, the partial thick gel overlay comprises a polymer selected from the group consisting of collagen I, collagen IV, fibronectin and fibricol. In one embodiment, the scoring further comprises a geometrical matrix. In one embodiment, the geometrical metric comprises a branching density defined by the equation ΣLₙ/Area of RO_{I}. In one embodiment, the geometrical metric comprises a porosity defined by the equation Σ Aₙ/Area of RO_{I}. In one embodiment, the differentiation stage comprises an increased BC network quality as determined by a decrease in average BC radius. In one embodiment, the differentiation stage comprises an increased bile canaliculi network quality as determined by an increase in branching density. In one embodiment, the differentiation stage comprises an increased bile canaliculi network quality as determined by an increase in porosity. In one embodiment, the scoring determines said differentiation stage with said partial thick gel overlay. In one embodiment, the scoring further comprises a shape analysis parameter. In one embodiment, the shape analysis parameter is selected from the group consisting of bile canaliculi circularity, bile canaliculi length and bile canaliculi solidity. In one embodiment, the shape analysis parameter determines a maturity stage of said bile canaliculi. In one embodiment, the microchannel further comprises a gel underlay. In one embodiment, the gel underlay comprises a hepatic stellate cell.

In one embodiment, the present invention contemplates a microfluidic device, comprising: a) a microchannel comprising a membrane, said membrane having a surface; b) an underlay gel layer disposed on said surface; c) a cell layer adhered to said underlay gel layer; and d) an overlay gel layer deposited on said cell layer. In one embodiment, the cell layer is a hepatocyte cell layer. In one embodiment, the device further comprises a molecular extracellular matrix layer disposed between said surface and said underlay gel layer. In one embodiment, the underlay gel layer comprises stellate hepatic cells.

In one embodiment, the present invention contemplates a method, comprising: a) providing; i) a microfluidic device comprising a microchannel, said microchannel comprising a surface; ii) a gel matrix solution comprising at least one polymer at a concentration of <0.5 mg/ml; and iii) a instrument comprising a fluid; b) loading said gel matrix solution into said microchannel; c) polymerizing said gel matrix solution overnight; d) flushing an unpolymerized gel solution matrix from said microchannel with said instrument; and e) forming an anchored three dimensional partial gel layer on said surface, wherein said partial gel layer comprises a flat surface and has a relatively homogenous thickness. In one embodiment, the gel is contained within the microchannel, i.e. the microchannel is closed (other than having inlet and outlet ports). In one embodiment, the instrument is selected from the group consisting of a manual syringe and an automatic pipettor. In one embodiment, the flushing comprises a shear velocity of approximately 187.5 μl/sec. In one embodiment, said partial gel layer comprises a thickness that ranges between 50-200 μm. In one embodiment, the at least one polymer is selected from the group consisting of collagen I and fibronectin. In one embodiment, the method further comprises calculating said thickness with a thresholding algorithm.

In one embodiment, the present invention contemplates a method, comprising: a) providing; i) a microfluidic device comprising a microchannel, said microchannel comprising a surface; ii) a gel matrix solution comprising at least one polymer at a concentration of <0.5 mg/ml; and iii) a instrument comprising a fluid; b) loading said gel matrix solution into said microchannel; c) polymerizing said gel matrix solution overnight; d) flushing an unpolymerized gel solution matrix from said microchannel with said instrument; and e) forming an anchored three dimensional partial thick gel layer on said surface, wherein said partial thick gel layer comprises a flat surface and has a relatively homogenous thickness. In one embodiment, the instrument is selected from the group consisting of a manual syringe and an automatic pipettor. In one embodiment, the flushing comprises a shear velocity of approximately 187.5 μl/sec. In one embodiment, the partial thick gel layer comprises a thickness that ranges between 50-200 μm. In one embodiment, the at least one polymer is selected from the group consisting of collagen I and fibronectin. In one embodiment, the method further comprises calculating said thickness with a thresholding algorithm.

In one embodiment, the present invention contemplates a method, comprising: a) providing; i) a microfluidic device comprising a microchannel, said microchannel comprising a surface; ii) a gel matrix solution comprising at least one polymer at a concentration of >0.5 mg/ml; and iii) a fluid comprising a lower viscosity than said gel matrix solution; b) seeding said surface with a cell layer; b) loading said gel matrix solution into said microchannel; c) polymerizing said gel matrix solution for five seconds; d) flushing said gel solution matrix from said microchannel to create a partial gel overlay comprising a central lumen, wherein a wall of said partial gel overlay coats said seeded cells. In one embodiment, the wall has a thickness ranging between approximately 50 μm-200 μm. In one embodiment, the method further comprises a gel underlay between said surface and said cell layer. In one embodiment, the gel underlay has a thickness of approximately 25 μm. In one embodiment, the lumen is homogenous, continuous and of a constant diameter. In one embodiment, the cell layer is a hepatocyte cell layer. In one embodiment, the polymer is collagen I. In one embodiment, the method further comprises differentiating said hepatocyte cells to express highly developed bile canaliculi as compared to a gelatinous protein mixture secreted by Engelbreth-Holm-Swarm mouse sarcoma cells. In one embodiment, the hepatocyte cells have reduced lipid accumulation as compared to a gelatinous protein mixture secreted by Engelbreth-Holm-Swarm mouse sarcoma cells. In one embodiment, the method further comprises a stellate cell layer embedded within said underlay, wherein said hepatocyte cells have reduced lipid accumulation. In one embodiment, the method further comprises incubating said cell layer with ethanol, wherein said hepatocyte cells have reduced lipid accumulation. In one embodiment, the stellate cell layer is an activated hepatic stellate cell layer. In one embodiment, the stellate cell layer improves bile canaliculi development in the presence of said ethanol. In one embodiment, the improvement is determined by quantitative metrics selected from the group consisting of increased branching density, increased porosity and decreased canal radius.

In one embodiment, the present invention contemplates a method, comprising: a) providing; i) a microfluidic device comprising a microchannel, said microchannel comprising a surface; ii) a gel matrix solution comprising at least one polymer at a concentration of >0.5 mg/ml; and iii) a fluid comprising a lower viscosity than said gel matrix solution; b) seeding said surface with a cell layer; b) loading said gel matrix solution into said microchannel; c) polymerizing said gel matrix solution for five seconds; d) flushing said gel solution matrix from said microchannel to create a partial thick gel overlay comprising a central lumen, wherein a wall of said partial thick gel overlay coats said seeded cells. In one embodiment, the wall has a thickness ranging between approximately 50 μm-200 μm. In one embodiment, the method further comprises a gel underlay between said surface and said cell layer. In one embodiment, the gel underlay has a thickness of approximately 25 μm. In one embodiment, the lumen is homogenous, continuous and of a constant diameter. In one embodiment, the cell layer is a hepatocyte cell layer. In one embodiment, the polymer is collagen I. In one embodiment, the method further comprises differentiating said hepatocyte cells to express highly developed bile canaliculi as compared to a gelatinous protein mixture secreted by Engelbreth-Holm-Swarm mouse sarcoma cells. In one embodiment, the hepatocyte cells have reduced lipid accumulation as compared to a gelatinous protein mixture secreted by Engelbreth-Holm-Swarm mouse sarcoma cells. In one embodiment, the method further comprises a stellate cell layer embedded within said underlay, wherein said hepatocyte cells have reduced lipid accumulation. In one embodiment, the method further comprises incubating said cell layer with ethanol, wherein said hepatocyte cells have reduced lipid accumulation. In one embodiment, the stellate cell layer is an activated hepatic stellate cell layer. In one embodiment, the stellate cell layer improves bile canaliculi development in the presence of said ethanol. In one embodiment, the improvement is determined by quantitative metrics selected from the group consisting of increased branching density, increased porosity and decreased canal radius.

In one embodiment, the present invention contemplates a microfluidic device, comprising: a) at least one microchannel and a membrane comprising a surface and a partial gel layer; b) at least one layer of an intestinal colonic epithelial cell adhered to said partial gel layer. In one embodiment, the said partial gel layer has a thickness of approximately 25 μm. In one embodiment, the gel is contained within the microchannel, i.e. the microchannel is closed (other than having inlet and outlet ports). In one embodiment, the partial gel layer comprises collagen IV and a gelatinous protein mixture secrete by Engelbreth-Holm-Swarm mouse sarcoma cells. In one embodiment, the membrane further comprises a colonic fibroblast cell layer. In one embodiment, the membrane further comprises an endothelial cell layer. In one embodiment, the epithelial cell layer and said colonic fibroblast layer are adhered to opposite surfaces of said membrane. In one embodiment, the fibroblast cell layer is adhered to said membrane underneath said epithelial cells. In one embodiment, the opposite membrane surfaces are in contact with the same culture media. In one embodiment, the opposite membrane surfaces are in contact with different culture media. In one embodiment, the colonic fibroblasts have migrated across said membrane. In one embodiment, the migrated colonic fibroblasts have formed an intestinal barrier.

In one embodiment, the present invention contemplates, a microfluidic device, comprising: a) at least one microchannel and a membrane comprising a partial gel layer comprising a lumen on a bottom surface of said membrane; b) a kidney glomerulus endothelial cell layer adhered to said lumen. In one embodiment, the partial gel layer is a gel layer. In one embodiment, the gel layer comprises a wall having a thickness ranging between 50-200 μm. In one embodiment, the gel layer comprises collagen I. fibronectin and collagen IV. In one embodiment, the gel layer further comprises a glomerulus mesangial cell layer. In one embodiment, the membrane further comprises a top surface adhered to a glomerulus podocyte cell layer. In one embodiment, the gel layer comprises a mesangial matrix. In one embodiment, the membrane further comprises a mesangial cell/podocyte cell basement membrane. In one embodiment, the membrane further comprises a mesangial cell/endothelial cell glycocalyx coat. In one embodiment, the membrane further comprises a podocyte cell/endothelial cell slit diaphragm.

In one embodiment, the present invention contemplates, a microfluidic device, comprising: a) at least one microchannel and a membrane comprising a partial gel layer comprising a lumen on a bottom surface of said membrane; b) a kidney glomerulus endothelial cell layer adhered to said lumen. In one embodiment, the partial gel layer is a thick gel layer. In one embodiment, the thick gel layer comprises a wall having a thickness ranging between 50-200 μm. In one embodiment, the thick gel layer comprises collagen I, fibronectin and collagen IV. In one embodiment, the thick gel layer further comprises a glomerulus mesangial cell layer. In one embodiment, the membrane further comprises a top surface adhered to a glomerulus podocyte cell layer. In one embodiment, the thick gel layer comprises a mesangial matrix. In one embodiment, the membrane further comprises a mesangial cell/podocyte cell basement membrane. In one embodiment, the membrane further comprises a mesangial cell/endothelial cell glycocalyx coat. In one embodiment, the membrane further comprises a podocyte cell/endothelial cell slit diaphragm.

The present invention is related to the field of imaging and image processing. In particular, the invention is related to imaging that supports the determination of cell membrane cilia beating frequency. For example, methods described herein encompass cilia beat frequency in the context of membrane region and/or distances between regions. Alternatively, the methods described here encompass cilia beat synchrony and correlation of beat frequency between cell membrane regions.

In one embodiment, the present invention contemplates a method, comprising: a) providing: i) a cell culture device comprising at least one chamber comprising a plurality of ciliated cells; ii) an imaging device comprising a modified light filter configured to image at least a portion of said plurality of ciliated cells; and iii) a processor comprising an image acquisition and analysis algorithm system wherein said processor is in electronic communication with said imaging device; b) imaging at least a portion of said plurality of ciliated cells to create an image digital file representing a region of said channel; and c) processing said image digital file with said image acquisition and analysis algorithm system of said processor to measure at least one ciliary biomarker in said region. In one embodiment, the chamber is a well. In one embodiment, the chamber is a channel. In one embodiment, the channel is a microchannel. In one embodiment, the cell culture device is a microfluidic device. In one embodiment, the cell culture device is a transwell device. In one embodiment, the method further comprises step d) identifying at least one symptom of a disease with said measuring of said at least one ciliary biomarker. In one embodiment, the plurality of ciliated cells comprises a plurality of differentiated ciliated epithelial cells. In one embodiment, the plurality of ciliated cells comprises a layer on said channel. In one embodiment, the ciliary biomarker is selected from the group consisting of ciliary beat frequency, ciliary beat frequency variability, ciliary beat frequency synchronization, ciliary beat frequency correlation, ciliary beat frequency uniformity and ciliary beat frequency temporal profile. In one embodiment, the modified light filter is configured to optimize and standardize an incidence angle. In one embodiment, the modified light filter is configured to increase contrast, clarity and data content extraction amount of said image digital file. In one embodiment, the image acquisition and analysis algorithm system comprises a plurality of pre-processing step algorithms to reduce noise and enhance said measuring of said ciliary biomarker. In one embodiment, the image acquisition and analysis algorithm system comprises a plurality of image processing algorithms wherein at least two algorithms are selected from the group consisting of motion detection, edge detection, particle tracking, Fourier Transformation, particle image velocimetry and orientation-sensitive algorithms. In one embodiment, the image acquisition and analysis algorithm system comprises an image processing algorithm configured to measure ciliary structure. In one embodiment, the image acquisition and analysis algorithm system comprises an image processing algorithm configured to measure ciliary beat motion. In one embodiment, the image acquisition and analysis algorithm system comprises an image processing algorithm configured to measure ciliary fluid transport. In one embodiment, the image acquisition and analysis algorithm system comprises an image processing algorithm configured to measure mucociliary transport. In one embodiment, the ciliary biomarker is a mucociliary transport biomarker. In one embodiment, the mucociliary transport biomarker is selected from the group consisting of single cell cilia kinematics, beat angle, cilia polarity, beat coordination; flow properties, horizontal flow velocity, vertical flow velocity, straightness, circulation, tissue-level kinematics, metachronal wave lengths and directionality. In one embodiment, the ciliated cells are derived from a healthy patient. In one embodiment, the ciliated cells are derived from a diseased patient. In one embodiment, the diseased patient exhibits at least one symptom of a respiratory disease. In one embodiment, the respiratory disease is selected from the group consisting of an inflammatory disease, a genetic ciliopathy and an acquired ciliopathy. In one embodiment, the respiratory disease is a mucociliary transport disease.

In one embodiment, the present invention contemplates a method, comprising: a) providing: i) a cell culture device comprising at least one chamber comprising a plurality of ciliated cells; ii) an imaging device comprising a modified light filter configured to image at least a portion of said plurality of ciliated cells; iii) a processor comprising an image acquisition and analysis algorithm system wherein said processor is in electronic communication with said imaging device; and iv) an agent; b) imaging at least a portion of said plurality of ciliated cells to create a first image digital file representing a first region of said channel; c) processing said first image digital file with said image acquisition and analysis algorithm system of said processor to measure at least one ciliary biomarker in said region; d) introducing said agent to said plurality of ciliated cells to create a plurality of treated ciliated cells; e) imaging at least a portion of said plurality of treated ciliated cells to create a second image digital file representing said first region of said channel; and f) processing said second image digital file with said image acquisition and analysis algorithm system of said processor to measure a change in at least one ciliary biomarker in said first region. In one embodiment, the chamber is a well. In one embodiment, the chamber is a channel. In one embodiment, the channel is a microchannel. In one embodiment, the cell culture device is a microfluidic device. In one embodiment, the cell culture device is a transwell device. In one embodiment, the agent is a drug. In one embodiment, the agent is an infectious agent. In one embodiment, the plurality of ciliated cells comprises a plurality of differentiated ciliated epithelial cells. In one embodiment, the plurality of ciliated cells comprises a layer on said channel. In one embodiment, the ciliary biomarker is selected from the group consisting of ciliary beat frequency, ciliary beat frequency variability, ciliary beat frequency synchronization, ciliary beat frequency correlation, ciliary beat frequency uniformity and ciliary beat frequency temporal profile. In one embodiment, the modified light filter is configured to optimize and standardize an incidence angle. In one embodiment, the modified light filter is configured to increase contrast, clarity and data content extraction amount of said image digital file. In one embodiment, the image acquisition and analysis algorithm system comprises a plurality of pre-processing step algorithms to reduce noise and enhance said measuring of said ciliary biomarker. In one embodiment, the image acquisition and analysis algorithm system comprises a plurality of image processing algorithms wherein at least two algorithms are selected from the group consisting of motion detection, edge detection, particle tracking, Fourier Transformation, particle image velocimetry and orientation-sensitive algorithms. In one embodiment, the image acquisition and analysis algorithm system comprises an image processing algorithm configured to measure ciliary structure. In one embodiment, the image acquisition and analysis algorithm system comprises an image processing algorithm configured to measure ciliary beat motion. In one embodiment, the image acquisition and analysis algorithm system comprises an image processing algorithm configured to measure ciliary fluid transport. In one embodiment, the image acquisition and analysis algorithm system comprises an image processing algorithm configured to measure mucociliary transport. In one embodiment, the ciliary biomarker is a mucociliary transport biomarker. In one embodiment, the mucociliary transport biomarker is selected from the group consisting of single cell cilia kinematics, beat angle, cilia polarity, beat coordination; flow properties, horizontal flow velocity, vertical flow velocity, straightness, circulation, tissue-level kinematics, metachronal wave lengths and directionality. In one embodiment, the ciliated cells are derived from a healthy patient. In one embodiment, the ciliated cells are derived from a diseased patient. In one embodiment, the diseased patient exhibits at least one symptom of a respiratory disease. In one embodiment, the respiratory disease is selected from the group consisting of an inflammatory disease, a genetic ciliopathy and an acquired ciliopathy. In one embodiment, the respiratory disease is a mucociliary transport disease. In one embodiment, the ciliated cells comprise human primary airway epithelial cells derived from a human cystic fibrosis patient. In one embodiment, the drug comprises a cytokine. In one embodiment, the drug comprises a steroid. In one embodiment, the infectious agent is a virus.

In one embodiment, the present invention contemplates a method, comprising: a) providing: i) a cell culture device comprising at least one chamber comprising a plurality of ciliated cells; ii) an imaging device comprising a modified light filter configured to image a region of said plurality of ciliated cells; and iii) a processor comprising an image acquisition and analysis algorithm system wherein said processor is in electronic communication with said imaging device; b) imaging said region of said plurality of ciliated cells to create an image digital file; and c) processing said image digital file with said image acquisition and analysis algorithm system of said processor to measure a variability of at least one ciliary biomarker in said region. In one embodiment, the at least one ciliary biomarker variability measurement comprises cilia rate variability along a specific distance. In one embodiment, the at least one ciliary biomarker variability measurement comprises ciliary beat frequency variability. In one embodiment, the chamber is a well. In one embodiment, the chamber is a channel. In one embodiment, the channel is a microchannel. In one embodiment, the cell culture device is a microfluidic device. In one embodiment, the cell culture device is a transwell device. In one embodiment, the method further comprises step d) identifying at least one symptom of a disease with said measuring of said at least one ciliary biomarker. In one embodiment, the plurality of ciliated cells comprises a plurality of differentiated ciliated epithelial cells. In one embodiment, the plurality of ciliated cells comprises a layer on said channel. In one embodiment, the ciliary biomarker is selected from the group consisting of ciliary beat frequency, ciliary beat frequency variability, ciliary beat frequency synchronization, ciliary beat frequency correlation, ciliary beat frequency uniformity and ciliary beat frequency temporal profile. In one embodiment, the modified light filter is configured to optimize and standardize an incidence angle. In one embodiment, the modified light filter is configured to increase contrast, clarity and data content extraction amount of said image digital file. In one embodiment, the image acquisition and analysis algorithm system comprises a plurality of pre-processing step algorithms to reduce noise and enhance said measuring of said ciliary biomarker. In one embodiment, the image acquisition and analysis algorithm system comprises a plurality of image processing algorithms wherein at least two algorithms are selected from the group consisting of motion detection, edge detection, particle tracking, Fourier Transformation, particle image velocimetry and orientation-sensitive algorithms. In one embodiment, the image acquisition and analysis algorithm system comprises an image processing algorithm configured to measure ciliary structure. In one embodiment, the image acquisition and analysis algorithm system comprises an image processing algorithm configured to measure ciliary beat motion. In one embodiment, the image acquisition and analysis algorithm system comprises an image processing algorithm configured to measure ciliary fluid transport. In one embodiment, the image acquisition and analysis algorithm system comprises an image processing algorithm configured to measure mucociliary transport. In one embodiment, the ciliary biomarker is a mucociliary transport biomarker. In one embodiment, the mucociliary transport biomarker is selected from the group consisting of single cell cilia kinematics, beat angle, cilia polarity, beat coordination; flow properties, horizontal flow velocity, vertical flow velocity, straightness, circulation, tissue-level kinematics, metachronal wave lengths and directionality. In one embodiment, the ciliated cells are derived from a healthy patient. In one embodiment, the ciliated cells are derived from a diseased patient. In one embodiment, the diseased patient exhibits at least one symptom of a respiratory disease. In one embodiment, the respiratory disease is selected from the group consisting of an inflammatory disease, a genetic ciliopathy and an acquired ciliopathy. In one embodiment, the respiratory disease is a mucociliary transport disease.

In one embodiment, the present invention contemplates a method, comprising: a) providing: i) a cell culture device comprising at least one chamber comprising a plurality of ciliated cells; ii) an imaging device comprising a modified light filter configured to image a region of said plurality of ciliated cells; iii) a processor comprising an image acquisition and analysis algorithm system wherein said processor is in electronic communication with said imaging device; and iv) an agent; b) imaging at least a portion of said plurality of ciliated cells in said region to create a first image digital file; and c) processing said first image digital file with said image acquisition and analysis algorithm system of said processor to measure a variability of at least one ciliary biomarker in said region; d) introducing said agent to said plurality of ciliated cells to create a plurality of treated ciliated cells; e) imaging at least a portion of said plurality of treated ciliated cells in said region to create a second image digital file; and f) processing said second image digital file with said image acquisition and analysis algorithm system of said processor to measure a change in variability of said at least one ciliary biomarker in said region. In one embodiment, the at least one ciliary biomarker variability measurement comprises cilia rate variability along a specific distance. In one embodiment, the at least one ciliary biomarker variability measurement comprises ciliary beat frequency variability. In one embodiment, the chamber is a well. In one embodiment, the chamber is a channel. In one embodiment, the channel is a microchannel. In one embodiment, the cell culture device is a microfluidic device. In one embodiment, the cell culture device is a transwell device. In one embodiment, the agent is a drug. In one embodiment, the agent is an infectious agent. In one embodiment, the plurality of ciliated cells comprises a plurality of differentiated ciliated epithelial cells. In one embodiment, the plurality of ciliated cells comprises a layer on said channel. In one embodiment, the ciliary biomarker is selected from the group consisting of ciliary beat frequency, ciliary beat frequency variability, ciliary beat frequency synchronization, ciliary beat frequency correlation, ciliary beat frequency uniformity and ciliary beat frequency temporal profile. In one embodiment, the modified light filter is configured to optimize and standardize an incidence angle. In one embodiment, the modified light filter is configured to increase contrast, clarity and data content extraction amount of said image digital file. In one embodiment, the image acquisition and analysis algorithm system comprises a plurality of pre-processing step algorithms to reduce noise and enhance said measuring of said ciliary biomarker.

In one embodiment, the image acquisition and analysis algorithm system comprises a plurality of image processing algorithms wherein at least two algorithms are selected from the group consisting of motion detection, edge detection, particle tracking, Fourier Transformation, particle image velocimetry and orientation-sensitive algorithms. In one embodiment, the image acquisition and analysis algorithm system comprises an image processing algorithm configured to measure ciliary structure. In one embodiment, the image acquisition and analysis algorithm system comprises an image processing algorithm configured to measure ciliary beat motion. In one embodiment, the image acquisition and analysis algorithm system comprises an image processing algorithm configured to measure ciliary fluid transport. In one embodiment, the image acquisition and analysis algorithm system comprises an image processing algorithm configured to measure mucociliary transport. In one embodiment, the ciliary biomarker is a mucociliary transport biomarker. In one embodiment, the mucociliary transport biomarker is selected from the group consisting of single cell cilia kinematics, beat angle, cilia polarity, beat coordination; flow properties, horizontal flow velocity, vertical flow velocity, straightness, circulation, tissue-level kinematics, metachronal wave lengths and directionality. In one embodiment, the ciliated cells are derived from a healthy patient. In one embodiment, the ciliated cells are derived from a diseased patient. In one embodiment, the diseased patient exhibits at least one symptom of a respiratory disease. In one embodiment, the respiratory disease is selected from the group consisting of an inflammatory disease, a genetic ciliopathy and an acquired ciliopathy. In one embodiment, the respiratory disease is a mucociliary transport disease. In one embodiment, the ciliated cells comprise human primary airway epithelial cells derived from a human cystic fibrosis patient. In one embodiment, the drug comprises a cytokine. In one embodiment, the drug comprises a steroid. In one embodiment, the infectious agent is a virus.

In one embodiment, the present invention contemplates, a method, comprising: a) providing: i) a cell culture device comprising at least one chamber comprising a plurality of ciliated cells; ii) an imaging device comprising a modified light filter configured to image a region of said plurality of ciliated cells; and iii) a processor comprising an image acquisition and analysis algorithm system wherein said processor is in electronic communication with said imaging device; b) imaging said region of said plurality of ciliated cells to create an image digital file; and c) processing said image digital file with said image acquisition and analysis algorithm system of said processor to measure a correlation between a first ciliary biomarker and a second ciliary biomarker in said region. In one embodiment, the correlation is a cilia rate variability correlation. In one embodiment, the correlation is a cilia beat frequency correlation. In one embodiment, the correlation is a temporal correlation. In one embodiment, the temporal correlation identifies that said first ciliary biomarker and said second ciliary biomarker are not synchronized. In one embodiment, the correlation is an orientational order correlation. In one embodiment, the orientational order correlation identifies that said first ciliary biomarker and said second ciliary biomarker are in the same direction. In one embodiment, the chamber is a well. In one embodiment, the chamber is a channel. In one embodiment, the channel is a microchannel. In one embodiment, the cell culture device is a microfluidic device. In one embodiment, the cell culture device is a transwell device. In one embodiment, the method further comprises step d) identifying at least one symptom of a disease with said measuring of said at least one ciliary biomarker. In one embodiment, the plurality of ciliated cells comprises a plurality of differentiated ciliated epithelial cells. In one embodiment, the plurality of ciliated cells comprises a layer on said channel. In one embodiment, the ciliary biomarker is selected from the group consisting of ciliary beat frequency, ciliary beat frequency variability, ciliary beat frequency synchronization, ciliary beat frequency correlation, ciliary beat frequency uniformity and ciliary beat frequency temporal profile. In one embodiment, the modified light filter is configured to optimize and standardize an incidence angle. In one embodiment, the modified light filter is configured to increase contrast, clarity and data content extraction amount of said image digital file. In one embodiment, the image acquisition and analysis algorithm system comprises a plurality of pre-processing step algorithms to reduce noise and enhance said measuring of said ciliary biomarker. In one embodiment, the image acquisition and analysis algorithm system comprises a plurality of image processing algorithms wherein at least two algorithms are selected from the group consisting of motion detection, edge detection, particle tracking, Fourier Transformation, particle image velocimetry and orientation-sensitive algorithms. In one embodiment, the image acquisition and analysis algorithm system comprises an image processing algorithm configured to measure ciliary structure. In one embodiment, the image acquisition and analysis algorithm system comprises an image processing algorithm configured to measure ciliary beat motion. In one embodiment, the image acquisition and analysis algorithm system comprises an image processing algorithm configured to measure ciliary fluid transport. In one embodiment, the image acquisition and analysis algorithm system comprises an image processing algorithm configured to measure mucociliary transport. In one embodiment, the ciliary biomarker is a mucociliary transport biomarker. In one embodiment, the mucociliary transport biomarker is selected from the group consisting of single cell cilia kinematics, beat angle, cilia polarity, beat coordination; flow properties, horizontal flow velocity, vertical flow velocity, straightness, circulation, tissue-level kinematics, metachronal wave lengths and directionality. In one embodiment, the ciliated cells are derived from a healthy patient. In one embodiment, the ciliated cells are derived from a diseased patient. In one embodiment, the diseased patient exhibits at least one symptom of a respiratory disease. In one embodiment, the respiratory disease is selected from the group consisting of an inflammatory disease, a genetic ciliopathy and an acquired ciliopathy. In one embodiment, the respiratory disease is a mucociliary transport disease.

In one embodiment, the present invention contemplates a method, comprising: a) providing: i) a cell culture device comprising at least one chamber comprising a plurality of ciliated cells; ii) an imaging device comprising a modified light filter configured to image a region of said plurality of ciliated cells; iii) a processor comprising an image acquisition and analysis algorithm system wherein said processor is in electronic communication with said imaging device; and iv) an agent; b) imaging at least a portion of said plurality of ciliated cells in said region to create a first image digital file; c) processing said first image digital file with said image acquisition and analysis algorithm system of said processor to measure a correlation between first ciliary biomarker and a second ciliary biomarker in said region; d) introducing said agent to said plurality of ciliated cells to create a plurality of treated ciliated cells; e) imaging at least a portion of said plurality of treated ciliated cells in said region to create a second image digital file; and f) processing said second image digital file with said image acquisition and analysis algorithm system of said processor to measure a change in correlation between said first ciliary biomarker and said second ciliary biomarker in said region. In one embodiment, the correlation is a cilia rate variability correlation. In one embodiment, the correlation is a cilia beat frequency correlation. In one embodiment, the correlation is a temporal correlation. In one embodiment, the temporal correlation identifies that said first ciliary biomarker and said second ciliary biomarker are not synchronized. In one embodiment, the correlation is an orientational order correlation. In one embodiment, the orientational order correlation identifies that said first ciliary biomarker and said second ciliary biomarker are in the same direction. In one embodiment, the chamber is a well. In one embodiment, the chamber is a channel. In one embodiment, the channel is a microchannel. In one embodiment, the cell culture device is a microfluidic device. In one embodiment, the cell culture device is a transwell device. In one embodiment, the agent is a drug. In one embodiment, the agent is an infectious agent. In one embodiment, the plurality of ciliated cells comprises a plurality of differentiated ciliated epithelial cells. In one embodiment, the plurality of ciliated cells comprises a layer on said channel. In one embodiment, the ciliary biomarker is selected from the group consisting of ciliary beat frequency, ciliary beat frequency variability, ciliary beat frequency synchronization, ciliary beat frequency correlation, ciliary beat frequency uniformity and ciliary beat frequency temporal profile. In one embodiment, the modified light filter is configured to optimize and standardize an incidence angle. In one embodiment, the modified light filter is configured to increase contrast, clarity and data content extraction amount of said image digital file. In one embodiment, the image acquisition and analysis algorithm system comprises a plurality of pre-processing step algorithms to reduce noise and enhance said measuring of said ciliary biomarker. In one embodiment, the image acquisition and analysis algorithm system comprises a plurality of image processing algorithms wherein at least two algorithms are selected from the group consisting of motion detection, edge detection, particle tracking, Fourier Transformation, particle image velocimetry and orientation-sensitive algorithms. In one embodiment, the image acquisition and analysis algorithm system comprises an image processing algorithm configured to measure ciliary structure. In one embodiment, the image acquisition and analysis algorithm system comprises an image processing algorithm configured to measure ciliary beat motion. In one embodiment, the image acquisition and analysis algorithm system comprises an image processing algorithm configured to measure ciliary fluid transport. In one embodiment, the image acquisition and analysis algorithm system comprises an image processing algorithm configured to measure mucociliary transport. In one embodiment, the ciliary biomarker is a mucociliary transport biomarker. In one embodiment, the mucociliary transport biomarker is selected from the group consisting of single cell cilia kinematics, beat angle, cilia polarity, beat coordination; flow properties, horizontal flow velocity, vertical flow velocity, straightness, circulation, tissue-level kinematics, metachronal wave lengths and directionality. In one embodiment, the ciliated cells are derived from a healthy patient. In one embodiment, the ciliated cells are derived from a diseased patient. In one embodiment, the diseased patient exhibits at least one symptom of a respiratory disease. In one embodiment, the respiratory disease is selected from the group consisting of an inflammatory disease, a genetic ciliopathy and an acquired ciliopathy. In one embodiment, the respiratory disease is a mucociliary transport disease. In one embodiment, the ciliated cells comprise human primary airway epithelial cells derived from a human cystic fibrosis patient. In one embodiment, the drug comprises a cytokine. In one embodiment, the drug comprises a steroid. In one embodiment, the infectious agent is a virus.

In one embodiment, the present invention contemplates a method, comprising: a) providing: i) a cell culture device comprising at least one chamber comprising a plurality of diseased ciliated cells; ii) an imaging device comprising a modified light filter configured to image at least a portion of said plurality of diseased ciliated cells; and iii) a processor comprising an image acquisition and analysis algorithm system wherein said processor is in electronic communication with said imaging device; b) imaging at least a portion of said plurality of diseased ciliated cells to create an image digital file representing a region of said channel; and c) processing said image digital file with said image acquisition and analysis algorithm system of said processor to measure at least one ciliary biomarker in said region. In one embodiment, the diseased ciliated cells were exposed to an agent. In one embodiment, the diseased ciliated cells were derived from a diseased patient. In one embodiment, the agent is a drug. In one embodiment, the agent is an infectious agent. In one embodiment, the infectious agent is a virus. In one embodiment, the drug comprises a cytokine. In one embodiment, the drug comprises a steroid. In one embodiment, the diseased patient exhibits at least one symptom of a respiratory disease. In one embodiment, the respiratory disease is selected from the group consisting of an inflammatory disease, a genetic ciliopathy and an acquired ciliopathy. In one embodiment, the respiratory disease is a mucociliary transport disease. In one embodiment, the ciliated cells comprise human primary airway epithelial cells derived from a human cystic fibrosis patient. In one embodiment, the diseased cells are induced to express a disease phenotype. In one embodiment, the expressed disease phenotype is genetically induced. In one embodiment, the expressed disease phenotype is agent-induced. In one embodiment, the method further comprises comparing said diseased ciliary cell biomarker measurement to a healthy ciliary cell biomarker measurement. In one embodiment, the correlating diagnoses a disease. In one embodiment, the diseased cell ciliary biomarker measurement comprises a variability measurement. In one embodiment, the diseased cell ciliary biomarker measurement comprises a correlation measurement. In one embodiment, the at least one diseased ciliary biomarker variability measurement comprises cilia rate variability along a specific distance. In one embodiment, the at least one diseased ciliary biomarker variability measurement comprises ciliary beat frequency variability. In one embodiment, the correlation is a cilia rate variability correlation. In one embodiment, the correlation is a cilia beat frequency correlation. In one embodiment, the correlation is a temporal correlation. In one embodiment, the temporal correlation identifies that said first ciliary biomarker and said second ciliary biomarker are not synchronized. In one embodiment, the correlation is an orientational order correlation. In one embodiment, the orientational order correlation identifies that said first ciliary biomarker and said second ciliary biomarker are in the same direction. In one embodiment, the plurality of diseased ciliated cells comprises a plurality of differentiated diseased ciliated epithelial cells. In one embodiment, the plurality of diseased ciliated cells comprises a layer on said channel. In one embodiment, the ciliary biomarker is selected from the group consisting of ciliary beat frequency, ciliary beat frequency variability, ciliary beat frequency synchronization, ciliary beat frequency correlation, ciliary beat frequency uniformity and ciliary beat frequency temporal profile. In one embodiment, the modified light filter is configured to optimize and standardize an incidence angle. In one embodiment, the modified light filter is configured to increase contrast, clarity and data content extraction amount of said image digital file. In one embodiment, the image acquisition and analysis algorithm system comprises a plurality of pre-processing step algorithms to reduce noise and enhance said measuring of said ciliary biomarker. In one embodiment, the image acquisition and analysis algorithm system comprises a plurality of image processing algorithms wherein at least two algorithms are selected from the group consisting of motion detection, edge detection, particle tracking, Fourier Transformation, particle image velocimetry and orientation-sensitive algorithms. In one embodiment, the image acquisition and analysis algorithm system comprises an image processing algorithm configured to measure ciliary structure. In one embodiment, the image acquisition and analysis algorithm system comprises an image processing algorithm configured to measure ciliary beat motion. In one embodiment, the image acquisition and analysis algorithm system comprises an image processing algorithm configured to measure ciliary fluid transport. In one embodiment, the image acquisition and analysis algorithm system comprises an image processing algorithm configured to measure mucociliary transport. In one embodiment, the ciliary biomarker is a mucociliary transport biomarker. In one embodiment, the mucociliary transport biomarker is selected from the group consisting of single cell cilia kinematics, beat angle, cilia polarity, beat coordination; flow properties, horizontal flow velocity, vertical flow velocity, straightness, circulation, tissue-level kinematics, metachronal wave lengths and directionality. In one embodiment, the chamber is a well. In one embodiment, the chamber is a channel. In one embodiment, the channel is a microchannel. In one embodiment, the cell culture device is a microfluidic device. In one embodiment, the cell culture device is a transwell device.

In one embodiment, the present invention contemplates a method, comprising: a) providing: i) a plurality of cell culture devices, wherein each of said plurality of cell culture devices comprise at least one chamber with a plurality of ciliated cells; ii) an imaging device comprising a modified light filter configured to image at least a portion of said plurality of ciliated cells in each of said plurality of cell culture devices; iii) a processor comprising an image acquisition and analysis algorithm system wherein said processor is in electronic communication with said imaging device; and iv) a plurality of agents; b) imaging at least a portion of said plurality of ciliated cells in each of said plurality of cell culture devices to create a first set of image digital files representing a first region of said chambers; c) processing said first set of image digital files with said image acquisition and analysis algorithm system of said processor to measure at least one ciliary biomarker in said first regions; d) introducing a single agent of said plurality of agents into each of said plurality of cell culture devices to create a plurality of treated ciliated cells; e) imaging at least a portion of said plurality of treated ciliated cells in each of said plurality of cell culture devices to create a second set of image digital file representing said first region of said chamber; and f) processing said second set of image digital files with said image acquisition and analysis algorithm system of said processor to measure a change in said at least one ciliary biomarker in said first region of each of said plurality of cell culture devices; and g) identifying a candidate agent based on said change in said at least one ciliary biomarker. In one embodiment, the candidate agent is a candidate therapeutic agent. In one embodiment, the candidate agent is a candidate research agent. In one embodiment, the candidate research agent modulates a biochemical mechanism of said at least one ciliary biomarker. In one embodiment, the candidate agent is a drug. In one embodiment, the candidate agent is a nucleic acid sequence. In one embodiment, the candidate agent is an amino acid sequence. In one embodiment, the drug comprises a cytokine. In one embodiment, the drug comprises a steroid. In one embodiment, the nucleic acid sequence is a ribonucleic acid sequence. In one embodiment, the amino acid sequence is a Cas9 sequence. In one embodiment, the cell ciliary biomarker measurement comprises a variability measurement. In one embodiment, the cell ciliary biomarker measurement comprises a correlation measurement. In one embodiment, the at least one ciliary biomarker variability measurement comprises cilia rate variability along a specific distance. In one embodiment, the at least one ciliary biomarker variability measurement comprises ciliary beat frequency variability. In one embodiment, the correlation is a cilia rate variability correlation. In one embodiment, the correlation is a cilia beat frequency correlation. In one embodiment, the correlation is a temporal correlation. In one embodiment, the temporal correlation identifies that said first ciliary biomarker and said second ciliary biomarker are not synchronized. In one embodiment, the correlation is an orientational order correlation. In one embodiment, the orientational order correlation identifies that said first ciliary biomarker and said second ciliary biomarker are in the same direction. In one embodiment, the plurality of ciliated cells comprises a plurality of differentiated ciliated epithelial cells. In one embodiment, the plurality of ciliated cells comprises a layer on said channel. In one embodiment, the ciliary biomarker is selected from the group consisting of ciliary beat frequency, ciliary beat frequency variability, ciliary beat frequency synchronization, ciliary beat frequency correlation, ciliary beat frequency uniformity and ciliary beat frequency temporal profile. In one embodiment, the modified light filter is configured to optimize and standardize an incidence angle. In one embodiment, the modified light filter is configured to increase contrast, clarity and data content extraction amount of said image digital file. In one embodiment, the image acquisition and analysis algorithm system comprises a plurality of pre-processing step algorithms to reduce noise and enhance said measuring of said ciliary biomarker. In one embodiment, the image acquisition and analysis algorithm system comprises a plurality of image processing algorithms wherein at least two algorithms are selected from the group consisting of motion detection, edge detection, particle tracking, Fourier Transformation, particle image velocimetry and orientation-sensitive algorithms. In one embodiment, the image acquisition and analysis algorithm system comprises an image processing algorithm configured to measure ciliary structure. In one embodiment, the image acquisition and analysis algorithm system comprises an image processing algorithm configured to measure ciliary beat motion. In one embodiment, the image acquisition and analysis algorithm system comprises an image processing algorithm configured to measure ciliary fluid transport. In one embodiment, the image acquisition and analysis algorithm system comprises an image processing algorithm configured to measure mucociliary transport. In one embodiment, the ciliary biomarker is a mucociliary transport biomarker. In one embodiment, the mucociliary transport biomarker is selected from the group consisting of single cell cilia kinematics, beat angle, cilia polarity, beat coordination; flow properties, horizontal flow velocity, vertical flow velocity, straightness, circulation, tissue-level kinematics, metachronal wave lengths and directionality. In one embodiment, the chamber is a well. In one embodiment, the chamber is a channel. In one embodiment, the channel is a microchannel. In one embodiment, the cell culture device is a microfluidic device. In one embodiment, the cell culture device is a transwell device.

In one embodiment, the present invention contemplates, a method, comprising: a) providing a microphysiological device comprising: i) a microfluidic device comprising at least one channel, said at least one channel layered with a plurality of differentiated ciliated epithelial cells; ii) an imaging device comprising a modified light filter configured to image said plurality of differentiated ciliated epithelial cells; iii) a processor comprising an image acquisition and analysis algorithm system wherein said processor is in electronic communication with said imaging device; b) imaging said plurality of differentiated ciliated epithelial cells to create an image digital file; c) analyzing said image digital file with said image acquisition and analysis algorithm system to identify at least one ciliary biomarker; and d) diagnosing at least one symptom of a respiratory disease with said identified at least one ciliary biomarker. In one embodiment, the image digital file comprises a cell membrane ciliated region. In one embodiment, the cell membrane ciliated region is of a single cell. In one embodiment, the cell membrane ciliated region comprises a discrete distance. In one embodiment, the ciliary biomarker is selected from the group consisting of ciliary beat frequency, ciliary beat frequency variability, ciliary beat frequency synchronization, ciliary beat frequency correlation, ciliary beat frequency uniformity and ciliary beat frequency temporal profile. In one embodiment, the modified light filter is configured to optimize and standardize an incidence angle. In one embodiment, the modified light filter is configured to increase contrast, clarity and data content extraction amount of said image digital file. In one embodiment, the image acquisition and analysis algorithm system comprises a plurality of pre-processing step algorithms to reduce noise and enhances the measurability of said ciliary biomarkers. In one embodiment, the image acquisition and analysis algorithm system comprises a plurality of image processing algorithms wherein at least two algorithms are selected from the group consisting of motion detection, edge detection, particle tracking, Fourier Transformation, particle image velocimetry and orientation-sensitive algorithms. In one embodiment, the image acquisition and analysis algorithm system comprises an image processing algorithm configured to measure ciliary structure. In one embodiment, the image acquisition and analysis algorithm system comprises an image processing algorithm configured to measure ciliary beat motion. In one embodiment, the image acquisition and analysis algorithm system comprises an image processing algorithm configured to measure ciliary fluid transport. In one embodiment, the image acquisition and analysis algorithm system comprises an image processing algorithm configured to measure mucociliary transport. In one embodiment, the ciliary biomarkers are mucociliary transport biomarkers. In one embodiment, the mucociliary transport biomarkers are selected from the group consisting of single cell cilia kinematics, beat angle, cilia polarity, beat coordination; flow properties, horizontal flow velocity, vertical flow velocity, straightness, circulation, tissue-level kinematics, metachronal wave lengths and directionality. In one embodiment, the differentiated epithelial cells are derived from a healthy patient. In one embodiment, the differentiated epithelial cells are derived from a diseased patient. In one embodiment, the diseased patient exhibits at least one symptom of a respiratory disease. In one embodiment, the respiratory disease is selected from the group consisting of an inflammatory disease, a genetic ciliopathy and an acquired ciliopathy. In one embodiment, the respiratory disease is a mucociliary transport disease.

In one embodiment, the present invention contemplates a method, comprising: a) providing a microphysiological device comprising: i) a microfluidic device comprising at least one channel, said at least one channel layered with a plurality of differentiated ciliated epithelial cells; ii) an imaging device comprising a modified light filter configured to image said plurality of differentiated ciliated epithelial cells; iii) a processor comprising an image acquisition and analysis algorithm system wherein said processor is in electronic communication with said imaging device; b) imaging said plurality of differentiated ciliated epithelial cells to create an image digital file; c) analyzing said image digital file with said image acquisition and analysis algorithm system to identify at least one ciliary biomarker; and d) diagnosing at least one symptom of a respiratory disease with said identified at least one ciliary biomarker. In one embodiment, the image digital file comprises a cell membrane ciliated region. In one embodiment, the cell membrane ciliated region is of a single cell. In one embodiment, the cell membrane ciliated region comprises a discrete distance. In one embodiment, the ciliary biomarker is selected from the group consisting of ciliary beat frequency, ciliary beat frequency variability, ciliary beat frequency synchronization, ciliary beat frequency correlation, ciliary beat frequency uniformity and ciliary beat frequency temporal profile. In one embodiment, the modified light filter is configured to optimize and standardize an incidence angle. In one embodiment, the modified light filter is configured to increase contrast, clarity and data content extraction amount of said image digital file. In one embodiment, the image acquisition and analysis algorithm system comprises a plurality of pre-processing step algorithms to reduce noise and enhances the measurability of said ciliary biomarkers. In one embodiment, the image acquisition and analysis algorithm system comprises a plurality of image processing algorithms wherein at least two algorithms are selected from the group consisting of motion detection, edge detection, particle tracking, Fourier Transformation, particle image velocimetry and orientation-sensitive algorithms. In one embodiment, the image acquisition and analysis algorithm system comprises an image processing algorithm configured to measure ciliary structure. In one embodiment, the image acquisition and analysis algorithm system comprises an image processing algorithm configured to measure ciliary beat motion. In one embodiment, the image acquisition and analysis algorithm system comprises an image processing algorithm configured to measure ciliary fluid transport. In one embodiment, the image acquisition and analysis algorithm system comprises an image processing algorithm configured to measure mucociliary transport. In one embodiment, the ciliary biomarkers are mucociliary transport biomarkers. In one embodiment, the mucociliary transport biomarkers are selected from the group consisting of single cell cilia kinematics, beat angle, cilia polarity, beat coordination; flow properties, horizontal flow velocity, vertical flow velocity, straightness, circulation, tissue-level kinematics, metachromal wave lengths and directionality. In one embodiment, the differentiated epithelial cells are derived from a healthy patient. In one embodiment, the differentiated epithelial cells are derived from a diseased patient. In one embodiment, the diseased patient exhibits at least one symptom of a respiratory disease. In one embodiment, the respiratory disease is selected from the group consisting of an inflammatory disease, a genetic ciliopathy and an acquired ciliopathy. In one embodiment, the respiratory disease is a mucociliary transport disease.

In one embodiment, the present invention provides a device comprising a membrane having an extracellular matrix coating on top of one surface of said membrane, wherein said coating is overlain with a gel between 20 microns-500 microns in thickness. In one embodiment, said gel comprises an extracellular matrix protein. In one embodiment, said extracellular matrix of said coating is a different type than said extracellular matrix of said gel. In one embodiment, said gel is a continuous layer. In one embodiment, said gel provides a continuous layer of approximately 100 microns. In one embodiment, said gel provides a continuous homogenous layer of approximately 30 microns. In one embodiment, said gel provides a continuous layer between 20-80 microns over at least 75% of said membrane surface. In one embodiment, said gel provides a continuous layer between 50-200 microns over at least 75% of said membrane surface. In one embodiment, said gel provides a partial gel layer covering 75%-100% of said membrane surface. In one embodiment, said extracellular matrix of said coating comprises Collagen I and wherein said extracellular matrix of said gel comprises Collagen IV. In one embodiment, said extracellular matrix of said coating comprises Collagen IV and wherein said extracellular matrix of said gel comprises Matrigel®. In one embodiment, said device is a Transwell device. In one embodiment, said device further comprises parenchymal cells seeded on top of said gel. In one embodiment, said device is a microfluidic device comprising at least one fluidic channel. In one embodiment, said microfluidic device comprises a second fluidic channel separated from said first fluidic channel by said membrane having a second surface facing said second channel. In one embodiment, said second channel comprises an extracellular matrix coating on top of said second surface wherein said coating is overlain with a gel between 20 microns-500 microns in thickness. In one embodiment, said device further comprises endothelial cells seeded in said second channel.

In one embodiment, the present invention provides a method, comprising a) providing a microfluidic device comprising a membrane having first and second surfaces; b) coating said membrane with an extracellular matrix protein coating on said first surface of said membrane, and c) overlaying said coating with a gel between 20 microns-500 microns in thickness. In one embodiment, said gel comprises an extracellular matrix protein. In one embodiment, said extracellular matrix protein of said coating is a different type than said extracellular matrix protein of said gel. In one embodiment, said gel is a continuous layer. In one embodiment, said gel provides a continuous layer of approximately 100 microns. In one embodiment, said gel provides a continuous homogenous layer of approximately 30 microns. In one embodiment, said gel provides a continuous layer between 20-80 microns over at least 75% of said membrane surface. In one embodiment, said gel provides a continuous layer between 50-200 microns over at least 75% of said membrane surface. In one embodiment, said gel provides a partial gel layer covering 75%-100% of said membrane surface. In one embodiment, said extracellular matrix of said coating comprises Collagen I and wherein said extracellular matrix of said gel comprises Collagen IV. In one embodiment, said extracellular matrix of said coating comprises Collagen IV and wherein said extracellular matrix of said gel comprises Matrigel®. In one embodiment, said device is a Transwell device. In one embodiment, said microfluidic device comprises at least one fluidic channel. In one embodiment, said method further comprising d) seeding parenchymal cells on top of said gel. In one embodiment, said gel is contained within at least a portion of said microfluidic fluidic channel. In one embodiment, said microfluidic device comprises first and second channels. In one embodiment, said method further comprising seeding endothelial cells in said second channel. In one embodiment, said method further comprising detecting differentiated cells. In one embodiment, said differentiated cells are ciliated cells. In one embodiment, said ciliated cells are derived from a cystic fibrosis patient.

In one embodiment, the present invention provides a cell culture system, comprising: a) a microfluidic device comprising a membrane having an extracellular matrix coating on top of one surface of said membrane, wherein said coating is overlain with a gel between 20 microns-500 microns in thickness; b) a fluid; and c) flowing said fluid under pressure for a period of time so as to increase the gas carrying capacity of said fluid. In one embodiment, the gel is contained within the device, i.e. the device is closed (other than having inlet and outlet ports). In one embodiment, said membrane comprises a center area, wherein at least a portion of said gel in said center area remains. In one embodiment, said system further comprising a population of cells, and a step before or after step c), a step of seeding said cells into said device on top of said gel overlay. In one embodiment, said system further comprising repeating step c) at least once after seeding said cells. In one embodiment, after said pressurized flow, said system resuming normal culture. In one embodiment, said pressurized flow has a duration of time from 1-2 hours. In one embodiment, said gel comprises an extracellular matrix protein. In one embodiment, said extracellular matrix of said coating is a different type than said extracellular matrix of said gel. In one embodiment, said gel is a continuous layer. In one embodiment, said gel provides a continuous layer of approximately 100 microns. In one embodiment, said gel provides a continuous homogenous layer of approximately 30 microns. In one embodiment, said gel provides a continuous layer between 20-80 microns over at least 75% of said membrane surface. In one embodiment, said gel provides a continuous layer between 50-200 microns over at least 75% of said membrane surface. In one embodiment, said gel provides a partial gel layer covering 75%-100% of said membrane surface. In one embodiment, said extracellular matrix of said coating comprises Collagen I and wherein said extracellular matrix of said gel comprises Collagen IV. In one embodiment, said extracellular matrix of said coating comprises Collagen IV and wherein said extracellular matrix of said gel comprises Matrigel®. In one embodiment, said device is a Transwell device. In one embodiment, said device further comprises parenchymal cells seeded on top of said gel. In one embodiment, said device is a microfluidic device comprising at least one fluidic channel. In one embodiment, said microfluidic device comprises a second fluidic channel separated from said first fluidic channel by said membrane having a second surface facing said second channel. In one embodiment, said second channel comprises an extracellular matrix coating on top of said second surface wherein said coating is overlain with a gel between 20 microns-500 microns in thickness. In one embodiment, said device further comprises endothelial cells seeded in said second channel.

In one embodiment, the present invention provides a method of cell culture, comprising: a) providing: i) a device comprising a membrane having an extracellular matrix coating on top of one surface of said membrane, wherein said coating is overlain with a gel between 20 microns 500 microns in thickness; ii) a population of cells comprising cells capable of differentiating into ciliated cells; iii) a test substance; b) seeding said cell population on top of said gel so as to create seeded cells; c) culturing said seeded cells under conditions such that at least a portion of said cells differentiate wherein said differentiated cells comprise ciliated cells; d) contacting said ciliated cells with said test substance; and e) measuring a function of said ciliated cells. In one embodiment, said function is mucociliary clearance. In one embodiment, said function is measured using an assay selected from the group consisting of quantitative imaging of ciliary beat and mucus flow. In one embodiment, said test substance is selected from the group consisting of a microbe, a particulate, a chemical, a drug compound, and a therapeutic. In one embodiment, said cells are parenchymal cells. In one embodiment, said cells are from a cystic fibrosis patient. In one embodiment, said cells are derived from a cystic fibrosis patient. In one embodiment, said cells are diseased cells derived from a human biopsy. In one embodiment, said cells are human organoid cells. In one embodiment, said cells are human organoid cells derived from iPS cells. In one embodiment, said cells are derived from a patient for use in providing an individualized treatment for that patient. In one embodiment, said gel comprises an extracellular matrix protein. In one embodiment, said extracellular matrix of said coating is a different type than said extracellular matrix of said gel. In one embodiment, said gel is a continuous layer. In one embodiment, said gel provides a continuous layer of approximately 100 microns. In one embodiment, said gel provides a continuous homogenous layer of approximately 30 microns. In one embodiment, said gel provides a continuous layer between 20-80 microns over at least 75% of said membrane surface. In one embodiment, said gel provides a continuous layer between 50-200 microns over at least 75% of said membrane surface. In one embodiment, said gel provides a partial gel layer covering 75%-100% of said membrane surface. In one embodiment, said extracellular matrix of said coating comprises Collagen I and wherein said extracellular matrix of said gel comprises Collagen IV. In one embodiment, said extracellular matrix of said coating comprises Collagen IV and wherein said extracellular matrix of said gel comprises Matrigel®. In one embodiment, said device is a Transwell device. In one embodiment, said device further comprises parenchymal cells seeded on top of said gel. In one embodiment, said device is a microfluidic device comprising at least one fluidic channel. In one embodiment, said microfluidic device comprises a second fluidic channel separated from said first fluidic channel by said membrane having a second surface facing said second channel. In one embodiment, said second channel comprises an extracellular matrix coating on top of said second surface wherein said coating is overlain with a gel between 20 microns-500 microns in thickness. In one embodiment, said device further comprises endothelial cells seeded in said second channel.

In one embodiment, the present invention provides a method of cell culture, comprising: a) providing: i) a device comprising a membrane having an extracellular matrix coating on top of one surface of said membrane, wherein said coating is overlain with a gel between 25 microns-500 microns in thickness; ii) a population of cells comprising cells capable of differentiating into ciliated cells; iii) a test substance; b) seeding said cell population on top of said gel so as to provide seeded cells; c) culturing said seeded cells under an air-liquid interface (ALI) such that at least a portion of said cells differentiate, wherein said differentiated cells comprise ciliated cells; d) contacting said ciliated cells with said test substance; and e) measuring a function of said ciliated cells. In one embodiment, said culture is under an air-liquid interface (ALI) ranging from 1-30 days, up to a year. In one embodiment, said cilia are observed beating 6-8 days after initiation of an air-liquid interface (ALI). In one embodiment, said cilia are observed beating 8-21 days after initiation of an air-liquid interface (ALI). In one embodiment, said cilia are observed beating 25 days after initiation of an air-liquid interface (ALI). In one embodiment, said differentiated cells are selected from the group consisting of goblet cells and club cells. In one embodiment, said measuring is selected from the group consisting of quantitative imaging of ciliary beat and mucus flow. In one embodiment, said cells are parenchymal cells. In one embodiment, said cells are healthy human cells. In one embodiment, said cells are selected from the group consisting of primary human bronchial epithelial cells and primary human tracheal epithelial cells. In one embodiment, said cells are derived from a cystic fibrosis patient. In one embodiment, said cystic fibrosis cells comprise one or more mutations in a cystic fibrosis transmembrane conductance regulator (CFTR) gene resulting in an amino acid mutation selected from the group consisting of F508del, G551D; G1244E; G1349D; G178R; G551S; G970R; S1251N; S1255P; S549N; S549R; and R117H. In one embodiment, said cystic fibrosis cells have at least one mutant F508del allele. In one embodiment, said cystic fibrosis cells have two mutant F508del alleles. In one embodiment, said cystic fibrosis cells have at least one mutant F508del allele and at least one additional mutation in a CFTR protein. In one embodiment, said cystic fibrosis cells have a reduced number of cell surface cystic fibrosis transmembrane conductance regulator (CFTR) proteins. In one embodiment, said test substance increases said number of cell surface cystic fibrosis transmembrane conductance regulator (CFTR) proteins. In one embodiment, said cystic fibrosis cells have a reduced function selected from the group consisting of ion transport function, cilia function, mucus flow trajectory and mucociliary transport function. In one embodiment, said test substance increases at least one function. In one embodiment, said method further providing a second test substance for increasing at least one function. In one embodiment, said method further providing a third test substance for increasing at least one function. In one embodiment, said method further providing a fourth test substance for identifying an adverse substance interaction. In one embodiment, said method further providing a microbe, and a step after step b), contacting said parenchymal cells with said microbe. In one embodiment, said microbe is selected from the group consisting of a virus, a bacteria, a fungi, a phage and a protist. In one embodiment, said virus is a respiratory virus. In one embodiment, said bacteria is selected from the group consisting of Pseudomonas aeruginosa, Streptococcus pneumoniae, Haemophilus species, Staphylococcus aureus and Mycobacterium tuberculosis. In one embodiment, said fungi is an Aspergillus species. In one embodiment, said phage is selected from the group consisting of 14/1, ΦKZ, PNM and PT7. In one embodiment, said protist is selected from the group consisting of Tetrahymena thermophila, Tetrahymena pyriformis and Acanthamoebae polyphaga. In one embodiment, said cells are derived from a patient for use in providing an individualized treatment for that patient. In one embodiment, said differentiated cells provide a confluent layer of cells. In one embodiment, said gel comprises an extracellular matrix protein. In one embodiment, said extracellular matrix of said coating is a different type than said extracellular matrix of said gel. In one embodiment, said gel is a continuous layer. In one embodiment, said gel provides a continuous layer of approximately 100 microns. In one embodiment, said gel provides a continuous homogenous layer of approximately 30 microns. In one embodiment, said gel provides a continuous layer between 20-80 microns over at least 75% of said membrane surface. In one embodiment, said gel provides a continuous layer between 50-200 microns over at least 75% of said membrane surface. In one embodiment, said gel provides a partial gel layer covering 75%-100% of said membrane surface. In one embodiment, said extracellular matrix of said coating comprises Collagen I and wherein said extracellular matrix of said gel comprises Collagen IV. In one embodiment, said extracellular matrix of said coating comprises Collagen IV and wherein said extracellular matrix of said gel comprises Matrigel®. In one embodiment, said device is a Transwell device. In one embodiment, said device further comprises parenchymal cells seeded on top of said gel. In one embodiment, said device is a microfluidic device comprising at least one fluidic channel. In one embodiment, said microfluidic device comprises a second fluidic channel separated from said first fluidic channel by said membrane having a second surface facing said second channel. In one embodiment, said second channel comprises an extracellular matrix coating on top of said second surface wherein said coating is overlain with a gel between 20 microns-500 microns in thickness. In one embodiment, said device further comprises endothelial cells seeded in said second channel. In one embodiment, immune cells are provided, for adding to said device in said second channel. In one embodiment, immune cells are neutorphils.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

As used herein, the term “gel layer” refers to refers to a gelatinous material that is generally considered to have a thickness between approximately 10-100 microns, more preferably between 15-75 microns, and most preferably between approximately between 20-50 microns. Gel layers are easily measured using conventional measuring devices. Gel layer materials generally comprise noncellular tissue-specific and heterogeneous complex including but not limited to a 3D scaffold, i.e. a network of scaffold fibrous proteins such as collagens, laminin, fibronectin, tenascin, elastin, etc., and proteoglycans including glycosaminoglycans (carbohydrate polymers), such as small leucine-rich proteoglycans (SLRPs), modular proteoglycans and cell-surface proteoglycans, hyaluronic acid, typically a negatively charged, non-branched polymer composed of repeated disaccharides of glucuronic acid and N-acetylglucosamine. Gel layers may also comprise a synthetic poly(ethylene glycol) (PEG), poly(vinyl alcohol), and poly(2-hydroxy ethyl methacrylate), etc.

As used herein, the term “partial gel layer” refers to a gel layer that does not cover all the surfaces of a particular device and may only cover one or one less than all of the surfaces of a particular device. Further, a partial gel layer may not cover the entirely of any one surface of a particular device. For example, a partial gel layer may be discontinuous on a particular surface, have different thicknesses across a particular surface, or comprise gaps across a particular surface. For example, only a first surface of a membrane has a gel layer.

As used herein, “extracellular matrix” or “ECM”, refers to a gelatinous material that is generally considered a molecular layer, thereby having a thickness that is below the threshold of conventional measuring devices. ECM materials generally comprise noncellular tissue-specific and heterogeneous complex including but not limited to a 3D scaffold, i.e. a network of scaffold fibrous proteins such as collagens, laminin, fibronectin, tenascin, elastin, etc., and proteoglycans including glycosaminoglycans (carbohydrate polymers), such as small leucine-rich proteoglycans (SLRPs), modular proteoglycans and cell-surface proteoglycans, hyaluronic acid, typically a negatively charged, non-branched polymer composed of repeated disaccharides of glucuronic acid and N-acetylglucosamine. ECM derived from in vivo ECM may further contain attached molecules, such as enzymes, etc. In vitro, ECM may refer to at least one isolated scaffold component found in vivo, or at least one compound mimicking a scaffold component found in vivo, such as a synthetic poly(ethylene glycol) (PEG), poly(vinyl alcohol), and poly(2-hydroxy ethyl methacrylate), etc.

As used herein, “collagen” refers to a fibrous protein found in animals (mainly mammals) composed of three polypeptide chains, i.e. polymers, arranged in a triple helical conformation, with a primary structure that is mostly a repeating motif with glycine in every third position and in humans, proline or 4-hydroxyproline frequently preceding the glycine residue. Native collagen is typically insoluble, thus extraction methods are needed for isolation. Type I collagen differs from other collagens by its low lysine hydroxylation and low carbohydrate composition.

As used herein, “gelatin” refers to a form of collagen generally extracted from body parts of cattle, rats, pigs, fish, etc., as a heterogeneous mixture of water (or biological fluid)-soluble proteins of high average molecular masses. Gelatin proteins are extracted by boiling the relevant skin, tendons, ligaments, bones, etc. in water.

As used herein, “gel” generally refers to a polymer, including but not limited to collagen, either in the form of a powder, in solution, or as a partially networked compound or a fully networked compound forming a semi-solid, jelly-like material, e.g. a colloid in a more solid form than a solution. Gel may also refer to isolated ECM, both in solution and polymerized.

As used herein, “gel” or “gelling” or “gelation” may refer to the process of transformation from a liquid gel solution (sol) to a semi-solid, jelly-like material, which in turn the jelly-like material may also be referred to as a noun, i.e. “gel”. One form of a gel, wherein the liquid component is mostly water, biological fluid, or water based solution, e.g. buffer, media, etc., is referred to as a “hydrogel”.

A “hydrogel,” in addition to referring to a gel containing a high amount of water or biological fluid, also refers to a hydrophilic network of polymers producing an elastic structure. Polymers may be natural, naturally derived or synthetic, or combination thereof, such as polymers found in extracellular matrix in vivo.

As used herein, “jelly-like” in reference to a material has properties ranging from soft to hard.

As used herein, “polymer” refers to one large molecule comprising many smaller molecules of the same kind linked together, i.e. repeat units or monomers. Some polymers are produced in vivo and others are synthetically produced. A polymer may be linear with or without side chains or branches.

As used herein, “transglutaminase” or “TG” or “Tgase” refers to an enzyme (i.e. protein-glutaminase γ-glutamyltransferase, EC 2.3.2.13) capable of catalyzing post-translational modifications of proteins.

As used herein, “microbial transglutaminases” or “mTgs” refer to TG enzymes capable of deamidating/transamidating proteins, enabling cross-linkage of molecules. MTgs are produced endogenously or recombinantly by bacteria, e.g. Streptomyces mobaraensis, Escherichia coli, Bacillus subtilis, etc.

The term “in vivo” refers to the natural environment (e.g., within an organism, tissue, and/or a cell).

The term “ex vivo” refers to an environment outside an organism.

The term “in vitro” refers to an ex vivo environment that includes manipulation under artificially-created conditions (e.g., culture medium, cell culture, transfection, assay) and/or using laboratory equipment (e.g., flasks, test tubes, petri dishes, multiwell plates, etc.).

The term “culture medium” refers to a nutritive substance that is suitable to support maintenance and/or growth of cells in vitro (i.e., cell cultures). A culture medium includes, for example, liquid media, and three-dimensional media. A culture medium includes but is not limited to salts (e.g., sodium chloride), carbohydrates (e.g., sugar), proteins (e.g., serum), etc.

The term “three-dimensional media,” “3D media,” and “three-dimensional matrix” interchangeably refer to an artificially-created environment (e.g., Matrigel®, collagen, agar, etc.) in which biological cells are permitted to grow or interact with their surroundings in all three dimensions. This is in contrast to growing cells in “two-dimensional” (2D) monolayers (e.g., on a petri dish) because the 3D model more accurately models the in vivo cell environment.

“Viscous” generally refers to a substance in between a liquid and a solid, i.e. having a thick consistency. A “viscosity” of a fluid refers to a measure of its resistance to gradual defoiination by shear stress or tensile stress. For liquids, it corresponds to an informal concept of “thickness”; for example, honey has a much higher viscosity than water.

“Viscous fingering” refers in general to the formation of open spaces (e.g., voids or lumens) within a solid or semi-solid gel.

As used interchangeably herein, the terms “non-porous” and “non-permeable” refer to a material that does not allow any molecule or substance to pass through.

As used herein, the term “porous” generally refers to a material that is permeable or selectively permeable. The term “permeable” as used herein means a material that permits passage of a fluid (e.g., liquid or gas), a molecule, a whole living cell and/or at least a portion of a whole living cell, e.g., for formation of cell-cell contacts. The term “selectively permeable” as used herein refers to a material that permits passage of one or more target group or species, but act as a barrier to non-target groups or species. For example, a selectively-permeable membrane can allow passage of a fluid (e.g., liquid and/or gas), nutrients, wastes, cytokines, and/or chemokines from one side of the membrane to another side of the membrane, but does not allow whole living cells to pass therethrough. In some embodiments, a selectively-permeable membrane can allow certain cell types to pass therethrough but not other cell types.

The term, “immune cells” as used herein, refers to any cell that is involved in immunological pathways. The cells of the immune system are usually white blood cells categorized as lymphocytes (e.g., T-cells, B-cells and NK cells), neutrophils, and monocytes/macrophages. More specifically, immune cells may include, but are not limited to, B cells, dendritic cells, granulocytes, innate lymphoid cells (ILCs), megakaryocytes, monocyte, macrophages, myeloid-derived suppressor cells (MDSCs). natural killer cells (NK Cells) and/or peripheral blood mononuclear cells (PBMCs).

The term, “agent” or “compound” as used herein, refers to any molecule (e.g., small organic compounds, peptides, proteins, hormones etc.) that exert a biological effect when contacting a living biological cell. For example, an agent may induce effects including, but not limited to, inflammation, cell migration and/or recruitment. Exemplary agents as used herein may include, but are not limited to, viruses, cytokines, necrosis factors and/or interleukins.

The term “microfluidic” as used herein relates to components where moving fluid is constrained in or directed through one or more channels wherein one or more dimensions are 1 mm or smaller (microscale). Microfluidic channels may be larger than microscale in one or more directions, though the channel(s) will be on the microscale in at least one direction. In some instances the geometry of a microfluidic channel may be configured to control the fluid flow rate through the channel (e.g. increase channel height to reduce shear). Microfluidic channels can be formed of various geometries to facilitate a wide range of flow rates through the channels. Examples of microfluidic devices with microfluidic channels are provided in U.S. Pat. No. 8,647,861, hereby incorporated by reference.

The term “transwell” as used herein relates to a chamber supported by a substrate (e.g., a well plate) and is configured to support a removable insert having a bottom surface with a porous filter capable of growing a cell culture that is in contact with a cell culture media.

The term “chamber” as used herein relates to a structure configured to hold a fluid and is configured to grow a cell culture. For example, a chamber may be a well (e.g., a microarray well or a portion of a transwell). Alternatively, a chamber may be a channel (e.g., a microchannel). A chamber may optionally be open or closed or formed within a channel or microchannel.

“Channels” are pathways (whether straight, curved, single, multiple, in a network, etc.) through a medium (e.g., silicon) that allow for movement of liquids and gasses. Channels thus can connect other components, i.e., keep components “in communication” and more particularly, “in fluidic communication” and still more particularly, “in liquid communication.” Such components include, but are not limited to, liquid-intake ports and gas vents. Microchannels are channels with dimensions less than 1 millimeter and greater than 1 micron.

As used herein, the phrases “connected to,” “coupled to,” “in contact with” and “in communication with” refer to any form of interaction between two or more entities, including mechanical, electrical, magnetic, electromagnetic, fluidic, and thermal interaction. For example, in one embodiment, channels in a microfluidic device are in fluidic communication with cells and (optionally) a fluid source such as a fluid reservoir. Two components may be coupled to each other even though they are not in direct contact with each other. For example, two components may be coupled to each other through an intermediate component (e.g. tubing or other conduit).

As used herein, “respiratory virus” refers to a virus capable of infecting cells in the respiratory system, such as bronchial epithelial cells, examples including but not limited to parainfluenza virus, influenza virus, rhinovirus, coronaviruses, human respiratory syncytial virus, adenoviruses, etc.

As used herein, “parainfluenza virus” or “NV” refers to a virus that can cause both upper and lower respiratory infections including colds, bronchiolitis, bronchitis, croup and pneumonia. PIV-1 and PIV-2 are common causes of croup, whereas PIV-3 often causes lower respiratory tract infections (LRIs), such as bronchiolitis and pneumonia. Human parainfluenza virus may be referred to as “HPIV.”

As used herein, “rhinovirus” refers to any virus of a group of picornaviruses, including those that cause some forms of the common cold.

As used herein, “flu” refers to an infectious disease caused by an influenza virus in addition to other “flu-like” viruses, characterized by symptoms such as fever, muscle pain, headache, and inflammation of the mucous membranes in the respiratory tract The term “disease” or “medical condition”, as used herein, refers to any impainnent of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “inhibitory compound” as used herein, refers to any compound capable of interacting with (i.e., for example, attaching, binding etc.) to a binding partner under conditions such that the binding partner becomes unresponsive to its natural ligands. Inhibitory compounds may include, but are not limited to, small organic molecules, antibodies, and proteins/peptides.

The term “derived from” as used herein, refers to the source of a sample, a compound or a sequence. In one respect, a sample, a compound or a sequence may be derived from an organism or particular species. In another respect, a sample, a compound or sequence may be derived from a larger complex or sequence.

The term “sample” or “biopsy” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables). For example, a pulmonary sample may be collected by bronchoalveolar lavage (BAL) which comprises fluid and cells derived from lung tissues. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

The term “bind” as used herein, includes any physical attachment or close association, which may be permanent or temporary. Generally, an interaction of hydrogen bonding, hydrophobic forces, van der Waals forces, covalent and ionic bonding etc., facilitates physical attachment between the molecule of interest and the analyte being measuring. The “binding” interaction may be brief as in the situation where binding causes a chemical reaction to occur. That is typical when the binding component is an enzyme and the analyte is a substrate for the enzyme. Reactions resulting from contact between the binding agent and the analyte are also within the definition of binding for the purposes of the present invention.

In a related aspect, “device” is used to describe both arrays and microarrays, where the array or microarray may comprise other defined components including surfaces and points of contact between reagents.

Further, “substrate” is also a term used to describe surfaces as well as solid phases which may comprise the array, microarray or device. In some cases, the substrate is solid an may comprise PDMS.

The term “orientational order” as used herein refers to a parameter that evaluation the quantitative vector component of a ciliary biomarker. This term may also be used in the context of directionality, e.g., when the cilia are beating in a certain direction. An “orientational order correlation” evaluates whether two different regions of cilia share the same directionality vector for a particular ciliary biomarker.

### BRIEF DESCRIPTION OF THE DRAWINGS

Exemplary embodiments are illustrated in referenced figures. It is intended that the embodiments and figures disclosed herein are to be considered illustrative rather than restrictive. The file of this patent contains at least one drawing executed in color. Copies of this patent with color drawings will be provided by the Patent and Trademark Office upon request and payment of the necessary fee.

FIG. 1A shows an exemplary schematic of ECM molecular coating (thin cross-hatched layer) under parenchymal cells, such as hepatocytes, with a Matrigel® molecular coating (thin cross-hatched layer) above parenchymal cells in one embodiment of a fluidic device. A dotted line represents a membrane separating the parenchymal cells (and gels) from another type of cell layer, such as endothelial cells (dark rectangles) and white blood cells (blue stars).

FIG. 1B shows an exemplary schematic of a parenchymal cell layer (e.g., hepatocytes) with a 3D gel overlay (thick cross-hatched layer; less than 100 μm) and an ECM molecular coating (thin cross-hatched layer) located under the parenchymal cells. A dotted line represents a membrane separating the parenchymal cells (and gels) from another type of cell layer, such as endothelial cells (dark rectangles) and white blood cells (blue stars).

FIG. 1C shows an exemplary schematic of a parenchymal cell layer (e.g., hepatocytes) with a 3D gel underlay (thick cross-hatched layer) and 3D gel overlay (thick cross-hatched layer), both layers being less than 100 μm. A dotted line represents a membrane separating the parenchymal cells (and gels) from another type of cell layer, such as endothelial cells (dark rectangles) and white blood cells (blue stars).

FIG. 1D shows an exemplary schematic of a parenchymal cell layer (e.g., hepatocytes) with an ECM molecular coating (thin cross-hatched layer) below the parenchymal cells and a Matrigel® molecular coating above parenchymal cells (thin cross-hatched layer). A dotted line represents a membrane separating the parenchymal cells (and gels) from another type of cell layer, such as endothelial cells (dark rectangles), white blood cells (blue stars) and at least one additional cell type (red stars) such as Kupffer cells added to the lower cell layer.

FIG. 1E shows an exemplary schematic of a parenchymal cell layer (e.g., hepatocytes) with a 3D gel overlay of less than 100 μm below the parenchymal cells (thick cross-hatched layer) and an ECM molecular coating (thin cross-hatched layer) above parenchymal cells. A dotted line represents a membrane separating the parenchymal cells (and gels) from another type of cell layer, such as endothelial cells (dark rectangles) and white blood cells (blue stars). Additional cells may be added to the overly for migration (red stars).

FIG. 1F shows an exemplary schematic of a parenchymal cell layer (e.g., hepatocytes) with a 3D gel overlay below the parenchymal cells (thick cross-hatched layer) and a 3D gel underlay (thick cross-hatched layer) above parenchymal cells, both layers being less than 100 μm. A dotted line represents a membrane separating the parenchymal cells (and gels) from another type of cell layer, such as endothelial cells (dark rectangles) and white blood cells (blue stars). In some embodiments, at least one additional cell type (red stars) such as Kupffer cells are located on the same side of the membrane as the parenchymal cell layer. In some embodiments, additional cell types (red stars) are added with said 3D gel overly. In some embodiments, additional cell types (red stars) migrate into said 3D gel overly.

FIGS. 2A-C illustrate embodiments of an exemplary microfluidic device which may find use with the present invention.

FIG. 2A: Illustrates a perspective view of a microfluidic device with microfluidic channels in accordance with an embodiment.

FIG. 2B: Illustrates an exploded view of the device 200 in accordance with an embodiment, showing a microfluidic channel in a top piece 207 and a microfluidic channel in a bottom piece, separated by a membrane 208, in some embodiments the membrane is stretchable.

FIG. 2C: Shows cells in relation to device parts in a closed top chip, e.g. upper microchannel (1-blue); lower microchannel (2-red) and optional vacuum chamber (6). 1. Options include a liquid microchannel; air-liquid microchannel (upper); 2. Vascular channel (lower); 3. parenchymal cells, including but not limited to epithelial cells/tissue (e.g. liver, kidney, lung), other types of cells, reticular cells (e.g. lymph node), neuronal cells, pericytes astrocytes (e.g. brain); 4. Simulated capillaries (e.g. endothelial cells matching or compatible with the cells in the upper chamber); 5. Membrane, stretchable; and 6. Vacuum Channels. Arrows represent direction of fluid flow.

FIG. 3A illustrates an exploded view of one embodiment of a stretchable open top chip device (3000) demonstrating the layering of a fluidic top, top structure and bottom structure.

FIG. 3B illustrates a cut-away view of one embodiment of a stretchable open top chip device (3100) showing the regional placement of assay cells (e.g., epithelial cells, dermal cells and/or vascular cells), further demonstrating a lumen comprising an epithelial compartment and a stromal compartment in addition to a vascular compartment.

FIG. 3C illustrates a fully assembled view of one embodiment of a stretchable open top chip device.

FIG. 3D illustrates one embodiment of an exploded view of a stretchable open top chip device.

FIGS. 4A-B show one embodiment of an Open Top Chip.

FIG. 4A shows one embodiment of an assembled chip, showing the open top chambers above, and separated by a membrane from, the lower channel fluidics.

FIG. 4B shows exemplary exploded view of an open top chip comprising two systems on one chip, wherein the membrane is highlighted in order to illustrate the relationship of the assembled components.

FIGS. 5A-E shows exemplary results of cell layers attached to one channel of a fluidic device using a collagen I extra cellular matrix. Attached cells are colored red for showing a uniform cell layer.

FIG. 5A shows exemplary results of 2 mg/mL Collagen 1 gel; 4 5 min×25° C.; Syringe Pump.

FIG. 5B shows exemplary results of 2 mg/mL Collagen 1 gel; 30 min×37° C.; Manual.

FIG. 5C shows exemplary results of 1 mg/mL Collagen 1 gel; 16 hr×4° C.; Syringe Pump.

FIG. 5D shows exemplary results of 1 mg/mL Collagen 1 gel; 45 min×25° C.; Syringe Pump.

FIG. 5E 1 mg/mL; 30 min×37° C.; Syringe Pump.

FIG. 6 shows an exemplary gel contraction with a dilute gel concentration and ALI Culture. Gel compositions with 7 mg/ml Collagen I showed stability with only a minimal level of contraction after 2 weeks of culture. Gel stability was observed in the presence of both a basic growth media (e.g., DMEM) and a complex growth media (e.g., EM).

FIG. 7 shows exemplary data showing a concentration dependent limitation on fibroblast proliferation with increasing gel concentration. For example, a gel composition with 7 mg/ml of Collagen I prevents fibroblast overgrowth thereby resulting in increased gel contraction.

FIG. 8 shows exemplary data showing that a collagen IV coating improves intestinal cells attachment. For example, coating of the top surface of a gel with a collagen IV/Matrigel® ECM improves intestinal epithelium attachment.

FIG. 9A-B compares known partial gel layers to those contemplated herein.

FIG. 9A: A microchannel containing gel comprising an enclosed lumen created by the viscous fingering technique. The gel is in contact with all four sides of the microchannel The lumen has differing diameters along its length (Panel A compared with Panel B) and is occluded at various locations (Panel C) due to a lack of uniformity in gel formation

FIG. 9B: A microchannel comprising a partial gel layer having an unenclosed surface that is flat (Panel A) or concave (Panel B). The partial gel layer does not contact all four sides of the microchannel.

FIG. 10A-B presents an exemplary photomicrograph of transmigration and recruitment of an immune cell induced by an inflammatory stimuli.

FIG. 10A: A partial gel layer with a plurality of transmigrated fluorescently labeled PBMC cells induced and recruited by human rhinovirus or interleukin 13.

FIG. 10B: A close-up of a region in FIG. 9A that clearly shows several transmigrated PBMC cells detected at different levels of fluorescent intensity. These data are interpreted to demonstrate that the PBMCs emitting less intense and more diffuse fluorescence have penetrated the deepest into the partial gel layer.

FIG. 11 illustrates one embodiment of a lung-on-a-chip (e.g., Air-way chip, S1 Base Model) comprising a membrane (7 μm pores) with a mucociliary airway epithelium layer and a microvascular epithelial layer.

FIG. 12 presents an exemplary timeline employed to test the parameters of the S1 Base Model chip.

FIG. 13 presents an exemplary categorization of five (5) different parameters that were investigated in the development of the S1 Base Model chip.

FIG. 14 presents exemplary photomicrographs of cell biomarkers identifying specific score values for viability and differentiation during chip cell culture maturation. Cell death (1); Ciliation (2): Cell attachment (3); Movement/Shape (4); Cell overgrowth (5); cell invasion (6).

FIG. 15 presents exemplary data showing score values for cell viability & differentiation biomarkers comparing four (4) different gel coatings (e.g., Coating A, Coating B, Coating C and Coating D). Each coating can be seen to result in different score values that are independent of location in the cell culture device (e.g., Zoe).

FIG. 16 presents exemplary data utilizing the scoring method in accordance with FIGS. 14 and 15 that identifies Bovine Serum Albumin (BSA) as supporting the most viable and differentiated cell layer.

FIG. 17 presents exemplary data utilizing the scoring method in accordance with FIGS. 14 and 15 that shows that increased cell culture media flow rate does not affect cell viability and/or differentiation.

FIG. 18 presents exemplary data utilizing the scoring method in accordance with FIGS. 14 and 15 that shows that increased retinoic acid concentrations supports the best cell viability and/or differentiation.

FIG. 19 presents exemplary data utilizing morphological observation of several viability/differentiation biomarkers to compare the efficacy of several different cell culture media.

FIG. 20A-H presents prior art photomicrographs demonstrating the huge variability in differentiated bile canaliculi growth in culture. Arrows and white areas appearing as wiggly lines within tissues show bile canaliculi within cultures.

FIG. 21 presents prior art data where a correlation can be observed between the extent of BC network, and therefore biliary flow, and Cl_{biliary} values. Ghibellini, 2007.

FIG. 22 presents exemplary photomicrograph data showing viability and differentiation of lung cells using cilia development as a biomarker (6 chips, 1 donor). Red: Cilia (tubulin). Blue: Nuclei (DAPI). Green: Basal Cells (p63).

FIG. 23A-B presents exemplary photomicrograph data showing viability and differentiation of lung cells using mucus secretion and cilia development as a biomarker (6 chips, 1 donor).

FIG. 23A: Green: Goblet Cells (Muc5AC); Red: Basal Cells (p63); and Blue: Nuclei (DAPI).

FIG. 23B: Green: Club Cells (CC16); Red: Basal Cells (p63); Blue: Nuclei (DAPI)

FIG. 24 presents exemplary quantitative data for the photomicrographs in FIGS. 23A and 23B.

FIG. 25A-B presents exemplary photomicrograph data of a differentiated lung cells having a stable ciliary beat frequency (6 chips, 1 donor). 100% of chips are fully ciliated and have steady ciliary beat after twenty-one (21) days exposure to an ALI (6 chips, 1 donor).

FIG. 25A: A stable ciliated cell culture maintained at day 21 of exposure to an ALI.

FIG. 25B. Motion detection microscopy shows a synchronized cilia beat frequency.

FIG. 26 presents exemplary quantitative data for the photomicrographs in FIGS. 25A and 25B.

FIG. 27A-B presents exemplary photomicrograph data of a differentiated lung cells having mucociliary transport (6 chips, 1 donor). 100% of chips are fully ciliated and have mucociliary transport function after twenty-one (21) days exposure to an ALI (6 chips, 1 donor).

FIG. 27A: A differentiated lung cell culture having mucociliary transport function maintained at day 21 of exposure to an ALI using 1 μm diameter fluorescent beads.

FIG. 27B. Motion detection microscopy shows mucus flow trajectories.

FIG. 28 shows a representative photomicrograph of epithelial cells on bottom side of membrane at day 14 of exposure to an ALI using standard conditions.

FIG. 29 presents an illustrative photomicrograph showing a top view (left panel) and a side view (right panel) of a partial gel matrix exposed to a top channel embedded with epithelial cells that do not migrate to the bottom channel.

FIG. 30 presents exemplary data showing the percent gel layer coverage of various embodiments of collagen-based gelling fluids.

FIG. 31 presents an exemplary photomicrograph of the uniformity of collagen IV-Matrigel® partial gel layer created with hydrodynamic flushing.

FIG. 32A-B presents exemplary data showing average partial gel layer thickness.

FIG. 32A shows a photomicrograph showing the relative uniformity of a partial gel layer width along the membrane.

FIG. 32B presents quantitative data of the partial gel layer widths between the various gelling fluid compositions.

FIG. 33A-B presents exemplary data showing gel erosion patterns during hydrodynamic flushing.

FIG. 33A depicts significant manual syringe-induced gel erosion patterns in the distal channel region (L) and middle channel region (M) relative to the channel inlet port.

FIG. 33B depicts no automatic pipettor-induced gel erosion in any of the channel regions (L, M or R) relative to the channel inlet port.

FIG. 34 presents representative photomicrographs showing the consistency and uniformity of a partial gel layer formed with a collagen IV+Matrigel® gelling fluid. Arrows: Small empty patches.

FIG. 35 presents representative photomicrographs showing the unsuccessful formation of a partial gel layer with a 2% gelatin gelling fluid.

FIG. 36 presents representative photomicrographs showing the unsuccessful formation of a partial gel layer with a 5% gelatin gelling fluid.

FIG. 37 presents representative photomicrographs showing the unsuccessful formation of a partial gel layer with a collagen-I (175 μg/ml)+collagen-IV (1.175 μg/ml)+laminin (100 μg/ml)+fibronectin (50 μg/ml) gelling fluid.

FIG. 38 presents representative photomicrographs showing the unsuccessful formation of a partial gel layer with a collagen I (0.5 mg/mL)+collagen IV (300 μg/mL) gelling fluid.

FIG. 39A-B demonstrates improved cystic fibrosis cell differentiation in transwell cultures with a collagen I gel underlay.

FIG. 39A shows CF cells on a standard transwell culture plate with a collagen VI overlay coating.

FIG. 39B shows CF cells on a standard Transwell culture plate with a collagen VI overlay coating and collagen I gel underlay.

FIG. 40A-B demonstrates motion detection of ciliary beat by showing density of ciliation in transwell cultures.

FIG. 40A shows CF cells on a standard transwell culture plate with a collagen VI overlay coating.

FIG. 40B shows CF cells on a standard Transwell culture plate with a collagen VI overlay coating and collagen I gel underlay. Improved ciliary beat frequency and synchronization is seen.

FIG. 41 presents illustrative data regarding the problematic nature of culturing cystic fibrosis cells in an open-top microfluidic device. Arrows: Representative hole in the culture monolayer.

FIG. 42A-B presents exemplary data showing ciliation differentiation of cystic fibrosis cells in a microchannel comprising a mixed collagen I/collagen IV partial gel layer. 100% (6/6 chips) differentiated and became ciliated after 14 days exposure to an ALI.

FIG. 42A shows two representative CF-cell microchips with a differentiated cystic fibrosis cell monolayer.

FIG. 42B shows two representative CF-cell microchips under motion detection microscopy showing ciliation density. Cell beat frequency synchrony was also observed.

FIG. 43A-D presents exemplary photomicrographs demonstrating complete cell differentiation of cystic fibrosis cells in a microchannel comprising a mixed collagen I/collagen IV partial gel layer. 100% (6/6 chips) differentiated and became ciliated after 14 days exposure to an ALI. Achieved complete differentiation in healthy and CF Chips.

FIG. 43A: Orange: Cilia (tubulin); Green: Goblet cells (Muc5AC); Light blue: Basal cells (p63); Dark blue: Actin.

FIG. 43B: Green: Cilia (tubulin); Magenta: Basal cells (p63)

FIGS. 43C and 43D: shows representative 100% of CF chips fully differentiate in optimized conditions at 21 days ALI (3 chips, 1 donor) FIG. 43C shows Orange: Cilia (tubulin); Green: Goblet cells (Muc5AC); DAPI stained nuclei blue.

FIG. 43D shows club cells (CC-16) and ciliated cells—magenta (alpha tubulin stained) DAPI stained nuclei blue.

FIG. 44A-B presents exemplary data showing functional differences between mucous-secreting differentiated cystic fibrosis goblet cells and mucous-secreting differentiated healthy goblet cells. Example video analysis of mucociliary transport (here done on transwell). Mucus flow trajectories revealed by motion detection. Exemplary measure of mucociliary transport on CF-Chip and quantify velocities as a functional indicator of cilia function.

FIG. 44A: Mucociliary transport pattern of differentiated cystic fibrosis goblet cells.

FIG. 44B: Mucociliary transport pattern of differentiated healthy goblet cells.

FIG. 45A-d presents exemplary data showing functional differences between ciliated differentiated cystic fibrosis cells and differentiated healthy lung cells.

FIG. 45A: Ciliation of differentiated cystic fibrosis cells.

FIG. 45B: Ciliation of differentiated healthy lung cells.

FIGS. 45C and 45D: Mucociliary Transport on Day 25 ALI (1 um fluorescent beads).

FIG. 45C Mucociliary Transport

FIG. 45D: Mucus flow trajectories revealed by motion detection.

FIG. 46A-C presents exemplary data of BC network variability in standard Liver-Chips (e.g., without partial gel layers).

FIG. 46A: BC network adjacent to the outlet port.

FIG. 46B: BC network within access channel from outlet port to main channel.

FIG. 46C: BC network in the mid-section of the main channel.

FIG. 47 presents exemplary data showing that partial gel overlay degradation was greater in a mid-main channel location as compared to a port location and had a greater degradation rate under fluid flow conditions.

FIG. 48 presents exemplary data showing that BC network quality is positively correlated with partial gel layer thickness (e.g., adjacent to inlet/outlet ports).

FIG. 49A-B presents a schematic showing the relationship between gel underlays and overlays and BC network formation.

FIG. 49A: Asymmetric, spherical BC formation with only a partial gel underlay.

FIG. 49B: Symmetric, elongated BC formation with both a partial gel underlay and a partial gel overlay.

FIG. 50 presents exemplary photomicrograph data showing spherical BC networks and cholestasis with only a partial gel underlay as illustrated in FIG. 49A.

FIG. 51 presents exemplary photomicrographs showing significant improvement in BC network quality that correlate with increased partial gel layer thickness and reduced erosion as a function of different gel compositions in accordance with Table VII.

FIG. 52 presents exemplary data showing BC network branching density metrics for various BC networks differentiated on different gel compositions in accordance with Table VII.

FIG. 53 presents exemplary data showing BC network branching density metrics for various BC networks.

FIG. 54 presents exemplary data differentiating between immature BC networks and mature BC networks using a 3 D plot of circularity, length and solidity parameters.

FIG. 55 presents exemplary data showing that a collagen/fibronectin partial gel layer overlay develops superior BC networks as compared to conventional Matrigel® partial gel layer overlay using a shape analysis 3-D plot.

FIG. 56A-C presents illustrations comparing conventional cell culture compositions with improved cell culture compositions comprising a partial gel layer.

FIG. 56A: A conventional cell culture gel overlay composition.

FIG. 56B: An improved cell culture composition comprising a partial gel overlay and a partial gel underlay.

FIG. 56C: An improved cell culture composition comprising a partial gel overlay and a partial gel underlay with a stellate cell layer.

FIG. 57A-C presents exemplary data comparing crossectional views following the creation of partial gel layers using a manual syringe flush and an automatic pipettor flush.

FIG. 57A: Manual syringe creation of a partial gel layer photomicrographs from three locations (L,M,R) within a main microchannel. Notable discontinuity is present.

FIG. 57B: Low shear velocity automatic pipettor creation of a partial gel layer photomicrographs from three locations (L,M,R) within a main microchannel. Notable incomplete gel removal is present.

FIG. 57C: High shear velocity automatic pipettor creation of a partial gel layer photomicrographs from three locations (L,M,R) within a main microchannel. Notable complete gel removal and a continuous/homogeneous layer is present.

FIG. 58A-B presents exemplary data comparing top surface views following the creation of partial gel layers using a manual syringe flush and an automatic pipettor flush.

FIG. 58A: Manual syringe creation of a partial gel layer photomicrographs from three locations (L,M,R) within a main microchannel. Notable discontinuity is present.

FIG. 58B: High shear velocity automatic pipettor creation of a partial gel layer photomicrographs from three locations (L,M,R). A continuous/homogeneous layer is present.

FIG. 59A-C illustrates representative assessment parameters of gel layer coverage:

FIG. 59A: 2-D area: A molecular extracellular matrix on one microchannel surface;

FIG. 59B: 2-D/2 area: A 3-D gel layer on one microchannel surface;

FIG. 59C: 3-D area: A thick 3-D gel layer on multiple microchannel surfaces.

FIG. 60A-C presents representative photomicrographs of one embodiment of a rating scheme to assess partial gel layer coverage;

FIG. 60A: A microchannel with a discontinuous partial gel layer. Inset: 1=2-D area; 2=2-D/2 area; 3=3-D area in accordance with FIG. 59.

FIG. 60B: Magnified version of the microchannel of FIG. 60A.

FIG. 60C: Thresholding of FIG. 60B subtracting 2-D+2-D/2 areas showing gel layer gaps (red) within the 2-D regions (dark).

FIG. 61A-D presents exemplary photomicrographs of partial gel layer coverage assessments in accordance with FIG. 59 using different gel solution matrix compositions:

FIG. 61A: 0.5 mg/mL collagen I; 2-D/2 area rating.

FIG. 61B: 0.5 mg/mL collagen I+0.1 mg/mL fibronectin; 2-D/2 area rating.

FIG. 61C: 0.5 mg/mL collagen I+0.2 mg/mL collagen IV; 3-D area rating.

FIG. 61D: 0.4 mg/mL collagen IV+0.2 mg/mL Matrigel®; 3-D area rating.

FIG. 62A-B presents exemplary data showing differences in 2-D/2 area gel coverage is dependent upon gel composition.

FIG. 62A: Total amount of gel coverage in 2-D/2+3-D areas.

FIG. 62B: Thresholded amount of gel coverage in 2-D/2 gel area.

FIG. 63 presents exemplary data showing differences in gel layer thickness in 3-D gel areas.

FIG. 64 presents exemplary data showing an erosion/degradation pattern of a partial gel layer during nine (9) days of fluid flow.

FIG. 65 presents exemplary data showing a gel layer lumen without an erosion/degradation pattern during nine (9) days of fluid flow.

FIG. 66A-C presents exemplary data showing morphology of differentiated hepatocytes in a microchannel with a thick gel overlay.

FIG. 66A: A flat surfaced thick collagen I+MTG overlay of a hepatocyte cell layer with a collagen I+fibronectin underlay.

FIG. 66B: A flat surfaced thick collagen I+collagen IV overlay of a hepatocyte cell layer with a collagen I+fibronectin underlay.

FIG. 66C: A lumenized collagen I thick overlay of a hepatocyte cell layer with a collagen I underlay.

FIG. 67A-C presents exemplary data showing bile canaliculi (BC) development of differentiated hepatocytes in a microchannel with a thick gel overlay.

FIG. 67A: A flat surfaced thick collagen I+MTG overlay of a hepatocyte cell layer with a collagen I+fibronectin underlay.

FIG. 67B: A flat surfaced thick collagen I+collagen IV overlay of a hepatocyte cell layer with a collagen I+fibronectin underlay.

FIG. 67C: A lumenized collagen I thick overlay of a hepatocyte cell layer with a collagen I underlay.

FIG. 68A-B presents exemplary data showing that hepatic stellate cells embedded within a gel underlay further improve BC development with a lumenized thick gel overlay.

FIG. 68A: A photomicrograph showing the embedded hepatic stellate cells embedded within an gel underlay.

FIG. 68B: A photomicrograph showing elongated BC networks showing improved differentiation as compared to FIG. 67C.

FIG. 69 presents a representative experimental design to induce hepatic steatosis in an in vitro hepatocyte cell layer

FIG. 70A-C presents exemplary data demonstrating microchannel design-induced modulation of hepatocyte lipid accumulation.

FIG. 70A: Lipid accumulation in hepatocytes with a thin Matrigel® gel overlay (˜25 μm).

FIG. 70B: Lipid accumulation in hepatocytes with a thick collagen-I gel overlay (approximately ˜200 μm) without hepatic stellate cells.

FIG. 70C: Lipid accumulation in hepatocytes with a thick collagen I gel overlay (approximately ˜200 μm) with hepatic stellate cells.

FIG. 71A-F presents exemplary data demonstrating the effect of ethanol (0.08%), hepatic stellate cells (HSCs), and/or LPS on hepatocyte lipid accumulation with a thick collagen I gel overlay.

FIG. 71A: Lipid accumulation without ethanol, LPS or HSCs.

FIG. 71B: Lipid accumulation with HSCs but without ethanol and LPS.

FIG. 71C: Lipid accumulation with ethanol but without LPS and HSCs.

FIG. 71D: Lipid accumulation with ethanol and HSCs but without LPS.

FIG. 71E: Lipid accumulation with ethanol and LPS but without HSCs.

FIG. 71F: Lipid accumulation with ethanol, LPS and HSCs.

FIG. 72A-B presents exemplary data showing that HSCs are activated subsequent to ethanol+LPS incubation.

FIG. 72A: HSC expression of vimentin.

FIG. 72B: HSC expression of SMA.

FIG. 73A-C presents exemplary data showing that HSCs improve BC network development in hepatocytes with a thick gel layer overlay.

FIG. 73A: BC network development in a conventional thin Matrigel® overlay.

FIG. 73B: BC network development with a thick collagen I overlay.

FIG. 73C: BC network development with HSCs and a thick collagen I overlay.

FIG. 74A-F presents exemplary data demonstrating the effect of ethanol (0.08%), hepatic stellate cells (HSCs), and/or LPS on BC network development with a thick collagen I gel overlay.

FIG. 74A: BC network development without ethanol, LPS or HSCs.

FIG. 74B: BC network development with HSCs but without ethanol and LPS.

FIG. 74C: BC network development with ethanol but without LPS and HSCs.

FIG. 74D: BC network development with ethanol and HSCs but without LPS.

FIG. 74E: BC network development with ethanol and LPS but without HSCs.

FIG. 74F: BC network development with ethanol, LPS and HSCs.

FIG. 75 presents exemplary data showing BC network quantitative metrics of branching density, porosity and radius.

FIG. 76A-C presents exemplary data demonstrating development of embodiments of an ALD Liver-Chip.

FIG. 76A presents exemplary approaches for modeling human ALD/ASH by exposing the organotypic Liver-Chip to human relevant blood-alcohol concentrations (BAC). FIG. 76B Biomimetic hepatobiliary architecture was achieved through systematic optimization of the on-chip ECM scaffold using quantitative metrics of FIG. 76B(i) Biomimetic Bile Canaliculi (BC) bile canaliculi (BC) network integrity, FIG. 76B(ii) including branching density (higher values are better), and mean radius (more narrowly distributed values are better).

FIG. 76C(i) presents exemplary Lipid droplet accumulation in hepatocytes visualized using AdipoRed staining after administration of fat (oleic acid 1 μg/ml; positive control) or ethanol (0.08% and 0.16%) for 48 hours.

FIG. 76C(ii) presents exemplary digital pathology was used to quantify the number of lipid droplets per cell, and lipid droplet size (projected area). Data represent median+(min and max). *p<0.05; **p<0.01 versus control (Kruskal-Wallis and Dunnett's multiple comparisons test).

FIG. 77A-C presents exemplary data demonstrating assessment of liver toxicity and metabolic changes in one embodiment of an ALD/ASH Liver-Chip. Quantitative analysis of hepatic functional markers in the Liver-Chip after 48h of exposure to physiologically relevant BAC. Fluorometric assessment using ELISA of FIG. 77A cholesterol levels in effluent (Cholesterol release), FIG. 77B glycogen storage in cell lysate, and FIG. 77C albumin release in effluent.

FIG. 78A-G presents exemplary data demonstrating alterations in gene expression changes induced by physiologically-relevant ethanol concentrations. Differential gene expression analysis in hepatocytes from ethanol-treated (for 48h at ethanol concentrations of either 0.08% or 0.16%, see methods) and control Liver-Chips revealed significant differences in expression of genes related to FIG. 78A alcohol metabolism, FIG. 78B cholesterol metabolism, FIG. 78C glucose metabolism, FIG. 78D bile acid production and maintenance (i.e., cholestasis), e DNA damage, FIG. 78F cell cycle regulation, and FIG. 78G oxidative and metabolic stress. Adjusted p-values are demonstrated by bar color. Data from one experiment with 2-5 chips per condition.

FIG. 79A-D presents exemplary data demonstrating Ethanol and LPS-induced steatosis, oxidative stress, and cytokines release. Data was collected after 48h of exposure to gradually increasing concentrations of ethanol (0.08% and 0.16%) or ethanol+LPS.

FIG. 79A presents exemplary hepatic lipid accumulation: Representative images of AdipoRed staining in the Liver-Chip hepatocytes (above) and quantification of lipid droplet counts and size (below).

FIG. 79B presents exemplary oxidative stress: Representative images of MitoSox staining in the Liver-Chip hepatocytes (above) and quantification of ROS events (below).

FIG. 79C presents exemplary assessment of nuclei count per field of view (left) and proportion of hepatocytes with multiple nuclei per cell (polyploidy) (right).

FIG. 79D presents exemplary Cytokine release: Release of IL-1 beta, IL-6, and TNF-alpha (left to right) as measured by multiplexed immunoassays. Data from 2 (ethanol) or 1 (high-fat) independent experiments, minimally n=2 chips per condition, 5-8 images per chip where applicable. Data represent median±(min and max). *p<0.05; **p<0.01, ****p<0.0001 versus control (Kruskal-Wallis and Dunnett's multiple comparisons test).

FIG. 80A-B presents exemplary data demonstrating LPS-induced inflammation and cholestasis. Data was collected after 48h of exposure to gradually increasing concentrations of 0.08% ethanol or 0.08% ethanol+LPS.

FIG. 80A Representative images showing changes in MRP-2 bile canaliculi staining (green) in the Liver-Chip in response to ethanol alone or ethanol+LPS.

FIG. 80B Quantification of the percent of bile canaliculi radius, branching density, and porosity, as well as cholestatic area in the Liver-Chip. Data represent median±(min and max). *p<0.05; **p<0.01, ****p<0.0001 versus control (Kruskal-Wallis and Dunnett's multiple comparisons test).

FIG. 81A-B presents exemplary data demonstrating recovery of ethanol and ethanol+LPS induced phenotypes. 5 days post treatment stop.

FIG. 81A Quantification of polyploidy and FIG. 81B oxidative stress events in hepatocytes after 48h of exposure of the Liver-Chip to either ethanol or ethanol+LPS followed by 5 days of recovery. Data from one experiment, n=3 chips each. Data represent median±(min and max). *p<0.05; **p<0.01, ****p<0.0001 versus control (Kruskal-Wallis and Dunnett's multiple comparisons test).

FIG. 82A-E presents exemplary data demonstrating optimization of bile canaliculi (BC) network integrity.

FIG. 82A Schematic of a proposed relationship between the ECM and bile canaliculi (BC) integrity based on published work ref²⁷. Healthy BC above, Cholestatic, below. See. Table 8.

FIG. 82B Representative immunofluorescently stained images showing vastly different BC networks (green) of the tri-culture Liver-Chip under different ECM conditions.

FIG. 82C exemplary schematic diagram showing quantitative metrics (equations) of bile canaliculi network integrity. BC network integrity is improved when the average radius is narrowly distributed and branching density and porosity increase.

FIG. 82D Quantification of BC network integrity using metrics: Effects of different ECM conditions on the radius, branching density, and area fraction of BC networks. Data are from one experiment with n=3 chips per condition. Data represent median±(min and max), *p<0.05; **p<0.01, ****p<0.0001 versus control (Kruskal-Wallis and Dunnett's multiple comparisons test).

FIG. 82E Planar versus luminal ECM geometry: Cross-sectional image and thickness measurements comparing the planar ECM scaffold (here: ECM-B) created by standard membrane coating, and the luminal ECM scaffold (ECM-E) created by viscous fingering. Data are from one experiment with n=3 chips per condition. Data represent mean±(SEM).

FIG. 83A-B presents exemplary data demonstrating assessment of metabolic changes in one embodiment of an ALD/ASH Liver-Chip. Data was collected after 48h of exposure to gradually increasing concentrations of 0.08% or 0.16% ethanol. Fluorometric assessment using ELISA of FIG. 83A cholesterol levels in cell lysate and FIG. 83B glucose release in effluent.

FIG. 84A-C presents exemplary data demonstrating effect of donor variability on cellular responses to ethanol. FIG. 84A-B Quantification of cholesterol levels in Liver-Chips generated from different donors FIG. 84A-B after 48 h of ethanol exposure and FIG. 84C normalized to show fold-change in cholesterol in response to increasing concentrations of ethanol. Data represent median±(min and max). *p<0.05; **p<0.01; ***p<0.001; ****p<0.0001 versus control (Kruskal-Wallis and Dunnett's multiple comparisons test).

FIG. 85A-B presents exemplary data demonstrating differential Gene Expression analysis in ethanol-treated vs. control Liver-Chip hepatocytes. (Liver-Chips were treated for 48h at ethanol concentrations of either 0.08% or 0.16%, see methods herein).

FIG. 85A volcano plot illustrates the number of differentially expressed (DE) genes and how they stratify based on magnitude of change in ethanol exposed and control Liver-Chips. Red dots: genes that are significantly up- or down-regulated (adj. p-value<0.05 and |log₂ FoldChange|>1); black dots: non-DE genes.

FIG. 85B In total, 123 are differentially expressed, 87 are unregulated (red in heatmap) and 36 are downregulated (blue in heatmap) in the ethanol-exposed Liver-chips (0.08% and 0.16%). See, Table 9.

FIG. 86A-B presents exemplary data demonstrating LPS-induced ROS events, lipid accumulation and cholesterol release

FIG. 86A Cholesterol release in response to ethanol alone or ethanol+LPS.

FIG. 86B oxidative stress (ROS positive events) in response to ethanol alone, LPS alone, or ethanol+LPS. Data represent median±(min and max). *p<0.05; **p<0.01; versus control (Kruskal-Wallis and Dunnett's multiple comparisons test). FIG. 86C Representative bright field images of ethanol and ethanol+LPS treated hepatocytes in the Liver-Chips.

FIG. 87A-E presents exemplary data demonstrating Quad Liver-Chip responses to ethanol and ethanol+LPS treatment.

FIG. 87A Schematic illustrating the cellular organization in the quad-culture version of the Liver-Chip containing hepatic stellate cells (HSCs) in the 3D ECM scaffold.

FIG. 87B On-plate test of 3D ECM scaffolds (type ECM-D) for HSC culture. Immunofluorescent staining of α-smooth muscle actin (αSMA) reveals activation of HSCs in standard 2D culture (top row) whereas HSCs in 3D matrix remain quiescent (bottom row). FIG. 87C Immunofluorescent staining of vimentin and αSMA shows a quiescent (αSMA negative) HSC phenotype in the untreated Quad Liver-Chip.

FIG. 87D Representative images of AdipoRed staining showing lipid droplet accumulation (left) and quantification of lipid droplet count per cell and size (right) in the Quad Liver-Chip in response to either ethanol alone or ethanol+LPS.

FIG. 87E Fold-change of lipid droplet count per cell and size (compared to median value of untreated chips) in Quad Liver-Chips and Tri Liver-Chips. Data are from one experiment with n=2-3 chips per condition. Data represent average±(min and max). *p<0.05; **p<0.01; ***p<0.001; ****p<0.0001 versus control (ANOVA and Sidak's test).

FIG. 88A-D presents a representative illustration of a colon intestinal cells on a gel layer.

FIG. 88A: A colonic epithelial cell layer adhered to a membrane top surface.

FIG. 88B: A colonic epithelial cell layer adhered to a membrane top surface and a colonic fibroblast cell layer adhered to a membrane bottom surface.

FIG. 88C: A colonic epithelial cell layer adhered to a thin (partial) gel layer on a membrane top surface and an intestinal endothelial layer adhered to a membrane bottom surface.

FIG. 88D: A colonic epithelia cell layer adhered to a thin (partial) gel layer comprising colonic fibroblasts on a membrane top surface and intestinal endothelial cells adhered to a membrane bottom surface.

FIG. 89 present representative photomicrographs showing data that colonic fibroblasts migrate across the membrane from the endothelial layer to the epithelial layer after eight (8) days of culture.

FIG. 90 presents exemplary data showing intestinal barrier formation after four (4) days of culture between epithelial/endothelial cells or epithelial-fibroblast/endothelial cells as measured by apparent permeability coefficients (Pₐₚₚ).

FIG. 91 A-B presents exemplary data showing a lumenized thick gel layer overlay of glomerular endothelial cells in a bottom microchannel.

FIG. 91A: A longitudinal section view of the lumenized thick gel layer overlay.

FIG. 91B: A cross-sectional view of the lumenized thick gel layer overlay.

FIG. 92A-B presents a representation of glomerular type cell interactions that create a functional in vitro glomerulus.

FIG. 92A: Illustrative representation of the glomerular cell types and their interactions.

FIG. 92B: A representation of glomerulus endothelial cells, mesangial cells and podocyte cells within the bottom microchannel. Illustrative representation of the placement of glomerular cell types within a bottom microchannel either adhered to a membrane bottom surface or embedded within the thick gel layer overlay. Section 1: Positioning of mesangial cells to create a mesangial matrix. Section 2: Juxtapositions of mesangial cells and podocytes to create a basement membrane. Section 3: Juxtapositions of mesangial cells and endothelial cells to form a basement membrane. Section 4: Juxtapositions of podocyte cells and endothelial cells to create a slit diaphragm.

FIG. 93A-H present exemplary data collected from a microfluidic device have a channel of layers mucociliary bronchiolar epithelial cells (e.g., an Airway chip).

FIG. 93A: Schematic diagram of an Airway chip with a 3 μm pore PET membrane.

FIG. 93B: A differentiated airway epithelium exhibits continuous tight junctional connections on-chip, as demonstrated by Zo-1 staining (red). Scale bar, 20 μm.

FIGS. 93C-D: A well-differentiated human airway epithelium demonstrating goblet cells stained with MUC5AC (green) that demonstrates an extensive coverage of ciliated cells labelled with β-tubulin (green). Scale bar, 20 μm.

FIG. 93E: A lung blood microvascular endothelial monolayer formed on-chip contained continuous adherens junctions between adjacent cells, as indicated by VE-Cadherin staining (green). Scale bar, 10 μm. All images are representative of two to five independent experiments performed with cells from three different donors.

FIG. 93F: Graphic representation of cilia beating frequency (CBF) on-chip generated from a high speed recording of cilia beating in a representative field of view. Scale shows color coded CBF values in Hz. Scale bar, 20 μm.

FIG. 93G: The graph shows the cilia beating frequency measured in 5-10 random on-chip fields of view and each dot represents regions of ciliary beating. Data have been recorded in cells from three different donors and are presented as mean±SD.

FIG. 93H: Composite real time micrograph of a 5 sec recording of fluorescent microbeads path lines at the surface of the epithelium revealing the mucociliary activity of the differentiated hAECs. Scale bar, 20 μm.

FIG. 94A-D presents exemplary data of ciliary beating modeling on an Airway-Chip.

FIG. 94A: Immunofluorescence confocal views of differentiated airway epithelium cultured at air-liquid interface for 3 weeks on-chip in the absence (left) or presence (right) of IL-13 (100 ng/mL) for 7 days showing epithelium stained for the goblet cell marker MUC5AC (green) and nuclei (blue). Scale bar, 20 (applies to both views); images are representative of two independent experiments performed on cells from three different donors.

FIG. 94B: Total culture area covered by goblet cells in absence or presence of IL-13 (100 ng/mL) for 7 days.

FIG. 94C: Heat map of cilia beating frequency (CBF) on-chip generated from a high speed recording of cilia beating in a representative field of view in absence or presence of IL-13 (100 ng/mL). Scale shows color coded CBF values in Hz. Scale bar, 20 μm.

FIG. 94D: Graph showing Cilia beating frequency in absence or presence of IL-13 (100 ng/mL). Data represent mean±SEM and significance was determined by unpaired Student's; ***P<0.001, ****P<0.0001.

FIG. 95A-C present exemplary data showing a therapeutic modulation of IL-13 induced neutrophil recruitment and infiltration with a CXCR2 antagonist on an Airway chip.

FIG. 95A: Fluorescence imaging tracking neutrophil velocity at the endothelial surface in HRV-16 infected chips, in absence (left) or presence (right) of the CXCR2 antagonist. Scale shows color coded velocity values in μm/sec; Scale bar, 50 μm.

FIG. 95B: Quantification of neutrophil velocity in HRV16 infected chips with or without MK-7123 (10 μM).

FIG. 95C: Schematic diagram of neutrophil diapedesis.

FIG. 96 illustrates a multiscale profiling of mucociliated airway epithelia. Optical recordings at multiple scales in the small airway microphysiological system are analyzed to extract mechanical biomarkers of mucociliary transport. Biomarker profiles summarize the (*=statistically-significant) differences of test and control conditions, which can be used for disease modeling, diagnosis, and drug screening.

FIG. 97 presents an illustration of one work flow alternative for acquisition and analysis of mechanical biomarkers such as ciliary biomarkers. The work flow includes an integrated platform that enables real-time measurements of quantitative biomarkers of mucociliary transport in a small airway microphysiological system.

FIG. 98A-C presents exemplary data showing epithelial remodeling processes in a model of asthma.

FIG. 98A: IL-13 decreases CBF.

FIG. 98B: IL-13 decreases tissue-wide ciliary beat polarity.

FIG. 98C: IL-13 reduces uniform mucus flow straightness.

FIG. 99 presents exemplary data showing an optimized data acquisition of cilia motion. Left Panel: Motion detection acquired with standard filters and illumination leaves ciliated cells blurry. Right Panel: Clear trajectories of ciliary motion when optics are optimized.

FIG. 100A-D presents different methods for structural analysis and motion tracking.

FIG. 100A: CBF measurements through Fourier Transform,

FIG. 100B: ciliated cell density measurements through motion tracking,

FIG. 100C: ciliated cell planar beat polarity measurements through wavelet analysis

FIG. 100D: Vertical mucus flow velocity measurements through particle tracer tracking

FIG. 101 presents exemplary microphotographs of differentiated cells non-infected (left) or 6 days post rhinovirus infection (right). Cilia appear in green while nuclei were counterstained with DAPI (blue) (original magnification, 63×). The graph on the right display the cilia beating frequency recorded in non-infected and infected cultures.

FIG. 102 resents exemplary data showing the recapitulation of an impaired muco-ciliary function of CF tissues in vitro. Microphotographs of differentiated cells derived from healthy (left) and CF (right) donors. Fluorescent microbeads have been introduced in the apical channel of the Airway-Chip and imaged using a fluorescent microscope and high speed camera. Two seconds exposure.

FIGS. 103A-B presented exemplary data of cilia beating frequency with and without viral infection.

FIG. 103A shows beads moving across cilia-mucociliary elevator.

FIG. 103B shows quantification of cilia beat frequency between non-infected and HRV-16 infected chips. Cilia beating frequency (Hertz: Hz) comparing frequency measured in non-infected to infected chips.

FIG. 104A and FIG. 104E present exemplary photomicrographs of cilia beating frequency and immunostained Z-stacks.

FIG. 105A-B presents exemplary data of cilia beating before and after IL-13

FIG. 105A shows a panel of micrographs: FIG. 105(1) presents a CBF (cilia beating frequency) (HZ) colorized scale demonstrating cilia beating.

FIG. 105A(2) presents a colorized cilia beating frequency micrograph using a CBF scale shown in FIG. 105A(3).

FIG. 105A(4) shows a still shot from a video micrograph of mucociliary transport (i.e. mucociliary escalator) where the white dots are fluorescent microbeads moving across the upper surface of the epithelium.

FIG. 105B(1) and FIG. 105B(2) show micrographs demonstrating cilia beating frequency in colorized micrographs a CBF scale shown in FIG. 105B(3).

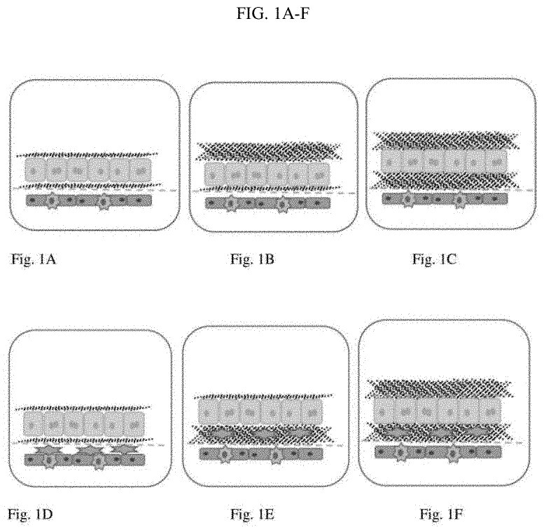

### DETAILED DESCRIPTION OF THE INVENTION

The present invention relates to the use of gels for cell cultures, including but not limited to microfluidic devices and transwell devices, for culturing cells, such as organ cells, e.g. airway cells, intestinal cells, etc., and co-culturing cells, (e.g. parenchymal cells and endothelial cells, etc). As one example, the use of gels results in improved lung cell cultures, such as when using transwells and microfluidic devices, (e.g. for culturing healthy airway epithelial cells, culturing diseased airway epithelial cells, e.g., CF epithelial cells that are ciliated). The present invention relates to fluidic devices, methods and systems for use with gel layers within a microfluidic device. In particular, a partial gel layer is disposed within a microchannel of a microfluidic device. For example, a partial gel layer has a thickness ranging between approximately 20-100 μm. A dilute partial gel layer of less than 100 μm may be formed from a polymer solution of 0.5 mg/ml. A cell-permeable partial gel layer having a thickness ranging between approximately 20-50 μm may be formed from a polymer solution of 1-3 mg/ml. A partial gel layer may be formed by a hydrodynamic shearing technique. Such thin-parital gel layers can support a variety of cell cultures, including but not limited to single cells, cell populations, cell layers, differentiated cell layers, and/or primary tissues. The present invention is related to the field of imaging and image processing. In particular, the invention is related to imaging that supports the determination of cell membrane cilia beating frequency. For example, methods described herein encompass cilia beat frequency in the context of membrane region and/or distances between regions. Alternatively, the methods described here encompass cilia beat synchrony and correlation of beat frequency between cell membrane regions.

In some embodiment, the present invention uses cells (e.g., parenchymal cells and/or vascular endothelial cells) cultured in the organ chips that can be isolated from a tissue or a fluid of subject using any methods known in the art, or differentiated from stems cells, e.g., embryonic stem cells, or iPSC cells, or directly differentiated from somatic cells. In some embodiments, stem cells can be cultured inside the organ chips and be induced to differentiate to organ-specific cells. Alternatively, the cells used in the organ chips can be obtained from commercial sources, e.g., Cellular Dynamics International, Axiogenesis, Gigacyte, Biopredic, InVitrogen, Lonza, Clonetics, C D I, and Millipore, etc.).

Non-limiting examples of cells contemplated for use in a microfluidic device for in vitro modeling of gastrointestinal tissue, include primary gastrointestinal epithelial cells, such as human biopsy derived gastrointestinal cells and cultures of primary gastrointestinal cells including but not limited to cells derived from: healthy intestinal tissue; inflammatory intestinal tissue; noninflammatory intestinal tissue, e.g. areas adjacent to inflammatory intestinal tissue; diseased intestinal tissue, e.g. Inflammatory bowel disease (IBD); Crohn's disease; colitis, ulcerative colitis (UC); etc.; diseased inflammatory intestinal tissue; noninflammatory intestinal tissue, e.g. areas adjacent to diseased inflammatory intestinal tissue; cancerous intestinal tissue; non-cancer intestinal tissue (e.g. tissue adjacent to cancerous tissue); cultures of expanded primary gastrointestinal epithelial cells; isolated intestinal crypts; cell populations enriched in a stem cell marker for intestinal stem cells, e.g. Leucine-rich repeat containing G protein-couple receptor (Lgr5+); epithelial cells derived from 3D intestinal enteroids; epithelial organoids; epithelial spheroids; stem cell enriched-spheroids; epithelial intestinal cells derived from induced pluripotent stem cells; fetal intestinal tissue; baby intestinal tissue; child intestinal tissue; adult intestinal tissue; intestinal tissue having known genetic mutations, intestinal tissue having unknown genetic mutations, and test intestinal tissue, etc. Biopsies may be obtained from any area of gastrointestinal tissue including but not limited to: mouth; esophagus; stomach, e.g. cardia, fundus, corpus, antrum, pylorus; small intestine tissue, e.g. duodenum, duodenojejunal flexure, jejunum, ileum, and terminal ileum; appendix; large intestine tissue, e.g. cecum, ascending colon, hepatic flexure, descending colon, sigmoid colon, rectum and anus; and areas around and including sphincters. Primary intestinal epithelial cells may also be obtained from commercial sources, e.g. Lonza Group Ltd.

In some embodiments, the cells used in the organ chips can be differentiated from the “established” cell lines that commonly exhibit poor differentiated properties (e.g., A549, CaCo2, HT29, etc.). These “established” cell lines can exhibit high levels of differentiation if presented with the relevant physical microenvironment (e.g., air-liquid interface and cyclic strain in lung, flow and cyclic strain in skin, lungs, etc.), e.g., in some embodiments of the organ chips.

In some embodiments, the cells used in the organ chips can be genetically engineered for various purposes, e.g., to express a fluorescent protein, or to modulate an expression of a gene, or to be sensitive to an external stimulus, e.g., light, pH, temperature and/or any combinations thereof.

### I. Extracellular Matrices in Microfluidic Cell Culture Chips.

Properties of extracellular matrices (ECMs) within, or associated with, a given tissue, cell layer, etc., in vivo can vary from one tissue to another (e.g. lungs versus skin versus bone) and even within the same tissue (e.g. renal cortex region versus renal medulla region). Differences in physiological states (e.g., normal cell versus cancerous cell) may also result in compositional ECM differences between the two cell types.

ECM is believed to be largely composed of several types of biopolymer fibers, such as collagen and fibrin, which are largely responsible for its mechanical properties through their strength and elasticity. Such an ECM fiber structure is believed to regulate cellular morphology, proliferation, migration, and gene expression. Many basic cell functions, including migration, proliferation, gene expression, and differentiation, may depend on how these mechanical forces deform and shape the surrounding extracellular matrix (ECM).

Fluidic devices used in cell culture typically have smaller numbers of cells within a smaller confined space, as opposed to conventional tissue culture plates. Thus, ECM used in fluidic devices may have an enhanced effect upon cell physiology and morphology than observed in plate cultures. Therefore, when providing a microfluidic device containing cells in contact with ECM for mimicking an organ, or a particular part of an organ, or a diseased state, the ECM should be specifically tailored in vitro for having matching properties for that in vivo part of the organ.

The temperatures and/or pH levels used in conventional ECM production methods may not align with optimum conditions for viability in cells seeded within, on top of, or underneath, gels. Consequently, these conventional methods may not achieve the necessary structural and/or mechanical properties of collagen gels (hydrogels), or other types of ECM gels to support cell growth in microfluidic cell cultures. For example, the lag time before polymerization begins and duration of polymerization are exemplary parameters considered for designing a gel for use with certain cells in a particular type of growth chamber, such as in channels of microfluidic chips. Further, ECM components and incubation protocols may need optimization by experimentation in order to maximize cell-viability.

Thus, it would also be useful to be able to manipulate ECM characteristics for matching in vivo ECM, including concentration and structure, found in disease states and in affected areas of tissues, such as for autoimmune diseases, cancer, etc.

Further, some ECM characteristics desirable for manipulation for use in microfluidic devices include gel strength and gel density, e.g. pore size and fiber density. Thus, in some embodiments, compositions and methods are provided for enhancing the strength of low-concentration (e.g., dilute) collagen gels used as ECM coatings, i.e. underlays, overlays, and for embedding cells in layers within microchannels of microfluidic devices.

Therefore, in some embodiments, compositions and methods are provided herein for providing gels whose polymerization characteristics provide temperatures, duration time for polymerization, structural properties or mechanical properties more conducive to optimal viability of cells. In some embodiments, gels are provided having polymerization and polymerized characteristics that are counterintuitive to those found with high concentration gels in microfluidic devices, i.e. contrary to common-sense expectations of characteristics of high concentration gels in microfluidic devices, but nevertheless true. Such counterintuitive gels, i.e. gels produced using enzymatic polymerization as described herein, in microfluidic devices provide polymerized gels having one or more characteristics of desired: strength, fiber diameter, structure, etc., for enhancing viability of cells coming in contact with the inventive gels in microfluidic devices as described herein.

A. Hydrogel ECMs.

Hydrogels generally refer to a cross-linked polymeric network comprising water or a biological fluid. Hydrogel mechanical properties in vitro provide stability for cells in contact with polymer networks in culture and may also influence cellular mechanotransduction (e.g, the conversion of mechanical information from the microenvironment into biochemical signaling), which in turn may regulate cellular behaviors like spreading, migration, and stem cell differentiation.

Numerous kinds of hydrogel/ECM materials may be used in vitro, including natural, synthetic and hybrid materials. Natural hydrogels may be made from ECM mixtures isolated from biopsies, or assembled using isolated natural materials such as Matrigel®, collagen, gelatin, fibronectin, hyaluronic acid, laminin, chitosan, and sodium alginate, etc. In addition, ECM-derived polymers like collagen, fibrin, HA, and laminin have ability to induce biological responses in cells. pubs.rsc.org/en/content/articlehtml/2012/cs/c1cs15203c-cit24. Further, other proteins such as gelatin, fibrin silk, and sugar beet pectin can easily form hydrogels owing to the intrinsic existence of sufficient tyrosine and phenylalanine, and feruloyl groups respectively. Synthetic materials may be used, such as polylactide, polylactide-co-glycolic acid copolymer, polyethylene glycol, polycaprolactone, and polyacrylamide. Hybrid hydrogel materials combine elements of synthetic and natural polymers. The source of hydrogel material influences hydrogel properties, including but not limited to, polymerization kinetics, hydrogel mechanics, and fiber structure. Further, the method by which collagen is extracted from tissue was shown to alter the molecular structure of the collagen fibrils as well as the kinetics of assembly.

Most collagen hydrogels are prepared using type I collagen, which comprises 90% of the protein in human connective tissues and is easily extracted from animal tissue with minimal contamination by other collagens or proteins. Type I collagen is a triple-helical protein formed of 67-nanometer (nm) periodic polypeptide chains with a total molecular weight around 300 kDa. Collagen fibrils self-assemble at neutral pH into bundled fibers typically 12-120-nm diameter that crosslink to produce a matrix structure that ultimately forms a hydrogel in the presence of a water-based solvent.

Collagen types II, III, and other constituents such as glycosaminoglycans may also be incorporated. Collagen sources for hydrogels include rat tail tendon, porcine skin, bovine skin, etc. Collagen is usually derived from solutions of acid or pepsin-solubilized type I collagen, often sourced from rat-tail tendon.

Gelatin is a common hydrogel material. Gelatin refers to a degraded product of collagen, having the same amino acid sequence as collagen but lacking the triple helical character.

Collagen-containing hydrogels, including Matrigel®, suffer from some drawbacks, shared by other natural materials, including low stiffness, where stiffness is the extent to which an object resists deformation in response to an applied force, limited long-term stability, and batch-to-batch variability. It is also difficult to produce collagen hydrogels with higher stiffnesses (>1 kPa) without extensive chemical crosslinking, which fundamentally alters the degradability of collagen fibrils. As a result, culturing cells in collagen hydrogels for long time periods results in significant contraction of the matrix, leading to a loss of cell viability.

Matrigel® in particular has drawbacks for use in microfluidic devices. Matrigel® refers to a basement membrane-derived preparation extracted from Engelbreth-Holm-Swarm (EHS) mouse sarcoma tumors that is primarily composed of laminin, type IV collagen, and entactin, with various other constituents including proteoglycans and growth factors. Drawbacks include Matrigel®'s tumorigenic origin, diverse composition, and batch-to-batch variability in terms of mechanical and biochemical properties, which in turn brings a level of uncertainty to cellular experiments.

Thus, it is desired to be able to produce and use hydrogels in microfluidic devices having specified physical properties, such as a certain concentration of polymers, mechanical strength, stiffness, stress relaxation, and degradation. Further, cell-scaffold interactions may induce a change in mechanical properties of the associated hydrogel. As one example, an increase in stiffness of blood vessels is associated with arterial disease.

B. Polymer Concentration Effects.

Control of collagen concentration of in vitro hydrogels may be accurately achieved using lyophilized collagen, as the reconstitution step permits a pre-determined and accurate weight measurement of dry collagen and volume measurement of solvent to obtain the stock solution concentration. Whether extracted in-house or obtained commercially, stock concentrations are most commonly in the range 1-5 mg/mL, lower than the collagen content of many native tissues.

This limitation has implications for the utility of collagen hydrogels as tissue mimics. Collagen concentration in vitro and in vivo influences tissue mechanical properties, thereby regulating cellular behavior. Collagen concentration plays a role in tumor malignancy, as cancerous tissue contains 9-45 mg/mL (0.9-4.5% wt.) collagen in the interstitium while normal tissue contains significantly less.

Although most tissue mimics were fabricated using hydrogels with low collagen content (<4 mg/mL), they have limited value for mimicking 3D tissues due to their nonphysiological strength and microstructure, which results in an inability to support microfabrication as bulk gels. Additionally, most commercially available formulations are provided at low concentrations or for providing low concentrated gels.

Along with collagen content, temperature of polymerization significantly affects hydrogel properties. Because reaction kinetics are temperature dependent, self-assembly of collagen molecules occurs more rapidly at higher temperature and results in fibers with a lower number of bundled fibrils, resulting in a less-ordered structure and consequently altered mechanical, structural, and transport properties.

C. Polymerization and Cross-Linking Effects.

The kinetic process of fiber polymerization into a hydrogel network is a multistep process including, but not limited to, fibril formation, fiber nucleation and development, and crosslinking. Fabrication parameters, such as the type of collagen, collagen source, extraction method and type of processing affects the kinetic polymerization of the in vivo gels. For example, pepsin-digested collagen produces fiber networks with long fibers and large pores as compared with undigested collagen such as that extracted from rat-tail tendon. As another example, acid-solubilized collagen polymerize are known to more rapidly than pepsin-digested collagens.

Mechanical properties of hydrogels may be defined by a filament network architecture and individual filament properties. To increase the filament properties, such as matrix stiffness or strain-stiffening, these filament networks can be modified by adding crosslinking agents that alter the network architecture, e.g. by increasing the unions between filaments.

Matrix stiffness may be increased by increasing one or both of the concentration of collagen deposition and increasing cross-linking of the polymers, e.g. adding or increasing the amount of an external crosslinking agent, i.e. added to a gel solution. Increasing cross-linking alone can result in increased matrix stiffness without changing an ECM architecture. As one example, areas around tumors may have higher ECM stiffness, thus decreasing cell migration through stiffer ECM. The addition of external crosslinking agents also affects fibrillogenesis. Fibrillogenesis refers to a development of fine fibrils normally present in collagen fibers of connective tissue.

Along with collagen source, content, concentration, type of induced crosslink, i.e. chemical or enzyme used to initiate crosslinking, the temperature of polymerization significantly affects hydrogel properties. This characteristic also impacts the viability, morphology and physiology of contacted cells during the polymerization stage. For the use of such preparations with cells, polymerization times may be kept short and used with non-toxic photopolymerization initiators (e.g. I2959 or lithium acylphosphinate salts) which are believed to minimize cell death and maintain overall cellular function. Because polymerization kinetics are temperature dependent, self-assembly of collagen molecules occurs more rapidly at higher temperature and results in fibers with a lower number of bundled fibrils, resulting in a less-ordered structure and consequently altered mechanical, structural, and transport properties. While previous studies used hydrogels polymerized at 37° C. to facilitate cell seeding and viability, polymerization at room temperature (20-26° C.) or below, down to 4° C., is not uncommon but generally inhibits inclusion of cells within the hydrogel.

Yang et al. and Raub et al. varied polymerization temperature between 4° C. and 37° C. to control fiber structure without necessitating variation in concentration and noted that polymerization at low temperatures produced the most desirable pore size for cellular proliferation. Similarly, Chrobak et al. found that gels formed at room temperature can be used to create microchannels with better stability and less degradation over time than those formed at 37° C.

Polymerization temperature is difficult to control accurately, as temperature-dependent fiber self-assembly initiates as soon as the solution is neutralized. Most groups work with solutions on ice or in an ice chamber to slow polymerization until the hydrogel is well mixed, at which time it is moved to a chamber at the appropriate polymerization temperature. Therefore, the exact temperature of the hydrogel during the initial stages of polymerization is heterogeneous and regulated by convection and conduction. These effects cannot be strictly controlled in practice; however, if fabrication procedures are consistent, mixing is performed rapidly, and the polymerizing solution is moved immediately from ice to a temperature-controlled chamber, then the properties of the final hydrogel are typically reproducible.

Hydrogels may be divided into at least two categories based on the physical nature or chemical nature of the cross-link junctions. Physical networks have transient junctions that arise from self-assembly, either polymer chain entanglements or physical interactions, such as ionic interactions, hydrogen bonds, or hydrophobic interactions. Physical crosslinks, created by chain entanglement or attraction, are usually weaker than chemical crosslinks. As one example, for conventional physical crosslinking of gelatin, an aqueous solution of several percent gelatin turns to a transparent elastic hydrogel upon cooling below 35° C. Crosslinking occurs via random coil gelatin molecules turning to the ordered triple helix conformation of collagen. However, the thermoreversibility of the hydrogel may results in melting at physiological temperature. Other physical crosslinking methods, such as plasma treatment, often result in low crosslinking extent of gelatin macromolecules. Synthetic polymers may be modified with various functional groups to enable physical crosslinking.

Photopolymerization using light induced cross-linking is widely used for hydrogel fabrications due to its biocompatibility, spatiotemporal controllability and covalent attachments between polymers. In some cases, polymer macromers are modified with photoreactive moieties, such as methacrylate or acrylate groups. A photoinitiator is then added to a photoreactive macromer solutions that are then crosslinked under UV or visible light. The photoinitiators generate free radicals that are transferred to the photoreactive carbon double bond groups in the modified macromers to start chain polymerization. Under high photo exposure, a photoinitiator will generate a large number of free radicals that may react with intracellular molecules to induce cell damage.

Chemical and enzymatic cross-linked networks have more permanent junctions. In fact, chemical and enzymatic induced crosslinked hydrogels in general have better performance and stability than physically crosslinked hydrogels due to stronger binding energy and substantially improved flexibility of the gel. In both examples of chemical crosslinking, gelation needs to occur fast enough to prevent the settling of cells during the encapsulation process.

Chemical (covalent) crosslinking of gelatin refers to the use of chemical reagents, such as glutaraldehyde, formaldehyde, 1-(3-dimethylaminopropyl)-3-ethyl-carbodimide hydrochloride, and genipin, as a chemical crosslinker (Tseng et al., 2013). Chemically crosslinked hydrogels comprising covalent bonds between the polymer chains are formed via radical polymerization and non-enzymatic or enzymatic cross-linking of complementary groups. However, the toxicity of chemical crosslinking agents and the gelling process limits cellular applications, in particular added to fluidic chips comprising live cells. For one example, small molecular chemical crosslinkers, e.g. glutaraldehyde and carbodiimides, are toxic and are not recommended to fabricate cell-laden hydrogels.

Therefore, the use of enzymatic crosslinkers is described herein as a preferred embodiment for providing polymerized gels in microfluidic devices. Some advantages of enzymatic hydrogelation include but is not limited to avoiding the production of undesirable by-products or cytotoxic compounds because enzymes work with nontoxic and specific substrate polymers.

In preferred embodiments, enzymatic crosslinking provides biomaterial crosslinking, for use in microfluidic devices, such as those described herein. The use of enzymatic cross-linked hydrogels enables choosing desired hydrogel properties tailored for different types of Organ-chips, biomedical purposes, etc. Enzyme-enabled crosslinking can be highly selective for a particular enzyme with polymerization achieved under mild physiological conditions.

Transglutaminase, horseradish peroxidase, and tyrosinase, are examples of crosslinkers for numerous kinds of proteins, including gelatin and collagen (Yung et al., 2007; Spurlin et al., 2009; Kuwahara et al., 2010; Taddei et al., 2013). Transglutaminase refers to an enzyme that catalyzes the crosslinking of proteins by creating covalent amide bonds between glutamine and lysine. Tissue transglutaminase (TG2) and microbial transglutaminase, where BTG refers to bacterial transglutaminase, refers to a calcium-dependent enzyme (EC 2.3.2.13) of the transglutaminase family, found in vivo within mammalian cells and produced endogenousely by microbes, respectively. Tyrosinase refers to an oxidase (EC 1.14.18.1) that may react spontaneously mainly via 1,4-additions with the side chains of lysine, tyrosine, histidine, and cysteine residues, depending on their abundance and accessibility on the target protein, to form covalent protein—protein crosslinks, i.e. junctions. Additional enzymes used for crosslinking include, Laccase (EC 1.10.3.2), and Lysyl oxidase (EC 1.4.3.13)/amine oxidase (EC 1.4.3.6) which crosslinks collagen and elastin. Horseradish peroxidase (HRP) can build networks between polymers by reactions of polymer (e.g. aniline derivatives) with phenol conjugates in the presence of hydrogen peroxide (H₂O₂). In part, by oxidative coupling of hydroxyphenylpropionic acid moieties lysyl oxidase (LOX).

Moreover, there is a wider range of enzymes that can be used for hydrogelation mechanisms including sortase, phosphopantetheinyl transferase, plasma amine oxidase, phosphatases, thermolysin, β-lactamase, and phosphatase/kinase, α-chymotrypsin, and other peroxidases.

Among these enzymes, horseradish peroxidase (HRP) may have biomedical applications due to fast gelation, controllable crosslinking density, and gelation rate. Besides, HRP has moderate substrate specificity and can easily be a tuned hydrogel network by adjusting precursor reactants. HRP-catalyzed reaction is performed with a broad array of polymers due to moderate substrate specificity of HRP. Natural-based polymers such as chitosan, hyaluronic acid (HA), alginate, collagen, silk fibrils, fibrin, gelatin, dextran, heparin, chondroitin sulfate, and their combination-formed hydrogel through HRP-catalyzed polymerization were cytocompatible and biodegradable.

HRP acts as anaerobic oxidase by means of a ferric-ferrous cycle of valency changes of peroxidase iron. The majority of HRP reactions use hydrogen peroxide (H2O2) as a reduction substrate. The formed complex oxidizes Ph moieties of rich polymers that results in gelation of polymers. After successive oxidation reactions, HRP returns to its original state and then can re-enter the crosslinking cycle. This enzyme catalyzes crosslinking of polymers in rich phenols (Ph), phenylamines, indoles, sulfonates, and other similar analogues to form hydrogel networks. Despite the cytocompatibility of HRP substrates involving in vivo applications, a major concern is a potential immunogenicity as HRP is not a mammalian derived peroxidase.

In contrast, transglutaminases are biocompatible crosslinking enzymes, which are naturally found on/in living organisms. Enzymatic activity of a transglutaminase was observed in many microorganisms and in plant and animal tissues. In fact, transglutaminases are enzymes that are commonly found inside and outside of a cell. Their location determines the versatility and diversity of their functions. Further, the presence of different transglutaminase forms is observed in one organism (Luciano and Arntfield 2012). Animal and plant transglutaminases manifest catalytic activity and biochemical properties similar to those of microbiological transglutaminases, despite having a lack of homology in the amino acid composition (Luciano and Arntfield 2012).

Transglutaminase is an enzyme system involved in protein-based hydrogel crosslinking used for tissue engineering approaches, because it can offer intimate integration between the in situ formed hydrogel and the native host tissue (Teixeira et al., 2012). Moreover, the hydrogel catalyzed by transglutaminase is mechanically stronger and more stable than that catalyzed by tyrosinase (Chen et al., 2003). However, owing to the relatively high price of these enzymes compared with chemical crosslinkers, the enzymatic crosslinking method for gelatin had been rarely used until microbial transglutaminase (mTG) was discovered. mTG, which is derived from streptomycetes bacteria, exhibits high specific activity over a wide range of temperature and pH and is Ca2+ independent. mTG is utilized in the food industry, enhancing the functional properties of protein-rich food through covalent crosslinking (Halloran et al., 2008; Wangtueai, Noomhorm & Regenstein, 2010).

The effect of tissue transglutaminase crosslinked collagen gels has been studied on different cell types such as bone marrow stromal cells, dermal fibroblasts and osteoblasts, finding that the cytotoxicity was under the detection limit. The relative tissue strength of gels crosslinked with different transglutaminase concentrations was measured by denaturation temperature, finding that crosslinking increases the collagen gel strength as opposed to other types of crosslinking agents whose use decreases gel strength. Transglutaminases may be calcium-independent, such as in microbial TGs, or require Ca²⁺ ions as a cofactor, such as for mammalian transglutaminases.

Some studies describe gelatin hydrogels crosslinked by mTG as a cell scaffold material (Paguirigan & Beebe, 2007; Yung et al., 2007; Kuwahara et al., 2010; Bode et al., 2011; De Colli et al., 2012; Bode et al., 2013; Da Silva et al., 2014). Thus, the use of transglutaminase for polymerizing gels for other applications is non-toxic and exerts no side-effects on several cell types cultured in other types of devices. However, its use in microfluidic devices and effects upon cells within these devices are not known.

The transfer of mTG genes to expression systems such as Escherichia coli has increased availability of mTG. Enzymes biosynthesised by bacteria are stable at a wide range of pH values, i.e. from 4.5 to 8.0. In addition, they do not require calcium ions to be activated, which is in contrast to transglutaminases of animal origin. This is a highly desirable property, from a practical point of view, for use in enzymatic preparation. The activity of transglutaminase increases in the presence of Co²⁺, Ba²⁺ and K⁺. Microbial transglutaminases are inhibited by Zn²⁺, Cu²⁺, Hg²⁺ and Pb²⁺ ions that bind to the thiol group of cysteine in the active centre (Macedo et al. 2010; Motoki and Seguro 1998; Ando et al. 1989).

Transglutaminases of microbiological origin have low molecular weight, as opposed to transglutaminases isolated from animal tissues. Microbiological transglutaminase is a single polypeptide with a molecular weight of approx. 38 kDa. It is composed of 331 amino acids, with an isoelectric point at pH 8.9 (Abd-Rabo et al. 2010; Ando et al. 1989). It is a simple monomeric protein (not a glycoprotein or lipoprotein) (Yokoyama et al. 2004).

Microbial transglutaminase (MTGase) (EC 2.3.2.13) catalyzes acyl transfer reactions, deamidation, and inter and intramolecular crosslinks between amino acid residues of glutamine and lysine. Transglutaminase catalyses the formation of isopeptide bonds between proteins. Its cross-linking property is widely used in various processes: to manufacture cheese and other dairy products, in meat processing, to produce edible films and to manufacture bakery products. Its application in foods alters the technological properties of food proteins, such as their emulsifying capacity, gelation, viscosity, water holding capacity, and foam formation and stability.

Transglutaminase has considerable potential to improve the firmness, viscosity, elasticity and water-binding capacity of food products.

Transglutaminase was isolated from Streptoverticillium sp. and Physarum polycephalum. As an extracellular enzyme, it is biosynthesised by Streptoverticillium sp. (Aidaroos et al. 2011), Streptoverticillium cinnamoneum subsp. cinnamoneum (Duran et al. 1998), Streptomyces netropsis (Yu et al. 2008), Streptoverticillium griseocarneum (Gerber et al. 1994), Streptoverticillium ladakanum (Ho et al. 2000) and Streptomyces lydicus (Færgemand and Qvist 1997).

The structure of a collagen hydrogel fiber may be imaged using scanning electron microscopy (SEM) and transmission electron microscopy for qualitative/semiquantitative analysis of network and fibril structures, respectively. Optical imaging techniques may also be used for fiber structure measurement of hydrated hydrogels, two-photon fluorescence (TPF) and second-harmonic generation (SHG), as well as confocal reflectance microscopy (CRM) and confocal fluorescence microscopy.

Fiber structure in collagen gels is complex and often defined quantitatively in terms of parameters, such as mean fiber diameter D, fiber density (or volume fraction), pore size P, degree of crosslinking (or number of crosslinks per fiber), and orientation Ø. These parameters can be used to help elucidate the cellular characteristics described previously; for example, fiber orientation describes anisotropy of the material and can therefore be a predictor of directionality of cell migration.

For use herein, a Sirius red colorimetric assay may be used to measure the quantity of soluble collagen

### II. Ciliated Biomimetic Cell Culture Models.

A biomimetically engineered small airway epithelium can be a powerful tool for studying ciliated airway physiology in vitro including, but not limited to, real-time measurements of mucus-cilia mechanics at multiple scales, i.e., at the level of cilia, ciliated cells, ciliated tissue, and mucus flow. Previous reports describe automated, randomized measurements of ciliary beat frequency to acquire this established biomarker in an unbiased manner. Benam et al., “Matched-comparative modeling of normal and COPD human airway responses to inhaled smoke in vitro” Cell Syst. (2016).

In some embodiments, the present invention contemplates an integrated optical acquisition and software analysis suite for measuring a panel of structural and functional ciliary biomarkers. In one embodiment, this data acquisition and analysis suite is capable of measuring mucociliary clearance in living tissue. In one embodiment, the data acquisition and analysis suite produces a biomarker panel comprising cilia parameters that span multiple spatial scales and include biomechanical metrics including, but not limited to, ciliary beat frequency and mucus flow rate. In one embodiment, the biomarker panel also includes biomechanical metrics including, but not limited to, ciliary beat angle, planar beat polarity, and vertical mucus flow velocity. For example, an optical imaging setup and video processing algorithms extract several quantitative biomarkers of ciliary beats. In one embodiment, such setup and processing algorithms are configured for both microfluidic and transwell formats of a microphysiological system. In one embodiment, the optical imaging and video processing provide qualitative and quantitative information regarding biomarkers including, but not limited to, ciliary beat frequency, tissue-wide polarity, mucus flow velocity, and ciliated cell density. These data may be processed in real time or saved to storage media for later analysis. In one embodiment, the present invention contemplates an algorithm for extracting cilia metrics on a single cell level. In one embodiment, the single cell cilia metrics include, but are not limited to, beat coordination, beat polarity, and beat angle. Alternatively, the algorithms provide for the visualization and analysis of horizontal and vertical mucus flow and circulation.

In one embodiment, the present invention contemplates a method of using biomarker panel for profiling and/or categorizing healthy and defective mucociliary clearance. In one embodiment, the method profiles a cell culture mimicking at least one respiratory disease including cystic fibrosis, viral infection and/or asthma. In one embodiment, ciliary biomarkers are used for profiling mucociliary transport in respirator tissue models including, but not limited to, small airway diseases, viral infection, and asthma. Although it is not necessary to understand the mechanism of an invention, it is believed that by measuring a panel of ciliary biomarkers it is possible to observe disease effects on mucociliary clearance, detect previously unknown effects, and establish biomarker profiles that are unique to each airway disease.

### III. Cellular Membrane Cilia Beating Dynamics.

In one embodiment, the present invention contemplates methods of measuring cell membrane cilia beating frequencies. In one embodiment, the measuring is performed by an optical flow analysis. In one embodiment, the optical flow analysis comprises detecting a plurality of beads that are cleared from a specific cell membrane area. In one embodiment, the optical flow analysis is a video image analysis.

In some embodiment, the present invention extracts measurements of ciliary biomarkers. Although it is not necessary to understand the mechanism of an invention it is believed that when cells are cultured in a microphysiological device as described herein, the in vitro ciliary biomarkers are equivalent in functional capacity as compared to in vivo. See, Table 1.

TABLE 1
Comparison Of In Vivo Ciliary Biomarkers To A Microfluidic
Airway-On-Chip.

| | | In microfluidic |
|---|---|---|
| Biomarkers | In Humans (in vivo) | Airway-on-Chip (in vitro) |
| Cilia beating frequency | 9-20 Hz | 9-19 Hz |
| Cilia length | ~6 μm | ~6 μm |
| Axoneme structure | 9 + 2 microtubule | 9 + 2 microtubule |
| Mucociliary velocity | 40-150 μm/sec | 40-100 μm/sec |
| % of ciliated cells | ~30% | ~20-30% |
| % of goblet cells | ~10-15% | ~10-20% |
| % of basal cells | ~6-30% | ~20% |

~= approximately.

### A. Ciliary Beating Frequency Analysis.

Cilia beating frequency (CBF) was measured by applying Fourier spectral analysis to high-speed video recordings of the ciliated surfaces. Using an inverted phase contrast microscope, the ciliated surfaces were recorded at 190-200 frames per second and at a resolution of 512 by 512 pixels, which corresponded to a field of view of 166 by 166 μm² (0.32 μm per pixel). Cilia beating was recorded at multiple randomly chosen fields of view (FOV) for each chip.

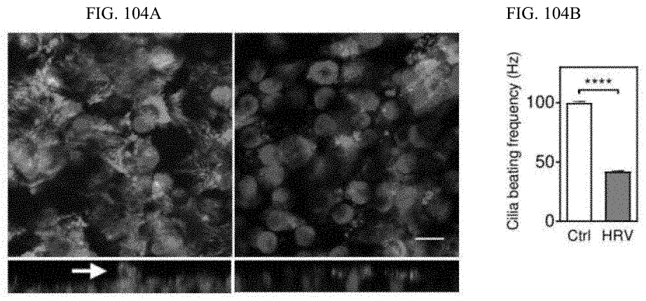

A first step of video processing was to identify regions of ciliary beating by computing a normalized standard deviation of an intensity value for each pixel across all frames. A standard deviation close to 1 corresponds to pronounced fluctuations in pixel brightness over time, indicating motion and hence ciliary beating. FIG. 104A-B.

In a further step, areas with ciliary motion were thresholded and the movie field of view was divided into 289 windows (ca. 10×10 μm² in size). For each window, a random set comprising 70% of all pixels with detected motion was further analyzed to detect the dominant beat frequency. For each sampled pixel, ciliary beat frequency was determined from a time-dependent fluctuation of pixel brightness reflecting the periodicities of the ciliary movement. A bandwidth filter (e.g., approximately 1 to 30 Hz) was applied to each pixel signal to remove noise. Further, a hamming window was applied to each pixel signal to reduce sampling artifacts. Subsequently, Fast Fourier Transform converted temporal pixel signals to pixel frequency domains thereby allowing computation of a pixel frequency power spectrum. Pixel signals that were identified without a dominant frequency component were discarded. For each window, all usable frequency power spectra were averaged across pixel signals to detect one dominant CBF per window. Thus, for each FOV, the number of measured CBFs was determined by the number of windows with usable signals (e.g., 289 windows).

B Cilia Beating Rate Variability Over a Specific Cell Membrane Area.

In one embodiment, the present invention contemplates a method to identify cilia rate variability within a specific membrane area or membrane space. In one embodiment, the present invention contemplates a method to identify cilia rate variability along a specific distance of a cell membrane. In one embodiment, cilia beating rate variability is displayed using heat maps. In one embodiment, the heat maps are colored heat maps. In one embodiment, cilia beating rate variability is displayed using scatter plots.

C. Cilia Beating Synchronization within a Specific Cell Membrane Area.

In one embodiment, the present invention contemplates a method to identify cilia synchronization within a specific membrane area or space. In one embodiment, the present invention contemplates a method to identify cilia synchronization along a specific distance of a cell membrane.

D. Cilia Beating Correlation Between Specific Cell Membrane Areas.

In one embodiment, the present invention contemplates a method for correlating cilia rate variability between at least two different specific membrane areas or membrane spaces. In one embodiment, the present invention contemplates a method for correlating cilia rate variability between at least two different specific distances of a cell membrane.

In one embodiment, the present invention contemplates the calculation of a correlation between at least two different cilia regions based upon orientational order parameters. For example, an orientational order parameter has been validated using both synthetic and experimental data. These parameters can be useful for quantifying the correlation of orientation of constructs such as cilia beat frequency and/or cilia beat direction. It would be expected that normal ciliated tissues would show strong positive correlation between regions for either ciliary biomarker. A further use of these orientational parameters would be to quantify the consistency of construct orientation on ciliated cells of the same or different densities. In general, the orientation parameter provides a quantitative tool to characterize tissues beyond co-localization or single construct orientation distribution. Such parameters have recently been reported in the context of correlations between different parameters of cardiac contractility. Drew et al., Multiscale Characterization of Engineered Cardiac Tissue Architecture” J Biomech Eng 138(11), 111003 (Oct. 21, 2016) (8 pages).

E. Cilia Beating Uniformity.

In one embodiment, the present invention contemplates a method for measuring cell membrane cilia beating uniformity. In one embodiment, the uniformity is measured within a specific cell membrane area. In one embodiment, the uniformity is correlated between at least two different specific cell membrane areas.

F. Cilia Beating Temporal Profiles.

In one embodiment, the present invention contemplates a method for measuring a cell membrane cilia beating frequency temporal profile. In one embodiment, the cilia beating frequency temporal profile correlates with a temporal mucus secretion profile. In one embodiment, the temporal mucus secretion profile comprises a temporal signature.

### IV. Ex Vivo Methods of Determining Therapeutic Effects Upon Ciliated Cells.

In one embodiment, the present invention contemplates a microfluidic device comprising at least one channel, wherein the channel is layer with a ciliated biological cell. In one embodiment, the ciliated biological cell is an immune cell and undergoes transmigration. In one embodiment, the ciliated biological cell undergoes sloughing. In one embodiment, the ciliated biological cell undergoes altered cilia beating frequencies. In one embodiment, the ciliated biological cell is a goblet cell and undergoes hyperplasia. In one embodiment, the ciliated biological cell is microvascular endothelial cell and undergoes an increased expression of adhesion molecules. In one embodiment, the ciliated biological cell is a microvascular endothelial cell and undergoes release of an inflammatory mediator.

In one embodiment, the present invention contemplates a method comprising real time, high resolution imaging and quantitative analysis of ex vivo inflammatory cells. In one embodiment, the imaging determines an efficacy of a CXCR2 antagonist to reduce inflammatory cell adhesion. In one embodiment, the imaging determines an efficacy of a CXCR2 antagonist to reduce inflammatory cell motility. In one embodiment, the imaging determines an efficacy of a CXCR2 antagonist to reduce inflammatory cell transmigration. In one embodiment, the inflammatory cell is a neutrophil. In one embodiment, the neutrophil is a human neutrophil. Rationale: Viral-induced exacerbations of asthma are a major cause of hospitalization and mortality. Our understanding of the complex mechanisms underlying asthma exacerbations is hindered by the lack of physiological relevance and cellular complexity of traditional in vitro systems and the limited human translatability of animal models.

In one embodiment, the present invention contemplates an ex vivo model of ciliated epithelial cells that can be used to evaluate various therapies, including, but not limited to neutrophil transepithelial migration and immunomodulatory therapy. For example, an ex vivo model of ciliated epithelial cells may comprise a fully-differentiated human mucociliary airway epithelium stimulated with IL-13.

Generation of a Th2 (T helper cell type 2) microenvironment through exogenous IL-13 stimulation may result in several biological responses of ciliated epithelial cells including, but not limited to, goblet cell hyperplasia, reduction of cilia beating frequency and vascular wall activation. In one embodiment, these biological effects may be studied and evaluated using a high resolution temporal analysis of secreted inflammatory markers. Microfluidic devices comprising channels layered with respiratory tissue epithelial cells may be perfused with human neutrophils to evaluate the effects of treatments on neutrophil transepithelial migration and/or neutrophil diapedesis.

A. Exemplary Methods for Measuring Mucociliary Clearance.

Organs-on-chips offer the opportunity to generate complex, microphysiological functional units of human tissue that recapitulate multicellular architecture, tissue-tissue interfaces, physiochemical microenvironment and vascular perfusion of the body. Bhatia et al., “Microfluidic organs-on-chips”, Nat. Biotechnol. 32:760-772 (2014). In one embodiment, the present invention contemplates a biomimetic ciliated epithelial cell chip that recapitulates the structure and function of a well-differentiated ciliated tissue. For example, a human mucociliary airway epithelium.

1. Beta-Tubulin Staining of Ciliated Cells.

In one embodiment, the present invention measures cilia on mucus producing cells.

2. Unidirectional Mucociliary Transport.

In one embodiment, the present invention measures a unidirectional ciliary movement within a specific region of a cell membrane. In one embodiment, the present invention measure a unidirectional ciliary movement along a specific distance of a cell membrane. In one embodiment, the present invention correlates unidirectional ciliary movement between at least two different specific regions of a cell membrane. In one embodiment, the present invention correlates unidirectional ciliary movement between at least two different distances along a cell membrane.

3. Ciliated Epithelial Cell Remodeling.

In one embodiment, the present invention contemplates a method for detecting a remodeled ciliated epithelial cell characterized by a two-fold increase in area covered by MUCSAC positive cells.

4. Effect of IFN-β on Cilia Beating Frequency.

In one embodiment, the present invention contemplates a method for contacting a ciliated cell with IFN-β to modulate ciliary beating frequency. In one embodiment, ciliary beating frequency variability is modulated. In one embodiment, ciliary beating frequency uniformity is modulated. In one embodiment, ciliary beating frequency correlations are modulated.

5. Effect of IL-13 on Cilia Beating Frequency.

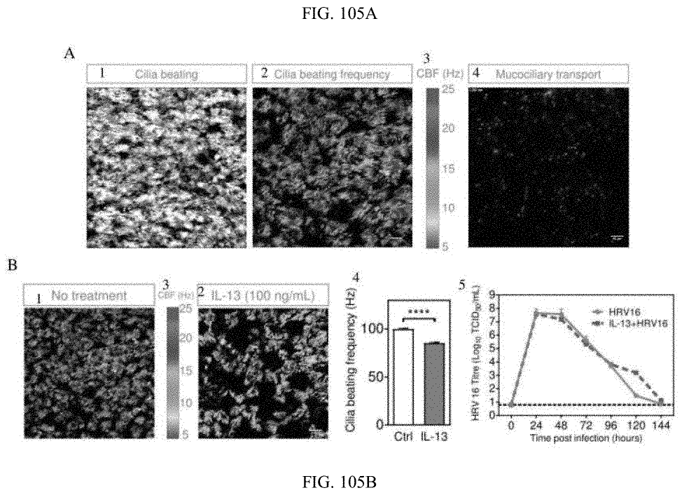

In one embodiment, the present invention contemplates a method for contacting a ciliated cell with IL-13 for approximately 6-8 hours to modulate ciliary beating frequency. In one embodiment, ciliary beating frequency variability is modulated. In one embodiment, ciliary beating frequency uniformity is modulated. In one embodiment, ciliary beating frequency correlations are modulated. See, FIG. 105A-B.

6. Transendothelial Migration of Ciliated Cells.

In one embodiment, the present invention contemplates a method for measuring the transendothelial migration of ciliated cells. In one embodiment, the transendothelial migration comprises a transcellular event. In one embodiment, the transendothelial migration comprises a paracellular migratory event.

7. Ciliary Beat Frequency Activation with an IL-13 Inhibitor.

In one embodiment, the present invention contemplates a method for reducing neutrophil adhesion using an IL-13 inhibitor. In one embodiment, the IL-13 inhibitor is a CXCR2 antagonist. In one embodiment, the IL-13 inhibitor is MK-7123. In one embodiment, the neutrophil adhesion is reduced by approximately seventy-five (75) percent (%). In one embodiment, the IL-13 recruits neutrophils by approximately two-fold.

B. Exemplary Methods for Personalized Medicine.

In principle, biopsy derived primary cells and organoid technology allows rapid ex vivo testing of drug responses on the affected tissue of individual patients. As a first example, the colon organoid-based CF test (Dekkers et al., 2013) can be read out in weeks after biopsy. The approach was applied for identification and successful treatment of patients with very rare CFTR mutations, who otherwise have no access to the recently introduced CF drugs (Dekkers, 2016). The feasibility of culturing various solid tumors directly from the patient in the form of tumor organoids (see above) holds a similar promise, yet the applicability of such an approach is less clear than in the case of the “single genetic lesion” CF organoids. Tumor organoids grow with unpredictable and often slower kinetics when compared to wild-type organoids, and—like the original tumors—display a heterogeneous genetic make-up. Ongoing trials will reveal the validity and applicability of tumor organoids in the assessment of drug response at the level of the individual patient.

### V. Mucociliary Dysfunction.

Mucociliary dysfunction is believed to play a role in many chronic respiratory disorders, such as cystic fibrosis, chronic bronchitis, primary ciliary dyskinesia, asthma and chronic obstructive pulmonary disease (COPD). Munkholm et al., “Mucociliary clearance: pathophysiological aspects” Clin. Physiol. Funct. Imaging 34:171-177 (2014). In addition, mucociliary dysfunction may also caused by acute viral or bacterial infection or by air pollutants such as ozone, aldehydes or cigarette smoke. Defective mucociliary transport lowers quality of life and can result in substantial morbidity in terms of dyspnoea, recurring sinopulmonary infections, and frequent and productive coughs. Asthma is believed to affect over 23 million US Americans and 13 million US Americans are diagnosed with COPD. Consequently, mucociliary complications represent a major burden to patients and the health care system. Ford et al., “COPD surveillance-—United States, 1999-2011” Chest 144:284-305 (2013); Pleis et al., “Summary health statistics for U.S. adults: National Health Interview Survey, 2008” Vital Health Stat. 10:1-157 (2009); and Bloom et al., “Summary health statistics for U.S. children: National Health Interview Survey, 2008” Vital Health Stat. 10:1-81 (2009).

These complications arise, in part, because mucociliary clearance may be a line of defense against inhaled pathogens and toxicants. For example, multiciliated cells and mucus-producing goblet cells line the airways from the nasal cavity down to the pulmonary alveoli. A coordinated beating of cilia, which are lubricated by an aqueous periciliary liquid, transports a top layer of viscous mucus and embedded particulate to the pharynx where this mix is swallowed. Acquired or genetic defects of this mucus transport can reduce clearance of mucus and therefore increase the retention of microorganism which can cause recurrent viral and bacterial infections. Robinson et al., “Regional mucociliary clearance in patients with cystic fibrosis” J. Aerosol Med. Off. J. Int. Soc. Aerosols Med. 13:73-86 (2000); Sagel et al., “Update of respiratory tract disease in children with primary ciliary dyskinesia” Proc. Am. Thorac. Soc. 8:438-443 (2011); and Knowles et al., “Mucus clearance as a primary innate defense mechanism for mammalian airways” J. Clin. Invest. 109:571-577 (2002). Permanent establishment of pathogens can start a destructive feedback mechanism known as “vicious circle hypothesis”. In COPD, mucociliary clearance is further impaired due to increased mucus secretion and disrupted ciliary activity, resulting in chronic inflammation and lung destruction. Sethi, S., “Infection as a comorbidity of COPD” Eur. Respir. J. 35:1209-1215 (2010). Treatment options specifically designed to enhance mucociliary transport are urgently needed. For more effective development of drugs and diagnostics, biomechanical biomarkers need to be identified that are sensitive to the structure-function relationships enabling airway cilia to coordinate transport mucus over three length scales. Knowles et al., “Mucus clearance as a primary innate defense mechanism for mammalian airways” J. Clin. Invest. 109:571-577 (2002).

Cellular biology research has provided some basic insight into the molecular and cytoskeletal basis of ciliary beating. Brooks et al., “Multiciliated cells” Curr. Biol. CB 24:R973-982 (2014). Cell-level biomarkers, such as ciliary beat frequency, cilia polarity, and ciliary beat patterns, have been suggested as cellular machinery to provide a basis for automated screening assays for disease and drug responses in cultured and biopsied cells. Raidt et al., “Ciliary beat pattern and frequency in genetic variants of primary ciliary dyskinesia” Eur. Respir. J. 44:1579-1588 (2014); Smith et al., “ciliaFA: a research tool for automated, high-throughput measurement of ciliary beat frequency using freely available software” Cilia 1:14 (2012); Christopher et al., “The Effects of Temperature and Anesthetic Agents on Ciliary Function in Murine Respiratory Epithelia” Front. Pediatr. 2 (2014); and Quinn et al., “Automated identification of abnormal respiratory ciliary motion in nasal biopsies” Sci. Transl. Med. 7:299ra124 (2015).

However, presently reported cell-level biomarkers are limited in their ability to predict the behavior of an intact ciliated tissue and its interaction with mucus. Indeed it is on a tissue-level, that many structural and functional features exist which may contribute to lung disease, including metachronal wave patterns, the ratio and distribution of ciliated versus non-ciliated areas, ciliated cell polarity, and vertical mucus flows. Yaghi et al., “Airway Epithelial Cell Cilia and Obstructive Lung Disease” Cells 5:40 (2016). These reports show that current in vitro cell culture models lack the complexity to recreate lung physiology at the tissue-level. Furthermore, animal models are less accessible to live imaging and also fail to replicate the pathophysiology of uniquely human diseases, such as asthma.

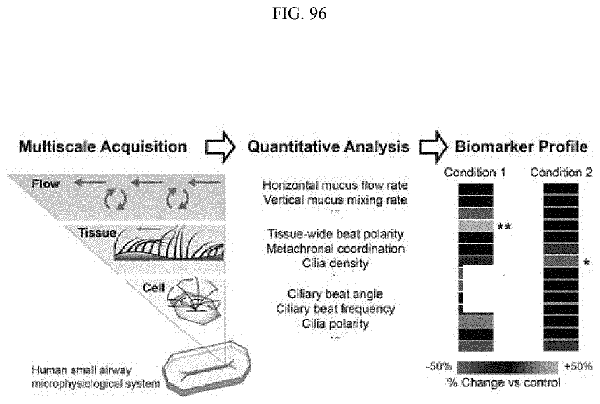

In one embodiment, the present invention contemplates a human small airway microphysiological system that models a variety of structural and functional aspects of human mucociliary transport. In one embodiment, the system creates and reports a readout of ciliary biomechanical biomarkers at multiple spatial scales. See, FIG. 96.

Although it is not necessary to understand the mechanism of an invention it is believed that, the resulting biomarker profiles measure natural variability between patient groups. In one embodiment, natural variability data is used in methods for identifying disease-specific signatures, patient-specific signatures, and drug-specific signatures. In other embodiments, natural variability data is used in methods for detecting correlations between biomarkers.

Although it is not necessary to understand the mechanism of an invention, it is believed that such biomarker correlations reveal causal mechanisms by which single cell mechanics are combined to achieve organ-level mucus clearance. To this end, a preliminary biomimetic approach has been reported for culturing human lung epithelium in a microphysiological system, in addition to implementing automated quantitative readouts of ciliary beat. Benam et al., “Small airway-on-a-chip enables analysis of human lung inflammation and drug responses in vitro: Nat. Methods (advance online publication) (2015); and Benam et al., “Matched-comparative modeling of normal and COPD human airway responses to inhaled smoke in vitro”. Cell Syst. (2016). These results suggest that tissue-wide cilia remodeling plays a role in treating inflammatory disease. In one embodiment, the present invention, contemplates a microphysiological system for developing and validating biomechanical biomarkers that profile mucociliary transport in healthy and diseased human airway epithelia.

A. Exemplary Profiling Mucociliary Transport in Diseased Airway Epithelium with Biomarkers.

Acquired and genetic cilia-related disorders and more broadly, human diseases involving cilia dysregulation, constitute a large range of pathologies with obvious mechanical factors. Mechanical factors in general refer to physiological mechanical barriers and fluid based mechanical factors associated with ciliary function, for example, lung diseases where mucociliary clearance constitutes a mechanical barrier mechanism against pathogen and exogenous particles. Tilley et al., “Cilia dysfunction in lung disease” Annu. Rev. Physiol. 77:379-406 (2015). Structure-function studies have helped identify the molecular targets that participate in regulating ciliary beating. Salathe, M., “Regulation of mammalian ciliary beating” Annu. Rev. Physiol. 69:401-422 (2007). However, the mechanics of such regulation—and its specific failure modes in disease—is relatively unknown.

Although it is not necessary to understand the mechanism of an invention, it is believed that ciliary biomarker profiling may provide valuable information on the fluid-mechanical factors associated with pathological processes. Specifically, mucociliary transport in healthy tissues and in three major classes of airway diseases with impaired mucociliary transport. These diseases include, but are not limited to, asthma, cystic fibrosis, and virus infection (e.g., influenza virus, rhinovirus and/or parainfluenza virus). These types of genetic cilia-related disorders/diseases, e.g. have genetic components, such as gene alleles associated with asthma and cystic fibrosis; and acquired cilia-related disorders/diseases, e.g. microbial infections, are not intended as separate disorder or disease platforms because there are genetic associations with acquired disorders/diseases. Further, patients having genetically based cilia-related disorders/diseases or acquired cilia-related disorders/diseases may have increased susceptibility to infection, as in secondary infections and opportunistic infections.

In one embodiment, the present invention contemplates a method for generating high-content, high-resolution biomarker profiles comprising disease-specific biomechanical signatures and correlations. In one embodiment, the profiles identify prognostics for staging and prediction of risk for developing respiratory diseases. In one embodiment, the profiles identify both intended and adverse effects of respiratory drug treatments. For example, long-term treatment with inhaled corticosteroids may negatively affect mucociliary transport as it increases the risk of developing airway infection. McKeever et al., “Inhaled corticosteroids and the risk of pneumonia in people with asthma: a case-control study” Chest 144:1788-1794 (2013).

Profiling mucociliary transport may identify impaired clearance of mucus which is a common symptom of many respiratory diseases. Hilding, A. C., “The relation of ciliary insufficiency to death from asthma and other respiratory diseases” J. Allergy 14:351-352 (1943). The Th2 cytokine IL-13 has been associated with cilia abnormalities. Ingram et al., “IL-13 in asthma and allergic disease: asthma phenotypes and targeted therapies” J. Allergy Clin. Immunol. 130:829-842-844 (2012). Among the effects of IL-13 stimulation on airway ciliated cells are induction of goblet cells hyperplasia and reduction of ciliated cell number. decrease of cilia beating frequency and impairment of ezrin apical localization. Laoukili et al., “IL-13 alters mucociliary differentiation and ciliary beating of human respiratory epithelial cells” J. Clin. Invest. 108:1817-1824 (2001); Thavagnanam et al., “Effects of IL-13 on mucociliary differentiation of pediatric asthmatic bronchial epithelial cells” Pediatr. Res. 69:95-100 (2011); and Gomperts et al., “IL-13 regulates cilia loss and foxj1 expression in human airway epithelium” Am. J Respir. Cell Mol. Biol. 37:339-346 (2007).

B. Exemplary Profiling Mucociliary Transport in a Model of Human Rhinovirus Infection.

Mucociliary transport is a fundamental defense mechanism of the respiratory tract against particulates and pathogens. Changes in cilia function as a result of cellular responses involving to particulates and pathogens are nonlimiting examples of acquired cilia-related disorders. Viral infection of the respiratory tract is often associated with secondary cilia dyskinesia and cilia ultrastructural defects. Mezey et al., “Mucociliary transport in allergic patients with antigen-induced bronchospasm” Am. Rev. Respir. Dis. 118:677-684 (1978); and Ingram et al., “IL-13 in asthma and allergic disease: asthma phenotypes and targeted therapies” J. Allergy Clin. Immunol. 130:829-842-844 (2012).

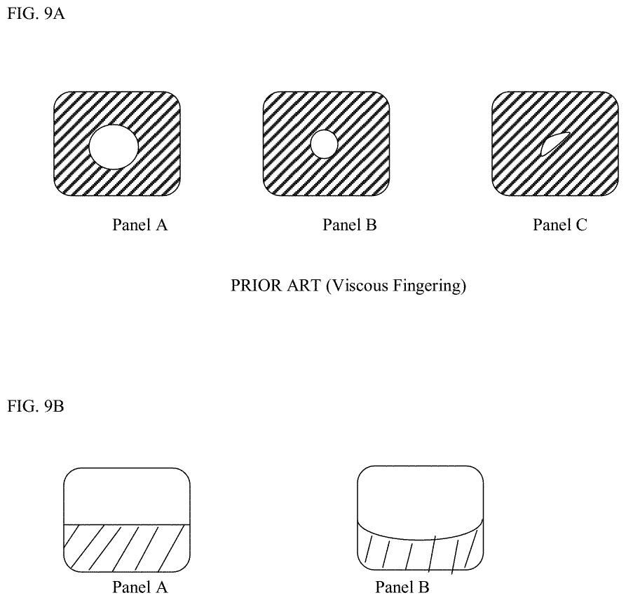

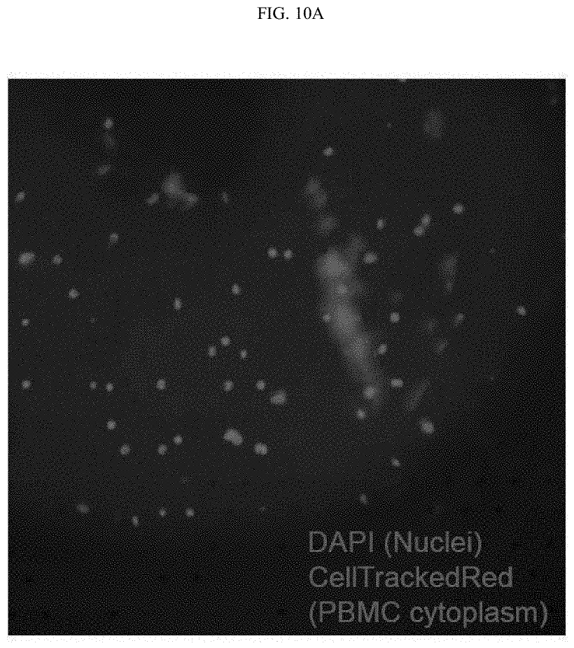

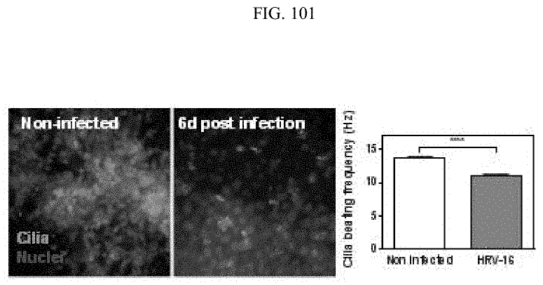

In one embodiment, the present invention contemplates a method for detecting biomarkers of acquired ciliary defects by quantifying the outcome of rhinovirus infection in well-differentiated airway epithelial cells. The data presented herein was collected using the presently disclosed microphysiological platform and indicates that rhinovirus infection of well-differentiated airway epithelial cells impairs cilia beating frequency and induces destruction of ciliated cells over time. See, FIG. 101 FIG. 9A-B. These data suggest that the presently disclosed cell culture platform and integrated imaging and data analysis system is well suited to profile the effects of viral infection on mucociliary transport using a biomechanical biomarker panel. Similar results to confirm rhinovirus can be obtained with immunofluorescence staining of infected cultures with an anti-dsRNA antibody (mabJ2) to detect replicating rhinovirus genome while cilia can be stained using an antibody against β-Tubulin. Jurgeit et al., “An RNA replication-center assay for high content image-based quantifications of human rhinovirus and coxsackievirus infections” Virol. J. 7:264 (2010).

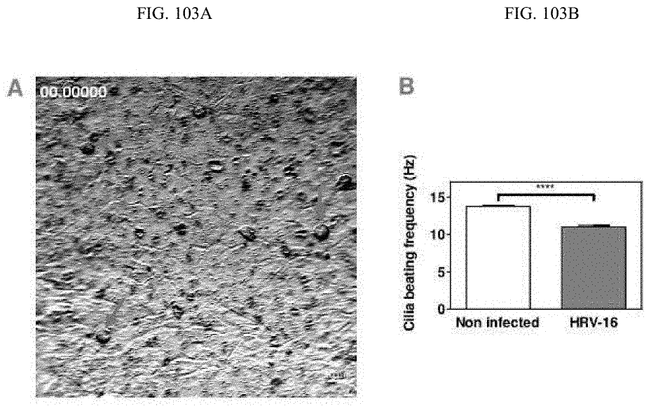

The data presented herein shows that rhinovirus infection induces ciliated cells rounding and reduces cilia beating frequency. For example, bead movement across a cilia-mucociliary elevator was measured. See, FIG. 103A. Additionally, cilia beating frequency was determined with and without viral infection (HRV-16). See, FIG. 103B.

C. Exemplary Profiling Mucociliary Transport in a Model of Cystic Fibrosis (CF).

Cystic fibrosis (CF) is a fatal genetic disease caused by at least one mutation in the CFTR (cystic fibrosis transmembrane conductance regulator) gene that regulates chloride and water transport across epithelia layers of organs and affects multiple organs including the lungs, intestine, liver, etc. Because of these defects, chloride ions cannot move into or out of the cells like they should. This can cause thick, sticky mucus to build up in organs, such as the lungs.

Cystic fibrosis (CF) is a genetic disease caused by a mutation of the CFTR gene. Cystic fibrosis (CF) is caused by a range of mutations in the cystic fibrosis transmembrane conductance regulator (CFTR) chloride channel formed by proteins that are normally expressed in epithelial cells of many organs, not just restricted to airway epithelial cells. CF patients have at least one copy of the F508del mutation in the cystic fibrosis transmembrane conductance regulator (CFTR) gene. This one mutation cause fewer CTFR proteins to reach the cell surface where the ion channel proteins are normally located for providing proper ion regulation into and out of a respiratory cell. Further, when CFTR proteins to reach the cell surface that do not open correctly for ion regulation. However, while F508 is a common mutations, there are other gene mutations associated with CF. Other types of CFTR Gene mutations are found in a population of CF patients, individually or in combinations, that may alter efficacy of treatments. Thus, CFTR gene mutations in airway cells that may find use in assertions with treatment efficacy, include but are not limited to F508del, G551D; G1244E; G1349D; G178R; G551S; G970R; S1251N; S1255P; S549N; S549R; R117H; etc. The first letter refers what is considered the normal amino acid while the second letter refers to the substituted amino acid. Substituted amino acids arise from mutations in corresponding nucleic acid base pairs controlling translation of the genetic code into a specified amino acid.

In the lung, the CFTR mutation leads to disrupted transport of chloride and sodium across the respiratory epithelium, altering the osmolar gradient, and resulting in accumulation of thick, dehydrated mucus. This dramatically decreases mucociliary clearance, causing airway clogging, infections and chronic inflammation. Flume et al., “Cystic fibrosis pulmonary guidelines: pulmonary complications: hemoptysis and pneumothorax” Am. J. Respir. Crit. Care Med. 182:298-306 (2010). No curative treatments are available today. The data presented herein demonstrates that the microphysiological platform and data imaging and analysis system as described herein recreates a differentiated CF epithelium in vitro from epithelial cells.

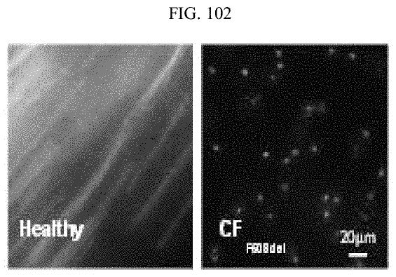

Furthermore, the system recapitulated the impaired mucociliary function of a CF epithelium in vitro. See, FIG. 102. Although it is not necessary to understand the mechanism of an invention, it is believed that such a highly differentiated CF epithelium with a physiologically reactive mucociliary transport system permits functional testing and validation of relevant biomechanical biomarkers associated with diagnosis and treatment. For example, immunofluorescence staining of CFTR can be performed in cultures comprising either healthy epithelial cells or CF epithelial cells to verify CFTR location at the apex of cells in healthy cultures and its absence in CF cultures. Cholon et al., “Modulation of endocytic trafficking and apical stability of CFTR in primary human airway epithelial cultures” Am. J. Physiol. Lung Cell. Mol. Physiol. 298:L304-14 (2010). Although it is not necessary to understand the mechanism of an invention, it is believed that the presently disclosed microphysiological platform can recapitulate CFTR misfolding in F508del patients and impaired CFTR transit to the apical membrane.

In one embodiment, the present invention contemplates a method for cultivating and differentiating human primary airway epithelial cells derived from human CF patients. In one embodiment, the epithelial cells comprise an F508del mutation (commercially available from Epithelix (Switzerland)), as one example. Other sources of both healthy and cystic fibrosis primary airway epithelial cells (HAECs) were tested for variability of cell quality from lot to lot, continuous availability, growth in Transwells and microfluidic devices, including but not limited to: BioIVT: ASTERAND™ Bioscience Bronchial Epithelial Cells—Cystic Fibrosis (primary cells from CF patients), D-HBE-CF—Diseased Human Bronchial/Tracheal Epithelial Cells from donors having Cystis Fibrosis (Lonza), and Diseased Bronchial/Tracheal Epithelial Cells, Primary, CF from Lifeline Cell Technology.

VI. Developing Embodiments of Healthy and Disease Modeling, e.g. with Gels.

Despite advances in cell culturing methods used for modeling healthy and diseased tissues and organs, many types of cells show fragileness in vitro. Such fragile, i.e. sensitive, cell types, include diseased cell types, e.g. cells that may not grow as robustly as healthy cells under the same culture conditions, or do not survive in cultures as long as healthy cells under the same culture conditions. Sensitivity include changes in growth in response to the use of different media formulations used for comparable healthy cell growth and/or differentiation, sensitivity to different types of growth surfaces, i.e. ECM, gels, overlays, etc. As one example, described herein, CF cells grown in the same medium as healthy airway cells may not show comparable ciliation when not grown on gels as described herein. Therefore, sensitivity is also referring generally to differences observed from culture to culture of the same cell types, e.g. a general lack of repeatability. As described herein, application of and use of gels surprisingly overcame many sensitivity issues so that a wider range of cell medium and cell types resulted in cultures having a greater amount of repeatability.

One hurdle encountered when developing gels for use with microfluidic devices was a technical problem that arose when using automated equipment for delivering pressurized fluid for eliminating bubbles from gel overlain microchannels, see herein for a more detailed method of removing bubbles. Pressurized fluid effects on gels were particularly evident when used for increasing the gas carrying capacity of flowing fluids, e.g. cell media fluids. Specifically, for thinner gel layers as described herein, pressurized fluid flow through microchannels for eliminating bubbles resulted in a substantial loss of gel areas, particularly in the middle area of the channel. At the same time gels remained about the same thickness at the ends of the channels. This loss of gel layer thickness resulted in variability of cell attachment for subsequently seeded cells. Moreover, when pressurized fluid was flowed over cells attached to gel layers, there was a loss of both cells and gels in the center region of the microfluidic channel. Because pressurized fluids are flowed through microchannels often just before or after cell seeding, and again (optionally) on approximately Day 5 of culture, this cell-gel loss also created a hole in the cell layer. Adjacent cells may or may not grow over the hole, depending upon the cells, size of hole, etc.

Such variations were exacerbated depending upon which type of gel was used because a variety of gels may be used because in general, each cell type may have a preference for a different gel type. Thus, there was a reduced level of repeatability of cell coverage in microfluidic chips which further caused a reduction in quality of cell cultures between duplicate chips.

Variability of chip cultures caused by a loss of gel coverage, combined with variability of cell viability from lot to lot, and commercial supplier to supplier, and variability of growth between cell culture media for a particular cell type from supplier to supplier, all combined to result in microfluidic devices having less than desired repeatability.

However, it was discovered that the use of an automatic pipetting and flushing systems described herein for layering partial and continuous gels within devices, along with a corresponding demonstration of increased gel coverage and retention over the length of the microchannel, resulted in a greater level of repeatability of gel coverage at desired thicknesses.

Further, it was also surprising that by using such gels for culturing cells, in particular fragile cells such as described herein, overcame many of the responses to variations to provide more robust and repeatable cell modeling conditions for both healthy and diseased cells. Advantages of providing more reliable and more robust tissue modeling including for use in testing treatment compounds to provide closer matches between in vitro results to how these medicines work in vivo.

Thus, advantages of using gels for cell culturing includes but is not limited to, differentiating CF cells into ALI instead of using liquid flooded cell cultures, thus having models of cells in more physiological conditions of ALI instead of using submerged cultures; overcoming at least some differences in growth of CF cells obtained from different commercial sources, overcoming differences in growth of CF cells cultured in different cell media formulations; having matured CF tissue for use as models of diseased tissue in the presence of infectious microbes, e.g. fungi, bacteria, and viruses; having a diseased CF model showing impaired mucus clearance in vitro for more accurate assessments of drug efficacy and testing of compounds for treating CF disease.

In both Transwell and microfluidic cultures differentiating CF cells did not progress well on merely an ECM coating (e.g., Col-IV), where differentiation was sporadic and few cells developed beating cilia. Surprising discoveries included observing that in contrast, a (3D) gel layer (e.g., Col-I overlay) on top of an ECM coating (e.g., Col-IV), differentiating CF cells developed into numerous cells having visible beating cilia. Further, differentiation was repeatable and reliable in contrast to not using such gel layers.

Therefore, advantages of using partial gels for disease modeling using fragile cells associated with disease, as compared to healthy cells, include but are not limited to, less variation in cell quality, i.e. increased repeatability, and more robust differentiated cell layers as observed when comparing cultures of commercial primary CF cells from different sources or patients, less difference in results depending upon the type of media used, i.e. cell media is not restricted to merely one formulation of supplier, etc.

Thus, in preferred embodiments, at least one gel, including partial gels as described herein, are used for growing healthy cells and diseased cells in Transwell devices and microfluidic devices. Examples of healthy cell and diseased cells include but are not limited to any particular type of organ cell, such as parenchymal cells and endothelial cells, e.g. epithelial cells as described herein and endothelial cells as described herein.

In one embodiment of CF airway cell cultures, robust differentiation resulting in ciliated cells was obtained without the presence of endothelial cells, including the presence of differentiation associated biomarkers. In one embodiment of CF airway cell cultures, co-cultures of CF epithelial cells are contemplated with cells embedded in gels, e.g. stromal cells, fibroblast cells, etc. In one embodiment of CF airway cell cultures, seeding endothelial cells in an opposing channel for co-culturing airway cells for healthy and diseased tissue modeling is contemplated. In some embodiments, the use of endothelial cells is contemplated, as described herein.

Thus, contemplated comparative measures of differentiation include but are not limited to comparative RNA expression of mature cell type specific markers between cultures without endothelial cells to cultures with endothelial cells. Such comparative cell types may also be made between healthy cells vs. diseased cell cultures, and cultures contacted with treatment compounds including test compounds for determining alterations in cell phenotypes.

As one example of disease modeling, devices comprising cells derived from patient biopsies, where the patient is at risk of, or diagnosed with, a disease referred to generally as “diseased cells”, is cultured in vitro for use with individualized drug testing. In some embodiments, such diseased cells may be genetically analyzed for determining the presence of one disease allele where the matching allele is not associated with disease, i.e. heterozygous, or whether two identical disease alleles are present, i.e. homozygous. Additionally, alleles associated with the disease allele may be identified as contributing to a healthy or disease genotype. Such genetic information may then be used for associating in vitro efficacy of tested drugs to predications of in vivo efficacy. In some embodiments, such association may provide information for clinical use in determining amounts of drug per treatment, along with predicting duration of treatment over time.

As merely one example whose general description may be applied to other respiratory diseases, respiratory airway cells derived from patient biopsies may be obtained commercially, as described herein, or directly from patient biopsies in the laboratory. In some embodiments, where a disease mutation is known, healthy cells may be engineered to express the mutation on different genetic backgrounds.

Such diseased cells are seeded into devices comprising gels, as described herein, wherein parital gels contact a membrane within a culturing device. Such seeded devices may then be used for testing drug treatments by contacting cells with test compounds, including but not limited to known test compounds and unknown test compounds. Exemplary drug treatments for testing on CF airway epithelium include but are not limited to known compounds, such as those offered for sale by Vertex corporation, i.e. TRIKAFTA®; SYMDEKO®; KALYDECO® (e.g. comprises a mixture of elexacaftor/tezacaftor/ivacaftor with ivacaftor; elexacaftor/tezacaftor/ivacaftor; and ivacaftor, respectively), ORKAMBI® (lumacaftor/ivacaftor. Elexacaftor and tezacaftor bind to different places on F508del-CTFR proteins to aid moving more proteins reach the cell surface. Lumacaftor brings more CFTR proteins to the cell surface. Ivacaftor helps those proteins at the cell surface to stay open longer. Further, experimental in vitro trials may include cells derived from patients with CF homozygous for the F508del Mutation in the CFTR Gene. Further, in some embodiments, experimental in vitro trials may include cells derived from patients with CF heterozygous for the F508del Mutation and a second gene mutation predicted to provide an enhanced response to a compound, such as Ivacaftor. Together, these 3 types of components help F508del-CFTR proteins function better as a CF treatment.

Moreover, efficacy of such disease treatments may be tested in vitro in the presence of other medications or chemicals, i.e. for in vitro testing drug interactions. Other medications or chemicals that may interact with treatments that may be added to cell cultures for identifying potential adverse effects, e.g. adverse effects of CF treatments in the presence of other chemical compounds, include but are not limited to as prescription and over-the-counter medicines, vitamins, homeopathic herbal supplements (e.g. St. John's wort (Hypericum perforatum)) that patients may be taking as self mendicants or prescribed for underlying conditions (i.e. conditions related to kidney, liver, pregnancy, etc.) before or during CF targeted drug treatments. Additional examples include, antibiotics such as rifampin (RIFAMATE®, RIFATER®) or rifabutin (MYCOBUTIN®), seizure medicines such as phenobarbital, carbamazepine (TEGRETOL®, CARBATROL®, EQUETRO®), or phenytoin (DILANTIN®, PHENYTEK®; antifungal medicines including ketoconazole (such as NIZORAL®), itraconazole (such as SPORANOX®), posaconazole (such as NOXAFIL®), voriconazole (such as VFEND®), or fluconazole (such as DIFLUCAN®); immunosuppressant medicines: cyclosporine, everolimus (Zortress®), sirolimus (Rapamune®), or tacrolimus (Astagraf XL®, Envarsus XR®, Prograf®, Protopic®); sedatives and anti-anxiety medicines: triazolam (Halcion®) or midazolam (Dormicum®, Hypnovel®, and Versed®); antibiotics including telithromycin (such as KETEK®), clarithromycin (such as BIAXIN®), or erythromycin (such as ERY-TAB®); other medicines including rifampin, rifabutin, phenytoin, ciprofloxacin, etc.

In some embodiments, effluent of treated chips or direct fluidic linkage with other organ chips, such as kidney (renal) chips, liver chips, or direct test compound testing directly in cell culture devices comprising gels may also be done for detecting potential adverse reactions. In some embodiments, as examples, drug interactions with a biomarker, such as levels of Transaminase (ALT or AST), CYP3A, may also be used. In particular, biomarkers of elevated Transaminase (ALT or AST), and CYP3A may be monitored in relation to potential adverse reactions, e.g. hepatic impairment of function. Further, treatments that may be CYP3A inducers or inhibitors may induce adverse drug interactions. Thus as another example, cell culture devices as described herein, may be used for determining whether to reduce the amount of a treatment compound when co-administered with moderate CYP3A inhibitors (e.g., fluconazole and erythromycin; one or more components isolated from grapefruit juice or Seville oranges). Strong CYP3A inhibitors, such as ketoconazole, itraconazole, posaconazole, voriconazole, telithromycin, and clarithromycin, co-administration with strong CYP3A inducers, such as rifampin, rifabutin, phenobarbital, carbamazepine, phenytoin, and St. John's wort is not recommended monitoring are recommended when co-administering KALYDECO with sensitive CYP3A and/or P-gp substrates, such as digoxin, cyclosporine, and tacrolimus.

### VI. Sources of Cells.

As opposed to other models of healthy and diseased cells, the use of gels with cell culturing devices as described herein, overcome previous limitations of organoid derived models by the incorporation of multiple cell types, immune cells, microbial cells, innervation of cells, along with embodiments of fluidically linking organ devices, either by manual introduction of fluid from one device into another, or by a physical continuous fluidic linkage whereby fluid emanating from a first organ device enters into the fluid chamber of a second device, and so on.

A. Organoids.

Organoids are contemplated for use with devices described herein, for modelelin organ-specific acquired and hereditary diseases. Organoids can be initiated from the two main types of stem cells: (1) pluripotent embryonic stem (ES) cells and their synthetic induced pluripotent stem (iPS) cell counterparts and (2) organ restricted adult stem cells (aSCs). Both approaches exploit the high expansion potential, i.e. long term availability of stem cells for providing progeny cells in culture. For ES and iPS cells, here collectively termed pluripotent stem cells or PSCs. Organ restricted adult stem cells (aSCs) mimic various organ stem cell niches when used for providing organoids. Thus, both PSCs and aSCs may find use with methods and devices described herein by providing organoids that may be used directly or dissociated into single cells prior to seeding into devices. Such cells may be embedded within gels and seeded onto gels.

1. Liver Organoids.

In some embodiments, liver organoids may be derived from healthy donors and diseased patients. As merely on example, liver organoids from alpha 1-antitrypsin deficiency patients reproduced the deleterious effects of the mutant protein precipitates in hepatocytes, while the absence of mature biliary cells in liver organoids from an Alagille syndrome patient mirrored the in vivo biliary tree abnormalities (Huch et al., 2015). Liver organoids from dogs deficient in the copper-transporter COMMD1 mimicked the disease by accumulating toxic levels of copper, which could be salvaged by re-expression of wild-type COMMD1 protein (Nantasanti et al., 2015). Therefore, organoid derived cells may be used for providing liver disease models for use in testing treatment substances and other substances as described herein. In some embodiments, such

2. Lung Organoids.

Hogan and colleagues reported an early bronchiolar lung organoid culture protocol, involving Matrigel® supplemented with EGF, e.g. Single basal cells isolated from the trachea grew into “tracheospheres” consisting of a pseudostratified epithelium with basal cells and ciliated luminal cells. These organoids could be passaged at least twice. No mature Clara-, neuroendocrineor mucus-producing cells were observed (Rock et al., 2009). In a later study, this clonal 3D organoid assay was used to screen for factors controlling generation of ciliated versus secretory cells from basal cells. It was thus found that IL-6 treatment resulted in the formation of multiciliated cells at the expense of secretory and basal cells (Tadokoro et al., 2014). A human airway organoid representing the distal airways (“alveolospheres”) was grown.

Alveoli consist of gas-exchanging type I and surfactant-secreting type II cells. While both cell types originally derive from a common progenitor, it appears that, later in life, a rare self-renewing type II cell acts as the stem cell to regenerate the alveolar epithelium. Indeed, sorted type II cells remained proliferative in short-term culture and could generate type I cells (Desai et al., 2014; Treutlein et al., 2014). Alternative culture conditions allowed establishment of mouse and human alveolospheres from single type I as well as type 2 alveolar cells, containing both cell types in the same organoid. Having said this, these alveolosphere culture conditions are as yet not fully defined, requiring co-culture with non-epithelial cells (e.g., mouse lung fibroblasts) (Barkauskas et al., 2013; Jain et al., 2015).

B. Applications of Organoid Technology.

The description of uses of organoids, may also be generalized to cell cultures in general as described herein. In other words, examples of organoid uses for gene therapy. Etc. May be used for devices seeded with cells from other origins.

For both PCS- and aSC-based organoids can be initiated from single cells, cultured long-term and are amenable to cell-biological and molecular analyses. As such, they provide a window between cell lines and in vivo studies to studying basic gene functions and cellular processes, in addition to testing potential therapeutics, drug interactions and gene therapy. Organoids are contemplated for use in providing cells in order to model hereditary diseases. Further, organoid derived cells are contemplated for use in developing treatments for individuals, using that individual's tissues or cells, as one example of individualized medicine. Illustratively, Knoblich and colleagues identified a patient with a mutation in the CDK5RAP2 and severe microcephaly. The corresponding iPS cells made significant smaller “mini-brains,” containing only occasional neuroepithelial regions with signs of remature neural differentiation, a phenotype that could be rescued by reintroducing the CDK5RAP2 protein (Lancaster et al., 2013).

1. Gene Therapy.

In some embodiments, disease modeling may include gene therapy testing. As one example, CRISPR/Cas9 genome editing may be used to correct mutations, i.e. to restore the non disease associated amino acid(s) in the CFTR locus by homologous recombination in cultured respiratory epithelial cells, and in other epithelial cells harboring CFTR, such as intestinal stem cells of CF patients. A corrected allele in CFTR in cultured intestinal stem cells of CF patients was fully functional, as demonstrated in clonally expanded organoids (Schwank et al., 2013).

In some embodiments, gene therapy is contemplated for use to induce gene mutations to either induce disease or reduce disease in clonally expandable cell populations derived from patients.

2. Infectious Disease.

Since organoids—unlike cell lines—ideally represent all cellular components of a given organ, they are theoretically well suited for infectious disease studies, particularly of pathogens that are restricted to man and are dependent on specialized cell types. Thus, infectious diseases including opportunistic infections may be modeled using cell culture devices described herein containing gels. In one embodiment, cells seeded into culture may be derived from organoids. As one example, ciliated cells are affected by the presence of microbes, microbial infections in nonciliated cells and ciliated cells, including but not limited to respiratory microbes, such as bacteria, e.g. Pseudomonas. Aeruginosa, Haemophilus influenzae; virus such as respiratory syncytial virus (RSV); fungi such as Aspergillosis spp., phage such as14/1, ΦKZ, PNM and PT7; and protist such as Tetrahymena thermophila and Acanthamoebae polyphaga, etc.

In an illustrative application, iPS-derived lung organoids were generated from an otherwise healthy child who suffered life-threatening influenza and carried null alleles in the interferon regulatory factor 7 gene. These organoids produced less type I interferon and displayed increased influenza virus replication (Ciancanelli et al., 2015). In another example, human stomach organoids, grown from PSCs or aSCs, can be productively infected by Helicobacter pylori (Bartfeld et al., 2015; McCracken et al., 2014). As another example, Qian et al. developed a miniaturized spinning bioreactor to generate forebrain-specific organoids from human iPSCs, following the Lancaster/Knoblich protocol. These organoids recapitulate many features of cortical development, including the formation of a distinct human-specific outer radial glia cell layer. Infection of these developing forebrain organoids with Zika virus (ZIKV) resulted in the preferential infection of neural progenitors, resulting in cell death, decreased proliferation, and a reduced neuronal cell-layer volume, thus modeling ZIKV-associated microcephaly. (Qian et al., 2016). Thus, in some embodiments, brain cells are contemplated for use in testing for potential ZIKV antiviral drugs.

3. Organoid-Derived In Vitro Cystic Fibrosis Disease Modeling.

The most common CF mutation (approximately 70% of cases) is caused by a phenylalanine (F) deletion at position 508 (F508del). Human pluripotent stem cells can be directed to differentiate in vitro into CFTR-functional conducting airway epithelium, Wong, et al., “Directed differentiation of human pluripotent stem cells into mature airway epithelia expressing functional CFTR protein”. Nat Biotechnol. 2012 September; 30(9): 876-882.

As an illustrative example in general for other ograns, and for airway in particular, differentiation of human ESC and iPSC into definitive endoderm: Briefly, pluripotent stem cells were harvested, gently triturated into single cell suspensions and seeded onto transwells (0.4 μm pore size, Corning) pre-coated with human placental collagen Type IV, which was shown to support airway epithelial cell growth. The cells were immediately treated with 100 ng/ml Activin-A and 25 ng/ml WNT3A (R&D Systems) for 4 consecutive days in Endoderm Differentiation Media consisting of serum-free Knockout DMEM (Invitrogen) with Glutamax (Invitrogen), penicillin/streptomycin (GIBCO), 1 mM nonessential amino acids (GIBCO) and 0.5 mM mercaptoethanol. Subsequent differentiation steps were performed on the transwells.

As an illustrative example in general for other organs, and for airway in particular, differentiation of definitive endoderm into anterior foregut endoderm progenitors; For anterior foregut endoderm differentiation and especially embryonic lung progenitors, definitive endoderm cells were treated with 500 ng/ml FGF2 (Preprotech) and 50 ng/ml Sonic hedgehog (SHH, Cedarlane) for 5 days in Endoderm Differentiation Media.

As an illustrative example in general for other organs, and for airway in particular directed differentiation of foregut endoderm into mature lung cell fates is as follows. The cells were treated with 50 ng/ml FGF10, 50 ng/ml KGF (FGF7) and 5 ng/ml BMP4 (all R&D systems) for 5 days followed by 10 ng/ml FGF10, 10 ng/ml FGF7 and 10 ng/ml FGF18 (Sigma-Aldrich) for an additional 5 days. To differentiate the cells into mature airway epithelial cells, the cells were cultured in Bronchial Epithelial Growth Media (BEGM, Lonza) supplemented with FGF18 (10 ng/ml) for 10 days followed by Bronchial-Air Liquid interface (B-ALI, Lonza) media for an additional 15+ days. The cells were “air-lifted” and B-ALI media was only added to the bottom but not the top of the transwell. However, such directed differentiation is contemplated for use with gels as described herein. In one embodiment, foregut endoderm may be seeded into microfluidic devices having gels for inducing differentiation including under conditions of an ALI as described herein.

4. Generation and Characterization of Human iPSC Lines.

Briefly, the following is an exemplary published procedure that may be used for providing cells for use as described herein. Human skin fibroblasts (GM00997, GM04320) were obtained from the Coriell Cell repository (Coriell Institute for Medical Research) and HSC patient fibroblast the Hospital for Sick Children (Toronto, Canada) with informed consent. These fibroblasts were isolated from the donor skin biopsy as previously described²⁶. By 4 weeks of reprogramming obvious human ESC-like EGFP+ colony numbers were enriched under puromycin selection before picking and expansion. CF-iPSC lines with ESC-like morphology were further assessed for pluripotency marker expression (NANOG, TRA1-81, TRA1-60) by flow cytometry and immunofluorescence, and real-time qPCR to examine up-regulation of endogenous pluripotency genes (OCT4, SOX2, C-MYC and KLF4) and down-regulation of the exogenous retroviral transgenes. In addition, gene expression of other pluripotency biomarkers DNMT3B, REX1, TERC, TERT were also assessed. Karyotype analysis was performed to determine genetic stability. iPSC lines were subjected to in vitro embryoid body and in vivo teratoma assays for functional tests of pluripotency as previously described.

As one illustrative example, CF-iPSC may find use in methods and devices described herein. CF-iPSC generated by retroviral-mediated reprogramming characteristically resembled hESC and were functionally pluripotent. Directed differentiation of CF mutant iPSC into airway epithelial cells pluripotent stem cells silenced the reprogramming retroviral transgenes by reprogramming primary human fibroblasts using retroviruses containing the four pluripotency factors (OCT4, KLF4, C-MYC and SOX2)

In the F508del CF mutation, the mutant CFTR protein does not fold properly in the endoplasmic reticulum, preventing it from being properly trafficked to the plasma membrane.

Instead the mutant CFTR protein is rapidly targeted for degradation. Small molecules sometimes called “corrector” compounds (substances) may be effective in partially rescuing the trafficking defect. Thus, CF-iPSC-derived epithelial cells may be used to evaluate CF corrector substances. One example of a test substance is C18, an active analog of the small molecule VX-809 (used in phase II clinical trials) for promoting plasma membrane localization of F508del-CFTR in CF-iPSC-derived epithelial cells. Those cultures treated for 24 hours with C18 (10 μM) exhibited patches of cells expressing CFTR on their cell surface compared to no surface localized F508del-CFTR detected in control DMSO-treated CF-iPSC-cultures.

### VII. Data Imaging And Analysis System

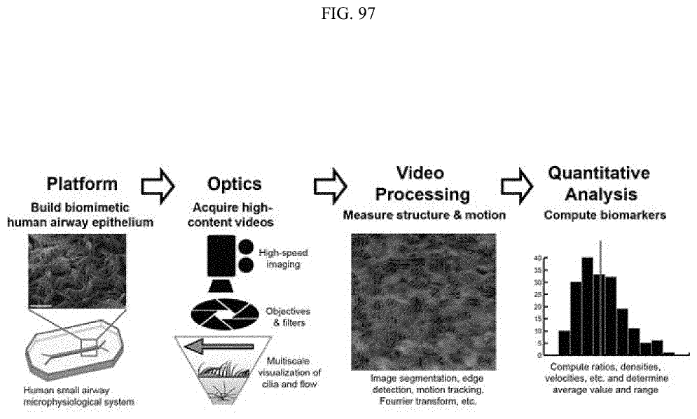

In one embodiment, the present invention contemplates a small airway microphysiological system configured for visualization and acquisition of ciliary beat and mucus flow at multiple scales, automated video processing to measure structures and kinematics, and quantitative analysis to compute quantitative biomarkers of mucociliary transport, such as densities, angles, velocities, frequencies, and rates. See, FIG. 97. In one embodiment, the data image and analysis system comprises a real-time analysis of mucociliary transport and cilia mechanics using a high-speed phase-contrast video-microscopy, followed by computational image processing and quantitative analysis of biomarkers

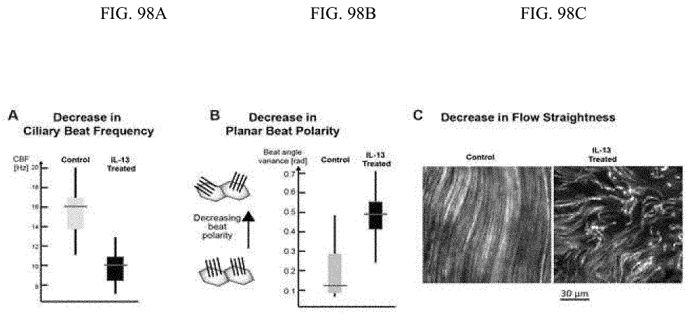

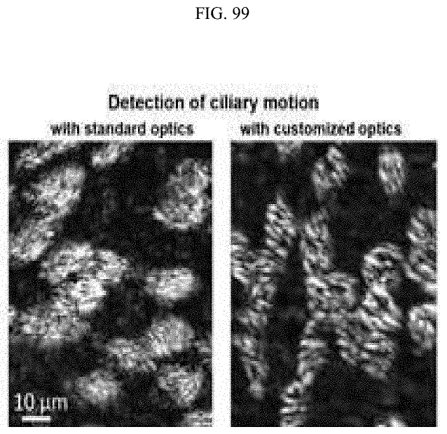

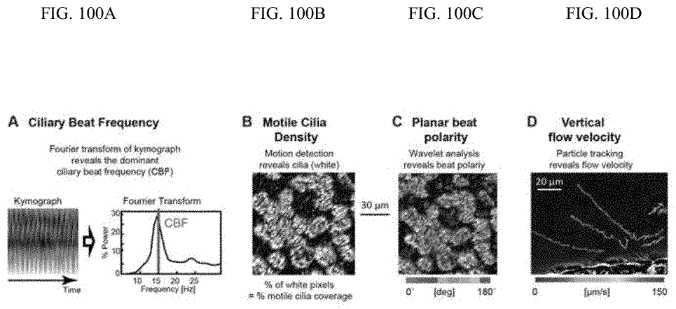

Data imaging and analysis includes, but is not limited to, comparing a biomarker profile of healthy cultures to models of three airway disease classes: inflammatory disease (e.g., asthma), genetic ciliopathy (e.g., cystic fibrosis), and acquired ciliopathy (e.g., viral infection). In vitro platforms have been developed with well differentiated human primary airway epithelial cells needed to recapitulate human airway physiology. Then, these platforms can be used for the acquisition and analysis of several structural and kinematic metrics known to reflect ciliary biomarkers including, but not limited to, mucociliary transport, ciliary beat frequency, planar cell polarity, ciliated cell density, and mucus flow velocity. Such metrics have been compared between healthy tissue and a model of asthma, where treatment with the TH2 cytokine IL-13 simulates an asthmatic epithelium. It is well-established that IL-13 treatment induces remodeling of the airway epithelium. For example, a multiscale analysis of healthy and IL-13 treated cultures have recapitulated known effects of airway remodeling, such as reduced cilia beating frequency and reduced ciliated cell density, the loss of tissue-wide cilia polarity (FIG. 98B) and loss of tissue-wide flow straightness. See, FIG. 98A-C.

A. Microphysiological Platform

Primary human epithelial airway cells are used to engineer fully differentiated, pseudostratified, airway epithelium formed at an air-liquid interface. For example, microphysiological platforms may include, but are not limited to, a microfluidic platform or a trans-well platform. In one embodiment, the platforms further comprise an optically clear side wall to allow for the optical imaging from both the top and the side. As such, either platform is well suited for studying real-time human mucociliary transport from a top-view perspective. Using such platforms, embodiments of the present invention contemplate methods for measuring ciliary biomarkers including, but not limited to, vertical flow transport, shape and angle of the ciliary beat cycle, and cilia polarity. It has been reported that such biomarkers are related to various diseases. Quinn et al., “Automated identification of abnormal respiratory ciliary motion in nasal biopsies” Sci. Transl. Med. 7:299ra124 (2015); Thomas et al., “Ciliary dysfunction and ultrastructural abnormalities are features of severe asthma” J. Allergy Clin. Immunol. 126:722-729.e2 (2010) and Rutland et al., “Random Ciliary Orientation” N. Engl. J. Med. 323:1681-1684 (1990).

B. Optical Acquisition

A range of optical setups for capturing cilia mechanics in live tissues have been described including, but not limited to, digital high-speed imaging, photomultipliers or photodiodes. Chilvers et al., “Analysis of ciliary beat pattern and beat frequency using digital high speed imaging: comparison with the photomultiplier and photodiode methods” Thorax 55:314-317 (2000). Despite advances in automated downstream analysis, few studies discuss illumination and filtering of the image during acquisition. In one embodiment, the present invention contemplates methods comprising optimizing and standardizing an incidence angle and optical filtering of a light source for increasing contrast, clarity and data content extraction amount. See, FIG. 99. In one embodiment, the present invention contemplates a modified filter design that is configured to be attached to a phase contrast microscope in conjunction with an integrated monochrome high-speed camera.

C. Video-Processing Algorithms

The current use of automated video processing for the unbiased and large-scale analysis and screening of ciliated tissue mechanics permitted a preliminary study of tissue-scale ciliary transport phenomena. Faubel et al., “Cilia-based flow network in the brain ventricles” Science 353:176-178 (2016). Using commonly used software (e.g., Matlab® and Python®), these algorithms (for example, Fast Fourier Transform for frequency measurements) have been improved upon and combined with novel algorithms. In one embodiment, the combined algorithms comprise a pre-processing step which reduces noise and enhances the measurability of the ciliary biomarkers. Petrou et al., Image Processing: The Fundamentals. (John Wiley & Sons, Ltd). Done manually in image processing software, this can be a highly variable and time-consuming process. As disclosed herein, the present invention contemplates a fully automation of all preprocessing steps, which may be facilitated through standardized acquisition techniques. In one embodiment, the image processing algorithms measure structures and motion of ciliary beat and fluid transport from pre-processed video recordings. In one embodiment, the algorithms analyze motion detection, edge detection, particle tracking, Fourier Transform, particle image velocimetry, and orientation-sensitive algorithms. See, FIG. 101; Nixon, M., “Feature Extraction and Image Processing for Computer Vision” Third Edition. (Academic Press, 2012); Nawroth et al., “A tissue-engineered jellyfish with biomimetic propulsion” Nat. Biotechnol. 30:792-797 (2012); and Thielicke et al., “PIVlab—Towards User-friendly, Affordable and Accurate Digital Particle Image Velocimetry in MATLAB” J. Open Res. Softw. 2 (2014).

As discussed above, automated algorithms have quantified tissue-level density of beating cilia, planar beat polarity, and beat frequency. In one embodiment, the present invention contemplates a method for automated extraction of additional metrics and biomarkers related to mucociliary transport. For example, these biomarkers include, but are not limited to, single cell cilia kinematics, beat angle, cilia polarity, beat coordination; flow properties, including horizontal and vertical flow velocity, straightness, and circulation; and tissue-level kinematics, including metachronal wave length and directionality. Guo et al., “Cilia beating patterns are not hydrodynamically optimal” Phys. Fluids 26: 091901 (2014); Ding et al., “Mixing and transport by ciliary carpets: a numerical study” J. Fluid Mech. 743:124-140 (2014); Wong et al., “Nature of the mammalian ciliary metachronal wave” J. Appl. Physiol. Bethesda Md. 1985 75:458-467 (1993); Smith et al., “Modelling mucociliary clearance” Respir. Physiol. Neurobiol. 163:178-188 (2008); Eloy et al., “Kinematics of the Most Efficient Cilium” Phys. Rev. Lett. 109:038101 (2012) and Sanderson et al., “Quantification of ciliary beat frequency and metachrony by high-speed digital video” Methods Cell Biol. 47:289-297 (1995). Representative mechanical biomarkers of mucociliary transport at multiple scales are summarized below. (Table 2)

TABLE 2
Exemplary list of biomechanical biomarkers of mucociliary transport.

| Cilia and Cell | Tissue-Level | |
|---|---|---|
| Level (1-50 μm) | (50-1000 μm) | Flow (>100 μm) |
| Ciliary beat frequency | Ciliated cell density | Vertical flow velocity |
| Cilia polarity | Ciliated cell distribution | Horizontal flow velocity |
| Ciliary beat angle | Planar beat polarity | Vertical flow circulation |
| Ciliary beat | Metachronal wave | Horizontal flow |
| coordination | length | straightness |
| Cilia length | Metachronal wave | |
| | directionality | |

D. Biomarker Analysis

In one embodiment, the present invention contemplates a method for analyzing ciliary biomarker measurements. In one embodiment, the analysis comprises a statistical analysis. In one embodiment, the biomarker measurements are derived from samples of healthy patients. In one embodiment, the biomarker measurements are derived from samples of diseased patients. In one embodiment, the diseased patient exhibits at least one symptom of a respiratory disease. In one embodiment, the method is performed using a pseudostratified tissue layer cultured with a human small airway microphysiological system. In one embodiment, the method measures the physiological variability and distribution of each biomarker. McDonald, J. H. Handbook of Biological Statistics. (Sparky House Publishing, 2014).

The effects of disease may be determined by measuring the same metrics in diseased tissues and healthy tissues and computing a percent change compared to the physiological mean value. For example, color-coded biomarker profiles can summarize and quantify the differences to the reference data. Alternatively, statistical significance of these differences may be determined by using a statistical test, for example, a multiple pairwise Student's T-test followed by Bonferroni-Holm correction. Armstrong, R. A., “When to use the Bonferroni correction” Ophthalmic Physiol. Opt. 34:502-508 (2014).

The following examples are exemplary methods that may find use for providing embodiments of healthy and disease models, as described herein. Such models may be used for testing drugs, e.g. test substances, such as treatment substances by Vertix, as described herein.

### Example I

### High Concentration Collagen Gels

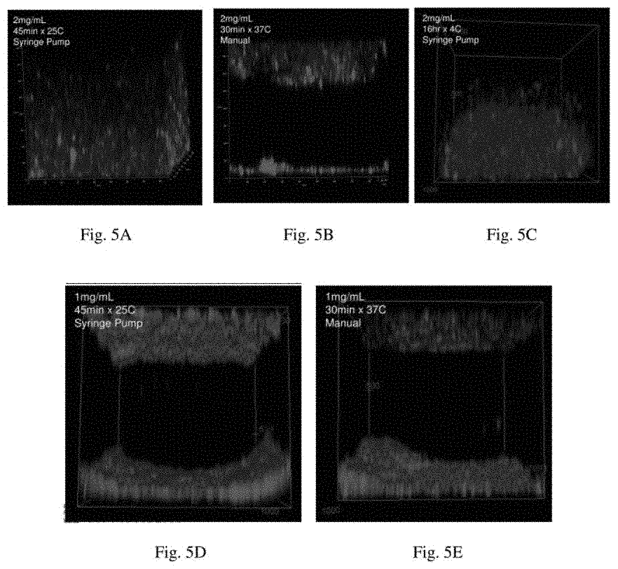

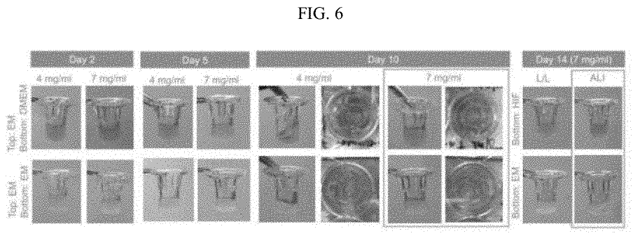

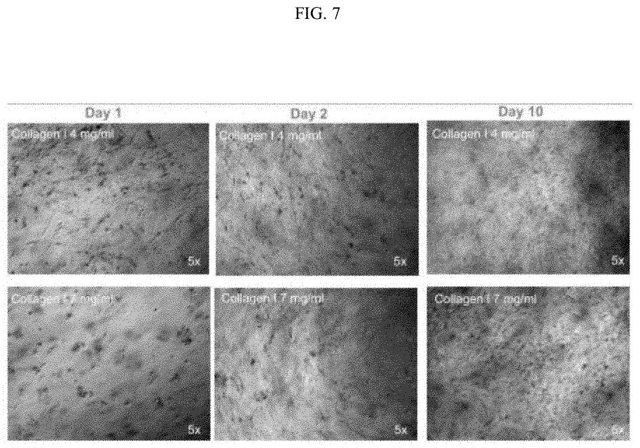

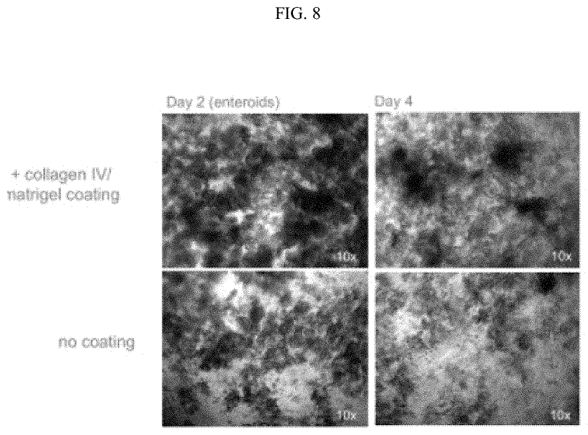

This example provides exemplary embodiments for coating chips using concentrations of collagen that are higher than indicated in previous methods. This method is exemplified in the thin 3D gels presented in FIG. 5A-E.

The following materials were used: Collagen I, including but not limited to bovine collagen I (e.g. from Advanced Biomatrix), Rat tail Collagen I, etc.; Fibronectin; Liver specific ECM, (e.g. TissueSpec® Matrix Hydrogel from Xylyx Bio East River BioSolutions, Inc., 760 Parkside Avenue, Brooklyn, N.Y., 11226 (formerly East River BioSolutions, formerly Matritek); another example from Xylyx Bio East River BioSolutions, Inc. is NativeCoat™ ECM Surface Coating; and an agent for increasing cross-linking, e.g. calcium-independent microbial transglutaminase (mTG), i.e. increasing cross-linking of at least one ECM component, including gels, at a lower concentration than without the use of the cross-linking agent.

In some preferred embodiments, said cross-linking is s calcium-independent microbial transglutaminase (mTG). In some embodiments, said mTG is added to a gel solution in the range of 1.5 mg/ml to 6 mg/ml. In some preferred embodiments, said mTG is added to a gel solution at 4 mg/ml in order to increase cross-linking of at least one gel component.

### Example II

### Hydrogel Preparation

Hydrogel preparation steps vary depending on whether cells are to be cultured on the surface or encapsulated within hydrogels, carefully select the appropriate protocol. TissueSpec® Matrix Hydrogel components include Matrix, Component A and Component B. Matrix is specific to a particular organ, such as liver. Thaw all components to 4° C. prior to use. Mix thoroughly by pipetting up and down between each step. Avoid introducing bubbles. Below are instructions to prepare 1 mL of TissueSpec® Matrix Hydrogel at a concentration of 6 mg/ml.

Check the pH of TissueSpec® matrix hydrogel preparations prior to adding your cells. pH values should range from 7.2-7.4 for cell viability and attachment. (East River Biosolutions-Rev. 6 Jun. 2018).

1. Matrix Hydrogels Cell Culture Preparations

Step 1. Working on ice, add 60 μL Component A to 600 μL Matrix and mix thoroughly by pipetting up and down. Avoid introducing bubbles.

Step 2. Add 70 μL Component B and mix thoroughly by pipetting up and down. Avoid introducing bubbles.

Step 3. Add 270 μL cell culture media to yield a final hydrogel concentration of 6 mg/mL. For other hydrogel concentrations, adjust by varying the volume of cell culture media.

Step 4. Add hydrogel mixture to the cell culture substrate (e.g., multi-well plate, petri dish) according to the experimental setup. Recommendation is approximately 150 μL/cm2. Refer to the Appendix for suggested volumes for multi-well formats. (East River BioSolutions, June 2018).

Step 5. Incubate at 37° C. in a humidified environment with 5% CO2 for 45 minutes to achieve gelation.

Note: A cell suspension at the desired concentration can be prepared at this time.

Step 6. After gelation, gently add cell suspension onto surface of the TissueSpec® Matrix Hydrogel.

Step 7. Culture cells according to standard cell culture protocols.

When replacing cell culture media, gently tilt multi-well plate, place pipette tip at the bottom edge of the well, and carefully aspirate cell culture media while ensuring hydrogel remains intact at the bottom of the well.

2. Matrix Hydrogel Encapsulation

Harvest or passage cells and prepare 270 μL cell suspension at a known desired cell concentration prior to hydrogel preparation. Optimization may be required.

Step 1. Working on ice, add 60 μL Component A to 600 μL Matrix and mix thoroughly by pipetting up and down. Avoid introducing bubbles.

Step 2. Add 70 μL Component B and mix thoroughly by pipetting up and down. Avoid introducing bubbles.

Step 3. Add 270 μL cell suspension to yield a final hydrogel concentration of 6 mg/mL.

Preparation of TissueSpec® Matrix Hydrogels at 6 mg/mL, final hydrogel concentration can be adjusted by varying the volume of cell suspension.

Step 4. Add hydrogel mixture containing cells to the cell culture substrate (e.g., multi-well plate, petri dish) according to your experimental setup. ˜150 μL/cm².

Step 5. Incubate at 37° C. in a humidified environment with 5% CO 2 for 45 minutes to achieve gelation and encapsulate cells within hydrogel.

Step 6. After gelation, gently add cell culture media onto TissueSpec® Matrix Hydrogel.

When replacing cell culture media, gently tilt multi-well plate, place pipette tip at the bottom edge of the well, and carefully aspirate cell culture media while ensuring hydrogel remains intact at the bottom of the well. For gene expression analysis, hydrogels can be dissociated with collagenase prior to proceeding with standard RNA isolation protocols. Please visit eastriverbio.com for detailed Supporting Protocols.

### Example IV

### Activating a Substrate Surface

Activation as previously established: sulfo-SANPAH diluted in buffer (5 mg/ml) Sulfo-SANPAH should be used with non-amine-containing buffers at pH 7-9 such as 20 mM sodium phosphate, 0.15M NaCl; 20 mM HEPES; 100 mM carbonate/bicarbonate; or 50 mM borate. Tris, glycine or sulfhydryl-containing buffers should not be used. 15 min in a nail star UV lamp. ECM: COL-I (100 ug/ml) and FN (25 ug/ml) on both top and bottom channel. Incubation: 2 h to overnight at 37° C.Sulfo-SANPAH (sulfosuccinimidyl-6-[4′-azido-2′-nitrophenylamino]hexanoate) having the formula of:

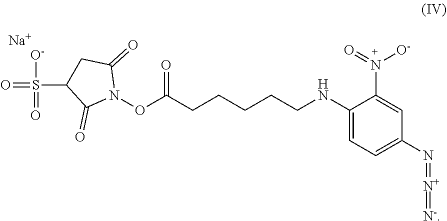

By way of example, sulfosuccinimidyl 6-(4′-azido-2′-nitrophenyl-amino) hexanoate or “Sulfo-SANPAH” (commercially available from Pierce) is a long-arm (18.2 angstrom) crosslinker that contains an amine-reactive N-hydroxysuccinimide (NHS) ester and a photoactivatable nitrophenyl azide.

Steps. Surface Activation with sulfo-SANPAH Solution (light and time sensitive):
a. Turn off light in biosafety hood.
b. Allow vial of sulfo-SANPAH powder to fully equilibrate to ambient temperature (to prevent condensation inside the storage container, as reagent is moisture sensitive).
c. Reconstitute the sulfo-SANPAH powder with a buffer solution.
d. Add 10 ml buffer into a 15 ml conical covered with foil.
e. Take 1 ml buffer from above conical and add to sulfo-SANPAH (5 mg) bottle, pipette up and down to mix thoroughly and transfer to same conical.
f. Repeat 3-5 times until all sulfo-SANPAH is fully mixed.

Sulfo-SANPAH Solution is single use only, discard immediately after finishing Chip activation, solution cannot be reused.
g. Wash channels.
h. Inject 200 μl of 70% ethanol into each channel and aspirate to remove all fluid from both channels.
i. Inject 200 μl of Cell Culture Grade Water into each channel and aspirate to remove all fluid from both channels.
j. Inject 200 μl of buffer used for the sulfo-SANPAH Solution into each channel and aspirate to remove all fluid from both channels.
k. Inject sulfo-SANPAH Solution (in buffer) in both channels.
l. Use a P200 and pipette 200 μl to inject sulfo-SANPAH Solution into each channel of each chip (200 μl should fill about 3 Chips (Both Channels)).
m. Inspect channels by eye to be sure no bubbles are present. If bubbles are present, flush channel with sulfo-SANPAH Solution until bubbles have been removed.
n. UV light activation of sulfo-SANPAH Solution.
o. Place Chips into UV light box.
p. UV light treat Chips for 20 min.
   While the Chips are being treated, prepare ECM Solution.
q. After UV treatment, gently aspirate sulfo-SANPAH Solution from channels via same ports until channels are free of solution.
r. Carefully wash with 200 μl of buffer used for the sulfo-SANPAH Solution through both channels and aspirate to remove all fluid from both channels.
s. Carefully wash with 200 μl of sterile DPBS through both channels.
t. Carefully aspirate PBS from channels and move on to: ECM-to-Chip.

### Example V

### Extracellular Matrix Preparation

Step 1. Calculate total volume of ECM solution needed to coat Chips. Volume required per Chip=50 μl/Channel.

Step 2. ECM diluent: PBS, prepared on ice.

Top Channel Coating: 50 μl Collagen IV (200 μg/ml) and Matrigel (100 μg/ml).
Bottom Channel Coating: 50 μl Collagen IV (200 μg/mi) and Fibronectin (30 μg/mi).

### Stock Concentrations

Collagen IV: 1 mg/ml (200 μl aliquots in −20° C.).
Fibronectin: 1 mg/ml (50 μl aliquots in 4° C.).
Matrigel®: 10 mg/ml (200 μl aliquots in −20° C.).

### Working Concentrations

Collagen IV: 200 ug/ml. Fibronectin: 30 ug/ml.

### ECM Channel Loading

Step 1. Place Chips in hood.
Step 2. Pipette 50 μl of Top Channel Coating into Top Channel—keep the pipette plunger depressed until you see fluid come out opposite end of the channel, then take another pipette tip (200 μl tip) to close the outlet port.

a. Once closed off, carefully remove the pipette tip, leaving the tip in the inlet port.

b. Aspirate excess fluid from the surface of Chip (avoid direct contact with the port).

Step 3. Repeat Step 2 but with Bottom Channel Coating into Bottom Channel.
Step 4. Incubate at 37° C. for a minimum of 2 hours up to overnight.

### Underlay Protocol

Prepare Collagen I, with or without Fibronectin:
In ICE: Using COLD TIPS (1 ml tips at the −20° C. freezer) add Collagen I (Advanced Biomatrix) to cold (in ice) cell culture media (tested in WEM, DMEM and Advanced DMEM media) containing 2% FBS (fetal bovine serum) so we have a final concentration of 0.5 mg/ml of Collagen I. Final pH most be check and if need must be adjusted using NaOH 10M to reach pH 7.0 to 7.4.

### Add Gel

Step 1. Load Bottom channel with media and leave tip in. Bottom channel most be “blocked” with tips containing media other wise the gel of the top channel will move to the bottom channel.
Step 2. Leave an empty 200 ul tip in 1 side of the top channel.
Step 3. Using a cold 200 uL tips add 80 ul of the underlay ECM to the top channel of the tall channel chip and leave the tip connected to the channel.
Step 4. Incubator for 4 hours to Overnight. Longer the incubation better (more homogenous and flat) will be the gel.

### Add Cells

Live cells can be added to the underlay gel.
Stellate cells preparation: Final 1.10⁴ to 1.10⁵ cells/ml of gel
ECM containing cell preparation:
Step 1 Cold media
Step 2 Collagen or other ECM
Step 3 Cold media containing cells.

Mix well with a pipette. Avoid adding air bubbles to the mixture.

### Example VI

### Cell Layering on Gel Underlays/Overlays

This example provides embodiments for layering cells on top of gel underlays and/or under gel overlays. In one embodiment, cells are layered on top of gel underlays. In one embodiment, cells are covered with a gel overlay, i.e. located under a gel overlay. For one nonlimiting example, a gel may be located (as an underlay) under hepatocytes

Day 0 of Coating Device Surface (after Chip Coating with Sulfo-SANPAH in Buffer:

### Day 0: Cells Seeding in the Top of the Underlay Gel.

Before cells seeding on the top of the both channel (top and bottom should be washed 2× using warm media (same cell culture media that will be used for seeding cells in that channel). After washing, the channels are ready for cell seeding. Keep chips with media on all times (do not let it any of the channels thy). If underlay gels contain cells also keep the chips at 37° C. and avoid out of the incubator time.

Cells may be seeded as previously established for the tall channel chip. In one embodiment, hepatocytes are seeded 3.5 ml/ml, using a 35 ul per channel chip.

### Day 1: Overlay Using Gel Protocol

In ICE: Using COLD TIPS (1 ml tips prepared in a −20° C. freezer) add Collagen I (Advanced Biomatrix) to cold (in ice) cell culture media (e.g. WEM, DMEM and Advanced DMEM media, etc.) containing for example, 2% FBS (fetal bovine serum) providing a final concentration of 0.5 mg/ml of Collagen I. pH is then checked for a pH target range from 7.0 to 7.4. For adjusting the pH to be in the target range, NaOH 10M is used. Gel is then added to the chip:

Step 1—Load Bottom channel with media and leave tip in. Thus, a bottom channel is “blocked” with tips containing media otherwise the gel of the top channel will move to the bottom channel.
Step 2—Leave an empty 200 μl tip in one side of the top channel.
Step 3—Using cold 200 μl tips, add 80 μl of the underlay ECM to the top channel of the tall channel chip and leave the tip connected to the channel.
Step 4—Incubator for 4 hours to overnight. Longer incubation times provide a more homogenous and flat) gel.

### Day 2

Second overlay option—In one embodiment, Matrigel® may be added as a second overlay option (e.g. on the top of the first overlay). F or one example, Matrigel® is added at 0.25 mg/ml in media containing 2% FBS.

### Example VII

### Partial Gel Layer Preparation

This example outlines the collagen gel solution and coating steps to create a thin, partial gel layer on a membrane on a top channel.

### 1. Reagents

1.1 ER1

1.2 ER2

1.3 70% Ethanol

1.4 PBS

1.5 PBS++

1.6 10×DMEM

1.7 1N NaOH

1.8 Collagen I (BD Biosciences #A1048301)

1.9 Ice

1.10 Reconstitution buffer

### 2. Procedure

2.1 Incubation of chips
2.1.1 For ideal results, incubate chips in a humidified tissue culture incubator at 37° C., 5% CO2 for 24-48 hours prior to seeding cells

2.2 ER1 treatment for the chips
2.2.1 Wash chips with ethanol, ER2
2.2.2 Add ER1 and UV for 20 min
2.2.3 Wash with ER2 then PBS
2.2.4 Leave PBS in the channel until ready to add ECM

2.3 Preparation of gel solution (1 mg/mL Rat Tail Collagen I)
2.3.1 Start with 878.6 ul PBS++ on ice in an Eppendorf tube
2.3.2 Add 9.8 ul of each 10×DMEM and Recon Buffer
2.3.3 Add 3.9 ul of 1 N NaOH and check pH to be a little over 8
2.3.4 When ready to use, add 97.94 ul of Collagen I and mix well—note: check collagen concentration, current is 10.21 mg/mL. Check pH to be 7-7.5

2.4 Chip Coating
2.4.1 Aspirate PBS from the channels and pipette PBS++ into the bottom channel
2.4.2 Slowly add collagen 1 solution into the top channel, to avoid bubbles in the channels
2.4.3 Allow the gel to crosslink at room temperature for 45 minutes
2.4.4 Add a 200 ul pipette tip to the outlet port of the top and bottom channels
2.4.5 Using a 200 ml pipette tip filled with PBS++, starting with the top channel, pipette in very slowly (7 sec per channel). Once gel ejects into the outlet pipette tip, stop flushing and release the inlet pipette tip to allow gravity to enable flow across the channel.

### Example, VIII

### Preparation of Stained Cells

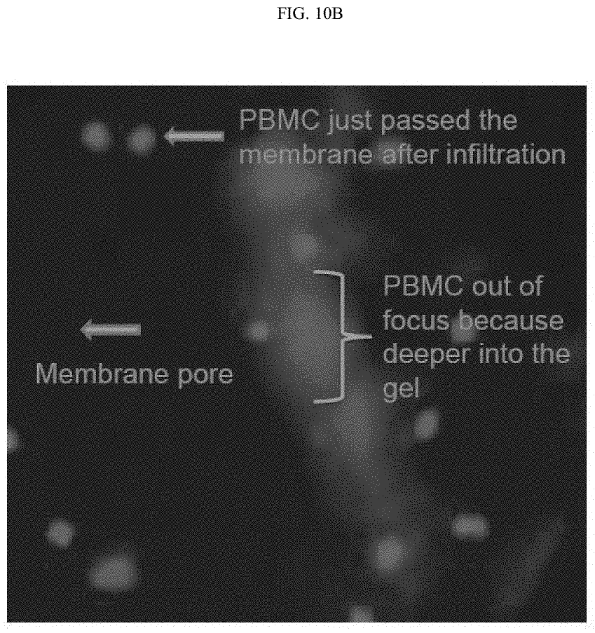

This example describes the staining of cells prepared on a partial gel layer (1:1:8) using rat tail collagen (8-11 mg/ml), 10× reconstitution buffer and 10×EMEM buffer. See, FIG. 10A-B.

A microfluidic channel was overlaid with a partial gel layer and seeded with epithelial cells (HSAEC; LifeLine) and cultured for a few weeks until the cells differentiated using ALI medium. Once differentiated, the cells were either infected with HRV for 24 hours or placed in contact with IL-13 and CXCL2.

PMN polymorphonucleates (e.g., PBMCs) were isolated using percol 65%. Then the PBMCs were stained with CellTracker Red® in RMPI 1640 buffer+10% FBS and perfused (using a pump) overnight to allow for gel penetration. The flow was then removed to allow the stain to settled by gravity.

Before imaging, the cells were fixed and stained with DAPI.

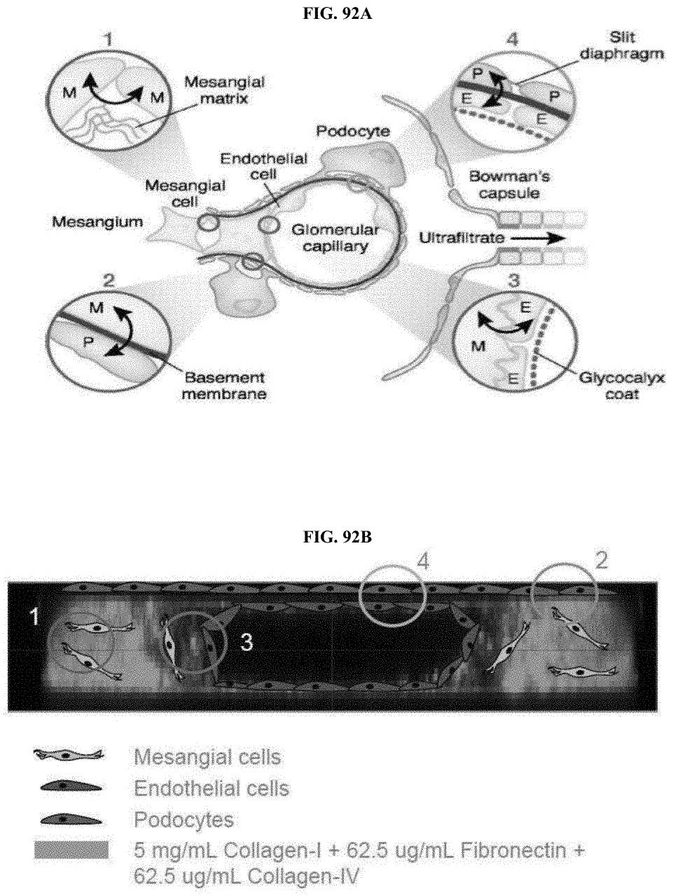

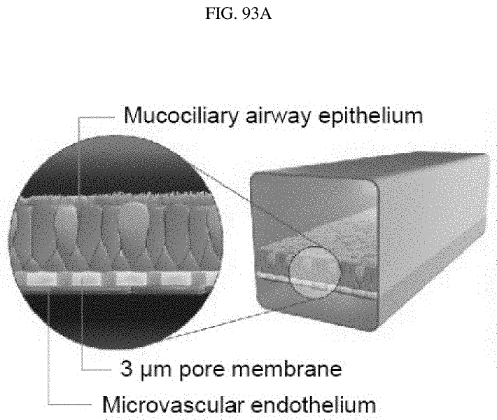

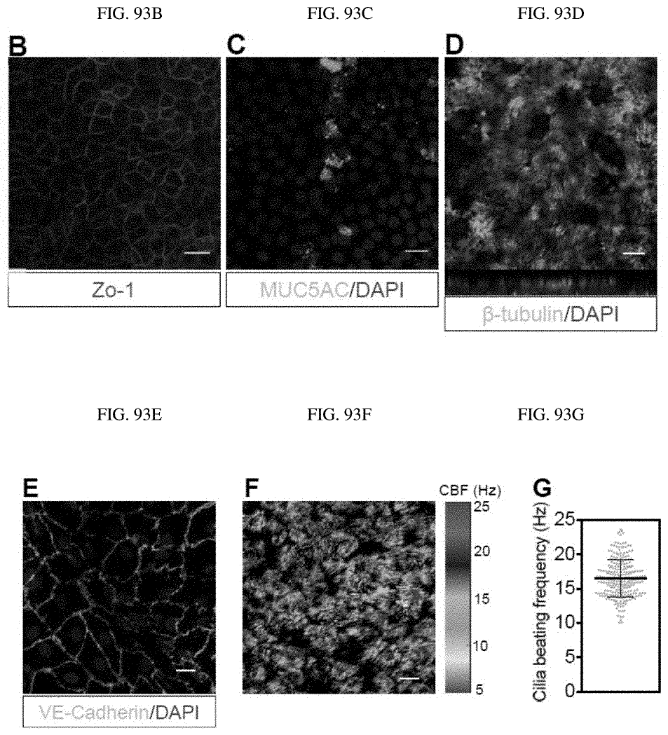

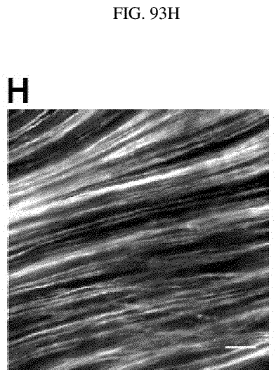

The following Examples I-III relate to FIGS. 93A-H-105A-B.

### Example I

### A Mucociliary Bronchiolar Epithelial Model

A microfluidic device comprising a channel separated by a membrane with increased pore size (3.0 μm vs 0.4 μm) was fabricated by previously published methods. This device allows immune cells to transmigrate from a vascular microchannel to a ciliated epithelial lumen and thus replicate inflammatory infiltrates. Human primary airway epithelial cells (hAECs) were cultured and differentiated at an air liquid interface (ALI) for 21 days on top of the collagen-coated 3 μm pore membrane while differentiation medium was continuously perfused at 60 μL/h through the bottom channel. Human primary endothelial cells were then seeded on the opposite side of the membrane and cultured under similar flow rate until they become confluent to create a tissue-tissue interface. FIG. 93A.

Establishment of a well-differentiated mucociliary bronchiolar epithelium on one side of the 3 μm pore membrane and a confluent pulmonary microvascular endothelium on the opposite side was confirmed by immunofluorescence (hereinafter referred to as “Airway Chips”). FIGS. 93B-F.

Using high-speed, real-time microscopy, cilia were observed to be actively beating in a synchronized fashion at a frequency of 16.35 (±2.6) Hz. FIG. 93F, FIG. 93G. This ciliary beating pattern generated a regional unidirectional mucociliary transport visualized by recorded trajectories of rapidly moving fluorescent microbeads diluted in PBS and introduced in the top channel. FIG. 93H. Supporting these observations were observations of tumbling small plugs of cell debris trapped in mucus. The measured bead velocity was typically ˜100 μm/sec, which is strikingly close to that reported in human airways. See, Table 3.

TABLE 3
Comparison of structure and function between a human airway
epithelium in vivo and the human Airway Chip.

| Parameters | Human airway | Airway Chip (SD) |
|---|---|---|
| Mucociliary velocity | 40-150 μm/s | 103.5 μm/s (±46.1) |
| Cilia beating frequency (Hz) | 9-20 Hz | 16.35 (±2.6) |
| Ciliated cells (%) | ~30 | 29.3 (±1.9) |
| Goblet cells (%) | ~10-15 | 18.4 (±1.2) |
| Basal cells (%) | ~6-30 | 10.4 (±3.8) |

### Example II

### Effect of IL-13 on Ciliated Cell Beating Frequency

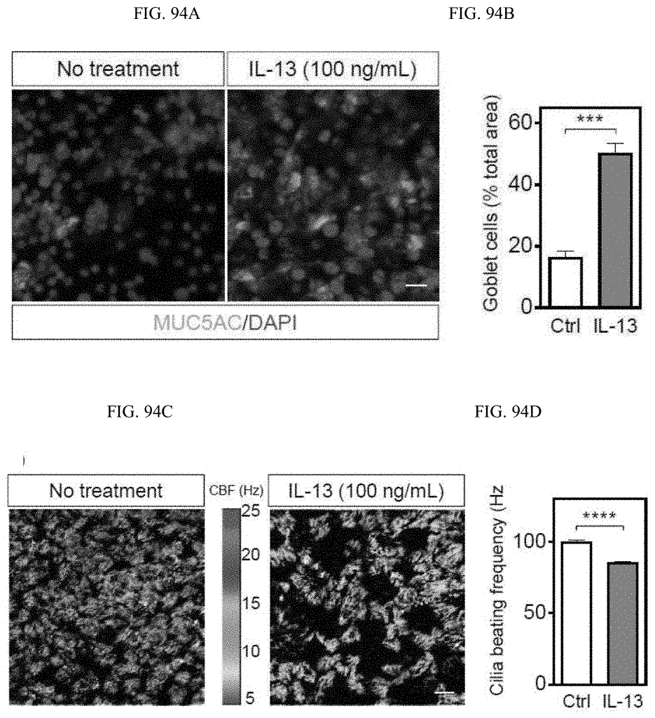

A microfluidic device comprising a mucociliary bronchiolar epithelium layer, prepared in accordance with Example I, was contacted with IL-13 (100 ng/mL) for 7 days. The data showed a significant increase in the number of goblet cells (16.5% total area vs 50.3%; p<0.01). FIG. 94A, 94B. The data also showed a decrease in cilia beating frequency (14.4% reduction; p<0.001). FIG. 94C, 94D.

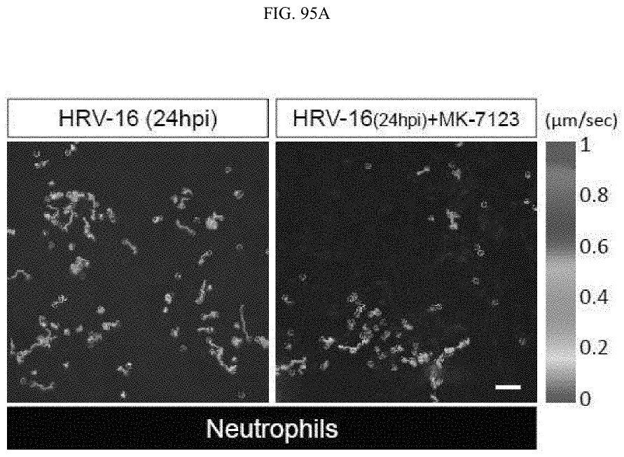

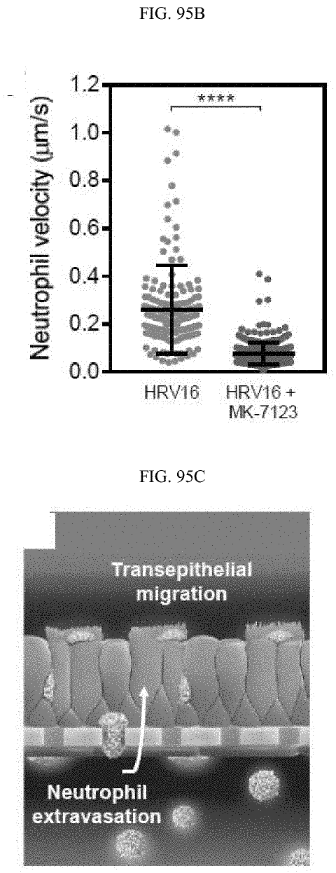

This response was accompanied by a reduction in neutrophil velocity. FIG. 95A, 95B. Also observed was a significant twofold increase in neutrophil recruited between HRV infected chips and infected chips in presence of IL-13 (953 vs 1999; p<0.001). FIG. 95A. Real time fluorescence imaging of circulating human neutrophils revealed that many endothelium bound neutrophils, transmigrated from the vascular channel through the large 3 μm pores of the membrane, into the epithelium chamber where they adhered to the epithelial surface. FIG. 95C.

### Example III

### Ciliated Biological Cell Differentiation On-Chip

Cells were cultured and differentiated as previously described. Benam et al., “Small airway-on-a-chip enables analysis of human lung inflammation and drug responses in vitro”, Nat. Methods 13:151-7 (2016). Briefly, hAECs were seeded in the Airway Chips (prepared in accordance with Example I) on a human placenta collagen IV-coated 3 μm pore polyester membrane at a density of 3×10⁶ cells/mL and left to attach for 2 h. Five days post seeding, air liquid interface (ALI) was introduced and Airway chips were perfused basally at 60 μL/h for 3 weeks until full differentiation. Epithelium integrity, extensive cilia beating coverage and apical mucus secretion were used for quality control.

When epithelial differentiation was fully reached, hMVECs or HUVECs were seeded onto the opposite side of the membrane, in the vascular channel at a density of 1×10⁷ cells/mL and cultured under flow for 3-4 days in endothelial growth media (EGM2-MV, Lonza, USA). The epithelial channel of each chip was gently rinsed 5× with DMEM. After the final wash, and every 24h thereafter, the epithelial surface was washed with 50 μL of DMEM.

### VIII. Microfluidic Chips, Devices and Systems

Microfluidic chips, devices, and systems contemplated for use include but are not limited to chips described in Bhatia and Ingber, “Microfluidic organs-on-chips.” Nature Biotechnology, 32(8):760-722, 2014; U.S. Pat. No. 8,647,861, Organ mimic device with microchannels and methods of use and manufacturing thereof, herein incorporated in its entirety, for some examples.

A. Closed Top Microfluidic Chips

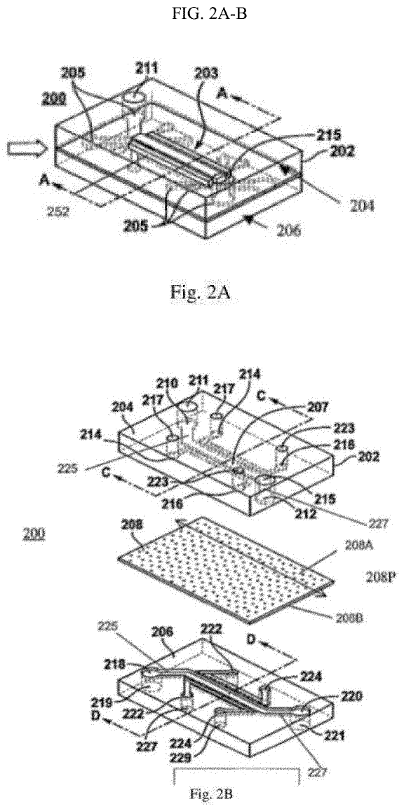

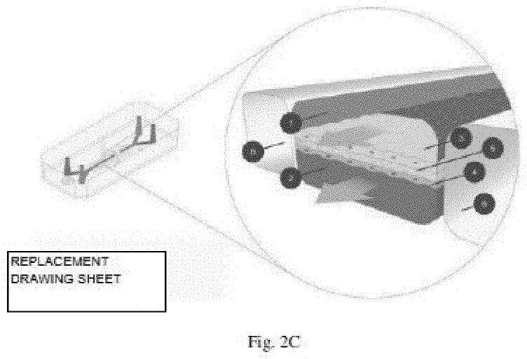

In some embodiments, the present invention contemplates a closed-top fluidic device, e.g. exemplary schematics in FIGS. 2A-C. Such closed-top microfluidic devices comprise organ-on-chips, such as fluidic devices comprising one or more cells types for the simulation of one or more of the function of organ components. In other embodiments, the present invention contemplates closed-top liver-on-chips, kidney-on-chips, e.g. proximal tubule-kidney-on-chips, lung-on-chips, etc., see, e.g. schematic in FIG. 2C. The present disclosure also relates to lymph node-on-chips, and BBB (blood brain barrier)-on-chips, which may also use a fluidic device such as depicted schematically in FIGS. 2A-C.

FIGS. 2A-B illustrates a perspective view of the devices in accordance with some embodiments described herein. For example, as shown in FIGS. 2A-2B, the device 200 can include a body 202 comprising a first structure 204 and a second structure 206. A body 202 can be made of an elastomeric material, although the body can be alternatively made of a non-elastomeric material, or a combination of elastomeric and non-elastomeric materials. It should be noted that the microchannel design 203 is only exemplary and not limited to the configuration shown in FIGS. 2A-2B.

While operating chambers 252 (e.g., as a pneumatics means to actuate the membrane 208, see the International Appl. No. PCT/US2009/050830 for further details of the operating chambers, the content of which is incorporated herein by reference in its entirety) are shown in FIGS. 2A-2B, they are not required in all of the embodiments described herein. In some embodiments, the devices do not comprise operating chambers on either side of the first chamber and the second chamber. In other embodiments, the devices described herein can be configured to provide other means to actuate the membrane, e.g., as described in the International Pat. Appl. No. PCT/US2014/071570, the content of which is incorporated herein by reference in its entirety.

In some embodiments, various organ chip devices described in the International Patent Application Nos. PCT/US2009/050830; PCT/US2012/026934; PCT/US2012/068725; PCT/US2012/068766; PCT/US2014/071611; and PCT/US2014/071570, the contents of each of which are incorporated herein by reference in their entireties, can be modified to form the devices described herein. For example, the organ chip devices described in those patent applications can be modified in accordance with the devices described herein.

The first structure 204 and/or second structure 206 can be fabricated from a rigid material, an elastomeric material, or a combination thereof. As used herein, the term “rigid” refers to a material that is stiff and does not bend easily, or maintains very close to its original form after pressure has been applied to it. The term “elastomeric” as used herein refers to a material or a composite material that is not rigid as defined herein. An elastomeric material is generally moldable and curable, and has an elastic property that enables the material to at least partially deform (e.g., stretching, expanding, contracting, retracting, compressing, twisting, and/or bending) when subjected to a mechanical force or pressure and partially or completely resume its original form or position in the absence of the mechanical force or pressure. In some embodiments, the term “elastomeric” can also refer to a material that is flexible/stretchable but does not resume its original form or position after pressure has been applied to it and removed thereafter. The terms “elastomeric” and “flexible” are interchangeably used herein. In some embodiments, the material used to make the first structure and/or second structure or at least the portion of the first structure 204 and/or second structure 206 that is in contact with a gaseous and/or liquid fluid can comprise a biocompatible polymer or polymer blend, including but not limited to, polydimethylsiloxane (PDMS), polyurethane, polyimide, styrene-ethylene-butylene-styrene (SEBS), polypropylene, polycarbonate, cyclic polyolefin polymer/copolymer (COP/COC), or any combinations thereof.

As used herein, the term “biocompatible” refers to any material that does not deteriorate appreciably and does not induce a significant immune response or deleterious tissue reaction, e.g., toxic reaction or significant irritation, over time when implanted into or placed adjacent to the biological tissue of a subject, or induce blood clotting or coagulation when it comes in contact with blood.

Additionally or alternatively, at least a portion of the first structure 204 and/or second structure 206 can be made of non-flexible or rigid materials like glass, silicon, hard plastic, metal, or any combinations thereof.

The membrane 208 can be made of the same material as the first structure 204 and/or second structure 206 or a material that is different from the first structure 204 and/or second structure 206 of the devices described herein. In some embodiments, membrane 208 was made out of polyethylene terephthalate (PET). PET membranes have limited optical clarity, thus limiting optical readouts. PET membrane pores are randomly distributed having pore diameters ranging from 3-10 microns. In some embodiments, PET membrane pores are 0.4 microns in diameter. In some embodiments, the membrane 208 can be made of a rigid material. In some embodiments, the membrane is a thermoplastic rigid material. Examples of rigid materials that can be used for fabrication of the membrane include, but are not limited to, polyester, polycarbonate or a combination thereof. In some embodiments, the membrane 208 can comprise a flexible material, e.g., but not limited to PDMS. In some embodiments, the membrane 208 is made of optically clear PDMS, having regular pore distribution of 7 μm diameter pores. Additional information about the membrane is further described below.

In some embodiments, the first structure and/or second structure of the device and/or the membrane can comprise or is composed of an extracellular matrix polymer, gel, and/or scaffold. Any extracellular matrix can be used herein, including, but not limited to, silk, chitosan, elastin, collagen, proteoglycans, hyaluronic acid, collagen, fibrin, and any combinations thereof. The device in FIG. 2A can comprise a plurality of access ports 205. In addition, the branched configuration 203 can comprise a tissue-tissue interface simulation region (membrane 208 in FIG. 2B) where cell behavior and/or passage of gases, chemicals, molecules, particulates and cells are monitored.

FIG. 2B illustrates an exploded view of the device in accordance with an embodiment. In one embodiment, the body 202 of the device 200 comprises a first outer body portion (first structure) 204, a second outer body portion (second structure) 206 and an intermediary membrane 208 configured to be mounted between the first and second outer body portions 204, 206 when the portions 204, 206 are mounted to one another to form the overall body.

The first outer body portion or first structure 204 can have a thickness of any dimension, depending, in part, on the height of the first chamber 204. In some embodiments, the thickness of the first outer body portion or first structure 204 can be about 1 mm to about 100 mm, or about 2 mm to about 75 mm, or about 3 mm to about 50 mm, or about 3 mm to about 25 mm. In some embodiments, the first outer body portion or first structure 204 can have a thickness that is more than the height of the first chamber by no more than 5 mm, no more than 4 mm, no more than 3 mm, no more than 2 mm, no more than 1 mm, no more than 500 microns, no more than 400 microns, no more than 300 microns, no more than 200 microns, no more than 100 microns, no more than 70 microns or less. In some embodiments, it is desirable to keep the first outer body portion or first structure 204 as thin as possible such that cells on the membrane can be visualized or detected by microscopic, spectroscopic, and/or electrical sensing methods.

The second outer body portion or second structure 206 can have a thickness of any dimension, depending, in part, on the height of the second chamber 206. In some embodiments, the thickness of the second outer body portion or second structure 206 can be about 50 μm to about 10 mm, or about 75 μm to about 8 mm, or about 100 μm to about 5 mm, or about 200 μm to about 2.5 mm. In one embodiment, the thickness of the second outer body portion or second structure 206 can be about 1 mm to about 1.5 mm. In one embodiment, the thickness of the second outer body portion or second structure 206 can be about 0.2 mm to about 0.5 mm. In some embodiments, the second outer first structure and/or second structure portion 206 can have a thickness that is more than the height of the second chamber by no more than 5 mm, no more than 4 mm, no more than 3 mm, no more than 2 mm, no more than 1 mm, no more than 500 microns, no more than 400 microns, no more than 300 microns, no more than 200 microns, no more than 100 microns, no more than 70 microns or less. In some embodiments, it is desirable to keep the second outer body portion or second structure 206 as thin as possible such that cells on the membrane can be visualized or detected by microscopic, spectroscopic, and/or electrical sensing methods.

In some embodiments, the first chamber and the second chamber can each independently comprise a channel. The channel(s) can be substantially linear or they can be non-linear. In some embodiments, the channels are not limited to straight or linear channels and can comprise curved, angled, or otherwise non-linear channels. It is to be further understood that a first portion of a channel can be straight, and a second portion of the same channel can be curved, angled, or otherwise non-linear. Without wishing to be bound by a theory, a non-linear channel can increase the ratio of culture area to device area, thereby providing a larger surface area for cells to grow. This can also allow for a higher amount or density of cells in the channel.

FIG. 2B illustrates an exploded view of the device in accordance with an embodiment. As shown in FIG. 2B, the first outer body portion 204 includes one or more inlet fluid ports 210 preferably in communication with one or more corresponding inlet apertures 211 located on an outer surface of the body 202. The device 100 is preferably connected to the fluid source 104 via the inlet aperture 211 in which fluid travels from the fluid source 104 into the device 100 through the inlet fluid port 210.

Additionally, the first outer body portion or first structure 204 can include one or more outlet fluid ports 212 in communication with one or more corresponding outlet apertures 215 on the outer surface of the first structure 204. In some embodiments, a fluid passing through the device 200 can exit the device to a fluid collector or other appropriate component via the corresponding outlet aperture 215. It should be noted that the device 200 can be set up such that the fluid port 210 is an outlet and fluid port 212 is an inlet.

In some embodiments, as shown in FIG. 2B, the device 200 can comprise an inlet channel 225 connecting an inlet fluid port 210 to the first chamber 204. The inlet channels and inlet ports can be used to introduce cells, agents (e.g., but not limited to, stimulants, drug candidate, particulates), airflow, and/or cell culture media into the first chamber 204.

The device 200 can also comprise an outlet channel 227 connecting an outlet fluid port 212 to the first chamber 204. The outlet channels and outlet ports can also be used to introduce cells, agents (e.g., but not limited to, stimulants, drug candidate, particulates), airflow, and/or cell culture media into the first chamber 204.

Although the inlet and outlet apertures 211, 215 are shown on the top surface of the first structure 204 and are located perpendicular to the inlet and outlet channels 225, 227, one or more of the apertures 211, 215 can be located on one or more lateral surfaces of the first structure and/or second structure such that at least one of the inlet and outlet apertures 211, 215 can be in-plane with the inlet and/or outlet channels 225, 227, respectively, and/or be oriented at an angle from the plane of the inlet and/or outlet channels 225, 227.

In another embodiment, the fluid passing between the inlet and outlet fluid ports can be shared between the first chamber 204 and second chamber 206. In either embodiment, characteristics of the fluid flow, such as flow rate, fluid type and/or composition, and the like, passing through the first chamber 204 can be controllable independently of fluid flow characteristics through the second chamber 206 and vice versa.

In some embodiments, while not necessary, the first structure 204 can include one or more pressure inlet ports 214 and one or more pressure outlet ports 216 in which the inlet ports 214 are in communication with corresponding apertures 217 located on the outer surface of the device 200. Although the inlet and outlet apertures are shown on the top surface of the first structure 204, one or more of the apertures can alternatively be located on one or more lateral sides of the first structure and/or second structure. In operation, one or more pressure tubes (not shown) connected to an external force source (e.g., pressure source) can provide positive or negative pressure to the device via the apertures 217. Additionally, pressure tubes (not shown) can be connected to the device 200 to remove the pressurized fluid from the outlet port 216 via the apertures 223. It should be noted that the device 200 can be set up such that the pressure port 214 is an outlet and pressure port 216 is an inlet. It should be noted that although the pressure apertures 217, 223 are shown on the top surface of the first structure 204, one or more of the pressure apertures 217, 223 can be located on one or more side surfaces of the first structure 204.

Referring to FIG. 2B, in some embodiments, the second structure 206 can include one or more inlet fluid ports 218 and one or more outlet fluid ports 220. As shown in FIG. 2B, the inlet fluid port 218 is in communication with aperture 219 and outlet fluid port 220 is in communication with aperture 221, whereby the apertures 219 and 221 are located on the outer surface of the second structure 206. Although the inlet and outlet apertures are shown on the surface of the second structure, one or more of the apertures can be alternatively located on one or more lateral sides of the second structure.

As with the first outer body portion or first structure 204 described above, one or more fluid tubes connected to a fluid source can be coupled to the aperture 219 to provide fluid to the device 200 via port 218. Additionally, fluid can exit the device 200 via the outlet port 220 and outlet aperture 221 to a fluid reservoir/collector or other component. It should be noted that the device 200 can be set up such that the fluid port 218 is an outlet and fluid port 220 is an inlet.

In some embodiments, the second outer body portion and/or second structure 206 can include one or more pressure inlet ports 222 and one or more pressure outlet ports 224. In some embodiments, the pressure inlet ports 222 can be in communication with apertures 227 and pressure outlet ports 224 are in communication with apertures 229, whereby apertures 227 and 229 are located on the outer surface of the second structure 206. Although the inlet and outlet apertures are shown on the bottom surface of the second structure 206, one or more of the apertures can be alternatively located on one or more lateral sides of the second structure. Pressure tubes connected to an external force source (e.g., pressure source) can be engaged with ports 222 and 224 via corresponding apertures 227 and 229. It should be noted that the device 200 can be set up such that the pressure port 222 is an outlet and fluid port 224 is an inlet.

In some embodiments where the operating channels (e.g., 252 shown in FIG. 2A) are not mandatory, the first structure 204 does not require any pressure inlet port 214, pressure outlet port 216. Similarly, the second structure 206 does not require any pressure inlet port 222 or pressure outlet port 224.

In an embodiment, the membrane 208 is mounted between the first structure 204 and the second structure 206, whereby the membrane 208 is located within the first structure 204 and/or second structure 206 of the device 200. In an embodiment, the membrane 208 is a made of a material having a plurality of pores or apertures therethrough, whereby molecules, cells, fluid or any media is capable of passing through the membrane 208 via one or more pores in the membrane 208. As discussed in more detail below, the membrane 208 in one embodiment can be made of a material which allows the membrane 208 to undergo stress and/or strain in response to an external force (e.g., cyclic stretching or pressure). In one embodiment, the membrane 208 can be made of a material, which allows the membrane 208 to undergo stress and/or strain in response to pressure differentials present between the first chamber 204, the second chamber 206 and the operating channels 252. Alternatively, the membrane 208 is relatively inelastic or rigid in which the membrane 208 undergoes minimal or no movement.

In some embodiments where the device simulates a function of a tissue, such as a lymph node, the membrane can be rigid.

The first chamber 204 and/or the second chamber 206 can have a length suited to the need of an application (e.g., a physiological system to be modeled), desirable size of the device, and/or desirable size of the view of field. In some embodiments, the first chamber 204 and/or the second chamber 206 can have a length of about 0.5 cm to about 10 cm. In one embodiment, the first chamber 204 and/or the second chamber 206 can have a length of about 1 cm to about 3 cm. In one embodiment, the first chamber 204 and/or the second chamber 206 can have a length of about 2 cm.

The width of the first chamber and/or the second chamber can vary with desired cell growth surface area. The first chamber 204 and the second chamber 206 can each have a range of width dimension between 100 microns and 50 mm, or between 200 microns and 10 mm, or between 200 microns and 1500 microns, or between 400 microns and 1 mm, or between 50 microns and 2 mm, or between 100 microns and 5 mm. In some embodiments, the first chamber 204 and the second chamber 206 can each have a width of about 500 microns to about 2 mm. In some embodiments, the first chamber 204 and the second chamber 206 can each have a width of about 1 mm.

In some embodiments, the widths of the first chamber and the second chamber can be configured to be different, with the centers of the chambers aligned or not aligned. In some embodiments, the channel heights, widths, and/or cross sections can vary along the length of the devices described herein.

The heights of the first chamber and the second chamber can vary to suit the needs of desired applications (e.g., to provide a low shear stress, and/or to accommodate cell size). The first chamber and the second chamber of the devices described herein can have the same heights or different heights. In some embodiments, the height of the second chamber 206 can be substantially the same as the height of the first chamber 204.

In some embodiments, the height of at least a length portion of the first chamber 204 (e.g., the length portion where the cells are designated to grow) can be substantially greater than the height of the second chamber 206 within the same length portion. For example, the height ratio of the first chamber to the second chamber can be greater than 1:1, including, for example, greater than 1.1:1, 1.5:1, 2:1, 2.5:1, 3:1, 3.5:1, 4:1, 4.5:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1. In some embodiments, the height ratio of the first chamber to the second chamber can range from 1.1:1 to about 50:1, or from about 2.5:1 to about 50:1, or from 2.5 to about 25:1, or from about 2.5:1 to about 15:1. In one embodiment, the height ratio of the first chamber to the second chamber ranges from about 1:1 to about 20:1. In one embodiment, the height ratio of the first chamber to the second chamber ranges from about 1:1 to about 15:1. In one embodiment, the height ratio of the first chamber to the second chamber can be about 10:1.

The height of the first chamber 204 can be of any dimension, e.g., sufficient to accommodate cell height and/or to permit a low shear flow. For example, in some embodiments, the height of the first chamber can range from about 100 μm to about 50 mm, about 200 μm to about 10 mm, about 500 μm to about 5 mm, or about 750 um to about 2 mm. In one embodiment, the height of the first chamber can be about 150 um. In one embodiment, the height of the first chamber can be about 1 mm.

The height of the second chamber 206 can be of any dimension provided that the flow rate and/or shear stress of a medium flowing in the second chamber can be maintained within a physiological range, or does not cause any adverse effect to the cells. In some embodiments, the height of the second chamber can range from 20 μm to about 1 mm, or about 50 μm to about 500 μm, or about 75 μm to about 400 μm, or about 100 μm to about 300 μm. In one embodiment, the height of the second chamber can be about 150 μm. In one embodiment, the height of the second chamber can be about 100 μm.

The first chamber and/or the second chamber can have a uniform height along the length of the first chamber and/or the second chamber, respectively. Alternatively, the first chamber and/or the second chamber can each independently have a varying height along the length of the first chamber and/or the second chamber, respectively. For example, a length portion of the first chamber can be substantially taller than the same length portion of the second chamber, while the rest of the first chamber can have a height comparable to or even smaller than the height of the second chamber.

In some embodiments, the first structure and/or second structure of the devices described herein can be further adapted to provide mechanical modulation of the membrane. Mechanical modulation of the membrane can include any movement of the membrane that is parallel to and/or perpendicular to the force/pressure applied to the membrane, including, but are not limited to, stretching, bending, compressing, vibrating, contracting, waving, or any combinations thereof. Different designs and/or approaches to provide mechanical modulation of the membrane between two chambers have been described, e.g., in the International Patent App. Nos. PCT/US2009/050830, and PCT/US2014/071570, the contents of which are incorporated herein by reference in their entireties, and can be adapted herein to modulate the membrane in the devices described herein.

In some embodiments, the devices described herein can be placed in or secured to a cartridge. In accordance with some embodiments of some aspects described herein, the device can be integrated into a cartridge and form a monolithic part. Some examples of a cartridge are described in the International Patent App. No. PCT/US2014/047694, the content of which is incorporated herein by reference in its entirety. The cartridge can be placed into and removed from a cartridge holder that can establish fluidic connections upon or after placement and optionally seal the fluidic connections upon removal. In some embodiments, the cartridge can be incorporated or integrated with at least one sensor, which can be placed in direct or indirect contact with a fluid flowing through a specific portion of the cartridge during operation. In some embodiments, the cartridge can be incorporated or integrated with at least one electric or electronic circuit, for example, in the form of a printed circuit board or flexible circuit. In accordance with some embodiments of some aspects described herein, the cartridge can comprise a gasketing embossment to provide fluidic routing.

In some embodiments, the cartridge and/or the device described herein can comprise a barcode. The barcode can be unique to types and/or status of the cells present on the membrane. Thus, the barcode can be used as an identifier of each device adapted to mimic function of at least a portion of a specific tissue and/or a specific tissue-specific condition. Prior to operation, the barcode of the cartridge can be read by an instrument so that the cartridge can be placed and/or aligned in a cartridge holder for proper fluidic connections and/or proper association of the data obtained during operation of each device. In some embodiments, data obtained from each device include, but are not limited to, cell response, immune cell recruitment, intracellular protein expression, gene expression, cytokine/chemokine expression, cell morphology, functional data such as effectiveness of an endothelium as a barrier, concentration change of an agent that is introduced into the device, or any combinations thereof.

In some embodiments, the device can be connected to the cartridge by an interconnect adapter that connects some or all of the inlet and outlet ports of the device to microfluidic channels or ports on the cartridge. Some examples interconnect adapters are disclosed in U.S. Provisional Application No. 61/839,702, filed on Jun. 26, 2013, and the International Patent Application No. PCT/US2014/044417 filed Jun. 26, 2014, the contents of each of which are hereby incorporated by reference in their entirety. The interconnect adapter can include one or more nozzles having fluidic channels that can be received by ports of the device described herein. The interconnect adapter can also include nozzles having fluidic channels that can be received by ports of the cartridge.

In some embodiments, the interconnect adaptor can comprise a septum interconnector that can permit the ports of the device to establish transient fluidic connection during operation, and provide a sealing of the fluidic connections when not in use, thus minimizing contamination of the cells and the device. Some examples of a septum interconnector are described in U.S. Provisional Application No. 61/810,944 filed Apr. 11, 2013, the content of which is incorporated herein by reference in its entirety.

A membrane 208 is oriented along a plane 208P parallel to the x-y plane between the first chamber 204 and the second chamber 206. It should be noted that although one membrane 208, more than one membrane 208 can be configured in devices which comprise more than two chambers.

A membrane separating a first chamber and a second chamber in devices described herein can be porous (e.g., permeable or selectively permeable), non-porous (e.g., non-permeable), rigid, flexible, elastic or any combinations thereof. Accordingly, the membrane 208 can have a porosity of about 0% to about 99%. As used herein, the term “porosity” is a measure of total void space (e.g., through-holes, openings, interstitial spaces, and/or hollow conduits) in a material, and is a fraction of volume of total voids over the total volume, as a percentage between 0 and 100% (or between 0 and 1). A membrane with substantially zero porosity is non-porous or non-permeable.

As used interchangeably herein, the terms “non-porous” and “non-permeable” refer to a material that does not allow any molecule or substance to pass through.

In some embodiments, the membrane can be porous and thus allow molecules, cells, particulates, chemicals and/or media to migrate or transfer between the first chamber 204 and the second chamber 206 via the membrane 208 from the first chamber 204 to the second chamber 206 or vice versa.

As used herein, the term “porous” generally refers to a material that is permeable or selectively permeable. The term “permeable” as used herein means a material that permits passage of a fluid (e.g., liquid or gas), a molecule, a whole living cell and/or at least a portion of a whole living cell, e.g., for formation of cell-cell contacts. The term “selectively permeable” as used herein refers to a material that permits passage of one or more target group or species, but act as a barrier to non-target groups or species. For example, a selectively-permeable membrane can allow passage of a fluid (e.g., liquid and/or gas), nutrients, wastes, cytokines, and/or chemokines from one side of the membrane to another side of the membrane, but does not allow whole living cells to pass therethrough. In some embodiments, a selectively-permeable membrane can allow certain cell types to pass therethrough but not other cell types.

Membrane permeability to individual matter/species can be determined based on a number of factors, including, e.g., material property of the membrane (e.g., pore size, and/or porosity), interaction and/or affinity between the membrane material and individual species/matter, individual species size, concentration gradient of individual species between both sides of the membrane, elasticity of individual species, and/or any combinations thereof.

A porous membrane can have through-holes or pore apertures extending vertically and/or laterally between two surfaces 208A and 208B of the membrane (FIG. 2B), and/or a connected network of pores or void spaces (which can, for example, be openings, interstitial spaces or hollow conduits) throughout its volume. The porous nature of the membrane can be contributed by an inherent physical property of the selected membrane material, and/or introduction of conduits, apertures and/or holes into the membrane material.

In some embodiments, a membrane can be a porous scaffold or a mesh. In some embodiments, the porous scaffold or mesh can be made from at least one extracellular matrix polymer (e.g., but not limited to collagen, alginate, gelatin, fibrin, laminin, hydroxyapatite, hyaluronic acid, fibroin, and/or chitosan), and/or a biopolymer or biocompatible material (e.g., but not limited to, polydimethylsiloxane (PDMS), polyurethane,styrene-ethylene-butylene-styrene (SEBS), poly(hydroxyethylmethacrylate) (pHEMA), polyethylene glycol, polyvinyl alcohol and/or any biocompatible material described herein for fabrication of the device first structure and/or second structure) by any methods known in the art, including, e.g., but not limited to, electrospinning, cryogelation, evaporative casting, and/or 3D printing. See, e.g., Sun et al. (2012) “Direct-Write Assembly of 3D Silk/Hydroxyapatite Scaffolds for Bone Co-Cultures.” Advanced Healthcare Materials, no. 1: 729-735; Shepherd et al. (2011) “3D Microperiodic Hydrogel Scaffolds for Robust Neuronal Cultures.” Advanced Functional Materials 21: 47-54; and Barry III et al. (2009) “Direct-Write Assembly of 3D Hydrogel Scaffolds for Guided Cell Growth.” Advanced Materials 21: 1-4, for examples of a 3D biopolymer scaffold or mesh that can be used as a membrane in the device described herein.

In some embodiments, a membrane can comprise an elastomeric portion fabricated from a styrenic block copolymer-comprising composition, e.g., as described in the International Pat. App. No. PCT/US2014/071611, can be adopted in the devices described herein, the contents of each of which are incorporated herein by reference in its entirety. In some embodiments, the styrenic block copolymer-comprising composition can comprise SEBS and polypropylene.

In some embodiments, a membrane can be a hydrogel or a gel comprising an extracellular matrix polymer, and/or a biopolymer or biocompatible material. In some embodiments, the hydrogel or gel can be embedded with a conduit network, e.g., to promote fluid and/or molecule transport. See, e.g., Wu et al. (2011) “Omnidirectional Printing of 3D Microvascular Networks.” Advanced Materials 23: H178-H183; and Wu et al. (2010) “Direct-write assembly of biomimetic microvascular networks for efficient fluid transport.” Soft Matter 6: 739-742, for example methods of introducing a conduit network into a gel material.

In some embodiments, a porous membrane can be a solid biocompatible material or polymer that is inherently permeable to at least one matter/species (e.g., gas molecules) and/or permits formation of cell-cell contacts. In some embodiments, through-holes or apertures can be introduced into the solid biocompatible material or polymer, e.g., to enhance fluid/molecule transport and/or cell migration. In one embodiment, through-holes or apertures can be cut or etched through the solid biocompatible material such that the through-holes or apertures extend vertically and/or laterally between the two surfaces of the membrane 208A and 208B. It should also be noted that the pores can additionally or alternatively incorporate slits or other shaped apertures along at least a portion of the membrane 208 which allow cells, particulates, chemicals and/or fluids to pass through the membrane 208 from one section of the central channel to the other.

The pores of the membrane (including pore apertures extending through the membrane 208 from the top 208A to bottom 208B surfaces thereof and/or a connected network of void space within the membrane 208) can have a cross-section of any size and/or shape. For example, the pores can have a pentagonal, circular, hexagonal, square, elliptical, oval, diamond, and/or triangular shape.

The cross-section of the pores can have any width dimension provided that they permit desired molecules and/or cells to pass through the membrane. In some embodiments, the pore size of the membrane should be big enough to provide the cells sufficient access to nutrients present in a fluid medium flowing through the first chamber and/or the second chamber. In some embodiments, the pore size can be selected to permit passage of cells (e.g., immune cells) from one side of the membrane to the other. In some embodiments, the pore size can be selected to permit passage of nutrient molecules. In some embodiments, the width dimension of the pores can be selected to permit molecules, particulates and/or fluids to pass through the membrane 208 but prevent cells from passing through the membrane 208. In some embodiments, the width dimension of the pores can be selected to permit cells, molecules, particulates and/or fluids to pass through the membrane 208. Thus, the width dimension of the pores can be selected, in part, based on the sizes of the cells, molecules, and/or particulates of interest. In some embodiments, the width dimension of the pores (e.g., diameter of circular pores) can be in the range of 0.01 microns and 20 microns, or in one embodiment, approximately 0.1-15 microns, or approximately 1-10 microns. In one embodiment, the pores have a width of about 7 microns.

In an embodiment, the porous membrane 208 can be designed or surface patterned to include micro and/or nanoscopic patterns therein such as grooves and ridges, whereby any parameter or characteristic of the patterns can be designed to desired sizes, shapes, thicknesses, filling materials, and the like.

The membrane 208 can have any thickness to suit the needs of a target application. In some embodiments, the membrane can be configured to deform in a manner (e.g., stretching, retracting, compressing, twisting and/or waving) that simulates a physiological strain experienced by the cells in its native microenvironment. In these embodiments, a thinner membrane can provide more flexibility. In some embodiments, the membrane can be configured to provide a supporting structure to permit growth of a defined layer of cells thereon. Thus, in some embodiments, a thicker membrane can provide a greater mechanical support. In some embodiments, the thickness of the membrane 208 can range between 70 nanometers and 100 μM, or between 1 μm and 100 μm, or between 10 and 100 μm. In one embodiment, the thickness of the membrane 208 can range between 10 μm and 80 μm. In one embodiment, the thickness of the membrane 208 can range between 30 μm and 80 μm. In one embodiment, the thickness of the membrane 208 can be about 50 μm.

While the membrane 208 generally have a uniform thickness across the entire length or width, in some embodiments, the membrane 208 can be designed to include regions which have lesser or greater thicknesses than other regions in the membrane 208. The decreased thickness area(s) can run along the entire length or width of the membrane 208 or can alternatively be located at only certain locations of the membrane 208. The decreased thickness area can be present along the bottom surface of the membrane 208 (i.e. facing second chamber 206), or additionally/alternatively be on the opposing surface of the membrane 208 (i.e. facing second chamber 204). It should also be noted that at least portions of the membrane 208 can have one or more larger thickness areas relative to the rest of the membrane, and capable of having the same alternatives as the decreased thickness areas described above.

In some embodiments, the membrane can be coated with substances such as various cell adhesion promoting substances or ECM proteins, such as fibronectin, laminin, various collagen types, glycoproteins, vitronectin, elastins, fibrin, proteoglycans, heparin sulfate, chondroitin sulfate, keratin sulfate, hyaluronic acid, fibroin, chitosan, or any combinations thereof. In some embodiments, one or more cell adhesion molecules can be coated on one surface of the membrane 208 whereas another cell adhesion molecule can be applied to the opposing surface of the membrane 208, or both surfaces can be coated with the same cell adhesion molecules. In some embodiments, the ECMs, which can be ECMs produced by cells, such as primary cells or embryonic stem cells, and other compositions of matter are produced in a serum-free environment.

In an embodiment, one can coat the membrane with a cell adhesion factor and/or a positively-charged molecule that are bound to the membrane to improve cell attachment and stabilize cell growth. The positively charged molecule can be selected from the group consisting of polylysine, chitosan, poly(ethyleneimine) or acrylics polymerized from acrylamide or methacrylamide and incorporating positively-charged groups in the form of primary, secondary or tertiary amines, or quaternary salts. The cell adhesion factor can be added to the membrane and is fibronectin, laminin, various collagen types, glycoproteins, vitronectin, elastins, fibrin, proteoglycans, heparin sulfate, chondroitin sulfate, keratin sulfate, hyaluronic acid, tenascin, antibodies, aptamers, or fragments or analogs having a cell binding domain thereof. The positively-charged molecule and/or the cell adhesion factor can be covalently bound to the membrane. In another embodiment, the positively-charged molecule and/or the cell adhesion factor are covalently bound to one another and either the positively-charged molecule or the cell adhesion factor is covalently bound to the membrane. Also, the positively-charged molecule or the cell adhesion factor or both can be provided in the form of a stable coating non-covalently bound to the membrane.

In an embodiment, cell attachment-promoting substances, matrix-forming formulations, and other compositions of matter are sterilized to prevent unwanted contamination. Sterilization can be accomplished, for example, by ultraviolet light, filtration, gas plasma, ozone, ethylene oxide, and/or heat. Antibiotics can also be added, particularly during incubation, to prevent the growth of bacteria, fungi and other undesired micro-organisms. Such antibiotics include, by way of non-limiting examples, gentamicin, streptomycin, penicillin, amphotericin and ciprofloxacin.

In some embodiments, the membrane and/or other components of the devices described herein can be treated using gas plasma, charged particles, ultraviolet light, ozone, or any combinations thereof.

Using the devices described herein, one can study biotransformation, absorption, as well as drug clearance, metabolism, delivery, and toxicity. The activation of xenobiotics can also be studied. The bioavailability and transport of chemical and biological agents across epithelial layers as in a tissue or organ, e.g., lung, and across endothelial layers as in blood vessels, such as for a BBB-on-chip, and across embodiments of skin epithelial layers for drug metabolism can also be studied. The acute basal toxicity, acute local toxicity or acute organ-specific toxicity, teratogenicity, genotoxicity, carcinogenicity, and mutagenicity, of chemical agents can also be studied. Effects of infectious biological agents, biological weapons, harmful chemical agents and chemical weapons can also be detected and studied. Infectious diseases and the efficacy of chemical and biological agents to treat these diseases, as well as optimal dosage ranges for these agents, can be studied. The response of organs in vivo to chemical and biological agents, and the pharmacokinetics and pharmacodynamics of these agents can be detected and studied using the devices described herein. The impact of genetic content on response to the agents can be studied. The amount of protein and gene expression in response to chemical or biological agents can be determined. Changes in metabolism in response to chemical or biological agents can be studied as well using devices described herein.

In some embodiments, the devices described herein (e.g., a Skin-on-Chip) can be used to assess the clearance of a test compound. For clearance studies, the disappearance of a test compound can be measured (e.g. using mass spec) in the media of the top chamber, bottom chamber, or both chambers (divided by a membrane comprising intestinal epithelial cells).

For example, in accordance to one aspect of the invention, a Skin-on-Chip drug-metabolizing performance can be measured by: i) disposing a substrate compound with known liver metabolites in the media of the top chamber, bottom chamber, or both chambers; and ii) measuring the amount of generated metabolite in the media of the top chamber, bottom chamber or both chambers (e.g. using mass spec). As is known in the art, the choice of the substrate and measured metabolite can help provide information on specific liver drug-metabolism enzymes (e.g. CYP450 isoforms, Phase II enzymes, etc.)

In some embodiments, the devices described herein (e.g., a Skin-on-Chip) can be used to assess the induction or inhibition potential of a test compound. For induction or inhibition studies a variety of tests are contemplated. For example, induction of CYP3A4 activity in the liver is one of main causes of drug-drug interactions, which is a mechanism to defend against exposure to drugs and toxin, but can also lead to unwanted side-effects (toxicity) or change the efficacy of a drug. A reliable and practical CYP3A induction assay with human hepatocytes in a 96-well format has been reported, where various 96-well plates with different basement membrane were evaluated using prototypical inducers, rifampicin, phenytoin, and carbamazepine. See Drug Metab. Dispo. (2010) November; 38(11):1912-6.

According to one aspect of the invention, the induction or inhibition potential of a test compound at a test concentration can be evaluated by: i) disposing the test compound in the media of the top chamber, bottom chamber or both chambers at the test concentration; ii) exposing the device for a selected period of time; and iii) assessing the induction or inhibition of enzymes by comparing performance to a measurement performed before the test compound was applied, to a measurement performed on a Skin-on-Chip that was subjected to a lower concentration of test compound (or no test compound at all), or both. In some embodiments, the performance measurement can comprise an RNA expression level. In some embodiments, the performance measurement comprises assessing drug-metabolizing capacity.

In some embodiments, the devices described herein (e.g., a Skin-on-Chip) can be used to identify in vivo metabolites of a test compound or agent, and optionally the in vivo ratio of these metabolites. According to one aspect of the invention, in vivo metabolites can be identified by: i) disposing a test compound or agent in the media of the top chamber, bottom chamber, or both chambers; and ii) measuring the concentration of metabolites in the media of the top chamber, bottom chamber, or both chambers. In some embodiments, the measuring of the concentration of metabolites comprises mass spectroscopy.

In some embodiments, the devices described herein (e.g., a Skin-on-Chip) can be used to identify the toxicity of a test compound or agent at a test concentration. According to one aspect of the invention, toxicity can be evaluated by i) disposing a test compound in the media of the top chamber, bottom chamber, or both chambers; and ii) measuring one or more toxicity endpoints selected from the list of leakage of cellular enzymes (e.g., lactose dehydrogenase, alanine aminotransferase, aspartate aminotransferase) or material (e.g., adenosine triphosphate), variation in RNA expression, inhibition of drug-metabolism capacity, reduction of intracellular ATP (adenosine triphosphate), cell death, apoptosis, and cell membrane degradation.

In one embodiment, the present invention contemplates a closed top organ-on-a-chip device without a gel (e.g., a bulk gel or a gel layer). In one embodiment, the closed to organ-on-a-chip device comprises: (i) a first structure defining a first chamber; (ii) a second structure defining a second chamber; and (iii) a membrane located at an interface region between the first chamber and the second chamber to separate the first chamber from the second chamber, the membrane including a first side facing toward the first chamber and a second side facing toward the second chamber, wherein the first and second chambers are enclosed. The first side of the membrane may have an extracellular matrix (ECM) composition disposed thereon, wherein the ECM composition comprises a coating layer. In some embodiments, the coating layer is an ECM layer. In one embodiment, the ECM layer is an overlay. In one embodiment the ECM layer is an underlay.

In one embodiment, the ECM layer is coated onto a surface of the membrane after functionalization of the membrane surface and before the chamber is seeded with cells. In some embodiments, when ECM is provided by living cells, the ECM coating is provided after seeding cells. In yet further embodiments, a membrane surface is coated by ECM before seeding cells, then seeded cells provide additional ECM on top of the first coating of ECM.

In one embodiment, the present invention contemplates a closed top organ-on-chip device comprising a gel. In one embodiment, the gel includes, but is not limited to, a gel layer, a bulk gel, a gel matrix and.or a hydrogel, etc. In one embodiment, the device comprises: (i) a first structure defining a first chamber; (ii) a second structure defining a second chamber; and (iii) a membrane located at an interface region between the first chamber and the second chamber to separate the first chamber from the second chamber, the membrane including a first side facing toward the first chamber and a second side facing toward the second chamber, wherein the first and second chambers are enclosed. In some embodiments, the device further comprises a gel. In some embodiments, the gel is a continuous layer. In some embodiments, the gel is a discontinuous layer (e.g., partial layer). In some embodiments, the gel is a layer of approximately the same thickness across the layer. In some embodiments, the gel has different thicknesses across the layer. In some embodiments, the gel is not layered on all surfaces of the device. In some embodiments, there are gaps in the gel layer. In some embodiments, only a first side of the membrane has a gel layer. In some embodiments, a gel is added to the first side of the membrane without an ECM layer. The first side of the membrane may have an extracellular matrix composition disposed thereon, wherein an extracellular matrix (ECM) composition comprises a coating layer. In some embodiments, an ECM coating layer is an overlay. In some embodiments, the gel layer is above the ECM coating layer. In some embodiments, the ECM coating layer is an underlay. In some embodiments, a gel layer overlays the ECM underlay.

In one embodiment, the ECM gel is added after coating a surface of the membrane, wherein the coating was made at the same time or after functionalization of the membrane surface, and before the chamber is seeded with cells. In some embodiments, when an ECM is provided by living cells, the ECM gel is added before seeding cells. In yet further embodiments, ECM gels contain cells, such that ECM gels and at least one cell type is added to a cell culture device.

Without being bound by theory, it is possible that the resulting gel is disposed on the bottom of the channel due to the effects of gravity. Alternatively, without being bound by theory, it is possible that the resulting gel is disposed along the membrane due to the porosity or texture of that region, i.e. that the membrane acts to anchor the gel during the flushing step. In preferred embodiments, a gel, such as a partial gel, is disposed along the membrane, e.g. for gelling onto the membrane surface, for providing a gel layer.

In some embodiments, texture or porosity is included in a region or surface of the channel, said texture or porosity causing the gel to be retained in that region or on that surface.

This can be very useful in preferentially controlling where the gel is disposed, which may be of value for engineering organ- or tissue level structure or controlling where cell populations reside (i.e. controlling the cytoarchitecture)”.

1. Closed Top Microfluidic Chips with a Gel Layer

In one embodiment, the present invention contemplates a closed top organ-on-chip device comprising a channel comprising a partial gel layer. Although it is not necessary to understand the mechanism of an invention it is believed that the device may be used for generating a more physiological relevant model of gastrointestinal tissue. In some embodiments, closed top organ-on-chip devices further comprises a three-dimensional (3-D) partial gel layer. In some embodiments, the partial gel layer can be formed by a process comprising (i) providing the first chamber filled with a polymerized gel matrix molecules; and (ii) flowing at least one or more pressure-driven fluid(s) through the polymerized matrix molecules to create a smooth surface partially extending through the first chamber. Thus, one or a plurality of lumens each extending through the first permeable matrix can be created.

In some embodiments as described herein, a partial gel layer can each independently comprise a hydrogel, an extracellular matrix gel, a polymer matrix, a monomer gel that can polymerize, a peptide gel, or a combination of two or more thereof. Methods to create a permeable polymer gel matrix are known in the art, including, e.g. but not limited to, particle leaching from suspensions in a polymer solution, solvent evaporation from a polymer solution, sold-liquid phase separation, liquid-liquid phase separation, etching of specific “block domains” in block co-polymers, phase separation to block-co-polymers, chemically cross-linked polymer networks with defined permeabilities, and a combination of two or more thereof.

Another example for making branched structures using fluids with differing viscosities is described in “Method And System For Integrating Branched Structures In Materials” to Katrycz, Publication number US20160243738, herein incorporated by reference in its entirety.

Regardless of the type of partial gel layers formed, cells can be attached to these partial gel layers. Thus, three-dimensional (3-D) partial gel layers may be used in several types of embodiments for closed top microfluidic chips, e.g. epithelial cells can be attached to outside, or within, the partial gel layer. In some embodiments, LPDCs may be added within, below, or on the opposite side of the partial gel layer. In some embodiments, stoma cells are added within the partial gel layer. In some embodiments, stomal cells are attached to the opposite side of the partial gel layer. In some embodiments, endothelial cells are located below the partial gel layer. In some embodiments, endothelial cells may be present within the partial gel layer.

In one embodiment, the present invention contemplates a multi-membrane organ-on-a-chip device comprising a partial gel layer on each of at least two membranes and at least three (3) channels. In one embodiment, the multi-membrane device is a lymph node organ-on-a-chip. In one embodiment, the multi-membrane device is an innervated brain-on-a-chip. U.S. Pat. No. 8,647,861, herein incorporated in its entirety. In one embodiment, the innervated brain-on-a-chip comprises a group of blood brain barrier cells. In one embodiment, the innervated brain-on-a-chip comprises a plurality of neuronal cells.

FIG. 7A illustrates a perspective view of an organ mimic device in accordance with an embodiment of the invention that contains three parallel microchannels separated by two porous membranes. As shown in FIG. 7A, the organ mimic device 800 includes operating microchannels 802 and an overall central microchannel 804 positioned between the operating microchannels 802. The overall central microchannel 804 includes multiple membranes 806A, 806B positioned along respective parallel x-y planes which separate the microchannel 804 into three distinct central microchannels 804A, 804B and 804C. The membranes 806A and 806B may be porous, elastic, or a combination thereof. Positive and/or negative pressurized media may be applied via operating channels 802 to create a pressure differential to thereby cause the membranes 806A, 806B to expand and contract along their respective planes in parallel. In fact, applying (flowing) pressurized media (fluids) across pressure differentials as used for removing bubbles from fluids within a microfluidic device, may induce unwanted effects under conditions for reducing air/gas bubble volumes.

It is nearly impossible to fill and operate microfluidic devices under bubble-free conditions. Thus, bubbles inadvertently introduced into a microfluidic system can significantly and negatively affect device operation. In addition, conditions for increasing the gas carrying capacity of fluid within a device may also negatively affect conditions within a device, especially when cells are present. When media passing by a bubble is unsaturated or under-saturated, it has the ability to take in/dissolve gas from the bubble. One can increase the amount or volume of gas that the media can consume (dissolve) by either actively removing the dissolved gas (degassing) or by increasing the fluid pressure. In one embodiment, both of these are done concurrently/simultaneously, with the increased pressure actually increasing the dissolved gas carrying capacity of the media. The greater the applied pressure, the greater the increase in media gas carrying capacity, the bigger/faster a bubble can be crushed. However, as described above, such procedures often removed overlaying gels, with or without cells, from at least the center regions of microfluidic channels. However, as described herein, the application and use of gels allowed bubble removal (crush) without significant removal of gel layers throughout the channel and especially within the middle region. Moreover, the application and use of gel layers and overlays comprising cells also allowed bubble removal (crush) without significant negative effects of cells lifting off of, and gels lifting off of, the membrane during culture time periods.

Details are provided as methods of removing bubbles from a microfluidic device where the flow is not stopped by combining pressure and flow to remove gas bubbles from a microfluidic device, in WO2018013646—REMOVING BUBBLES IN A MICROFLUIDIC DEVICE, herein incorporate by reference in its entirety. In one embodiment, gas is oxygen, nitrogen, carbon dioxide, or a mixture thereof, as typically used for culturing cells.

Briefly, in one embodiment, said microchannel is in fluidic communication with a first reservoir at a first end of said microchannel, and a second reservoir at a second end of said microchannel. In one embodiment, said first reservoir comprises fluid under a first pressure and said second reservoir comprises fluid under a second pressure, wherein said first pressure is greater than said second pressure. In one embodiment, said microchannel is in a perfusion manifold (e.g. containing the reservoirs). In one embodiment, said perfusion manifold is engaged with and in fluidic communication with a microfluidic chip. In one embodiment, said perfusion manifold comprises a skirt, said skirt comprising a side track engaging said microfluidic chip. In one embodiment, said microfluidic chip comprises one or more ports and said perfusion manifold is in fluidic communication with said microfluidic chip through said one or more ports. In one embodiment, the first pressure is greater by at least 0.5 kPa than the second pressure, where merely for one example, said first pressure is 21 kPa and said second pressure is 20 kPa. In one embodiment, said bubble is a gas bubble. In one embodiment, said gas is oxygen, nitrogen or a mixture thereof. In one embodiment, said bubble is an air bubble. In one embodiment, said flowing of fluid is at a flow rate of 40 uL/hr. In one embodiment, said flow rate is greater than 40 uL/hr. In one embodiment, said flow rate is 50 uL/hr. In one embodiment, said flow rate is between 50 and 75 uL/hr. In one embodiment, said microfluidic device comprises viable cells in said microchannel and said fluid comprises media supplied to said viable cells. In one embodiment, said media prior to step b) was degassed. In one embodiment, said media of step b) is unsaturated. In one embodiment, said media prior to step b) was not degassed. In one embodiment, step b is performed for less than one hour. In one embodiment, step b) is performed for at least one hour. In one embodiment, step b) is performed for 2 hours. In one embodiment, the method further comprises c) introducing fluid into said microchannel, wherein said fluid has not been degassed.

FIG. 7B illustrates a perspective view of an organ mimic device in accordance with an embodiment. As shown in FIG. 7B, the tissue interface device 900 includes operating microchannels 902A, 902B and a central microchannel 904 positioned between the microchannels 902. The central microchannel 904 includes multiple membranes 906A, 906B positioned along respective parallel x-y planes. Additionally, a wall 910 separates the central microchannel into two distinct central microchannels, having respective sections, whereby the wall 910 along with membranes 904A and 904B define microchannels 904A, 904B, 904C, and 904D. The membranes 906A and 906B are at least partially porous, elastic or a combination thereof.

The device in FIG. 7B differs from that in FIG. 7A in that the operating microchannels 902A and 902B are separated by a wall 908, whereby separate pressures applied to the microchannels 902A and 902B cause their respective membranes 904A and 904B to expand or contract. In particular, a positive and/or negative pressure may be applied via operating microchannels 902A to cause the membrane 906A to expand and contract along its plane while a different positive and/or negative pressure is applied via operating microchannels 902B to cause the membrane 906B to expand and contract along its plane at a different frequency and/or magnitude. Of course, one set of operating microchannels may be exposed to the pressure while the other set is not exposed to the pressure, thereby only causing one membrane to actuate. It should be noted that although two membranes are shown in the devices 800 and 900, more than two membranes are contemplated and can be configured in the devices.

In an example, shown in FIG. 7C, the device containing three channels described in FIG. 7A has two membranes 806A and 806B which are coated to determine cell behavior. In particular, membrane 806A is coated with a lymphatic endothelium on its upper surface 805A and with stromal cells on its lower surface, and stromal cells are also coated on the upper surface of the second porous membrane 805B and a vascular endothelium on its bottom surface 805C. Cells are placed in the central microchannel surrounded on top and bottom by layers of stromal cells on the surfaces of the upper and lower membranes in section 804B. Fluids, such as cell culture medium or blood, enter the vascular channel in section 804 C. Fluids, such as cell culture medium or lymph, enters the lymphatic channel in section 804A. This configuration of the device 800 allows cell growth and invasion into blood and lymphatic vessels during immune cell migration. In the example, one or more of the membranes 806A, 806B may expand/contract in response to pressure through the operating microchannels. Additionally or alternatively, the membranes may not actuate, but may be porous or have grooves to allow cells to pass through the membranes.

In some embodiments topside is referring to an upper surface of a membrane. In some embodiments, bottom side is referring to a lower surface of a membrane.

B. Open Top Microfluidic Chips

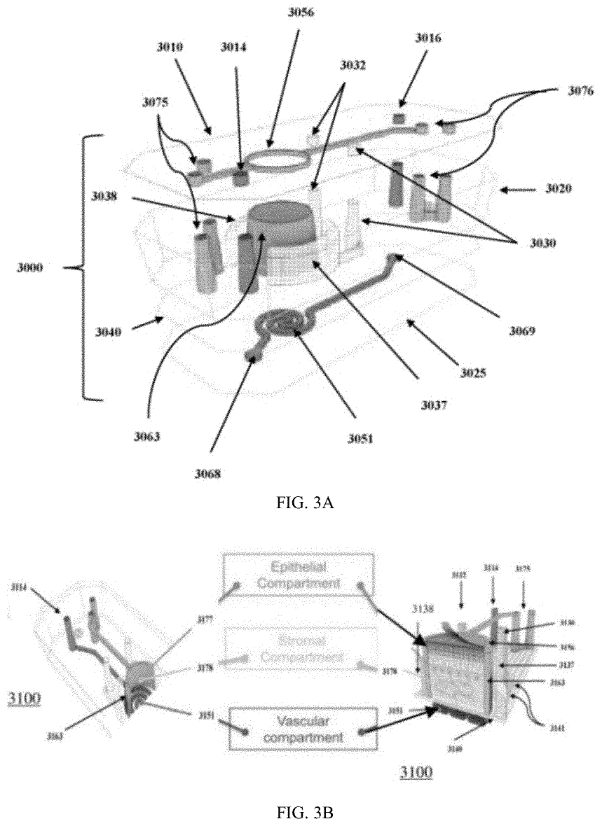

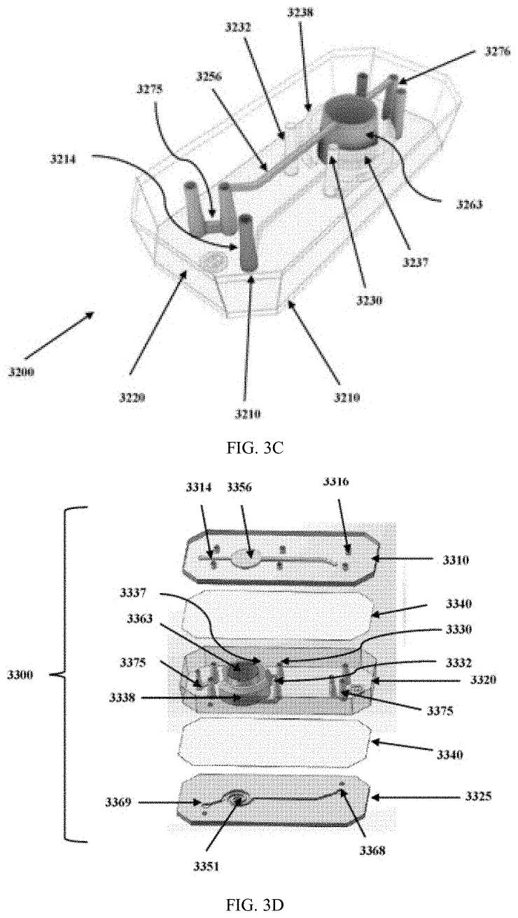

In one embodiment, the present invention contemplates open top organ-on-a-chip devices. See, e.g. schematics in FIGS. 3A-B through 4A-D. In one embodiment, the open top device comprises a skin-on-a-chip device, such as fluidic devices comprising one or more cells types for the simulation one or more of the function of skin components. U.S. Pat. No. 8,647,861 “Organ mimic device with microchannels and methods of use and manufacturing thereof” (herein incorporated by reference).

FIG. 3B shows an exemplary exploded view of one embodiment of an open-top chip device 1800, wherein a membrane 1840 resides between the bottom surface of the first chamber 1863 and the second chamber 1864 and the at least two spiral microchannels 1851. Open top microfluidic chips include but are not limited to chips having removable covers, such as removable plastic covers, paraffin covers, tape covers, etc.

In some embodiments of an open top device, it is desirable to include a cover that comprises sensors or actuators. For example, a cover can comprise one or more electrodes that can be used for measurement of electrical excitation. In some embodiments, such as where the device comprises a membrane (e.g., membrane 540), the one or more electrodes can be used to perform a measurement of trans-epithelial electrical resistance (TEER) across the cell layer. It may also be desirable to include one or more electrodes on opposite sides of the membrane 540. In some embodiments, the electrodes can be included in a bottom structure (e.g., bottom structure 525). In some embodiments, the bottom structure can be an open bottom with bottom electrodes included on a bottom cover that can be brought into contact with the bottom structure. The bottom cover may support any of the features or variations discussed herein in the context of a top cover, including, for example, removability, fluidic channels, multiple layers, clamping features, etc.

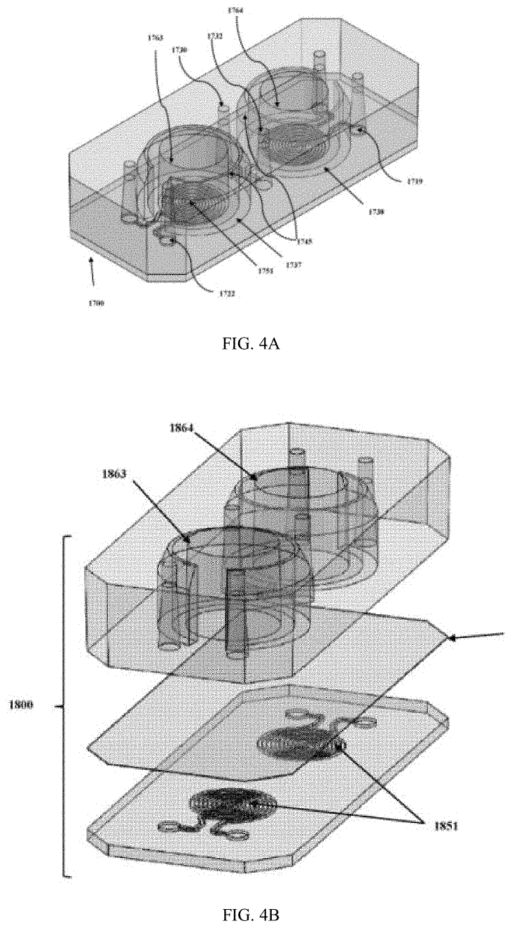

FIG. 3B shows exemplary schematic views of one embodiment of an open-top chip device in relation to exemplary cell compartments, e.g. epithelial, stromal and vascular. In one embodiment, the present invention contemplates a stretchable open top chip device 3100 comprising a chamber 3163 comprising an epithelial region 3177 and a dermal region 3178. In one embodiment, the epithelial region comprises an epithelial cell layer. In one embodiment, the dermal region comprises a dermal cell layer, wherein said epithelial cell layer adheres to the surface of the dermal cell layer. In one embodiment, the device further comprises a spiral microchannel 3151 in fluid communication with a fluid inlet port 3114, wherein the microchannel comprises a plurality of vascular cells, in one embodiment, a membrane 3140 is placed between the chamber dermal cell layer and the microchannel plurality of vascular cells. In one embodiment, the device further comprises an upper microchannel with a circular chamber 3156 connected to a fluid or gas port pair 3175. In one embodiment, the device further comprises a first vacuum port 3130 connected to a first vacuum chamber 3137 and a second vacuum port 3132 connected to a second vacuum chamber 3138. In one embodiment, the membrane 3140 comprises a PDMS membrane comprising a plurality of pores 3141, wherein said pores 3141 are approximately 50 μm thick, approximately 7 um in diameter, packed as 40 um hexagons, wherein each pore has a surface area of approximately 0.32 cm². Although it is not necessary to understand the mechanism of an invention, it is believed that the pore surface area contacts a gel layer (if present). FIGS. 4A and 4B.

FIG. 4C shows another exemplary schematic of an open top microfluidic chip showing embodiments of a stretchable open top chip device 3200. In one embodiment, the present invention contemplates a stretchable open top chip device 3200 comprising: i) a fluidic cover 3210 comprising an upper microchannel with a circular chamber 3256 configured with a first fluid or gas port pair 3275 and second fluid or gas port pair 3276; a fluid inlet port 3214, a fluid outlet port 3216, a first vacuum port 3230 and a second vacuum port 3232; ii) a top structure 3220 comprising a chamber 3263, a first vacuum chamber 3237 connected to the first vacuum port 3230, and a second vacuum chamber 3238, connected to the second vacuum port 3232, wherein the upper microchannel with a circular chamber 3256 seals with the top surface of the chamber 3263; and iii) a bottom structure 3225 layered underneath said top structure 3220. FIG. 4C.

Many of the problems associated with earlier systems can be solved by providing an open-top style microfluidic device that allows topical access to one or more parts of the device or cells that it comprises. For example, the microfluidic device can include a removable cover, that when removed, provides access to the cells of interest in the microfluidic device. In some aspects, the microfluidic devices include systems that constrain fluids, cells, or biological components to desired area(s). The improved systems provide for more versatile experimentation when using microfluidic devices, including improved application of treatments being tested, improved seeding of additional cells, and/or improved aerosol delivery for select tissue types.

It is also desirable in some aspects to provide access to regions of a cell-culture device. For example, it can be desirable to provide topical access to cells to (i) apply topical treatments with particulate matter, e.g. pigments, such as used in tattoo inks, liquid, such as pigment diluents used with tattoo inks, gaseous, solid, semi-solid, or aerosolized reagents, (ii) apply a tattoo, e.g. access for using a tattoo gun and a tattoo needle for wounding, for injecting pigments, etc., (iii) obtain samples and biopsies, or (vi) add additional cells or biological/chemical components.

Therefore, the present disclosure relates to fluidic systems that include a fluidic device, such as a microfluidic device with an opening that provides direct access to device regions or components (e.g. access to the gel region, access to one or more cellular components, etc.). Although the present disclosure provides an embodiment wherein the opening is at the top of the device (referred to herein with the term “open top”), the present invention contemplates other embodiments where the opening is in another position on the device. For example, in one embodiment, the opening is on the bottom of the device. In another embodiment, the opening is on one or more of the sides of the device. In another embodiment, there is a combination of openings (e.g. top and sides, top and bottom, bottom and side, etc.).

While detailed discussion of the “open top” embodiment is provided herein, those of ordinary skill in the art will appreciate that many aspects of the “open top” embodiment apply similarly to open bottom embodiments, as well as open side embodiments or embodiments with openings in any other regions or directions, or combinations thereof. Similarly, the device need not remain “open” throughout its use; rather, as several embodiments described herein illustrate, the device may further comprise a cover or seal, which may be affixed reversibly or irreversibly. For example, removal of a removable cover creates an opening, while placement of the cover back on the device closes the device. The opening, and in particular the opening at the top, provides a number of advantages, for example, allowing (i) the creation of one or more gel layers for simulating the application of topical treatments on the cells, tissues, or organs, or (ii) the addition of chemical or biological components such as the seeding of additional cell types for simulated tissue and organ systems. The present disclosure further relates to improvement in fluidic system(s) that improve the delivery of topicals, such as pigments, pigment diluents, such as used with tattoo inks, to simulated tissue and organ systems, such as simulated skin-tissues.

The present invention contemplates a variety of uses for these open top microfluidic devices and methods described herein. In one embodiment, the present invention contemplates a method of topically testing an agent (whether a drug, food, gas, or other substance) comprising 1) providing a) an agent and b) microfluidic device comprising i) a chamber, said chamber comprising a partial gel layer; ii) a cell layer in, on or under said gel layer, said gel layer positioned above iii) a porous membrane and under iv) a removable cover, said membrane in contact with v) fluidic channels; 2) removing said removable cover; and 3) topically contacting said cells in, on or under said gel matrix with said agent. In one embodiment, said agent is in an aerosol. In one embodiment, agent is in a liquid, gas, gel, semi-solid, solid, or particulate form. These uses may apply to the open top microfluidic chips described below and herein.

In one embodiment, the present invention contemplates an open-top chip device 1700 comprising: i) a first chamber 1763 and a second chamber 1764, wherein each chamber is surrounded by a deformable surface 1745; and ii) at least two spiral microchannels 1751 located on the bottom surface of the chambers, wherein each of the microchannels are in fluidic communication with an inlet port 1719 and an outlet port 1722 and are respectively configured with a first vacuum port 1730 or a second vacuum port 1732, such that each vacuum port is respectively connected to a first vacuum chamber 1737 or a second vacuum chamber 1738. FIG. 4A. An exploded view of the embodiment depicted FIG. 4B shows an open-top chip device 1800, wherein a membrane 1840 resides between the bottom surface of the first chamber 1863 and the second chamber 1864 and the at least two spiral microchannels 1851. FIG. 4B.

In one embodiment, the present invention contemplates a fully assembled stretchable open top microfluidic device 3600 comprising a fluidic cover 3610 comprising microfluidic channel 3608, a first vacuum port 3630 and a second vacuum port 3632, wherein the microfluidic channel 3608 terminates at either end an inlet port 3614 and an outlet port 3616, respectively.

A first cross-sectional view across plane A presents an open top microfluidic device 3700 in an assembled configuration comprising a fluidic cover 3710 attached to a membrane 3740, wherein the membrane 3740 overlays an open region 3704 (shown as hidden open region 3604) within a top structure 3720 that is attached to a bottom structure 3725. A second cross-section view across plane A presents an open top microfluidic device 3700 in a separated configuration where a fluidic top 3710 comprising a membrane 3740 is removed from top structure 3720 thereby providing access to an open region 3704, wherein a microfluidic channel 3608 is configured within the fluidic cover 3710.

A third cross-sectional view across plane A presents an open top microfluidic device 3800 in an assembled configuration comprising a fluidic cover 3810 attached to a membrane 3840, wherein the membrane 3840 overlays an open region 3804 (shown as hidden open region 3604) within a top structure 3820 that is attached to a bottom structure 3825. A fourth cross-section view across plane A presents an open top microfluidic device 3800 in a separated configuration where a fluidic top 3810 comprising a membrane 3840 is removed from top structure 3820 thereby providing access to an open region 3804, wherein a microfluidic channel 3608 is configured to traverse between fluidic cover 3810 and top structure 3820.

In one embodiment, the present invention contemplates a fully assembled stretchable open top microfluidic device 3600 comprising a fluidic cover 3610 comprising microfluidic channel 3608, a first vacuum port 3630 and a second vacuum port 3632, wherein the microfluidic channel 3608 terminates at either end and an inlet port 3614 and an outlet port 3616, respectively.

As another example, the use of an open-top chip allows electrical stimulation, e.g. using electrodes, and allows recording electrical measurements in real-time, e.g. recording TEER, e.g. epithelial layer, etc.

Additional embodiments of an open top chip.

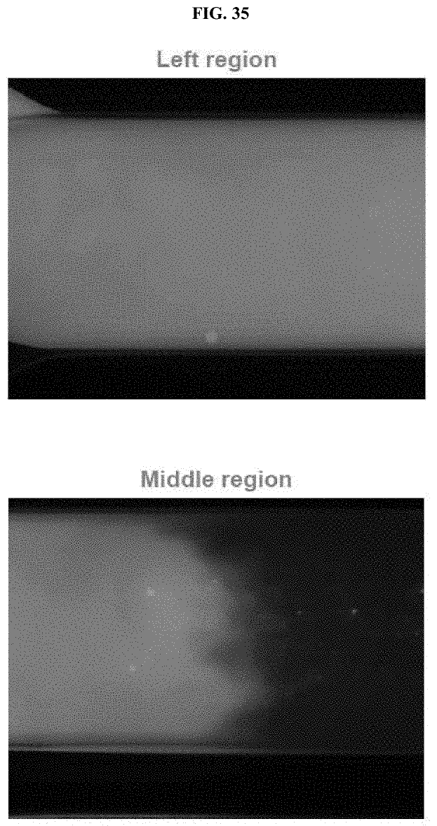

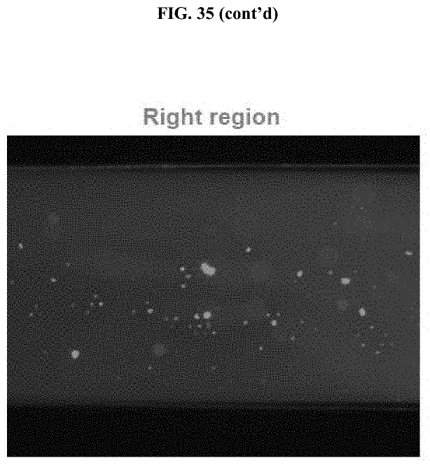

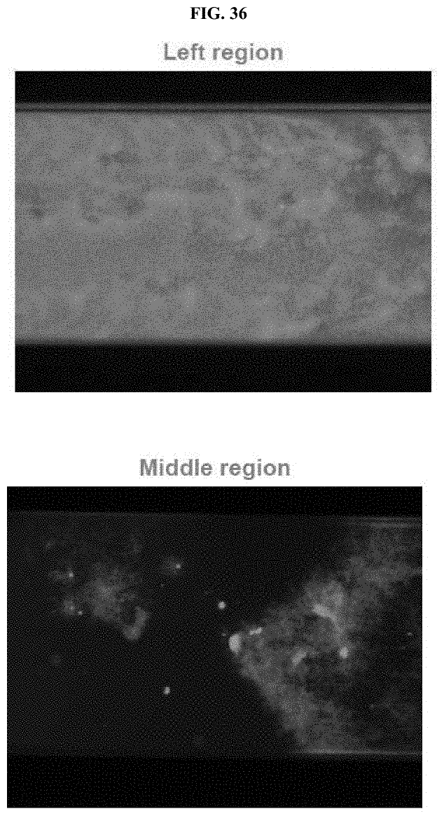

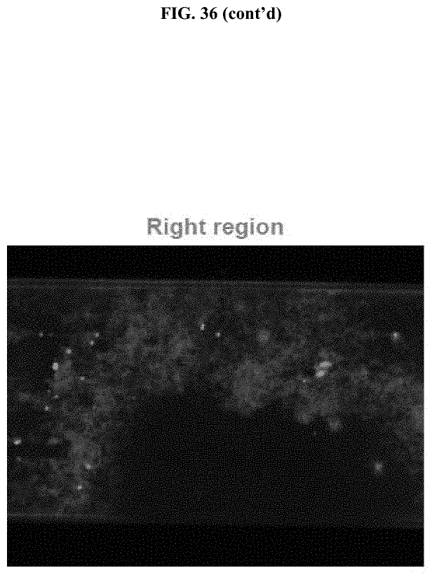

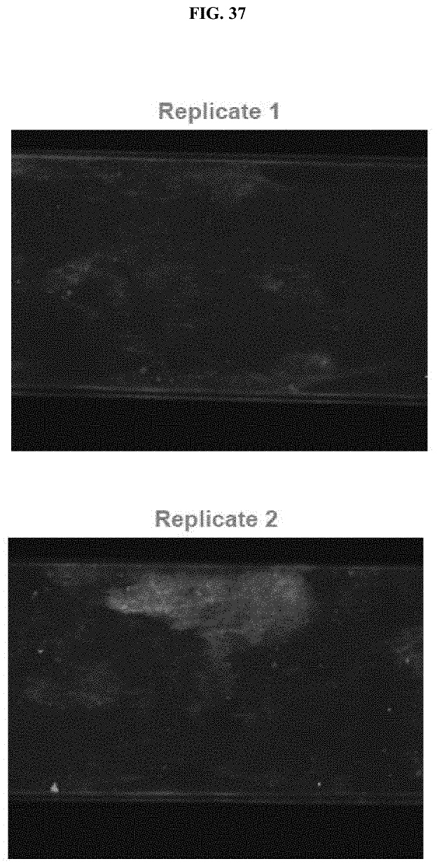

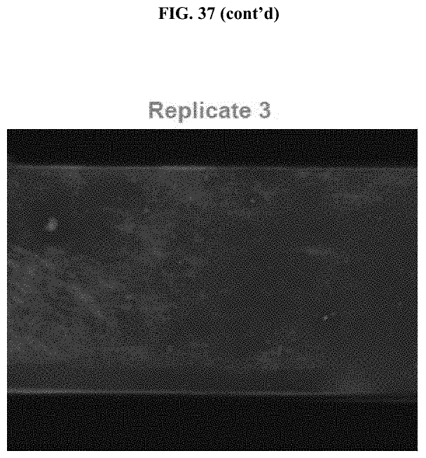

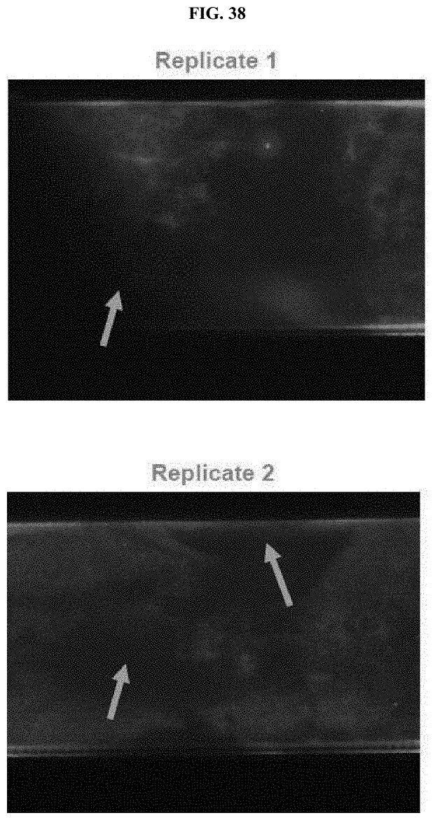

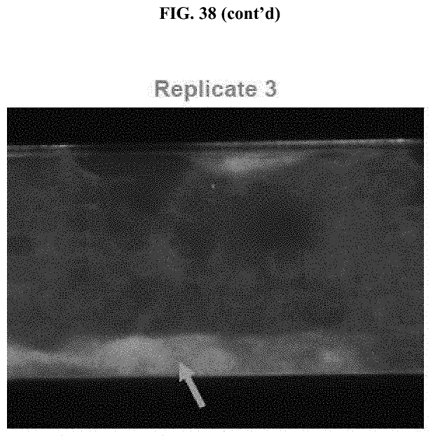

In one embodiment, the present invention contemplates a tall channel stretchable open top chip device 3500 comprising: i) a fluidic cover 3510 comprising an open region 3504; ii) a top structure 3520 comprising an upper microchannel 3534 attached to the fluidic cover 3510; iii) a bottom structure 3525 comprising a lower microchannel 3536 attached to the top structure 3520; and iv) a membrane 3540 layer between the bottom structure 3525 and the top structure 3520. In one embodiment, the open region 3504, upper microchannel 3534 and lower microchannel 3536 are configured to at least partially overlay each other. FIG. 35A and FIG. 35B. Although not intended to be limiting, the tall channel stretchable open top chip device 3500 may also comprise a vacuum port pair and/or inlet/outlet ports as shown and described above.

1. Open Top Microfluidic Chips Without Gels

In one embodiment, the present invention contemplates an open top organ-on-chip device without a gel, either as a bulk gel or a gel layer. Thus, the present invention also contemplates, in one embodiment, a layered structure comprising: i) fluidic channels; ii) a porous membrane; iii) a layer of cells attached to said membrane. In one embodiment, the device comprises a removable cover positioned above the cells.

Additional embodiments are described herein that may be incorporated into open top chips without gels.

2. Open Top Microfluidic Chips with Gels

Furthermore, the present disclosure contemplates improvements to fluidic systems that include a fluidic device, such as a microfluidic device with an open-top region that reduces the impact of stress that can cause the delamination of tissue or related component(s) (e.g., such as a gel layer). Thus, in a preferred embodiment, the open-top microfluidic device comprises a gel matrix. In one embodiment, the open-top microfluidic device does not contain a bulk gel.

In one embodiment, the present invention contemplates an open top microfluidic chip device comprising: i) fluidic channels; ii) a porous membrane: iii) a layer of cells attached to said membrane; and iv) a gel layer. In one embodiment, there is a removable cover over the cells. In one embodiment, the gel layer contacts the layer of cells. In one embodiment, the gel layer contacts the membrane. It is not intended that the present invention be limited to embodiments with only one gel or gel layer. In one embodiment, the device comprises at least two gel layers. In one embodiment, the gel layer is patterned. In one embodiment, the gel layer is not patterned. In one embodiment, a portion of the gel layer is patterned. It is not intended that the present invention be limited by the nature or components of the gel layer or gel coating. In one embodiment, gel layer comprises collagen. A variety of thickness is contemplated. In one embodiment of the layered structure, said gel layer is between 0.2 and 6 mm in thickness.

In one embodiment, the present invention contemplates a microfluidic device comprising: i) a channel comprising a partial gel layer; and ii) a porous membrane. In one embodiment, said membrane comprises cells. The projections, in one embodiment, project outward from the sidewalls. The projections, in another embodiment, project upward. The projects, in another embodiment, project downward. The projections can take a number of forms (e.g. a T structure, a Y structure, a structure with straight or curving edges, etc.). In some embodiments, there are two or more projections; in other embodiments, there are four or more projections to anchor the gel matrix. In one embodiment, the membrane is above said fluidic channels.

Partial gel layers may be used in several types of embodiments for open top microfluidic chips, e.g. epithelial cells or parenchymal cells can be attached to outside of the gel, or within the gel. In some embodiments, LPDCs may be added within the gel, below the gel, or above the gel. In some embodiments, stomal cells are added within the gel. In some embodiments, stomal cells are attached to the opposite side of the partial gel layer. In some embodiments, endothelial cells are located below the partial gel layer. In some embodiments, endothelial cells may be present within the partial gel layer.

IX. Partial Gel layer Filling Techniques

One technique known in the art to create a partial gel layer comprising at least one lumen is referred to as “viscous fingering”. One example of viscous fingering methods that may be used to form lumens, e.g. patterning lumens, is described by Bischel, et al. “A Practical Method for Patterning Lumens through ECM Hydrogels via Viscous Finger Patterning.” J Lab Autom. 2012 April; 17(2): 96-103. Author manuscript; available in PMC 2012 Jul. 16, herein incorporated by reference in its entirety. Viscous finger patterning methods have been used with microfluidic organ-on-chips, lumen structures patterned within a hydrogel. However, viscous fingering techniques are believed to not provide uniformity in lumen formation. The problem was addressed by discovering a gel staining technique that permitted visualization of viscous fingering gel formation irregularities (e.g., spotting, gaps, thickness differences). See, FIG. 9A. It should be noted that the viscous fingering technique necessarily created a gel layer that contacts all four sides of a microchannel, wherein the lumen is enclosed within the gel and does not face any microchannel wall.

By using this gel staining technique an empirical process was designed that identified the parameters necessary to create a uniform partial gel layer. Of interest, the following parameters were tested: i) collagen concentrations; ii) washout durations;

iii) washout agents; iv) polymerization gradient heating; v) directional gelification; vi) gel monomer solution deposition flow rate; and vii) opposing channel fluid flow.

The data collected from the above empirical study identified three techniques that provided partial gel layers having uniform characteristics. See, FIG. 9B. In one embodiment, the partial gel layers disclosed herein do not contact all four sides of a microchannel. In particular, partial gel layers may contact one or less than one of all the microchannel walls, such that the partial gel surface is adjacent to, but does not contact, at least one microchannel wall.

### Dilute Solution

Prepare a low concentration (non-gelling) rat tail collagen I solution (˜0.5 mg/ml).
Slowly inject the dilute collagen solution into a respective channel inlet port to independently fill one channel.
Allow gel to polymerize overnight into a layer approximately 100 microns thick.

### Wash-Out Flushing

Prepare a high concentration rat tail collagen I (1-3 mg/ml) solution.
Form a semi-solid pre-gel by allowing polymerization for 30-45 min
Wash out flushes unpolymerized gel and creates a smooth surface gel layer that is 20-30 microns thick

### Hydrodynamic Shearing

Prepare a high concentration rat tail collagen I (1-3 mg/ml) solution.
Form a solid gel by allowing polymerization for 30-45 min
Using an Eppendorf Explorer micropipettor, under a high flow rate, shear away polymerized gel until a smooth surface gel layer is formed that is 20-30 microns thick.

In one embodiment, Collagen I is used as an underlay or an overlay, with or without Fibronectin. For one example, bovine Collagen I, (e.g. from Advanced Biomatrix, San Diego, Calif.) was used at), at 0.5 mg/ml.

In one embodiment, Liver specific ECM is used as an underlay. For one example, Liver specific ECM from Xylyx Bio East River BioSolutions (Brooklyn, N.Y.) is used as an underlay. Instructions for Use, TissueSpec® Liver ECM Hydrogel Kit, has a suggested dilution: 250 μL ECM of matrix, 25 μL Component A, 25 μL Component B, 200 μL cell culture media for a total amount of 0.5 ml, hydrogel concentration of 4 mg/mL. Final hydrogel concentration can be adjusted by varying the volume of cell culture media. (Instructions for Use—TissueSpec® Liver ECM Hydrogel Kit, Revision: 12 Dec. 2018). However, for optimal use in a microfluidic Tall Channel chip, Liver specific ECM was tested from 3 to 10 times more diluted that suggested in the instructions, but the dilution was not adjusted merely by varying the amount of cell culture media, from the original protocol. Instead, in order to promote a homogenous and flat gel monolayer inside the Tall channel chip, the dilution was made by varying the amounts of the components added to the media.

More specifically, as used herein, the recommended dilution of TissueSpec® Liver ECM (Hydrogel Kit from Xylyx Bio East River BioSolutions: Revision: 12 Dec. 2018) was adapted for the Tall Channel chip as follows: TissueSpec® Liver ECM Hydrogel: 0.3 ml+Component A: 30 μl+Component B: 35 μl+Media 1.350 ml to a total of 4.635 ml.

### X. Chip Activation

A. Chip Activation Compounds

In one embodiment, bifunctional crosslinkers are used to attach one or more extracellular matrix (ECM) proteins. A variety of such crosslinkers are available commercially, including (but not limited to) the following compounds:

### ANB-NOS (N-5-azido-2-nitrobenzoyloxysuccinimide)

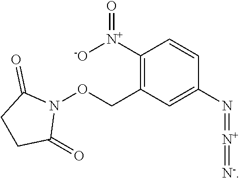

### Sulfo-SAND (sulfosuccinimidyl 2-[m-azido-o-nitrobenzamido]ethyl-1, 3′-dithiopropionate)

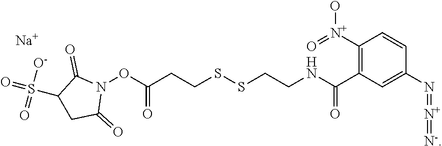

### SANPAH (N-succinimidyl-6-[4′-azido-2′-nitrophenylamino]hexanoate)

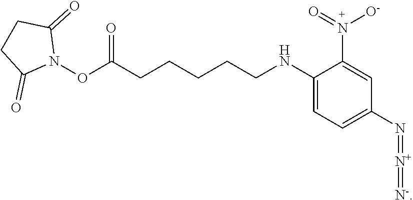

### Sulfo-SANPAH (sulfosuccinimidyl-6-[4′-azido-2′-nitrophenylamino]hexanoate)

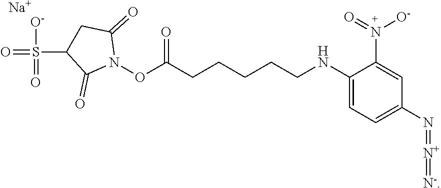

By way of example, sulfosuccinimidyl 6-(4′-azido-2′-nitrophenyl-amino) hexanoate or “Sulfo-SANPAH” (commercially available from Pierce) is a long-arm (18.2 angstrom) crosslinker that contains an amine-reactive N-hydroxysuccinimide (NHS) ester and a photoactivatable nitrophenyl azide. NHS esters react efficiently with primary amino groups (—NH₂) in pH 7-9 buffers to form stable amide bonds. The reaction results in the release of N-hydroxy-succinimide. When exposed to UV light, nitrophenyl azides form a nitrene group that can initiate addition reactions with double bonds, insertion into C—H and N—H sites, or subsequent ring expansion to react with a nucleophile (e.g., primary amines). The latter reaction path dominates when primary amines are present.

Sulfo-SANPAH should be used with non-amine-containing buffers at pH 7-9 such as 20 mM sodium phosphate, 0.15M NaCl; 20 mM HEPES; 100 mM carbonate/bicarbonate; or 50 mM borate. Tris, glycine or sulfhydryl-containing buffers should not be used. Tris and glycine will compete with the intended reaction and thiols can reduce the azido group.

For photolysis, one should use a UV lamp that irradiates at 300-460 nm. High wattage lamps are more effective and require shorter exposure times than low wattage lamps. UV lamps that emit light at 254 nm should be avoided; this wavelength causes proteins to photodestruct. Filters that remove light at wavelengths below 300 nm are ideal. Using a second filter that removes wavelengths above 370 nm could be beneficial but is not essential.

B. Exemplary methods of Chip Activation

### Prepare and Sanitize Hood-Working Space:

1. S-1 Chip Handling—Use aseptic technique, hold Chip using Carrier
a. Use 70% ethanol spray and wipe the exterior of Chip package prior to bringing into hood.
b. Open package inside hood
c. Remove Chip and place in sterile petri dish (6 Chips/Dish).
d. Label Chips and Dish with respective condition and Lot #.
   2. Surface Activation with Chip Activation Compound (light and time sensitive)
a. Turn off light in biosafety hood.
b. Allow vial of Chip Activation Compound powder to fully equilibrate to ambient temperature (to prevent condensation inside the storage container, as reagent is moisture sensitive).
c. Reconstitute the Chip Activation Compound powder with ER-2 solution.
   i. Add 10 ml Buffer, such as HEPES, into a 15 ml conical covered with foil.
   ii. Take 1 ml Buffer from above conical and add to chip Activation Compound (5 mg) bottle, pipette up and down to mix thoroughly and transfer to same conical.
   iii. Repeat 3-5 times until chip Activation Compound is fully mixed.
   iv. NOTE: Chip Activation Compound is single use only, discard immediately after finishing Chip activation, solution cannot be reused.
d. Wash channels.
   i Inject 200 ul of 70% ethanol into each channel and aspirate to remove all fluid from both channels
   ii. Inject 200 ul of Cell Culture Grade Water into each channel and aspirate to remove all fluid from both channels
   iiii. Inject 200 ul of Buffer into each channel and aspirate to remove fluid from both channels
e. Inject Chip Activation Compound Solution (in buffer) in both channels
   i. Use a P200 and pipette 200 ul to inject Chip Activation Compound/Buffer into each channel of each chip (200 ul should fill about 3 Chips (Both Channels))
   ii. Inspect channels by eye to be sure no bubbles are present. If bubbles are present, flush channel with Chip Activation Compound/Buffer until bubbles have been removed
f. UV light activation of Chip Activation Compound: Place Chips into UV light box
   i. UV light treat Chips for 20 min
   ii. While the Chips are being treated, prepare ECM Solution.
   iii. After UV treatment, gently aspirate Chip Activation Compound/Buffer from channels via same ports until channels are free of solution
   iv. Carefully wash with 200 ul of Buffer solution through both channels and aspirate to remove all fluid from both channels
   v. Carefully wash with 200 ul of sterile DPBS through both channels
   vi. Carefully aspirate PBS from channels and move on to: ECM-to-Chip.

### XI. Extracellular Matrix Layers

Some embodiments described herein relate to devices for simulating a function of a tissue, in particular a gastrointestinal tissue. In one embodiment, the device generally comprises (i) a first structure defining a first chamber; (ii) a second structure defining a second chamber; (iii) a membrane located at an interface region between the first chamber and the second chamber to separate the first chamber from the second chamber, the membrane including a first side facing toward the first chamber and a second side facing toward the second chamber, iv) a cell layer; and (iv) an extracellular matrix layer. In one embodiment, the ECM layer is overlay of the cell layer. In one embodiment, the ECM layer is an underlay of the cell layer. In one embodiment, the device comprises two ECM layers, an underlay ECM layer and an overlay ECM layer relative to said cell layer

To determine optimum conditions for cell attachment, the surface-treated material (e.g., APTES-treated or plasma-treated PDMS) can be coated with an ECM layer of different extracellular matrix molecules at varying concentrations. The ECM overlay is typically a “molecular coating,” meaning that it is done at a concentration that does not create a bulk gel. In some embodiments, an ECM overlay is used. In some embodiments, an ECM overlay is left in place throughout the co-culturing. In some embodiments, an ECM overlay is removed, e.g. when before seeding additional cells into a microfluidic device. In some embodiments, the ECM layer is provided by the cells seeded into the microfluidic device.

Although some cells described herein make their own ECM (e.g., epidermal epithelial cells), it is contemplated that ECM in predisease and diseased states may be found in areas around sites of cell growth. Further, the protein microenvironment provided by ECM also affects cells. Thus, it is contemplated that tissue-derived ECM may carry over a disease state. Therefore, in addition to the ECM described herein, ECM used in microfluidic devices of the present embodiments may be derived from or associated with areas in and around sites of cells. In one embodiment, a device comprising tissue-derived ECM may be used as described herein, to identity contributions to healthy or disease states affected by native ECM.

For example, ECM may be isolated from biopsies of healthy, non-disease and disease areas as tissue-derived ECM. Isolates for use may include cells within or attached or further processed to remove embedded cells for use in the absence of the cells.

Additional examples of ECM materials include but are not limited to Matrigel®, Cultrex®, ECM harvested from humans, etc.

### XII. Exemplary Media Formulations.

Several media formulations were tested initially on CF cell cultures in Transwell devices and microfluidic devices using standard ECM, i.e. Co-IV coating of functionalized membrane without a gel overlay. One exemplary formulation of DMEM/F12 plus Single Quots plus 4% serum, an optimized media promoted by the Cystic Fibrosis Foundation for augmented mucus production from CF cells in culture, resulted in stressed CF epithelium and epithelial cell death. Another formulation tested for airway epithelial cells was a 50:50 Dulbecco's Modified Eagle Medium (DMEM):SABM™ Small Airway Epithelial Basal Medium, plus SingleQuots™ (Lonza) plus HEPES Buffer, optionally 2% serum, optimized for healthy cell cultures and used in CF experiments. The DMEM:SABM™ medium formulation caused CF cell overproliferation and sparse ciliary beating. In part, these results led to experiments described herein for optimizing ECM substrate, e.g. gels. Subsequently, use of one embodiment of a gel, i.e. Col-I gel overlaying a Col-IV coating, resulted in high ciliary movement when using either one of these exemplary media formulations.

In other words, use of gels, such as partial gel layers or overlays, for culturing delicate CF cells overcame differences observed between media formulations. Therefore, these exemplary media formulations may be used for culturing both normal and diseased ciliated airway epithelial cells.

### XIII. Cell Migration: Immune Cell Transmigration and Recruitment

In one embodiment, the present invention contemplates a microfluidic device comprising at least one microchannel and a membrane, wherein a thin (partial) gel layer (e.g., without cells) is deposited on top of the membrane. In one embodiment, the thin (partial) gel layer prevents undesirable cell migration. In one embodiment, the microchannel further comprises a partial gel layer comprising a stromal cell layer. In one embodiment, the stromal cell layer is coated with a second thin (partial) layer (without cells). In one embodiment, the stromal cell layer is in contact with an epithelial cell layer or a parenchymal cell layer. In one embodiment, the gel layers can be of different compositions and/or thicknesses or all the same.

In one embodiment, the present invention contemplates a method comprising a microfluidic device comprising a channel with a partial gel layer and at least one immune cell.

In one embodiment, a first cell layer is disposed on an upper surface of the partial gel layer. In one embodiment, a second cell layer is disposed on a lower surface of the partial gel layer. In one embodiment, the method further comprises contacting the at least one immune cell with at least one inflammation-inducing stimuli. In one embodiment, the method further comprises transmigrating a first immune cell into said partial gel layer. In one embodiment, the channel comprises a membrane, the membrane having an upper surface and a lower surface. In one embodiment, the first cell layer is attached to the membrane upper surface. In one embodiment, the second cell layer is attached to the membrane lower surface. In one embodiment, a first extracellular matrix is disposed between said first cell layer and said membrane upper surface. In one embodiment, a second extracellular membrane is disposed between said second cell layer and the membrane lower surface. In one embodiment, the at least one inflammation-inducing stimuli includes, but is not limited to, cytokines, viruses, human rhinoviruses, chemokines, CXCL2 (also known as macrophage inflammatory protein 2 alpha (MIP2 alpha), interleukin 13, tissue necrosis factor-alpha (TNF-α), interleukin-1 (IL-1), IL-12, IL-18, interferon gamma (IFN-γ), and/or granulocyte-macrophage colony stimulating factor (GM-CSF). In one embodiment, the first immune cell is a T-cell or a peripheral blood mononuclear cells (PBMC). In one embodiment, the at least one inflammation-inducing stimuli recruits the transmigrated T-cell or PBMC. In one embodiment, the recruited T-cell or recruited PBMC induces transmigration of a second immune cell.

The data presented herein demonstrates that immune cells can be transmigrated through a partial gel layer. For example, a partial gel layer is shown with fluorescently labeled PBMC cells induced and recruited by human rhinovirus or interleukin 13. See, FIG. 10A. A close-up photomicrograph clearly shows several transmigrated PBMC cells detected a different levels of fluorescent intensity. These data are interpreted to demonstrate that the PBMCs emitting less intense and more diffuse fluorescence have penetrated the deepest into the partial gel layer. See, FIG. 10B. Upper Arrow: Indicates PBMC cells that have passed through the partial gel layer and into the membrane. Alternatively, the transmigrating cells have been visualized at various gel depths by imaging a different focal planes (data not shown).

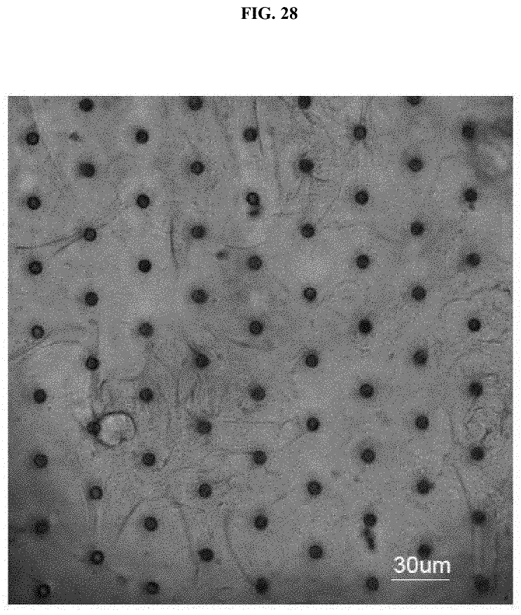

During cell culture of epithelial cells in a two-channel microfluidic system, it has been observed that the epithelial cells migrate from the membrane to the bottom channel under standard culture conditions. See, FIG. 28. In one embodiment, the present invention contemplates a method to prevent epithelial migration by first seeding endothelial cells on a membrane and culturing the endothelial cells to form endothelial barrier. Subsequently, epithelial cells are overseeded onto the endothelial layer to form an endothelial/epithelial co-culture layer.

Alternatively, several different types of partial gel matrices were compared for their respective ability to prevent top channel exposed epithelial cell from migrating through a membrane and into the bottom channel. See, Table below.

TABLE 4
Partial Gel Layer Screen To Prevent Cell Migration Through Membrane.

| | | Days | | | | | | | |
|---|---|---|---|---|---|---|---|---|---|
| | Chip | Post- | | Cell | | | | | |
| Condition | ID | ALI | Peeling | Death | Overgrowth | Movement | Ciliation | Invasion | Score |
| Matrigel ® + | 1.1 | 14 | 0 | 1 | 1 | 2.5 | 0 | 1 | 5.5 |
| Collagen IV | | | | | | | | | |
| Matrigel ® + | 1.2 | 14 | 1 | 1 | 1 | 2 | 0 | 0.5 | 5.5 |
| Collagen IV | | | | | | | | | |
| Matrigel ® + | 1.3 | 14 | 0 | 0 | 2 | 3 | 0 | 0 | 5 |
| Collagen IV | | | | | | | | | |
| Matrigel ® + | 1.4 | 14 | 2 | 0 | 2 | 3 | 0 | 1 | 8 |
| Collagen IV | | | | | | | | | |
| Matrigel ® + | 1.6 | 14 | 0 | 1 | 2 | 2.5 | 0 | 3 | 8.5 |
| Collagen IV | | | | | | | | | |
| Matrigel ® + | 1.1 | 14 | 0.5 | 0 | 1 | 2 | 0 | 0 | 3.5 |
| Collagen IV | | | | | | | | | |
| Matrigel ® + | 1.4 | 14 | 0 | 1 | 2 | 0.5 | 0 | 0 | 3.5 |
| Collagen IV | | | | | | | | | |
| Matrigel ® + | 1.6 | 14 | 2 | 0 | 0 | 2 | 0 | 0 | 4 |
| Collagen IV | | | | | | | | | |
| Matrigel ® + | 1.7 | 14 | 0 | 2 | 1 | 2 | 0 | 0 | 5 |
| Collagen IV | | | | | | | | | |
| Matrigel ® + | 1.8 | 14 | 1.5 | 1 | 1 | 2 | 1 | 2 | 8.5 |
| Collagen IV | | | | | | | | | |
| Matrigel ® + | 1.9 | 14 | 1 | 1 | 0 | 3 | 0 | 3 | 8 |
| Collagen IV | | | | | | | | | |
| Collagen IV | 2.3 | 14 | 0 | 1 | 1 | 2 | 0 | 1 | 5 |
| Collagen IV | 2.4 | 14 | 1 | 0 | 0 | 3 | 2 | 0 | 6 |
| Collagen IV | 2.5 | 14 | 0.5 | 0 | 1 | 1 | 0 | 0 | 2.5 |
| Collagen IV | 2.6 | 14 | 0 | 0 | 1 | 1 | 0 | 1 | 3 |
| Collagen IV | 2.9 | 14 | 1 | 0 | 2 | 2 | 0 | 0 | 5 |
| Collagen I + | 2.1 | 14 | 0 | 1.5 | 2 | 1 | | 0.5 | 5 |
| IV | | | | | | | | | |
| Collagen I + | 2.4 | 14 | 0 | 1.5 | 2 | 2.5 | 0 | 1.5 | 7.5 |
| IV | | | | | | | | | |
| Collagen I + | 2.5 | 14 | 2 | 1 | 2 | 2.5 | 0 | 0 | 7.5 |
| IV | | | | | | | | | |
| Collagen I + | 2.6 | 14 | 1 | 1.5 | 1 | 2.5 | 0 | 1 | 7 |
| IV | | | | | | | | | |
| Control | 3.1 | 14 | 2 | 1 | 2 | 1 | 0 | 3 | 9 |
| Control | 3.2 | 14 | 0 | 0 | 3 | 1 | 0 | 1 | 5 |
| Control | 3.3 | 14 | 2 | 0 | 2 | 3 | 0 | 1.5 | 8.5 |
| Control | 3.8 | 14 | 2 | 0 | 3 | 2 | 0 | 3 | 10 |
| Control | 3.1 | 14 | 0.5 | 0 | 0.5 | 1 | 0 | 0 | 2 |
| Control | 3.2 | 14 | 0 | 2 | 1 | 2 | 0 | 2 | 7 |
| Control | 3.3 | 14 | 0 | 2 | 0 | 2 | 0 | 2 | 6 |
| Control | 3.5 | 14 | 0 | 0.5 | 0 | 2 | 0 | 2 | 4.5 |

In this screen, a full epithelial cell migration (e.g., invasion) into the bottom channel of an S1 Base Model chip, is given a score of >1. For the Matrigel®+Collagen IV partial gel matrix, only one out of eleven chips had a full invasion score. For the Collagen IV only partial gel matrix, zero out of five chips had a full invasion score. For the Collagen I-Collagen IV partial gel matrix, one out of four chips had a full invasion score. Overall, only two out of twenty (e.g., 10%) of the partial gel matrix chips had a full invasion score. In contrast, six our of eight (80%) of the control chips (e.g., no partial gel layer) had a full invasion score. In conclusion, intact Collagen or Matrigel® partial gel layers prevent epithelial migration into the bottom channel. Although it is not necessary to understand an invention, it is believed that because Matrigel® is a naturally produced matrix, it is inherently ill-defined. As such, in embodiments of the present invention non-Matrigel® gels are preferred.

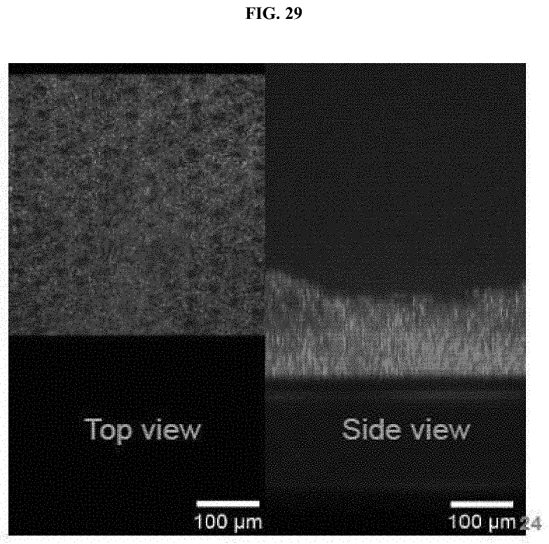

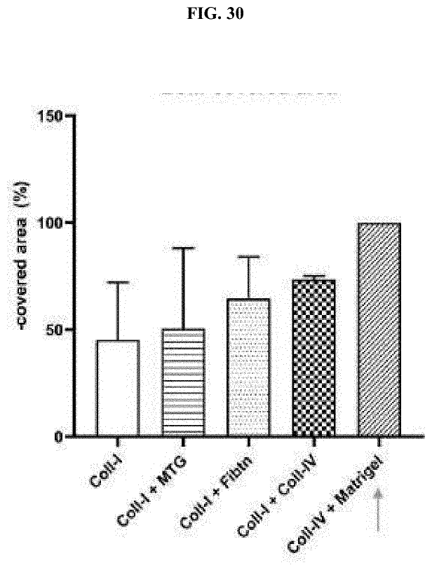

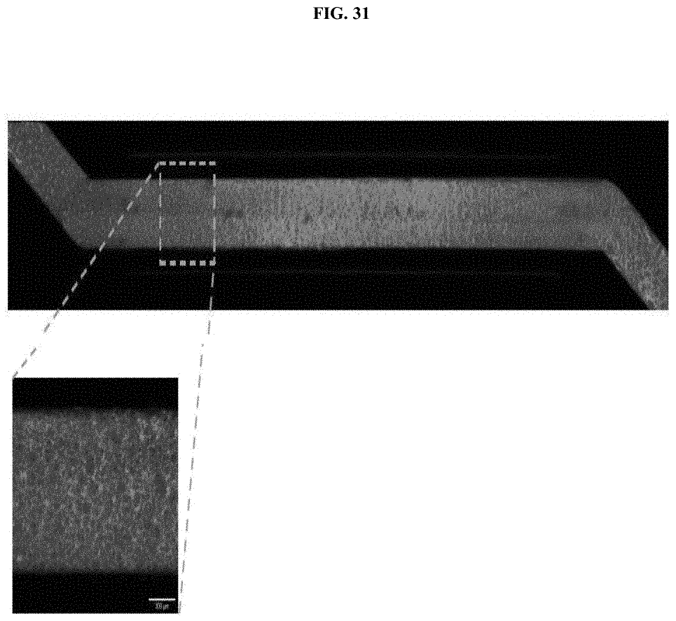

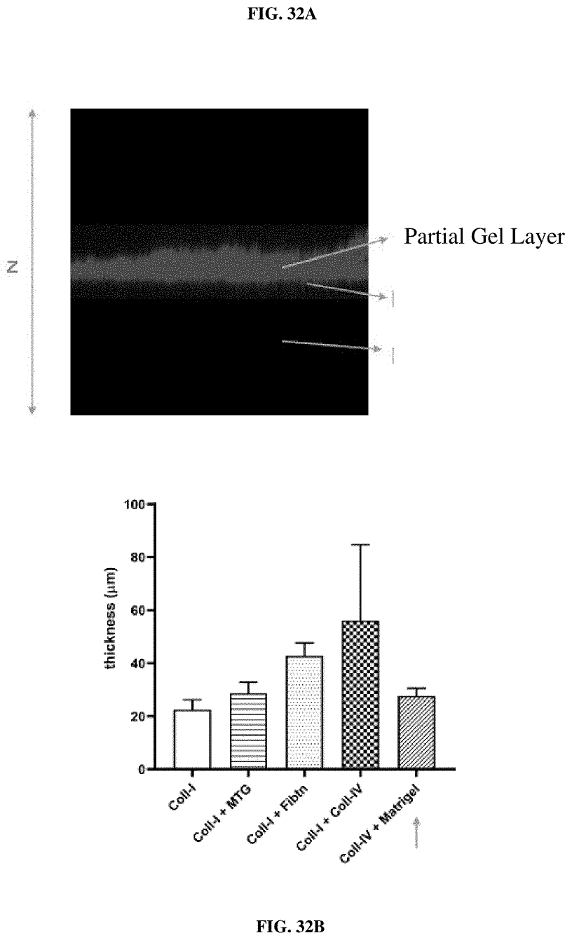

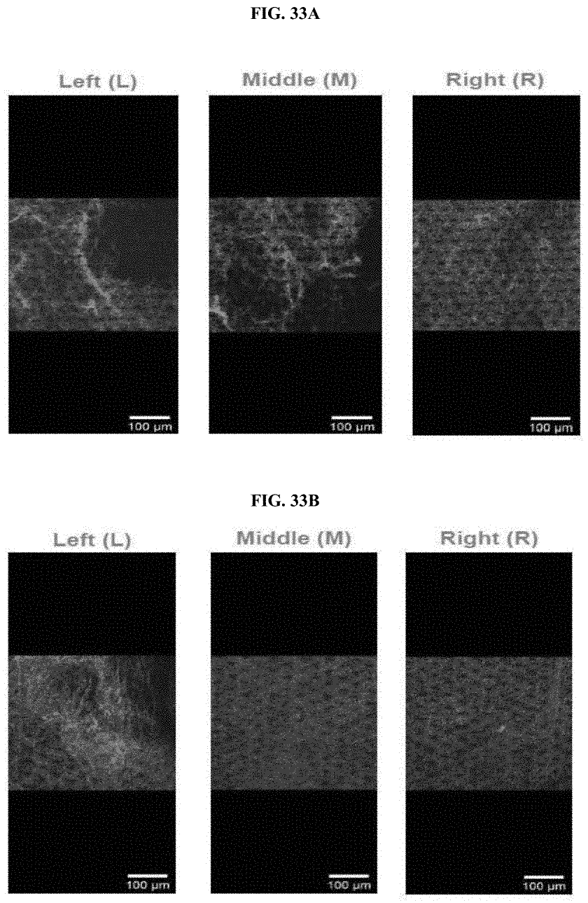

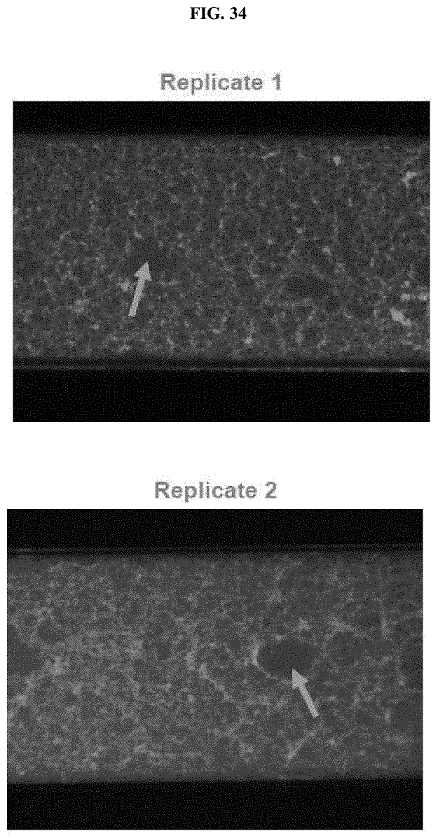

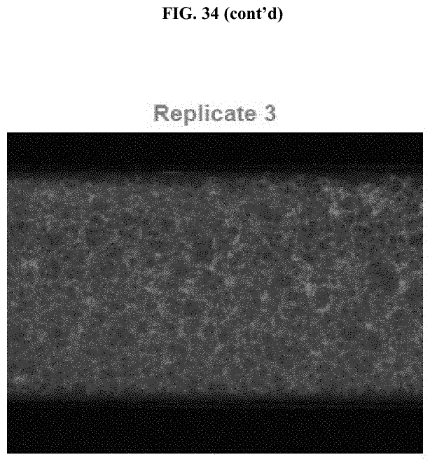

In one embodiment, the epithelial cells comprising the endothelial/epithelial co-culture layer do not migrate to the bottom channel. In one embodiment, the present invention contemplates a method to prevent epithelial migration by creating partial gel layer in the epithelial (top) channel into which fibroblasts and/or immune cells are embedded such that the epithelial cells do not migrate to the bottom channel. In one embodiment, the partial gel layer comprises a collagen IV-Matrigel® matrix. In one embodiment, the partial gel layer comprises a collagen IV-collagen I matrix. The data shows that three out of four cells on the gel matrix do not migrate, but all four cells do migrate in the control condition. Cells are visible in photomicrographs (top view and side view) of a partial gel layer exposed to a top channel. See, FIG. 29.

In one embodiment, the present invention contemplates a membrane comprising a plurality of pores, wherein a portion of the plurality of pores comprise a plug. Although it is not necessary to understand the mechanism of an invention, it is believed that such membrane pore plugs prevent undesirable cell migration through the membrane pores. In one embodiment, the plurality of plugs remain biologically useful wherein cellular biochemical signals (e.g., cell-cell communication) diffuses through the plugs. In one embodiment, the cellular biochemical signals induce cell migration through the membrane comprising a plurality of pore plugs. In one embodiment, the plurality of pores range in diameter between approximately 7 μm-20 μm. In one embodiment, the membrane pores are manufactured using ultraviolet (UV) laser ablation. In one embodiment, the membrane is disposed in a microfluidic device comprising a microchannel, said microchannel comprising a partial gel layer. In one embodiment, the membrane is disposed in a microfluidic device comprising an open-top microchannel.

### IXV. Diseased Cell Organ-On-a Chip Models

In one embodiment, the present invention contemplates a microfluidic device comprising a microchannel with a partial gel layer (e.g., an S1 Base Model Chip) configured to support a culture of sensitive diseased cells. (e.g., cystic fibrosis (CF) cells. In other embodiments, the microfluidic device supports other cell types (e.g. fibroblasts) to form stable interstitial tissue layer beneath a top sensitive diseased cell layer.

In one embodiment, the present invention contemplates a method comprising a microfluidic channel disposed with a layer of differentiated diseased cells. In one embodiment, the diseased cells comprise a plurality of cystic fibrosis lung cells. In one embodiment, the layer further comprises lung basal cells. In one embodiment, the layer further comprises fibroblast cells. In one embodiment, the fibroblast cells form a stable interstitial tissue layer beneath the differentiated diseased cells.

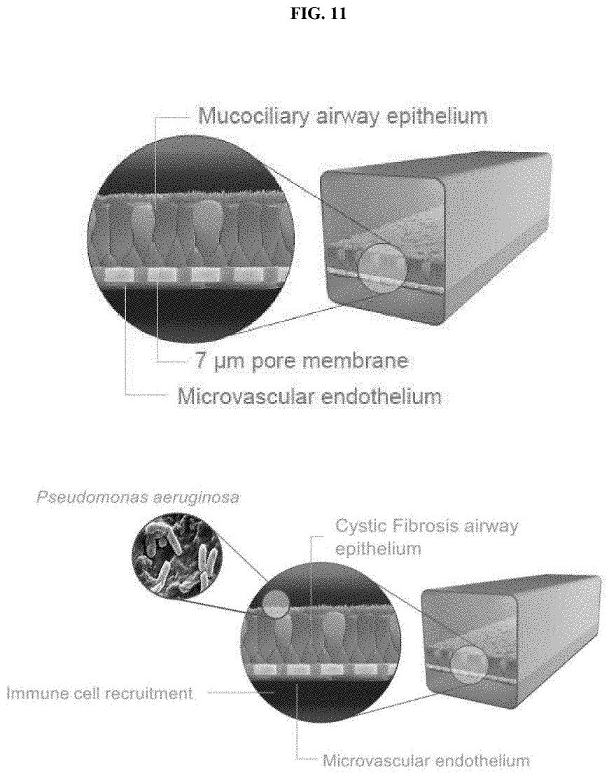

One diseased cell model of interest involves lung disease, e.g., cystic fibrosis (CF). In CF, the diseased cell type is a lung cell type. For example, the lung cell type is a ciliary cell. To improve the existing cell culture models, a lung-on-a-chip was designed comprising a membrane (7 μm pores) with a mucociliary airway epithelium layer and a microvascular epithelial layer. See, FIG. 11.

The development of a lung disease organ-on-a-chip model evaluated and solved a variety of problems in the art. See, Table 5.

TABLE 5
Challenges And Solutions To Develop A Lung Disease Chip
(e.g., S1 Base Model).

| Challenges | Approach | Solution / Achievement |
|---|---|---|
| No standardized | Establish QC procedure | Quantify morphological |
| Airway | based on literature and | markers during |
| epithelial | develop documentation | differentiation and document |
| cell culture | standards | daily |
| protocol or QC on | | Developed robust protocol |
| S1 PDMS Chips | | for PDMS S1 Chip |
| Epithelium | Optimize membrane | Double activation & BSA |
| delaminates | activation and ECM | coating |
| from membrane | coating | High seeding density & |
| | Optimize seeding cell | delay onset of flow until 6 h |
| | density and onset of | post seeding |
| | flow | |
| | Tested 10+ ECM types | |
| | and activation/ seeding/ | |
| | flow parameters | |
| Epithelium | Prevent shear stress: | Perform washes in a Zoe |
| transdifferentiates | Optimize tissue washes | culture module |
| into fibroblasts | Prevent thermal stress: | Dedicated incubators, |
| due to stress | Closely monitor | regular Zoe culture module |
| response | temperatures in Zoe | QC (quality control), and |
| | culture module and | real-time temperature |
| | incubator with real-time | sensors |
| | temperature sensors | |
| Incomplete or | Optimize differentiation | Pneumacult-ALI medium at |
| delayed epithelial | medium. we tested 10+ | 100% or 50% (mixed with |
| differentiation | media types and | standard DMEM/F12) |
| | combinations | |
| Frequent | Optimize pressure | Plug top channel reservoirs |
| submersion of top | differential between top | with 1 ml of medium each |
| channel when at | and bottom channel. | to in order to compensate |
| air-liquid interface | Modeled and tested | for hydrostatic pressure |
| | adding hydrostatic | crease in bottom channel |
| | pressure to top | due to waste buildup. |
| | channel | ADDITIONAL BENEFIT: |
| | | prevents dehydration of top |
| | | channel |
| Migration of | Strategy 1: 3D gel | Generated protocol for |
| epithelial cells into | layer in top channel. | adding continuous |
| bottom channel | Tested 7+ gel | ColIV/Matrigel ® gel and |
| | application protocols | ColV/ColIgel with |
| | and mixtures to | homogeneous thickness and |
| | achieve suitable | distribution in S1 without |
| | gel layer | blocking channel etc. |
| | | ColIV/Matrigel ® gel and |
| | | ColV/ColIgel on S1 prevent |
| | | epithelial migration into |
| | | bottom channel (1 donor, |
| | | n = 6) |
| | | ADDITIONAL BENEFIT of |
| | | Gel on S1 Chip: can be used |
| | | to embed fibroblasts and |
| | | resident immune cells & |
| | | helps diseased cells grow |
| Migration of | Strategy 2: Endothelial | Early seeding of endos |
| epithelial cells into | cell barrier in | prevents migration (1 donor, |
| bottom channel | bottom channel | n = 3) |
| No standardized | Optimize endothelial | Identified medium that |
| endothelial cell | seeding time point, cell | supports long-term co- |
| culture protocol | source, as well as | culture |
| | co-culture medium | |
| No quantification | Establish endpoints | Preliminary results: desired |
| of endpoints in | based on literature | cell types and CBF present |
| mature chip | | in 1 donor |

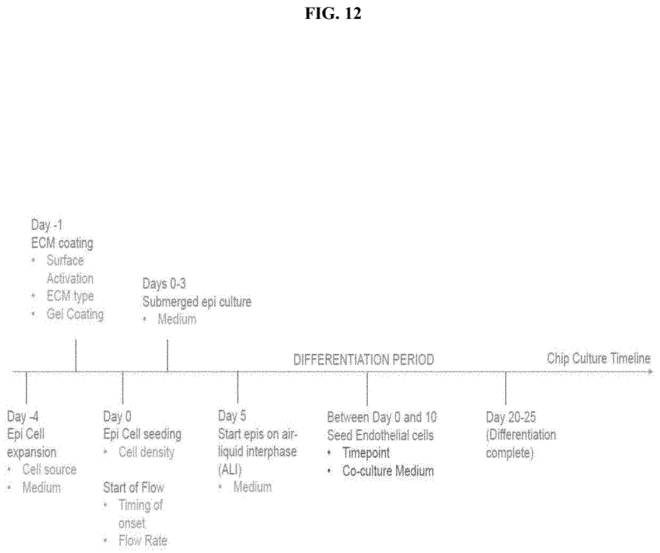

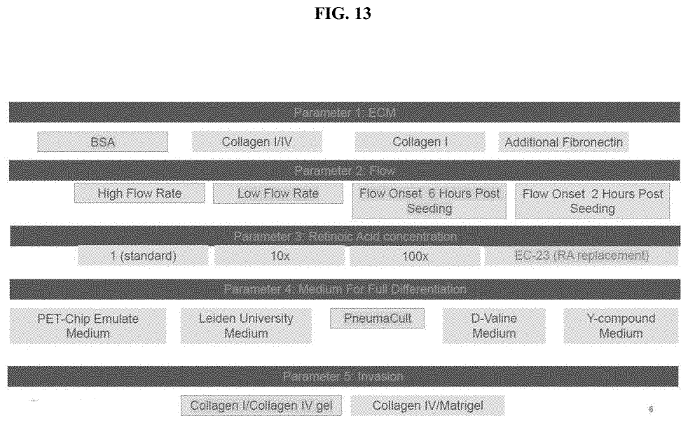

A testing protocol was then devised identifying the time points at which each of the above identified problems would be addressed. See, FIG. 12. The tested parameters were divided into five (5) different categories: i) extracellular membrane layers; ii) media flow characteristics; iii) retinoic acid characteristics; and iv) differentiation media; and v) invasion gel layers. See, FIG. 13.

In one embodiment, the extracellular membrane comprises bovine serum albumin. In one embodiment, the media flow characteristic comprises a low flow rate and/or flow initiate approximately six (6) hours after cell seeding. In one embodiment, the retinoic acid characteristics comprises EC-23, a retinoic acid substitute compound. In one embodiment, the differentiation media comprises PneumaCult®. In one embodiment, the invasion gel layer comprises a collagen I/collagen IV matrix gel layer. In one embodiment, the collagen I/collagen IV matrix gel layer is a partial collagen I/collagen IV matrix gel layer.

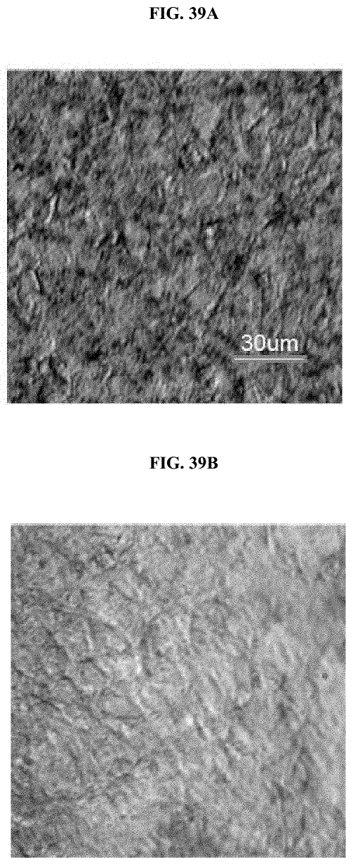

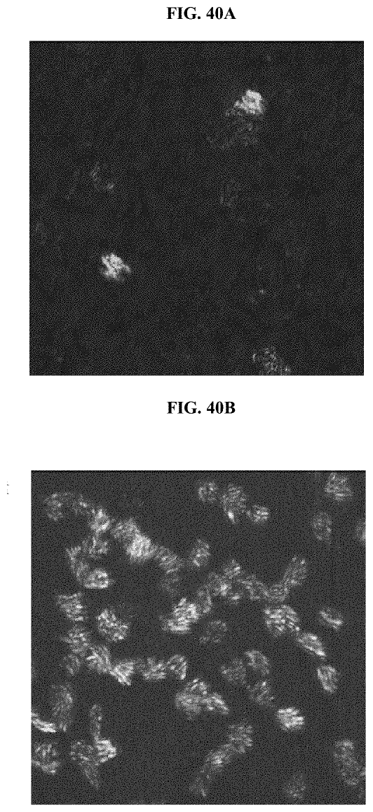

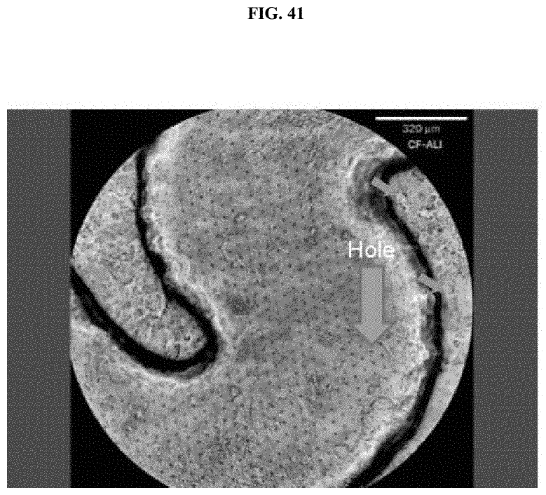

In one embodiment, the present invention contemplates a method for culturing a cystic fibrosis (CF) diseased lung cell. It was observed that cystic fibrosis cells have improved differentiation in transwell cultures when adding a collagen I gel underlay to a standard a collagen IV overlay. See, FIG. 39A-B. The collagen I gel underlay lead to higher ciliation (better differentiation) of CF cells. Improved synchrony of the ciliary beat frequency can also be detected with the collagen I gel underlay configuration. See, FIG. 40A-B. The development of a differentiation microfluidic cell culture model for cystic fibrosis cells has, until now, has been problematic. For example, preliminary data was collected using an “open-top” microfluidic device where the cells are exposed to provide easy access for observation. However, this approach has proved to be unstable, even when accompanied by collagen I gel underlays that provide acceptable results using a static transwell device. See, FIG. 41. These data show that the open-top microfluidic device comprising a cystic fibrosis cell culture developed holes (see, arrow). There were two observations that suggest possible explanations for this phenomenon. First, it was observed that the collagen 1 gel retracted at lower collagen I gel concentrations, and even at higher collagen I gel concentrations, the cystic fibrosis monolayer still exhibited hole formations. Either way, CF cells could not be maintained for more than 7 days with an open-top microfluidic device.

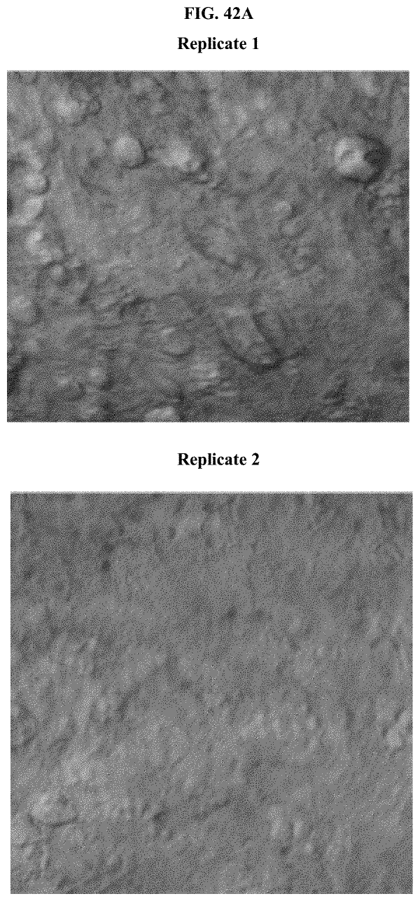

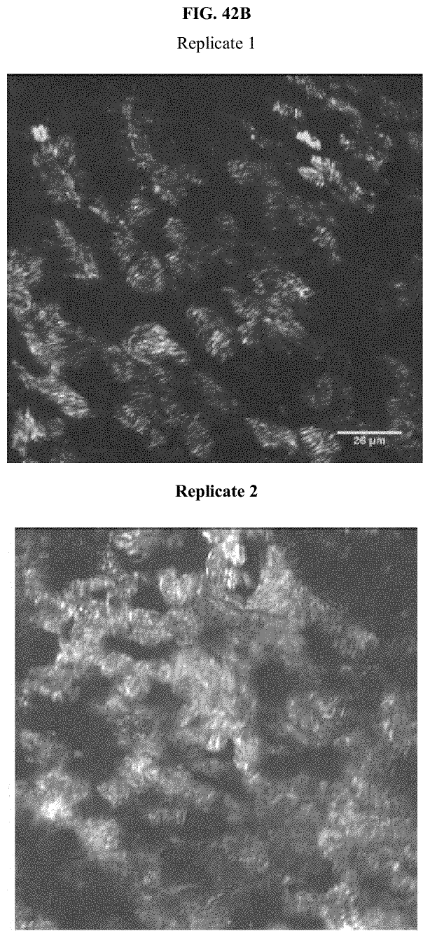

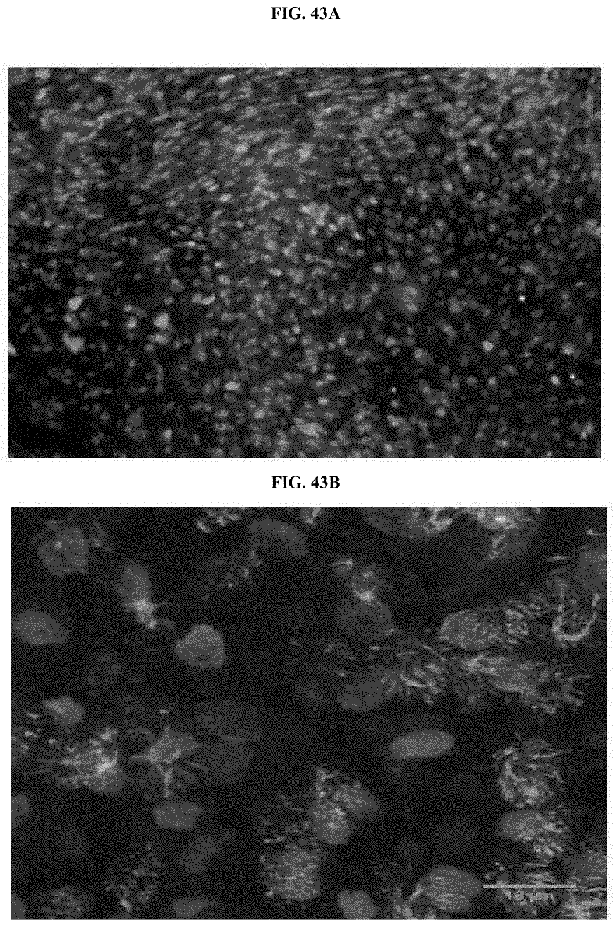

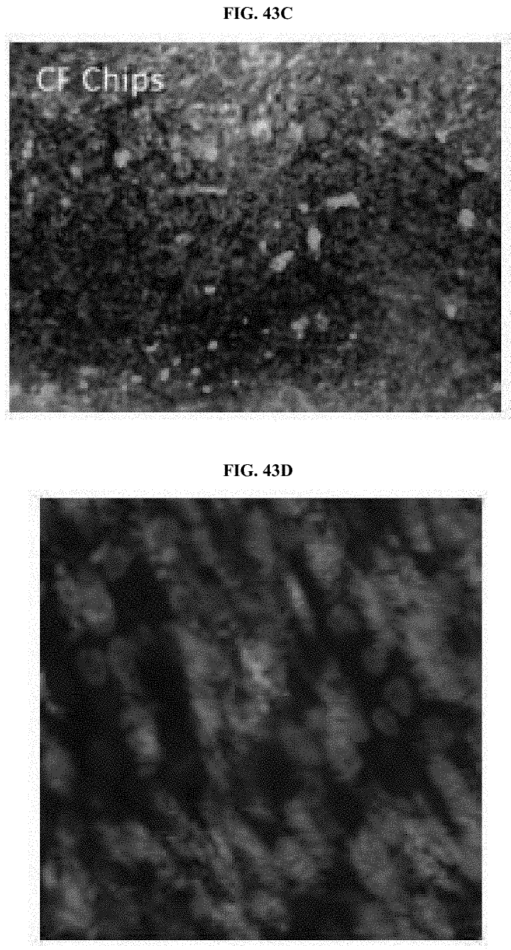

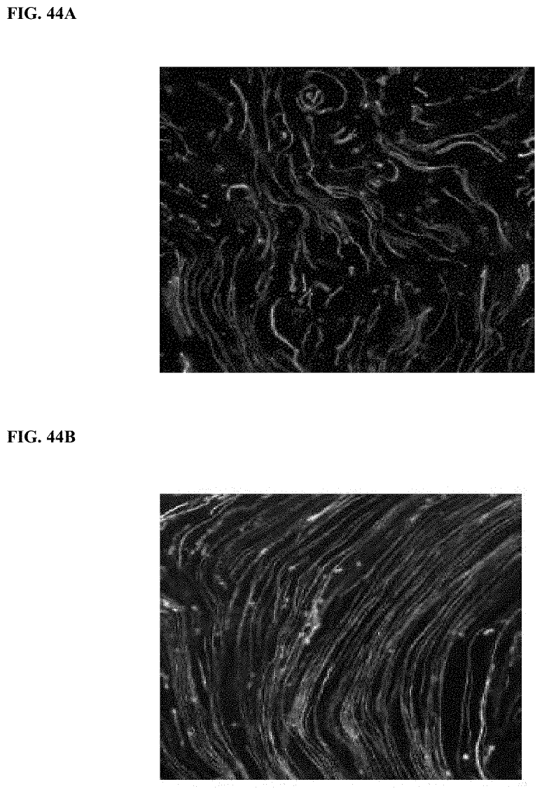

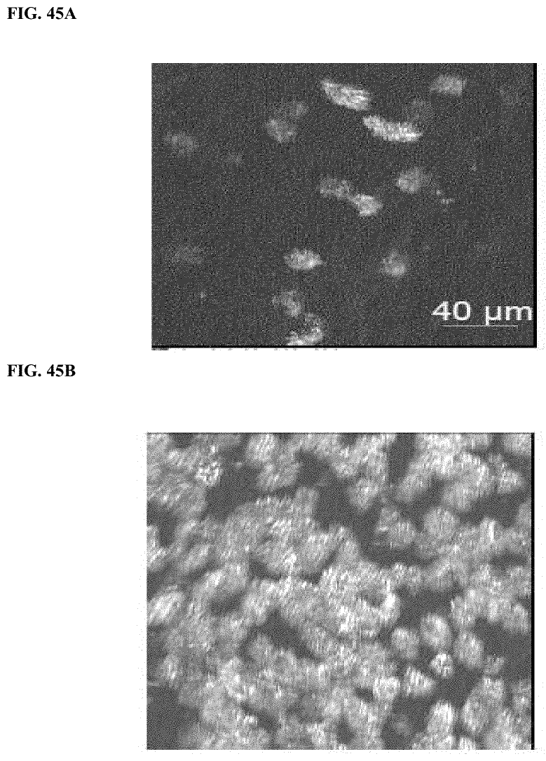

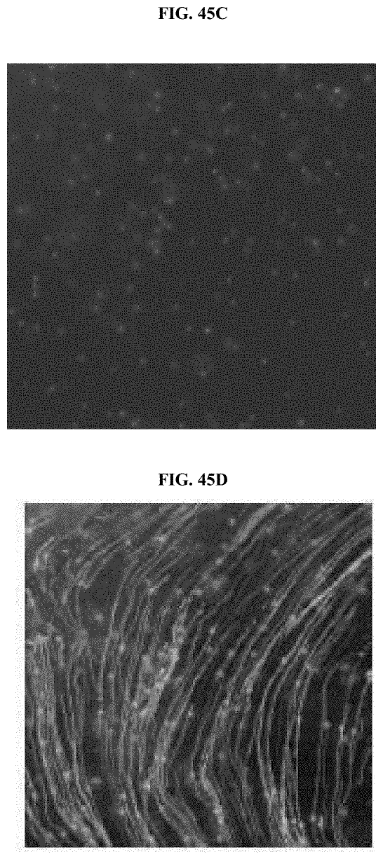

In one embodiment, the present invention contemplates a microfluidic device comprising at least one microchannel comprises a membrane coated with a mixed collagen 1/collagen IV partial gel layer and a cystic fibrosis cell layer. After fourteen (14) days exposure to an ALI, the cystic fibrosis cell layer had differentiated and become fully ciliated. See, FIG. 42A. Furthermore, when using motion detection microscopy, these ciliated differentiated cystic fibrosis cells exhibited synchronous ciliary beat frequencies. See, FIG. 42B. Further photomicroscopy staining procedures showed that these CF cultures had not only fully differentiated cilia, but mucus-secreting goblet cells and basal cells as well. See, FIGS. 43A and 43B. In one embodiment, these differentiated cystic fibrosis cells are used for functional studies in comparison to healthy cells. It was observed, that the differentiated goblet cells resulted in a reduced directedness of mucociliary transport when compared to healthy differentiated lung cells. See, FIGS. 44A and 44B. Similarly, it was observed that the differentiated cystic fibrosis cells has a reduced cilia density as compared to healthy differentiated lung cells. See, FIGS. 45A and 45B.

### XVI. Quality Control: Quantitative Measurement of Cell Viability & Differentiation

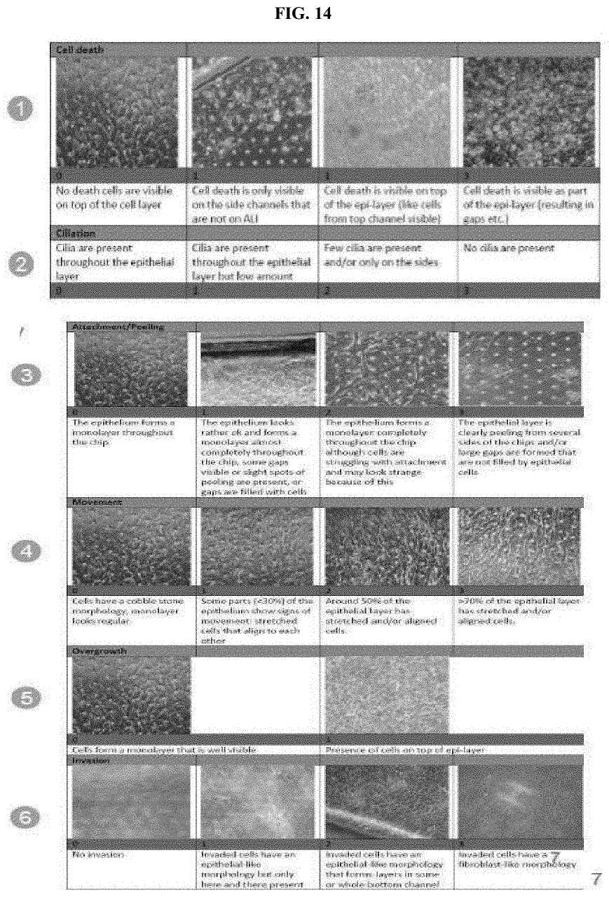

In one embodiment, the present invention contemplates a method comprising scoring at least one cell biomarker and determining a viable and differentiated cell. For example, specific quantitative scores have been assigned to a set of cell biomarkers for viability and differentiation during chip cell culture maturation includes, but is not limited to, cell death (1), ciliation (2) cell attachment (3) cell movement/shape (4), cell overgrowth (5), cell invasion (6). See, FIG. 14.

As depicted, the score value increases as the viability/differentiation relevance of the cell biomarker decreases. In one embodiment, the cell death biomarker score value increases in proportion to increased cell death. In one embodiment, the ciliation biomarker score value increases in proportion to deceased ciliation. In one embodiment, the cell attachment biomarker score value increases in proportion to decreased cell attachment. In one embodiment, the cell movement/shape biomarker score value increases with decreased cell movement/shape. In one embodiment, the cell overgrowth biomarker score value increases with increased cell overgrowth. In one embodiment, the cell invasion biomarker score value increases with increased cell invasion accompanied by a developing fibroblast morphology. In one embodiment, the present invention contemplates a cell biomarker score value of 0-2 that predicts cell viability and differentiation. In one embodiment, the present invention contemplates a cell biomarker score value of 3-5 that does not predict cell viability and differentiation.

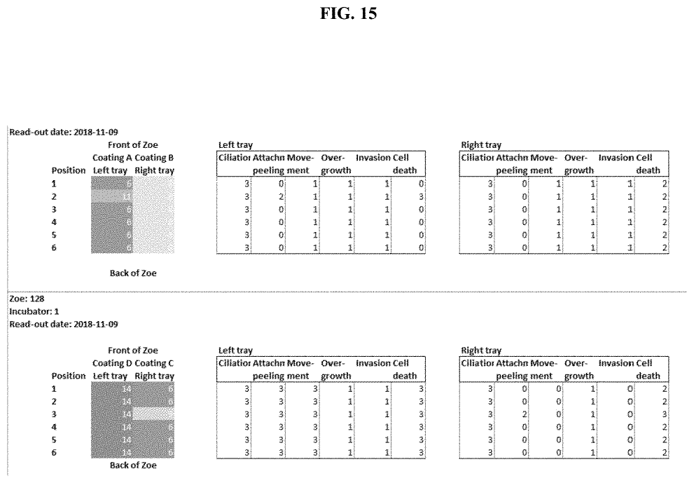

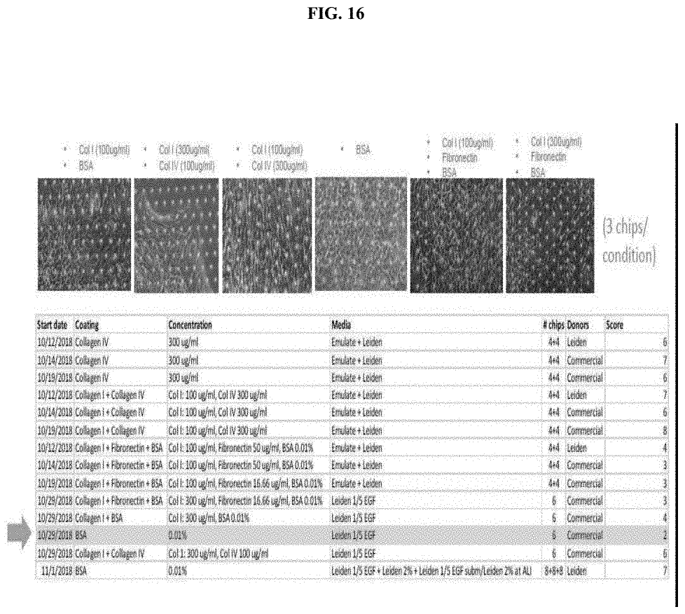

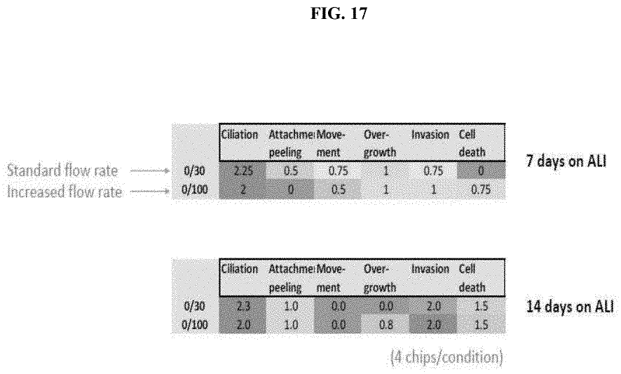

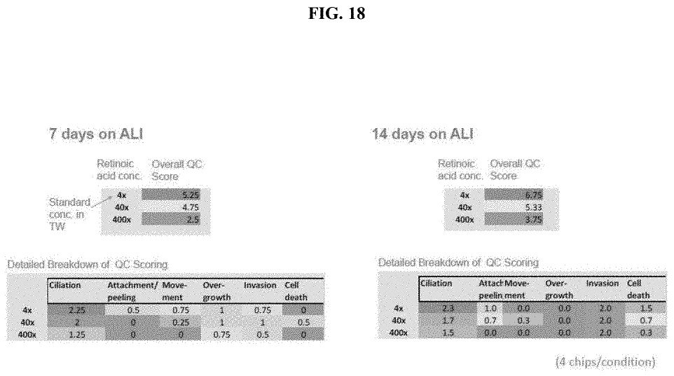

The data presented herein demonstrates the performance of the above method by comparing scored results in four (4) different gel coatings (e.g., Coating A, Coating B, Coating C and Coating D). Each coating can be seen to result in different score values that are independent of location in the cell culture device (e.g., Zoe). See, FIG. 15. This scoring method was utilized to compare fourteen (14) different types of gel layers of which Bovine Serum Albumin (BSA) was found to support the most viable and differentiated cells. See, FIG. 16. The scoring method was also applied to evaluate two different cell culture media flow rates. The data show that an increased flow rate does not affect either cell viability or differentiation parameters. See, FIG. 17. The scoring method was also applied to evaluate different retinoic acids. It is known that retinoic acid (RA) is readily absorbed by most microchip substrates. The data demonstrated that increased RA concentration improves the viability and differentiation of cells. See, FIG. 18.

Although it is not necessary to understand the mechanism of an invention, it is believed that an RA-related compound, EC-23, is minimally absorbed into microchip substrates and is also more light stable (an advantage when using transparent substrates) also supports cell viability and differentiation similar to increased RA concentrations (data not shown).

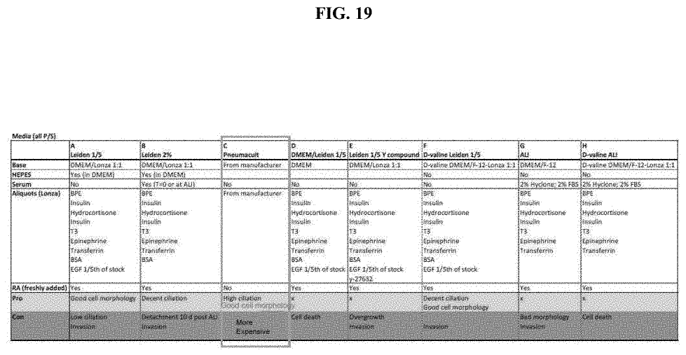

Several cell culture media were also compared using several viability/differentiation biomarkers described above. Morphological assessment found that Pneumacult® (up to 50% dilution) best supports full cell differentiation. See, FIG. 19.

A series of differentiation endpoints were selected to provide a basis for the above scoring method. These differentiation endpoints include, but are not limited to: i) epithelial cell type composition/cilia density; ii) epithelial ciliary beat frequency; iii) epithelial cytokines; iv) epithelial mucus production; v) epithelial morphology; and vi) endothelial cell integrity and function. See, Table 6.

TABLE 6
Representative Cellular Differentiation Endpoints.

| Endpoint | Acceptance Criteria | Reference |
|---|---|---|
| Epithelial cell type | Presence of basal, goblet, | (Amatngalim et al., 2018; |
| composition /cilia density | ciliated and club cells. | Rock et al., 2010; Yang et al., |
| | Ciliation 20-70%. | 2017) |
| Epithelial ciliary beat | Normal distribution; range | (Benam et al., 2016; Shah et |
| frequency | between 5-25 Hz | al., 2009) |
| Epithelial cytokines | 24 h collection of | (Amatngalim et al., 2016) |
| | Il-8 <3000 pg/ml | |
| Epithelial mucus production | Visible mucus production | (Benamet al., 2015) |
| Epithelial morphology | Confluent, ciliated | (Crystal et al., 2008) |
| | cobblestone epithelium; | |
| | pseudostratified; no migration | |
| | between channels | |
| Endothelial cell integrity and | Confluent tissue; Expression | (Cerutti and Ridley, 2017) |
| function | of ICAM-1 only in inflamed | |
| | condition; expression of ve- | |
| | cadherin (tight-junctions) | |

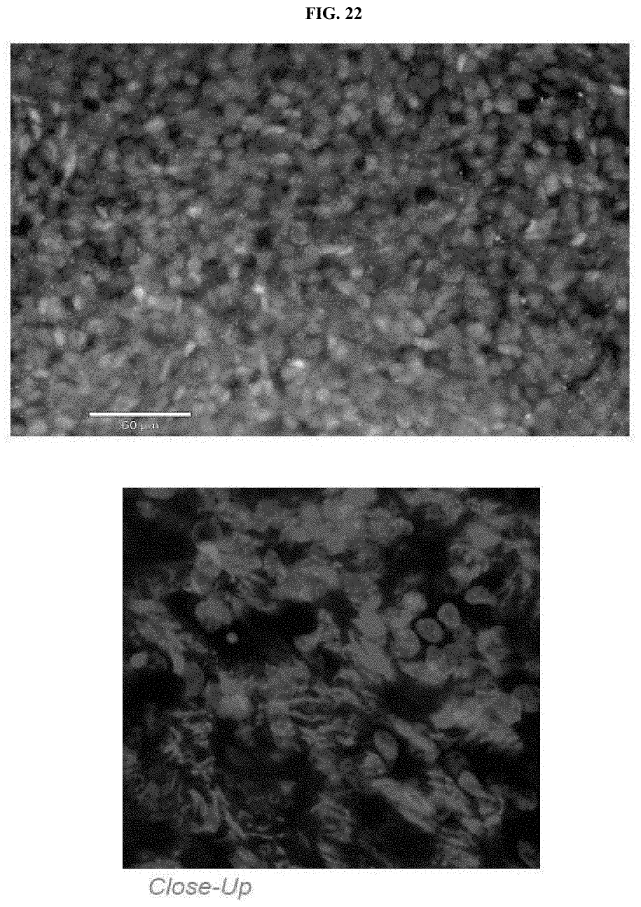

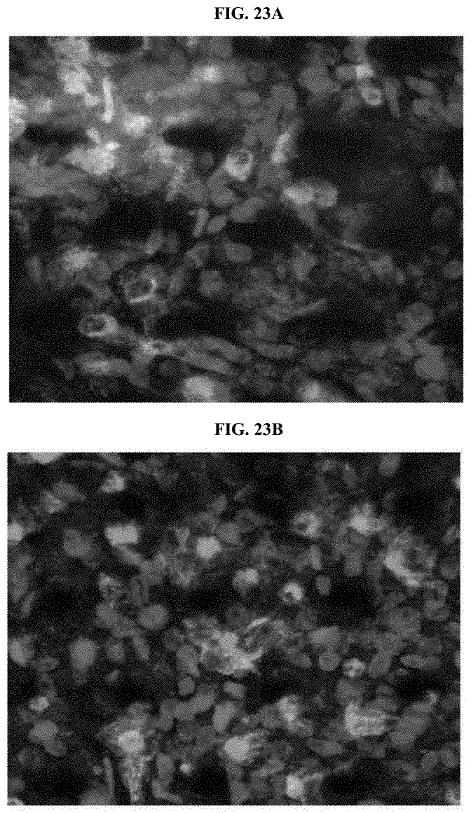

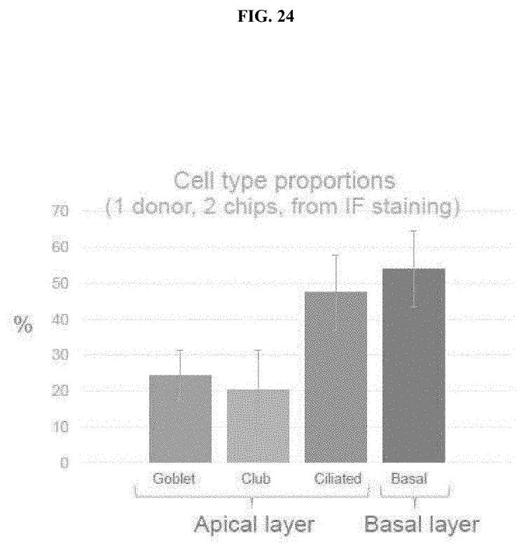

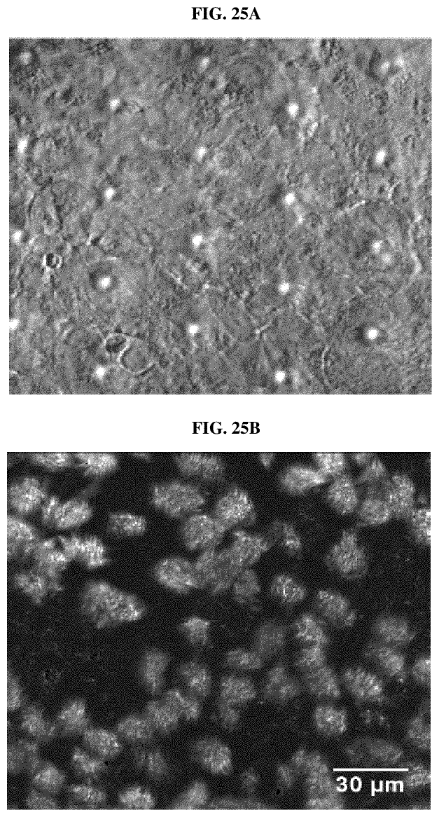

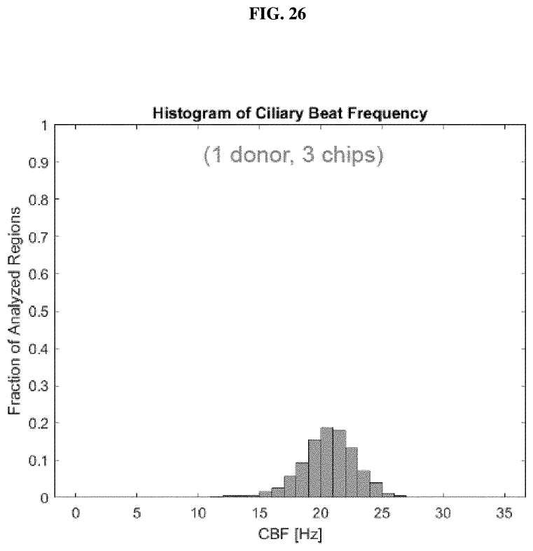

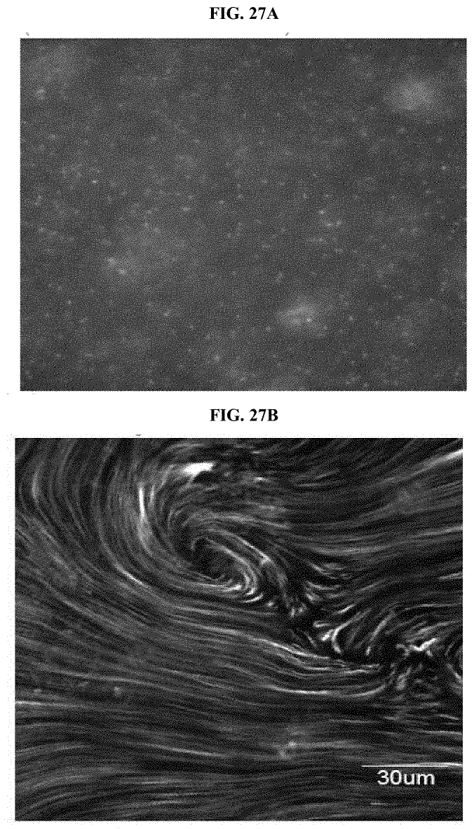

The endpoints may be used, for example, to identify differentiated, ciliated, cells. See, FIG. 22. These data show that lung cells are viable and fully differentiated in optimized conditions after fourteen (14) days of exposure to an ALI. The endpoints may be used, for example, to identify differentiated lung cells into mucus-secreting goblet cells and ciliated club cells. See, FIGS. 23A and 23B, respectively. Quantified data show that lung cells are viable and fully differentiated in optimized conditions after fourteen (14) days of exposure to an ALI. See, FIG. 24. After differentiation into a ciliated cell after seven (7) days exposure to an ALI, a stable ciliary beat frequency was obtained after twenty-one (21) days of exposure to an ALI. See, FIG. 25A. A motion detection scan shows cilia density and cilia beat frequency. See, FIG. 25B. Quantification of the data shows the uniformity of the beat frequency. See, FIG. 26. After differentiation into a ciliated cell after seven (7) days exposure to an ALI, mucociliary transport was obtained after twenty-one (21) days of exposure to an ALI as demonstrated using 1 nm diameter fluorescent beads. See, FIG. 27A. A motion detection scan shows the mucus flow trajectories induced by the cilia-induced mucociliary transport. See, FIG. 27B.

### XVII. Bile Canaliculi Metrics.

In one embodiment, the present invention contemplates a method comprising a microfluidic device having a microchannel with a partial gel layer and a hepatocyte layer. In one embodiment, the method further comprises differentiating bile canaliculi. In one embodiment, the method further comprises scoring the differentiated bile canaliculi. In one embodiment, the differentiated bile canaliculi have significantly improved scores as compared to bile canaliculi differentiated without a partial gel layer.

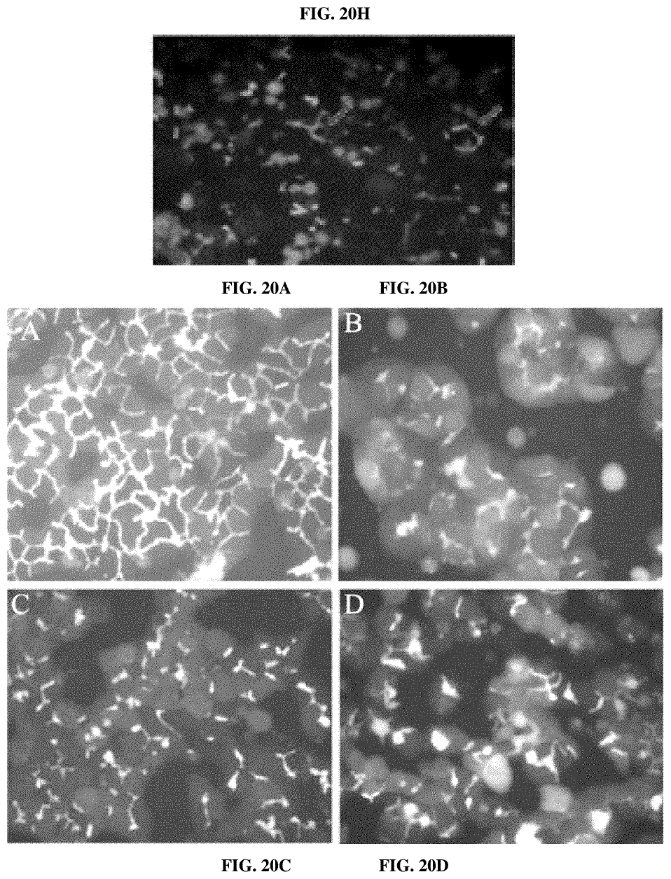

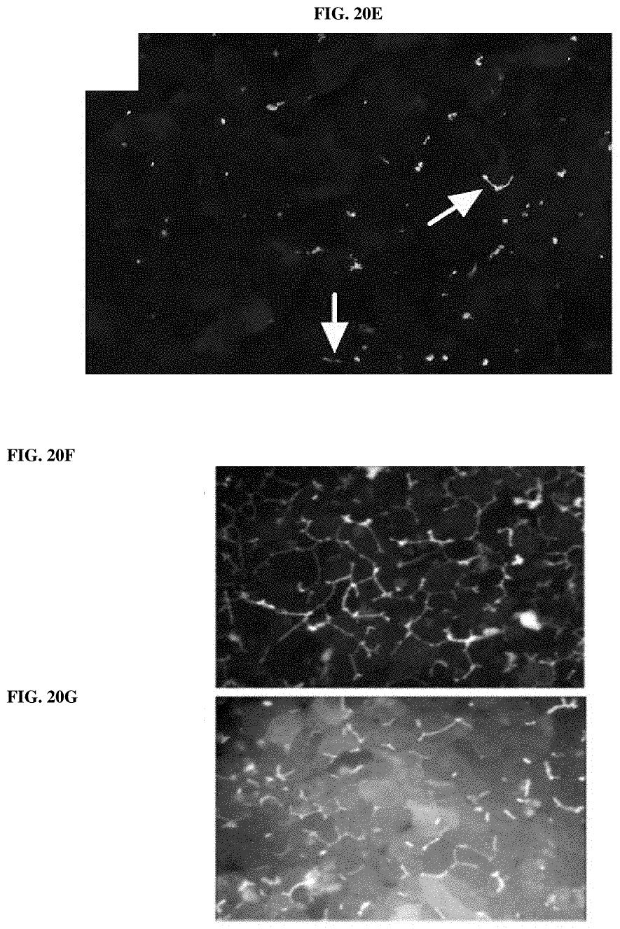

In vitro liver cultures reported in the literature have highly variable extent and architecture of bile canaliculi (BC) networks. To date, no quantitative metric or benchmark of evaluating BC integrity have been proposed or evaluated even though these measures are needed to assess liver function. Currently, subjective evaluations of BC network quality have been used to assess conditions such as tissue maturity, disease, repair, development, drug effects etc. Unfortunately, without a quantitative-based system, no two studies can be directly compared, hence there is now limited potential: i) to study diseases or toxicity (dose response) that affect BC network integrity in vitro since quantitative baseline is missing; ii) to optimize BC network in vitro as to match in vivo structure given lack of quantitative assessment; iii) to study effects of protocol, substrate, cell-cell interactions etc. on BC network formation in vitro given lack of quantitative assessment; and iv) to correlate BC network formation with functional readouts such as biliary clearance or with (pathological) biomarkers (=structure function relationships) in vitro given lack of quantitative assessment. As a result, there here are no tools nor metrics for quality control of BC network formation within and across studies which has resulted in a huge variability of BC networks in published work. See, FIG. 20A-H.

While many individual hepatic parameters that affect BC formation have been reported, there exists no quantification of the parameters that link BC structure to any liver function, such as biliary clearance, which results in an unknown structure function relationship. No quantitative study has been done on the relation between factors including, but not limited to: i) culture conditions, stress, or disease factors; ii) bile canaliculi formation; iii) transporter or other protein expression; and iv) biliary clearance or other liver function.

Human in vitro models of biliary clearance (Cl_{biliary}) should be useful for predicting: i) elimination of drugs via Cl_{biliary} enterohepatic circulation; ii) drug-drug interactions that effect Cl_{biliary}; and iii) drug induced liver injury (DILI) and hepatic diseases that affect Cl_{biliary}. A human cell culture model is needed because it has been reported that expression of transporters is species dependent and Cl_{biliary} in animal models are not always predictive. Grime, 2013. Conventional human liver culture+biliary clearance methods may predict a correct rank order of Biliary Clearance in various compounds, but such predictions are significantly lower than actual clearance rates and show great variability. Abe et al. (2009); and Ghibellini (2007). The reports found variabilities ranging between 5-30 fold between liver samples.

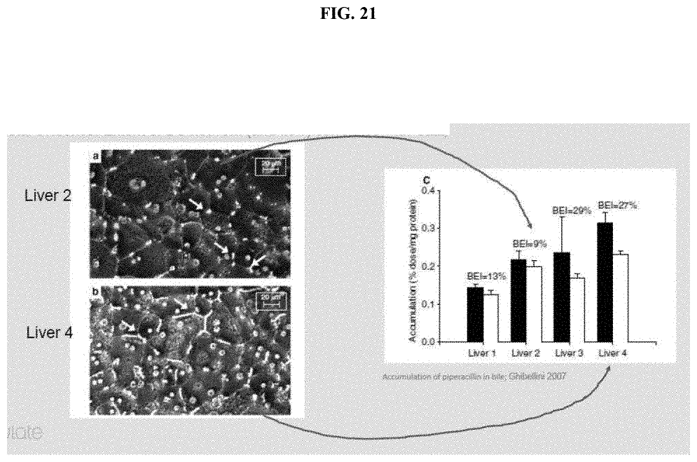

One possible explanation for this variability might be a result of differences in, or incomplete, bile canaliculi differentiation and development. See, FIG. 21. It has been reported that normalizing for absolute and relative expression levels of BC transporters improves in vitro Cl_{biliary} variability. (Li, 2010). Further, it has been noted that the extent of BC network, and therefore biliary flow, correlates with Cl_{biliary} values. See, FIG. 21; and Ghibellini, (2007). Because of these deficiencies in current methods, components that regulate Cl_{biliary}, such as bile flow dynamics and/or bile canaliculi integrity cannot be measured or controlled in vitro. For example: i) the relation between BC network and Cl_{biliary} has never been tested or connected to a mechanism e.g., BC density, presence of flow, expression of mature transporter profile etc.; and ii) a in vivo BC networks (e.g., a human BC network) have never been visualized and/or quantified.

In summary, there is a large variability and consistent underestimation of biliary clearance in state of art human in vitro models. Potential contributors to this effect includes, but are not limited to: i) a large variability of BC network formation in human in vitro models because there are no quality control metrics and no engineering control of BC formation; ii) no mechanistic understanding of relationship between BC network integrity and biliary clearance because only membrane canaliculi transporter density is known factor modulating biliary clearance and evidence that BC network formation is a factor in Cl_{biliary} is merely anecdotal; and iii) no in vitro biliary flow quantitative measurements which is a known factor in Cl_{Biliary} and a sensitive marker of DILI.

In one embodiment, the present invention contemplates a quantitative relationship between local ECM thickness an S1 chip and local BC formation. In one embodiment, the quantitative relationship shows that BC formation is strongly dependent on a partial gel overlay. Although it is not necessary to understand the mechanism of an invention, it is believed that a large degree of partial gel layer thickness is degraded in a constant flow configuration. It is further believed that the partial gel layer degradation can be prevented with improved gel compositions and/or coating methods that would result in improved BC network formations. In one embodiment, the method further comprises quantitative and analytical imaging for fixed (MRP2 stained) and live (CLF or CDFDA treated) in vitro liver cultures. In one embodiment, the method further comprises an image processing algorithm for semi-automatic quantification of BC network. In one embodiment, the image processing algorithm produces quantitative metrics (e.g., branching density, porosity and radius) that defines both extent/quantity and properties/quality of the BC network. Although it is not necessary to understand the mechanism of an invention, it is believed that quantitative metrics can distinguish between hepatic structural remodeling (e.g., in cholestasis) and lack losses in BC networks.

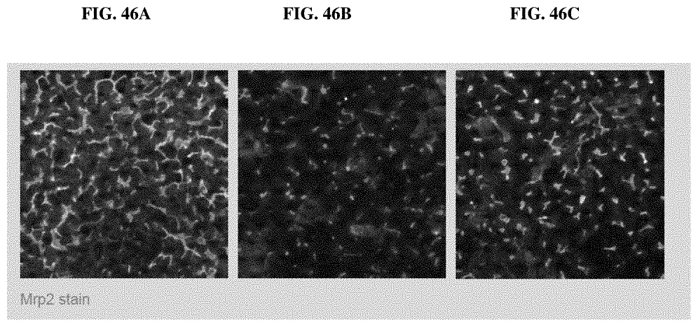

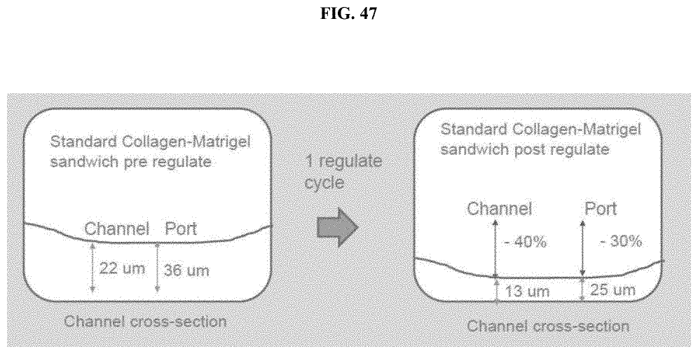

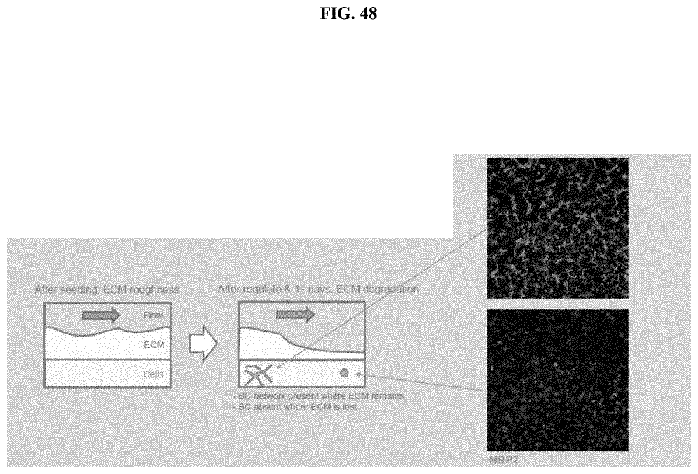

The data presented herein show that when using standard Liver-Chips (e.g., without partial gel layers) variability in BC networks were observed. See, FIG. 46A-C. When evaluating this data it was observed that the best BC networks developed near the inlet and/or outlet ports of the microchannel. The best quality BC networks are observed at, or near, the outlet port. See, FIGS. 46A & B. In contrast, the BC network in the midsection of the main channel is sparse and underdeveloped. See, FIG. 46C. Although it is not necessary to understand the mechanism of an invention, it is believed that fluid shear forces may be responsible for the spatial differences in BC network growth within a microchannel. For example, shear flow is 4-40 times higher in the mid-main channel compared to outlet ports. During normal operations the port locations have a shear of approximately 0.00001 dyn/cm² while the mid-main channel has a shear of approximately 0.00045 dyn/cm². These differences are even greater during a flush cycle (e.g., 0.0026 dyn/cm² versus 0.01038 dyn/cm²). Consequently, partial gel layer height was measured at several points along a microchannel. Five (5) locations (e.g., both ports and three equidistant positions within the main channel) were measured at both the center and the edge. The data show that the initial gels were thicker at the port locations and underwent less degradation than the microchannel locations. See, FIG. 47. Although it is not necessary to understand the mechanism of an invention, it is believed that channel diameter in various locations may partially explain gel deposition and degradation patterns. For example, it is believed that wider channel locations (e.g., port locations) have lower fluid velocities/shear as opposed to narrower channel locations (e.g., mid-mail channel locations) that would have higher fluid velocities/shear. The data further shows that partial gel layer thickness correlates with BC network formation. See, FIG. 48.

It has been reported that BC network formation depends on symmetric partial gel layer top-and-bottom scaffolding which provides the stress cues useful for BC elongation and positioning. Li et al., Nature Cell Biol. (2017). When only a gel scaffold underlay is used, the

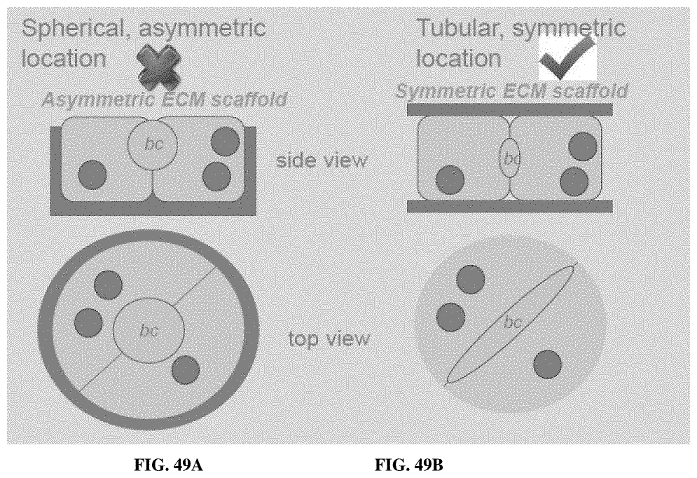

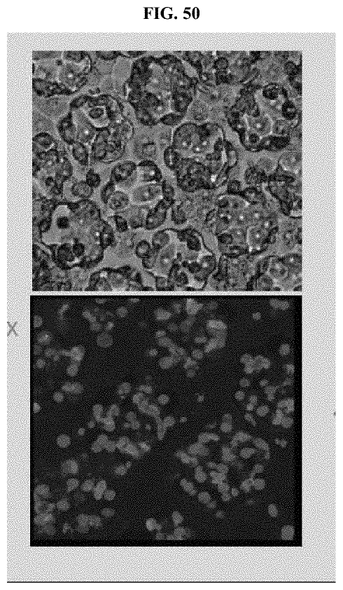

BC is spherical and is asymmetrically located within the cell layer. See, FIG. 49A. However, when a gel scaffold underlay and a gel scaffold overlay is used, the BC is elongated and is symmetrically located within the cell layer. See, FIG. 49B. Photomicrographs confirm this effect by demonstrating a “flower-pot” BC network development accompanied by cholestasis when cultured on only a partial gel underlay. See, FIG. 50.

In one embodiment, the present invention contemplates a method comprising providing a symmetric partial gel layer (e.g., scaffold), reducing fluid shear flow erosion of the partial gel layer and quantifying BC network formation. In one embodiment, the method further comprises controlling BC network formation. In one embodiment, fluid shear flow erosion was reduced with a gel composition including, but not limited to, collagen (I or IV)/fibronectin (FN), fibricol (Fib) collagen, Matritek® and Matrigel®. In some embodiment, the gel composition further comprised an MTG crosslinker. See, Table VII.

TABLE 7
Partial Gel Thickness/Erosion Parameters In Various Gel Compositions

| | | Height | Height | | |
|---|---|---|---|---|---|
| ECM | | pre | post | Delta | |
| conditions | | [um] | [um] | [um] ; % | Notes |
| C + FN − | Ch | 21.25 | 13.7 | −7.6; −36% | pre: filamentous distribution of |
| Matritek ^{®} | Port | 36.5 | 25.75 | −10.7; −30% | beads but homogeneous |
| | | | | | post: in channel, not in port, there |
| | | | | | are big blobs of “remodeled” beads |
| | | | | | sticking out twice as high as |
| | | | | | surrounding layer |
| C + FN | Ch | 40.8 | 38.75 | −2.05; −5% | Two well-defined layers |
| Fib | Port | 84.3 | 52 | −32.3; | Small clumps of beads, |
| Fib | | | | 38% | homogeneously distributed |
| | | | | | Post: some big clumps |
| C + FN | Ch | 36.2 | 23.6 | −12.6; −35% | Two well-defined layers |
| Matritek ^{®} | Port | 82.75 | 54.75 | −28; −32% | Some mixing of layers |
| Fib | | | | | Small clumps of beads, |
| | | | | | homogeneously distributed |
| C + FN | Ch | 31.6 | 23.6 | −2.8; −8% | Two well-defined layers |
| Matritek ^{®} | Port | 63 | 54.75 | −5.25; −8% | Very homogeneously distributed |
| Fib + FN | | | | | few clumps of beads |
| C + FN − | Ch | 21.25 | 13.7 | −7.6; −36% | pre: filamentous distribution of |
| Matritek ^{®} | Port | 36.5 | 25.75 | −10.7; −30% | beads but homogeneous |
| | | | | | post: in channel, not in port, there |
| | | | | | are big blobs of “remodeled” beads |
| | | | | | sticking out twice as high as |
| | | | | | surrounding layer |
| C + FN | Ch | 40.8 | 38.75 | −2.05; −5% | Two well-defined layers |
| Fib | Port | 84.3 | 52 | −32.3; | Small clumps of beads, |
| Fib | | | | 38% | homogeneously distributed |
| | | | | | Post: some big clumps |
| C + FN | Ch | 36.2 | 23.6 | −12.6; −35% | Two well-defined layers |
| Matritek ^{®} | Port | 82.75 | 54.75 | −28; −32% | Some mixing of layers |
| Fib | | | | | Small clumps of beads, |
| | | | | | homogeneously distributed |
| C + FN | Ch | 31.6 | 23.6 | −2.8; −8% | Two well-defined layers |
| Matritek ^{®} | Port | 63 | 54.75 | −5.25; −8% | Very homogeneously distributed |
| Fib + FN | | | | | few clumps of beads |

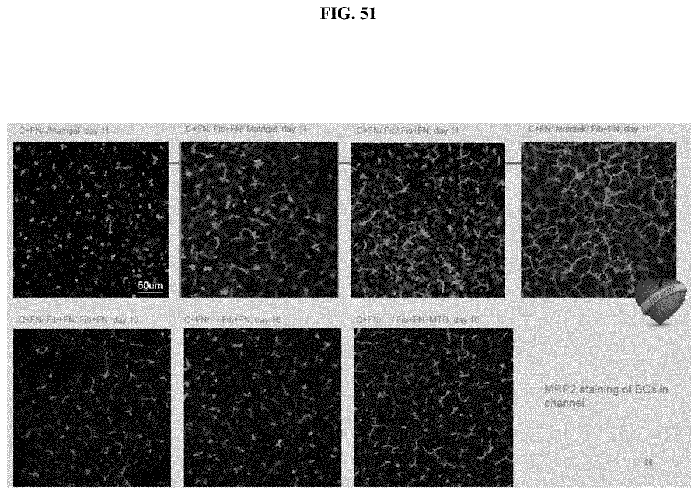

The data show that a collagen/fibronectin/Matrigel® overlay with a collagen/fibricol underlay provided the best results. This tabulated data is demonstrated in photomicrographs showing significant improvement of BC networks that correlate with increased partial gel layer thickness and reduced erosion (e.g. delta). See, FIG. 51.

In one embodiment, the present invention contemplates a method comprising calculating

BC network geometrical metrics. Meyer et al., (2017). In one embodiment, the geometrical metric comprises an average BC radius (ΣRₙ/N). In one embodiment, the geometrical metric comprises a branching density (ΣLₙ/Area of RO_{I}). In one embodiment, the geometrical metric comprises a porosity (Σ Aₙ/Area of RO_{I}). In one embodiment, increased BC network quality is determined by a decrease in average BC radius. I one embodiment, increased BC network quality is determined by an increase in branching density. In one embodiment, increased BC network quality is determined by an increase in porosity.

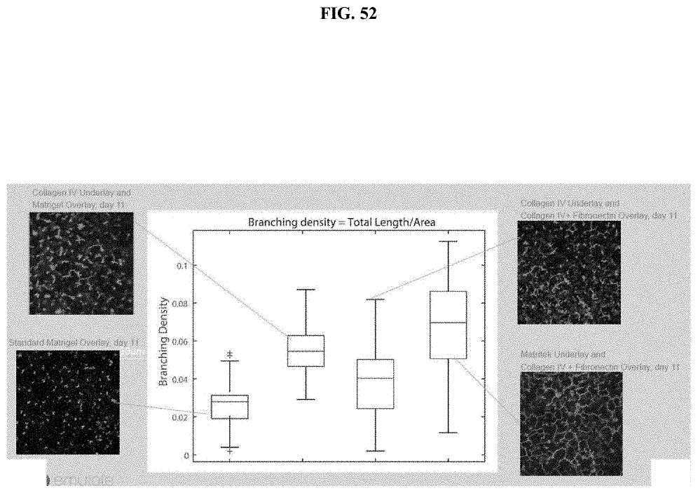

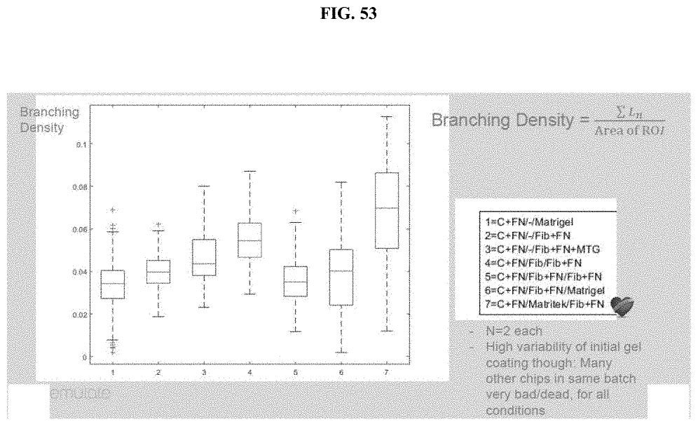

Representative branching density data was collected comparing four gel compositions identified in Table 7. It can be seen that the branching density metric reflected the observed visual BC network quality in the associated photomicrographs. The data demonstrate that branching density, and observed visual quality was highest in the collagen/fibronectin/Matrigel® overlay with a collagen/fibricol underlay configuration and correlates with the above gel thickness/erosion data. See, FIG. 52. A further assessment compared the branching density metric between seven different gel compositions. These data show that a collagen+fibronectin/Matritek/fibricol+fibronectin gel layer resulted in the highest BC network branching density metric. See, FIG. 53. These data demonstrate that by using a BC network analysis (e.g., radius, density and porosity), subtle differences between BC network quality and quantity can be distinguished and quantitated. The above metrics can be used alone or in combination with other statistical methods (e.g., regression models, principal component analysis, etc.) to identify population differences and to a provide statistical sample comparison.

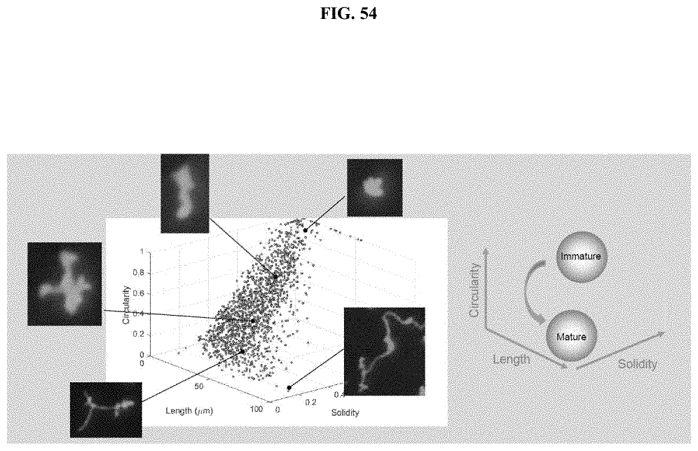

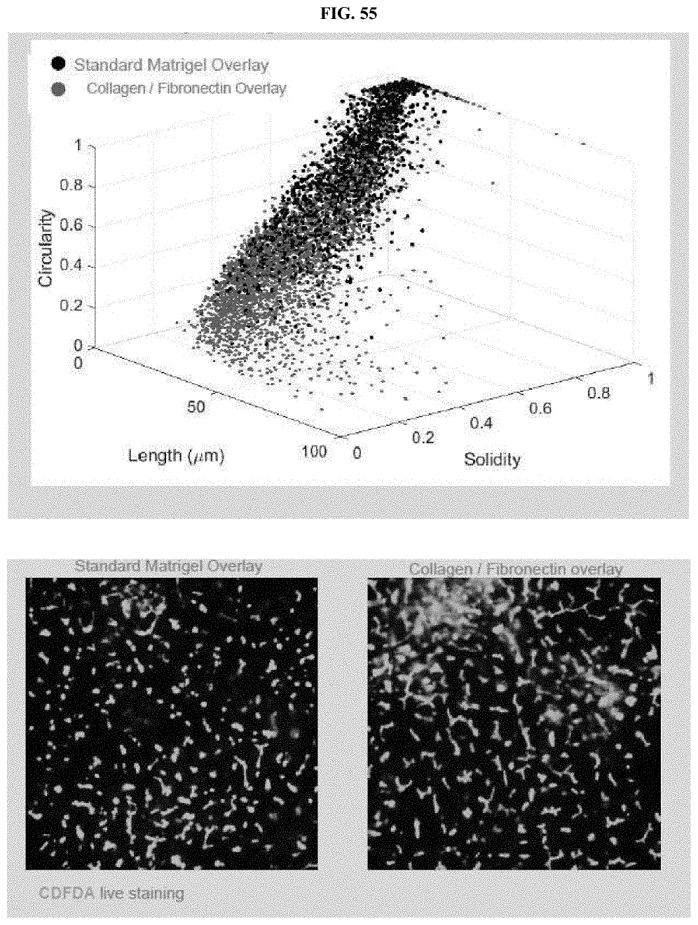

Alternatively, a BC network metric comprises a shape analysis to evaluate immature networks with little branching. These shape analysis parameters include, but are not limited to BC circularity, BC length and BC solidity. The data show that a shape metrics 3D plot of individual BC shape parameters measures the relative degree of BC network maturation. A 3 D plot of circularity, length and solidity clearly differentiates between immature BC networks and mature BC networks. See, FIG. 54. This shape analysis method was applied to compare the relative abilities of a collagen+fibronectin partial gel layer overlay to differentiate a BC network in comparison to a standard Matrigel® partial gel layer overlay. The data show a clear distinction in that the BC networks were vastly superior when the cells were cultured with a collagen+fibronectin overlay. See, FIG. 55.

In one embodiment, the present invention contemplates a BC network metric analysis method to determine cell parameters including, but not limited to health, toxicity, maturity, stress, disease, quantity, quality, tissue markers, gel layer integrity, cell types and cell factors. In one embodiment, the method further comprises standardizing tissues. In one embodiment, the method further comprises correlating functional readouts to assess structure-function relationships. In one embodiment, the method further comprises culturing an in vivo-like BC network. In one embodiment, the method further comprises measuring biliary flow in an in vitro BC network. In one embodiment, the method further comprises modulating biliary flow by changes in BC network formation. In one embodiment, the method further comprises standardizing phenotypic profiling. In one embodiment, the method further comprises integrating with genomic databases (e.g., Omics) to determine genotype/phenotype relationships. In one embodiment, the method further comprises developing treatment dose responses for DILI, liver damage and biliary function defects.

### XVIII. Three Dimensional Partial Gel Layers.

As discussed above, an empirical selection of one or more partial gel layers leads to unexpected and surprising improvements in cell differentiation during in vitro cell culture. Also, it has been observed that the addition of hepatic stellate cells to gel layer underlays of cultured hepatocytes. Both of these approaches significantly improved variability conventionally existing in cell culture microarchitecture such as BC network formation. In one embodiment, the present invention contemplates methods for improvement in the layering of partial gel layers (e.g., underlays or overlays). Although it is not necessary to understand the mechanism of an invention, it is believed that these improved methods provide reproducibility in cell development and differentiation, for example, by reestablishing hepatocyte polarity in culture.

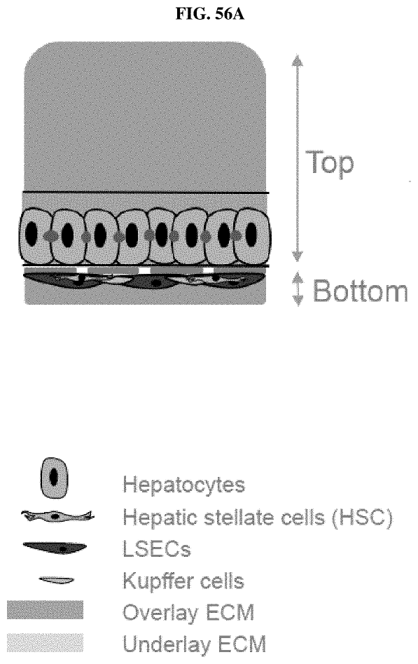

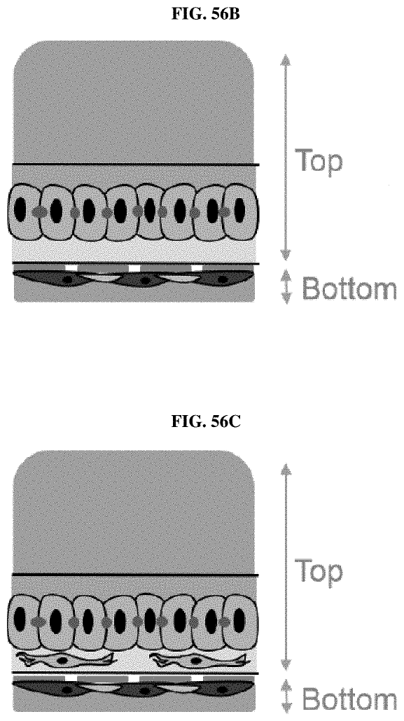

Currently, most cell culture compositions comprise a cell layer in between a single gel overlay and a standard molecular extracellular matrix. See, FIG. 56A. In one embodiment, the present invention contemplates an improved cell culture composition comprising a cell layer sandwiched between an overlay gel layer and an underlay gel layer, where the underlay gel layer covers a standard molecular extracellular matrix. See, FIG. 56B. In one embodiment, the improved cell culture composition further comprises a stellate cell layer embedded within the underlay gel layer. See, FIG. 56C. In one embodiment, the cell layer is a hepatocyte cell layer. In one embodiment the stellate cell layer is a hepatic stellate layer (HSC). Although it is not necessary to understand the mechanism of an invention, it is believed that the improved cell culture composition induces a reproducible and controllable bile canaliculi formation in the hepatocytes.

A. Partial Gel Layer Production with a Hydrodynamic Fluid Flush.

In one embodiment, the present invention contemplates a method for depositing a partial gel layer in a microchannel. In one embodiment, the method provides a robust creation of a 3D partial gel layer with a relatively homogeneous thickness. In one embodiment, the method comprises: i) polymerizing a gel matrix solution on a microchannel surface during an overnight (e.g., approximately twelve hours) incubation; ii) hydrodynamic flushing the microchannel surface with a fluid under conditions such that an anchored 3D partial gel layer remains on the microchannel surface. In one embodiment, the gel matrix solution comprises <0.5 mg/ml of a gel polymer (e.g., collagen). Although it is not necessary to understand the mechanism of an invention, it is believed that gel matrix solutions having a gel polymer of >0.5 mg/ml do not form partial gel layers. It is believed that the higher polymer concentration results in polymerized gel density that is not subject to fluid flushing after an overnight incubation.

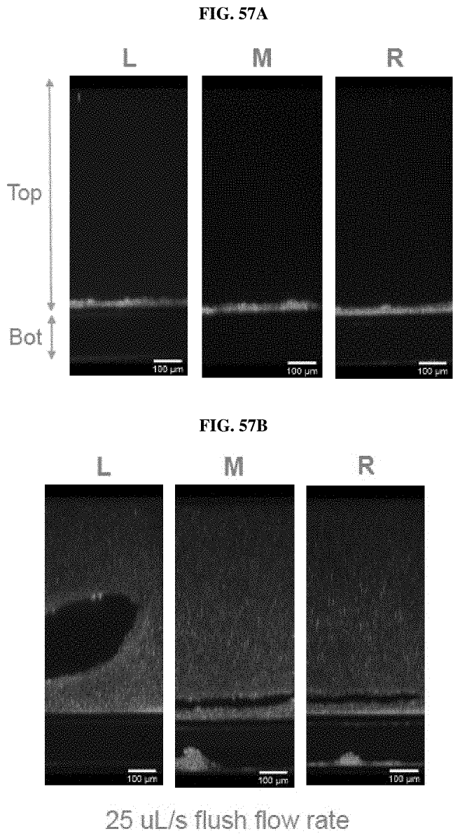

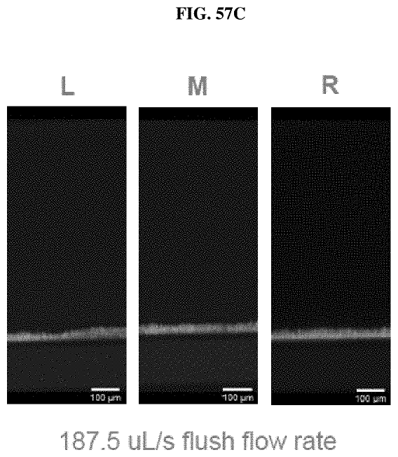

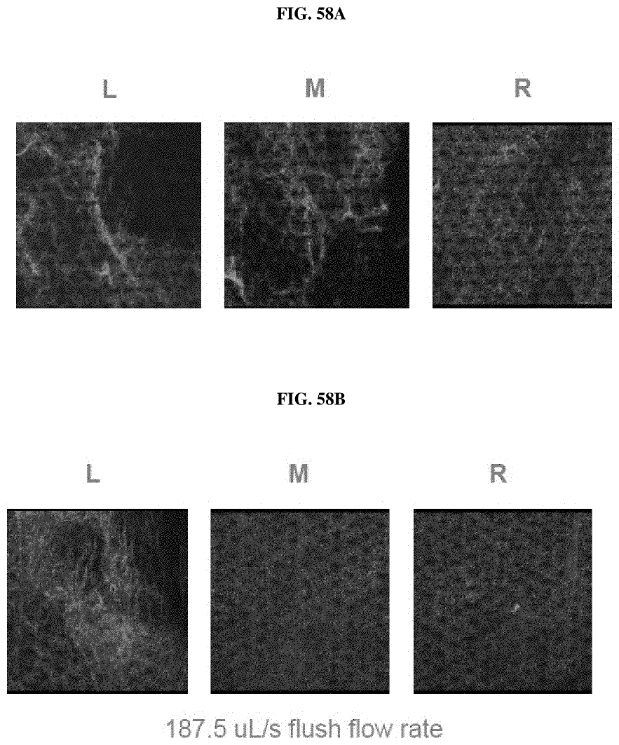

Different methods of hydrodynamic fluid flushing were compared for their ability to deposit a thick and homogeneous partial gel layer. In particular, a manual fluid flush (e.g., using a standard hand-held syringe) was compared to a commercially available, programmable, automatic pipettor (e.g., Eppendorf). The data demonstrates that an ability to deposit a thick and homogeneous partial gel layer is found using an automatic pipettor. In particular, a manual syringe flush resulted in discontinuous and non-homogeneous thin (partial) gel layers at three locations within a main microchannel of a microchip device (left, middle, right; L/M/R). See, FIGS. 57A and 58A (cross-sectional view and top view, respectively). When an automatic pipettor was used at a low shear velocity (25 μl/sec) incomplete flushing was observed that did not result in a partial gel layer with a smooth and flat surface. See, FIG. 57B (cross-sectional view. However, when the automatic pipettor was used at a high shear velocity (187.5 μl/sec) a thick, homogeneous partial gel layer with a smooth and flat surface was created. See, FIGS. 57C and 58B (cross-sectional view and top view, respectively).

B. Empirical Gel Composition Coverage Thresholding.

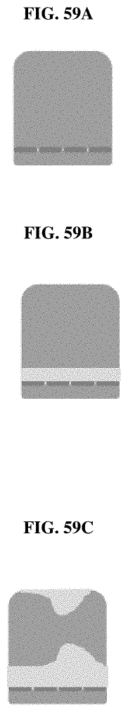

Despite the success of the above described hydrodynamic fluid flush method to create a partial gel layer, it was further to be determined if all gel solution matrix had equivalent polymerization properties. In fact, the data presented herein demonstrates that gel polymer composition is a determining factor in creating a partial gel layer having a smooth and flat surface. Assessment parameters of gel layer coverage includes, but is not limited to; i) molecular extracellular matrix (2-D area on one microchannel surface); ii) 3-D gel layer (2-D/2 area on one microchannel surface); and iii) thick 3-D gel layer (3-D area on multiple microchannel surfaces). See, FIG. 59A-C.

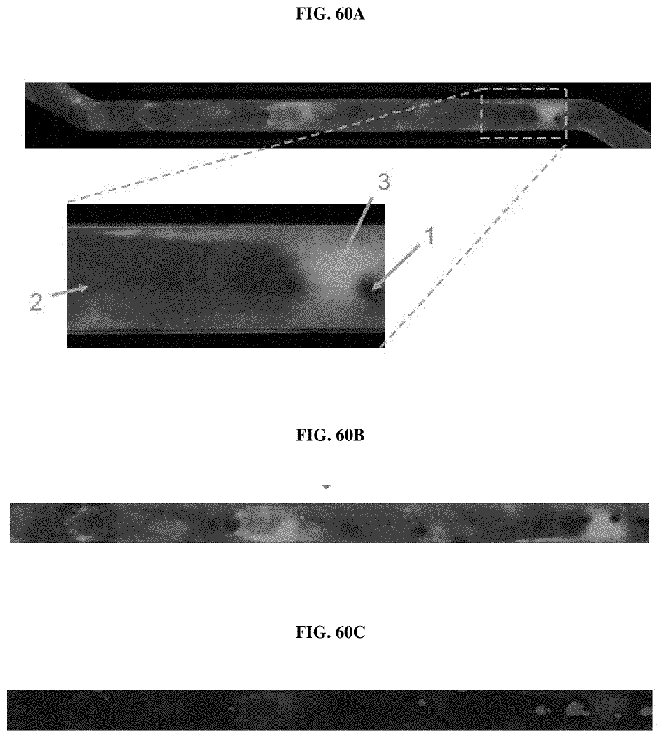

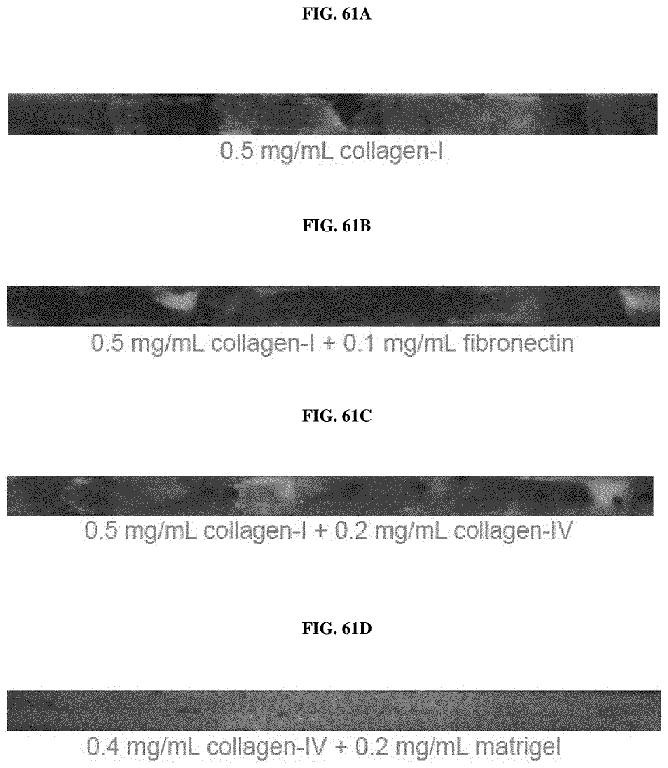

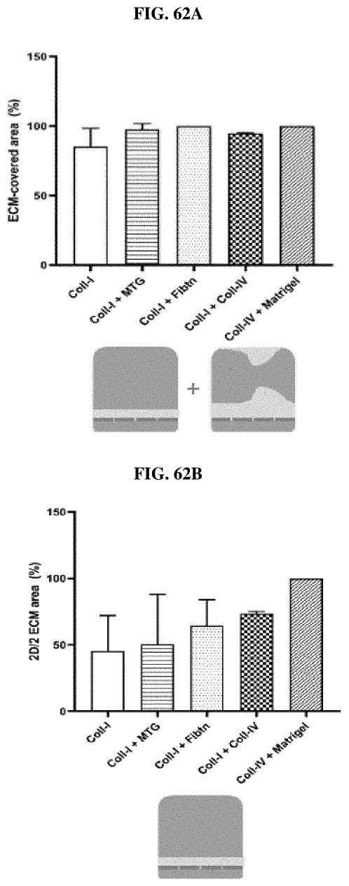

These assessment rating parameters were then used to identify specific areas on a discontinuous partial gel layer. See, FIG. 60A-C. In particular: i) Ranking #1 refers to a 2-D area; Ranking #2 refers to a 2-D/2 area; and iii) Ranking #3 refers to a 3-D area. See FIG. 60A. Once the areas are ranked, wherein a thresholding method combines the image intensities corresponding to Rankings #2 & #3 data and then subtracts the Ranking #3 data, thereby isolating the location of the Ranking #2 areas (dark regions) and gel layer gaps (e.g., red regions). See, FIG. 60C. These ranking parameters were then used to assess the partial gel layer coverage quality for several different gel solution matrices using a hydrodynamic flush method. See, FIG. 61A—D. The data show that the collagen I gel mixture and the collagen I+fibronectin gel mixture provided superior coverage, both having a 2-D/2 area Ranking #2 score. See, FIG. 61A and FIG. 61B. On the other hand, the collagen I+collagen IV gel mixture and the collagen IV+Matrigel® gel mixtures provided unsuitably thick partial gel layers, both having a 3-D area Ranking #3 score. See, FIGS. 61C and 61D. The thresholding algorithm was used to calculate coverage for partial gel layers deposited using several different gel solution matrices. First, the total gel coverage area was determined (e.g., 2-D/2 areas+3-D areas). The data show that among the five different gel compositions compared, all compositions deposited approximately the same amount of gel in the microchannel. See, FIG. 62A. However, when the 3-D areas were subtracted from this total amount, thereby reflecting only 2-D/2 areas, significant differences in gel coverage were observed between the gel compositions. These data show that collagen IV mixtures with either collagen I or Matrigel® showed the greatest coverage of 2-D/2 areas. See, FIG. 62B.

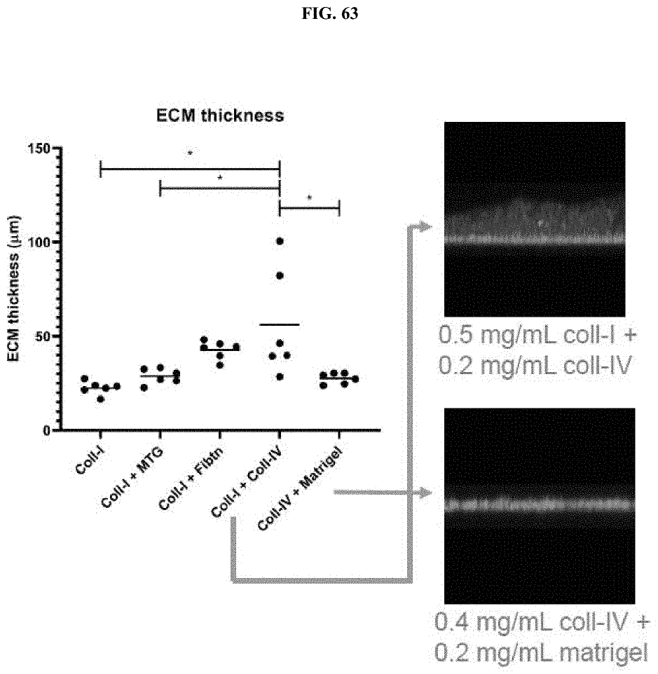

The thickness of each gel layer was also determined using the thresholding method. The data show that the collagen IV mixtures with either collagen I or Matrigel® showed the greatest gel layer thicknesses (e.g., ranging between approximately 25 μm-100 μm). See, FIG. 63.

C. Thick-Walled Lumens.

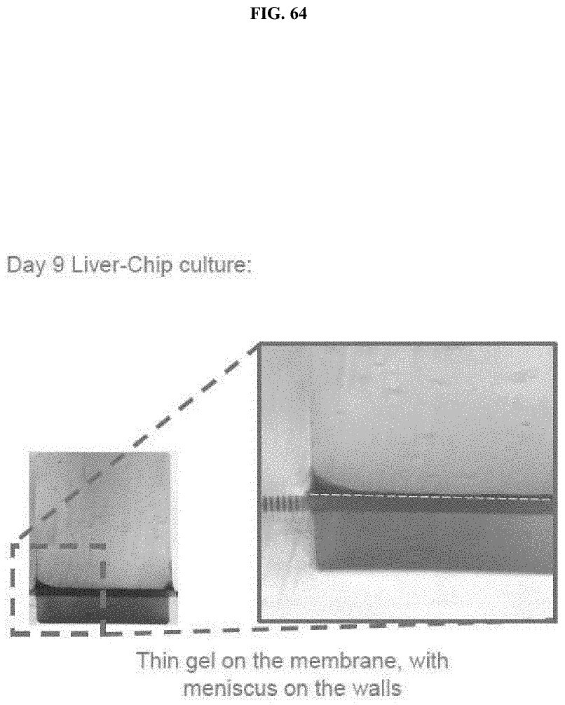

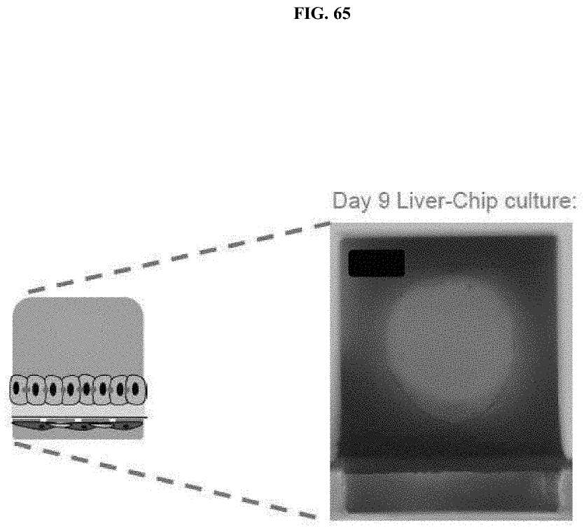

Some of the partial gel layers as presented herein have occasionally been observed to undergo a measureable amount of erosion/degradation during flow conditions. For example, after nine (9) days of fluid flow one partial gel layer demonstrated a thin middle layer with thicker sides, thereby forming a meniscus. See, FIG. 64. In one embodiment, the present invention contemplates a method for creating a stable gel layer with a lumen. In one embodiment, the stable gel layer does not undergo degradation when under fluid flow. See, FIG. 65.

The creation of this stable gel layer was a result of creating a partial gel layer using a viscous fingering technique. In one embodiment, a lumen is created within five (5) seconds of deposition of the high concentration gel polymer (e.g., >5 mg/ml). In one embodiment, the method creates a gel thickness between the interior lumen wall and microchannel surface of approximately 200 μm.

Although it is not necessary to understand the mechanism of an invention it is believed that a thick walled interior lumen within a gel-filled microchannel improved in vitro cell growth and differentiation.

1. Hepatocyte Growth and Differentiation.

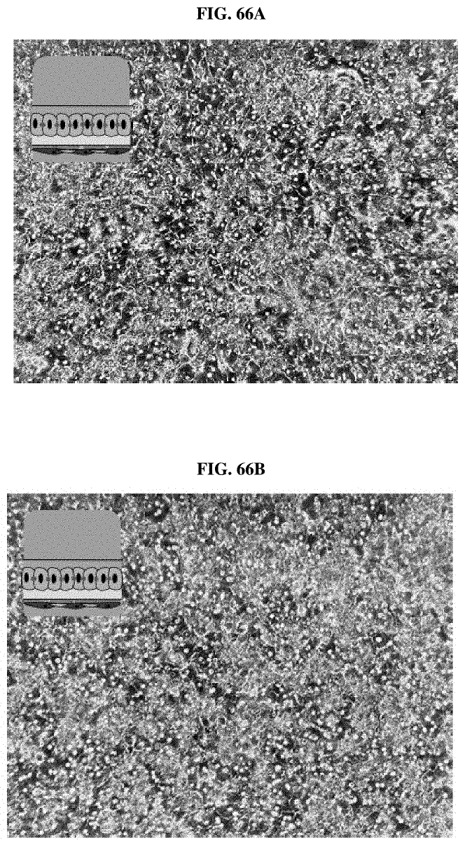

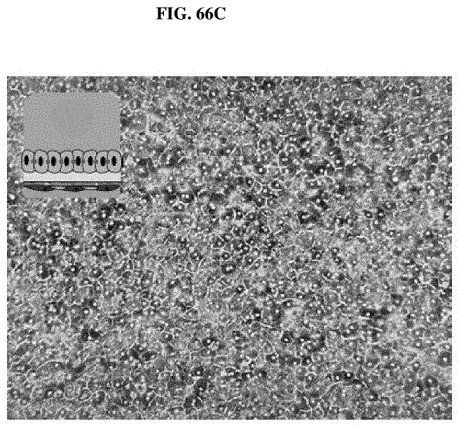

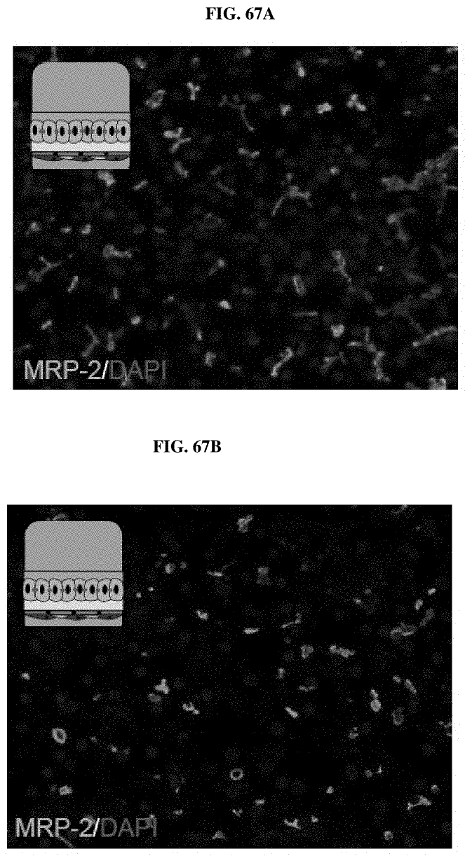

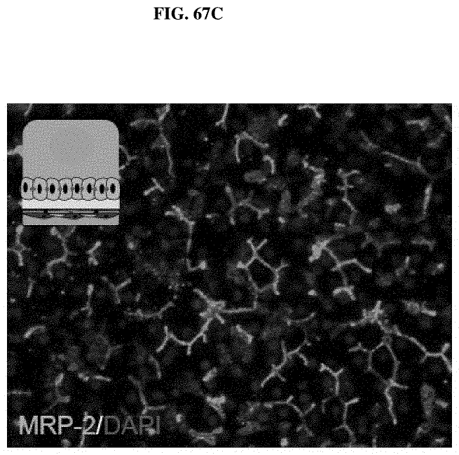

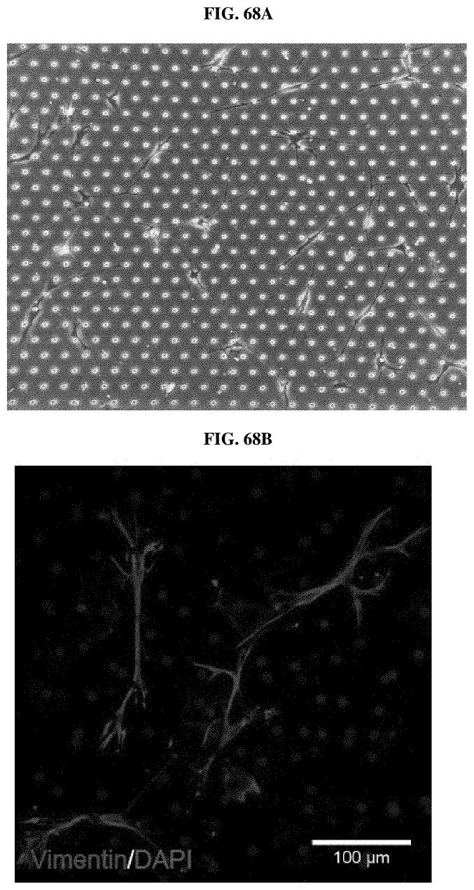

In one embodiment, the present invention contemplates a microchannel comprising a surface coated with a gel underlay, and a cell layer adhered to the gel underlay and a thick gel overlay. In one embodiment, the cell layer is a hepatocyte layer. In one embodiment, the thick gel overlay is approximately 50 μm-200 μm. In one embodiment, the gel underlay is approximately 25 μm. In one embodiment, the thick gel overlay comprises a homogenous, continuous lumen of a constant diameter. The data presented herein demonstrate the morphology of hepatocyte monolayers using the above described underlay/thick overlay configuration using a variety of gel compositions. Of interest, the morphology of a thick collagen I gel overlay with a lumen had better hepatocyte morphology than thick flat surfaced collagen I+fibronectin or collagen I+collagen IV partial gel overlays. See, FIGS. 66A-66C. A further study of these differentiated hepatocytes demonstrated developing bile canaliculi (BC) using the above described underlay/thick overlay configuration using a variety of gel compositions. Of interest, BC development in a thick collagen I gel overlay with a lumen appears superior to BC development using either a thick flat surfaced collagen I+fibronectin or collagen I+collagen IV partial gel overlays. See, FIGS. 67A-67C. BC development was further improved in the thick collagen I gel overlay with a lumen after embedding hepatic stellate cells (HSCs) in the gel underlay. See, FIGS. 68A and 68B.

2. Alcoholic Liver Disease

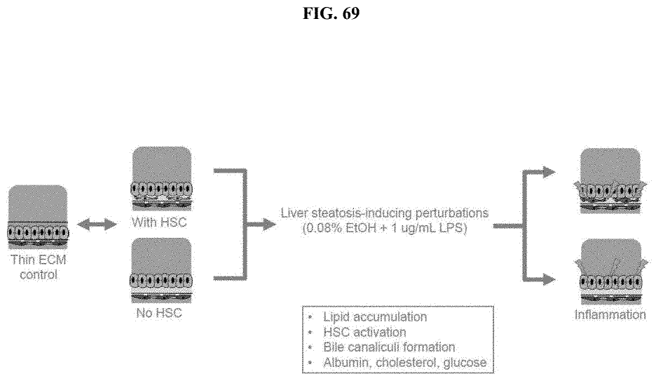

In one embodiment, the present invention contemplates a microchannel comprising a surface coated with a gel underlay, and a hepatocyte cell layer adhered to the gel underlay and a thick gel overlay. In one embodiment, the microchannel further comprises a physiologically relevant ethanol concentration. In one embodiment, the thick gel overlay is approximately 50 μm-200 μm. In one embodiment, the gel underlay is approximately 25 μm. In one embodiment, the thick gel overlay comprises a homogenous, continuous lumen of a constant diameter. The data presented herein demonstrate that the microchannel can be used as a model to induce alcoholic liver disease as measured by hepatic steatosis. Various ALD biological parameters may be measured including, but not limited to, lipid accumulation, hepatic stellate cell activation, BC network formation, albumin levels, cholesterol levels and/or glucose levels. See, FIG. 69.

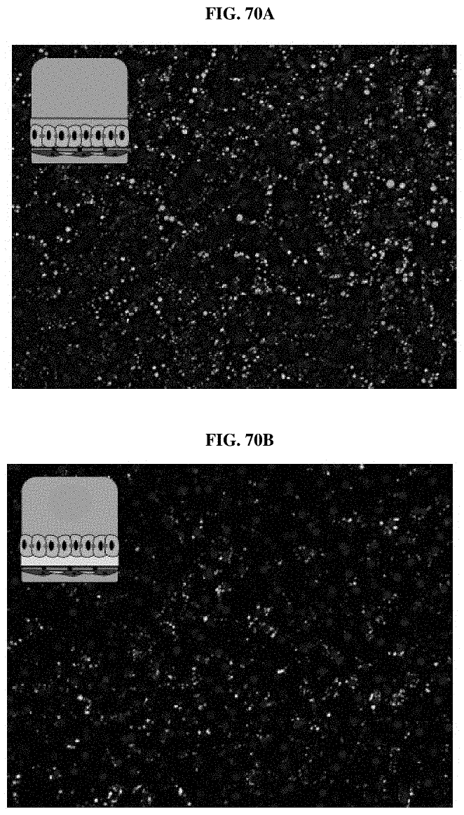

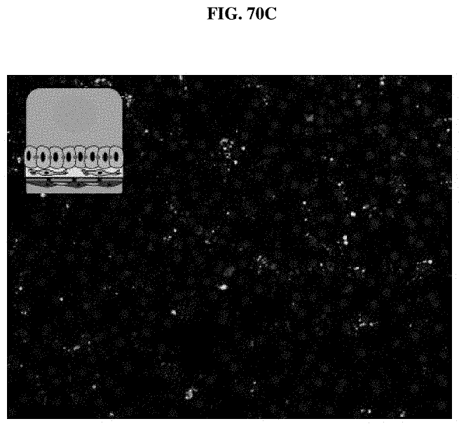

The design changes in the in vitro microchannel model was found to induce lipid accumulation in hepatocytes, without ethanol treatment. For example, in hepatocyte cells, it was surprisingly found that lipid accumulation was reduced when a thick gel layer overlay was deposited on the top surface of the hepatocyte cells as opposed to a thin gel layer overlay. In particular, the data presented herein shows that lipid accumulation in hepatocytes cultured on a conventional thin Matrigel® overlay, defined as a gelatinous protein mixture secreted by Engelbreth-Holm-Swarm mouse sarcoma cells) (0.2 mg/ml) is greater than with a thick collagen I overlay (5.0 mg/ml). See, FIG. 70A and FIG. 70B. Further reductions in lipid accumulation, without ethanol treatment, was observed when HSCs were co-cultured with the hepatocytes, thereby suggesting an HSC protective effect on hepatocyte lipid accumulation. See, FIG. 70B and FIG. 70C.

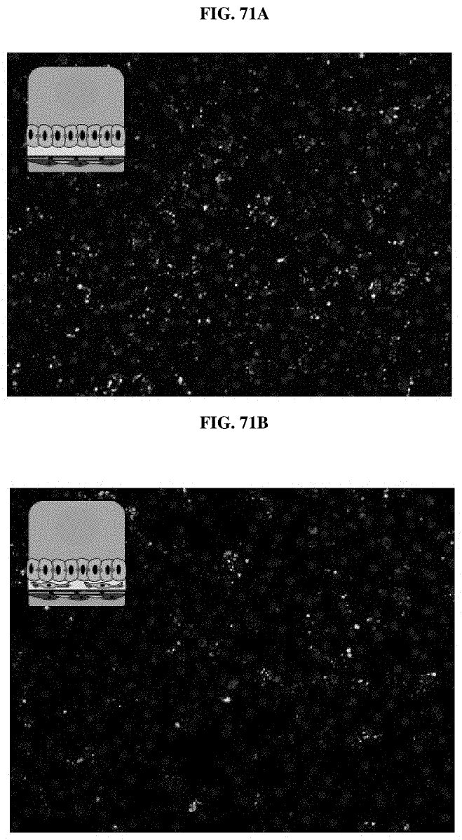

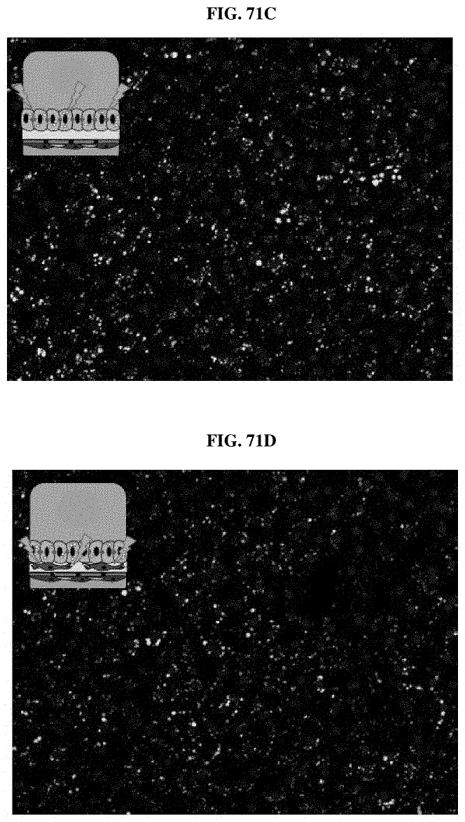

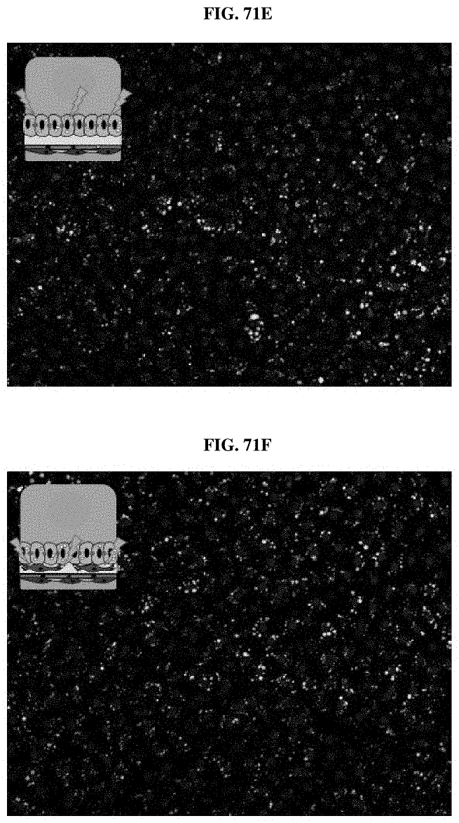

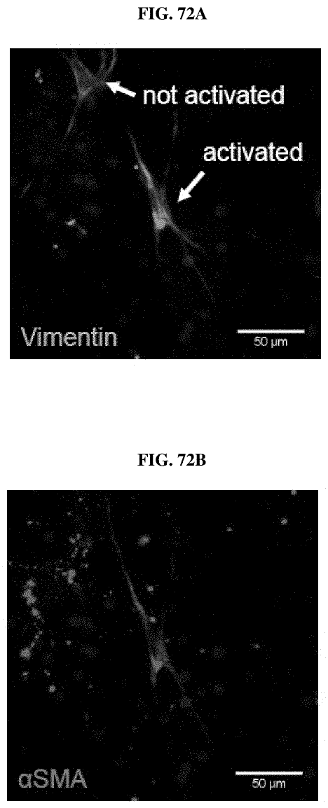

This phenomenon was further addressed by comparing lipid accumulation in hepatocytes cultured without HSCs but having thick gel overlays in the presence or absence of 0.08% ethanol and/or lipopolysaccharide (LPS). The data show that, when using thick collagen I overlays, the presence/absence of ethanol, LPS and/or HSCs resulted in significant changes in hepatocyte lipid accumulation. The data presented herein demonstrate that: i) HSCs reduce lipid accumulation in hepatocyte cells in the absence of both ethanol and LPS (see, FIGS. 71A and 71B); ii) HSCs reduce ethanol-induced lipid accumulation in hepatocyte cells in the absence of LPS (see, FIGS. 71C and 71D); and iii) HSCs have little effect on ethanol+LPS lipid accumulation in hepatocyte cells (see, FIGS. 71E and 71F). Following treatment with ethanol and LPS, hepatocyte cells were found to be activated by detecting expression of both vimentin and alpha-SMA. See, FIGS. 72A and 72B.

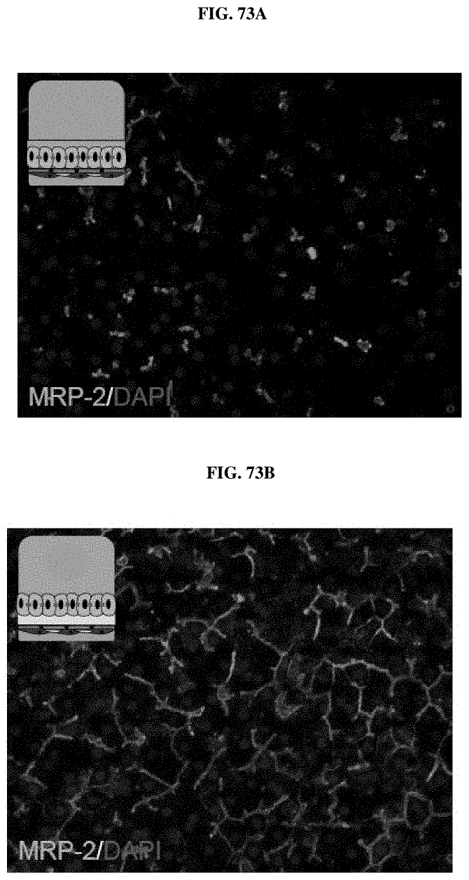

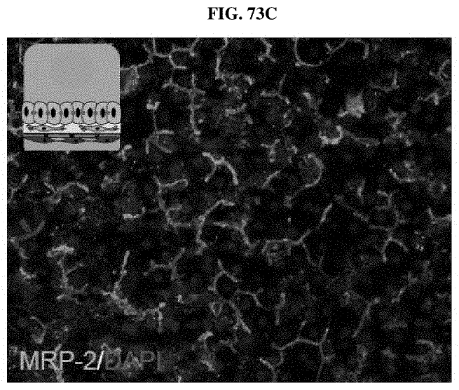

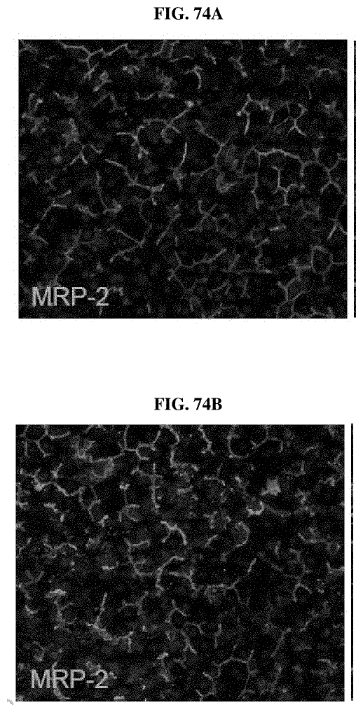

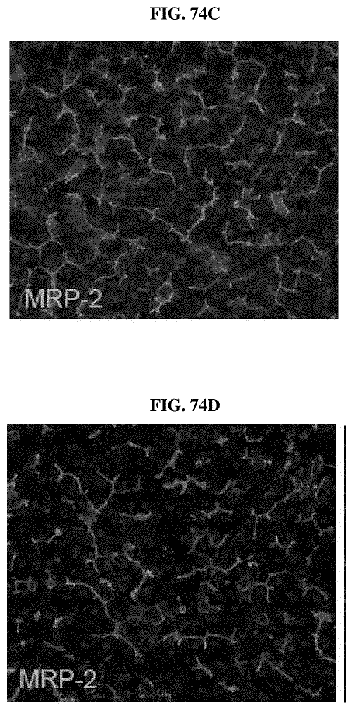

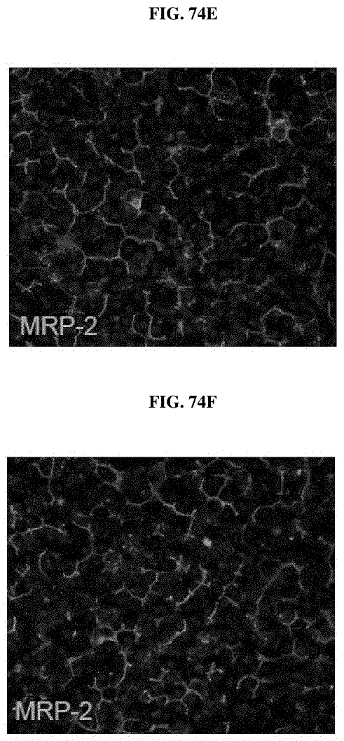

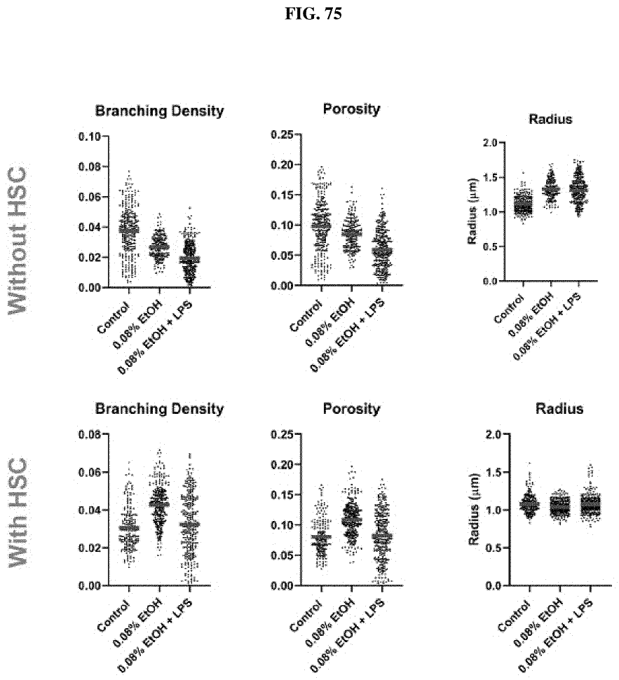

Similar to the above lipid accumulation data, the ability of HSCs to improve BC network development of a hepatocyte layer was further confirmed. In particular, the thick collagen I gel overlays were shown to have improved BC network as compared to standard thin Matrigel® overlays. See, FIG. 73A and FIG. 73B. However, when HSCs were added to the thick collagen I gel overlay composition a further improvement in BC network was observed. See, FIGS. 73B and 73C. Photomicrographs presented herein further demonstrate that: i) HSCs did not markedly effect BC network development in hepatocyte cells in the absence of both ethanol and LPS (see, FIGS. 74A and 74B); ii) HSCs improved ethanol-induced BC network development reductions in hepatocyte cells in the absence of LPS (see, FIGS. 74C and 74D); and iii) HSCs improve BC network development during ethanol+LPS in hepatocyte cells (see, FIGS. 74E and 74F). These morphological observations were confirmed by quantitative metrics (supra) measuring branching density, porosity and canal radius. These data show that ethanol reduced BC branching density and porosity while increasing BC radius. Each of these effects were reversed when HSCs were embedded within the gel underlay. A similar effect of HSCs was observed in hepatocyte cells that were incubated with both ethanol and LPS. See, FIG. 75.

3. NonAlcoholic Liver Disease (NASH).

Fatty liver disease (FLD) is a major public health burden that affects up to 30% of people in Western countries. FLD leads to progressive liver injury, comorbidities and increased mortality. Risk factors for developing FLD are obesity and alcohol consumption, both of which are growing in prevalence worldwide. There is an urgent need for human-relevant preclinical models to improve our understanding of FLD progression to steatohepatitis for development of sensitive noninvasive diagnostics and therapies.

Alcohol-induced liver disease (ALD) represents an ideal case for modeling FDL as ethanol exposure is a comparatively simple mechanism of the disease as opposed to the complexity of diet associated with obesity, diabetes and insulin resistance. Further, the similar characteristics in pathology progression and deterioration of liver function seen in alcoholic and non-alcoholic diet induced FLD, highlight the potential of an ALD microphysiological model for broad application in translational research.

As described herein, embodiments of a Liver-Chip, including embodiments of a Liver-Chip comprising a gel, to create an in vitro model of ALD that uses human relevant blood alcohol concentrations (BAC) and affords multimodal profiling of progressive human liver injury. Thus, ALD Liver-Chips, as described herein, recapitulate established FLD markers in response to ethanol in a concentration-dependent manner, including lipid accumulation as well as tissue and oxidative stress. We show that one embodiment of a gel ALD Liver-Chip supports the study of secondary insults, such as intestinal endotoxins, which as per the “second hit” hypothesis drive the progression from steatosis to steatohepatitis. Moreover, owed to new developments in the design, the ALD Liver-Chip enables novel in vitro quantitative readouts of alcoholic liver toxicity, such as structural changes of the bile canaliculi (BC) network.

In summary, we report development of a human ALD Liver-Chip, as a new platform for modeling progression of alcohol-induced liver injury with direct translation to clinical research.

### Introduction

Fatty liver disease (FLD) ref¹, is a growing global health problem that affects up to 30% of the general population in Western countries ref². The disease can be classified based on the causative trigger as either alcoholic fatty liver disease/alcoholic steatohepatitis (ALD/ASH) induced by excessive alcohol consumption, or as diet-induced non-alcoholic fatty liver disease/nonalcoholic steatohepatitis (NAFLD/NASH) ref¹. Despite their differences in etiology and epidemiology, both forms of FLD share several genetic susceptibility markers, including PNPLA3, TM6SF2, MBOAT7 refs^{3,4}, and both progress from simple hepatic steatosis to liver fibrosis, cirrhosis, and finally hepatocellular carcinoma refs⁵⁻¹⁰. As reported recently, the worldwide prevalence of NAFLD and AFLD was approximately 25% and 4.6%, respectively ref¹¹. Excessive use of alcohol, a major social problem ref¹², contributes substantially to the global burden of FLD. Specifically, of the 1% of the deaths due to liver cirrhosis, the end stage of FLD, 70-80% are directly due to alcohol abuse refs^{7,8}

This information highlights the urgency for the development of specific therapies and identification of noninvasive biomarkers of disease progression. A significant challenge in drug development for the obesity-associated NAFLD/NASH are the human-specific pathogenic, behavioral and environmental disease factors refs^{1,13}. Conversely, for ALD, alcohol intake is the established cause of clinical ASH. Thus, according to the American Gastroenterological Association and the European Association for the Study of the Liver, in patients presenting with FLD, a history of alcohol intake above 20 g/d for women and 30 g/d for men suffices for setting the diagnosis of ALD/ASH ref¹. Hence, we contemplated that ALD/may be the best approach for proof of concept studies on the capabilities of the Liver-Chip to model development and progression of FLD to ASH. Notably, no human in vitro models for alcohol-induced steatosis using clinically relevant blood alcohol concentrations (BAC) are currently available.

The great majority of the experimental studies on FLD have employed animal models that display aspects of the liver disease phenotype, but do not capture the spectrum of the metabolic and inflammatory responses found in the human patients refs^{14,15}. Similarly, in vitro models such as hepatocyte sandwich culture and liver spheroids may recapitulate several of the features of the liver diseases but are missing the dynamics of the tissue microenvironment and the associated cell-cell interactions promoted. A relevant example is given by the ethanol-induced alterations in hepatic cytochrome P450 and their role in the mechanisms driving liver damage¹⁶, that so far can only by studied in human microphysiological systems, as the expression of these enzymes was not maintained in standard sandwich liver cell culture beyond the early time of the culture ref¹⁷. Overuse of alcohol in humans induces systemic effects, such as compromised barrier function and increased gut permeability (“leaky gut”), a response subject to significant species-dependent variability ref¹⁸. Leaky gut and the ensuing inflammatory response due to the escape of bacterial endotoxins to the systemic circulation are suggested to act as the second factor, together with FLD, in the “two-hit” hypothesis for the development of ASH/NASH ref¹⁸.

Therefore, there is an urgency for developing in vitro platforms that more closely approach human organ physiology. Such models would enable basic research and translation to the clinic and eliminate the comorbidities of alcoholic patients, including a staggering number of deaths due to liver cirrhosis ref¹⁹. Because there is a need for in vivo platforms of human liver diseases, we developed a microphysiological system to enable the modeling of the pathogenesis and progression of ALD, based on clinically relevant endpoints.

A Liver-Chip, using primary human cells to recreate the liver sinusoid architecture, demonstrated its unique usefulness for assessment of drug safety and toxicity for humans ref¹⁷. Here, we advanced the design of a Liver-Chip model, including embodiments comprising a new type of gel layer, with new additional capabilities to recapitulate events in human ALD using alcohol at concentrations as in the blood (BAC) of human patients. Further, we evaluated the ability of the ALD Liver-Chip to model disease recovery through alcohol abstinence, or worsening of the phenotype in a two-hit approach by co-exposure to alcohol and the Gram negative bacterial endotoxin, lipopolysaccharide (LPS).

We report here our findings based on multimodal profiling of the histological, molecular, and functional tissue changes in response to ethanol alone, or in combination with LPS, or following abstinence from ethanol. These findings demonstrate the induction of ALD/ASH phenotypes supported by several metabolic stress-associated endpoints in the ALD Liver-Chip,

Moreover, embodiments of Liver chips may be used to observe induction of stress responses, e.g. increasing polyploidy of cells. Indicators of liver injury in ALD, include but are not limited to: increased TG accumulation; increased oxidative stress (ROS); decreased mitochondrial function; increased CYP2E1 activity; Lower NAD+/NADH ratio; Cell death—apoptosis, necroptosis; ER stress. Triglyceride (TG) accumulation refers to molecules representing the major form of storage and transport of fatty acids within cells and in the plasma. ER stress occurs when the capacity of folding associated molecules within the endoplasmic reticulum (ER) to fold proteins becomes saturated. ER stress may be caused by any one or more factors, including those that impair protein glycosylation or disulfide bond formation, or by overexpression of or mutations in proteins entering the secretory path

In addition, we report new readouts of liver injury, as disease-induced changes in the structure of the bile canaliculi (BC) network, which was not shown before in vitro. Although BC network structure was reported as a sensitive marker of toxicity in mice²⁰ and was also altered in human NAFLD patients ref²¹. For this purpose, we optimized the extracellular matrix (ECM) scaffolding to support robust formation of a highly branched BC network, and we captured the ethanol-induced disruptions of the BC network through novel quantitative imaging tools.

Together, results demonstrated the unique ability of the ALD Liver-Chip to model human relevant alcoholic steatosis and phenotypes previously missed by in vitro systems. We believe that the ALD Liver-Chip is a powerful platform for probing the mechanisms of steatosis onset and progression to steatohepatitis, and for supporting drug discovery by providing efficacy and safety endpoints in a patient-specific manner.

### Results: Development of the ALD/ASH Liver-Chip

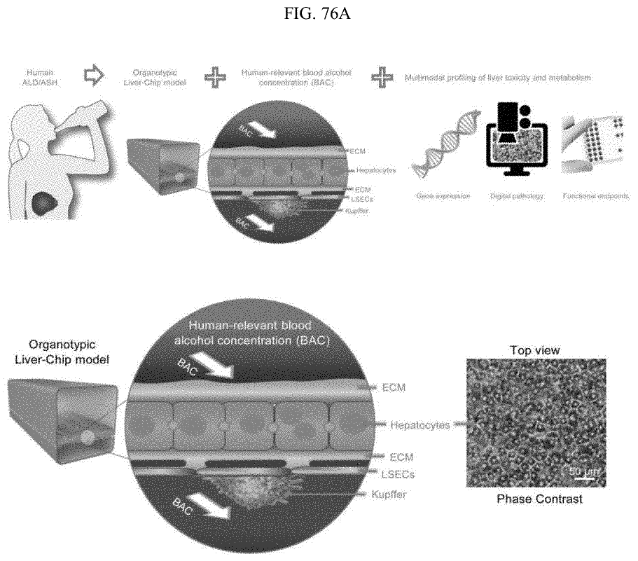

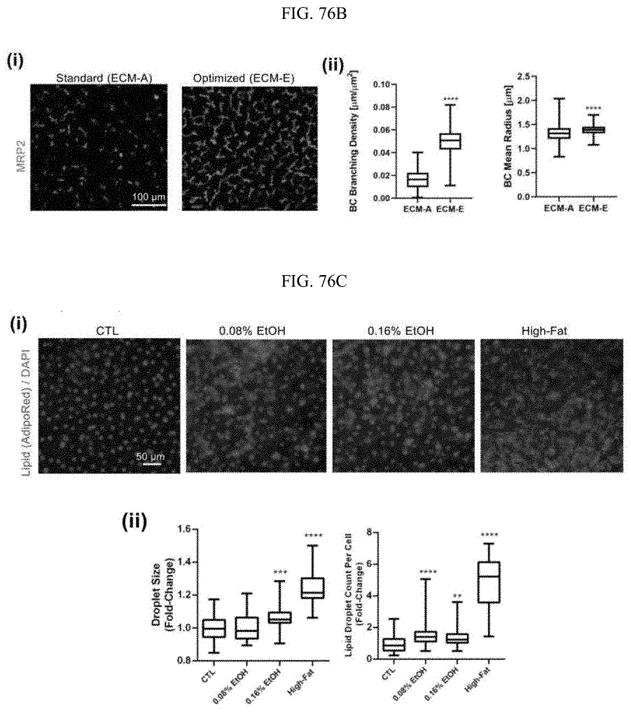

For modeling human ALD, was to continuously perfuse embodiments of a Liver-Chip, as an organotypic microphysiological system using primary human cells ref¹⁷, with ethanol doses within clinically relevant blood alcohol concentrations (BACs) (FIG. 76A), followed by multimodal phenotyping and functional analysis. As reported ref¹⁷, a Liver-Chip embodiment is made of polydimethylsiloxane (PDMS) and contains an upper channel (1 mm tall×1 mm wide) and a lower channel (0.2 mm tall×1 mm wide), which are separated by an ECM-coated, porous PDMS membrane to allow for cell-cell interactions.

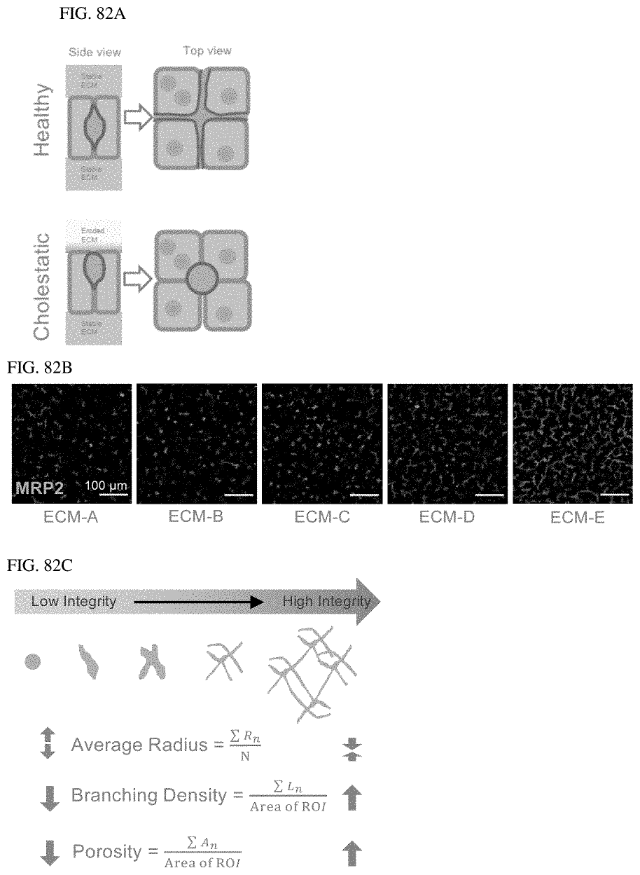

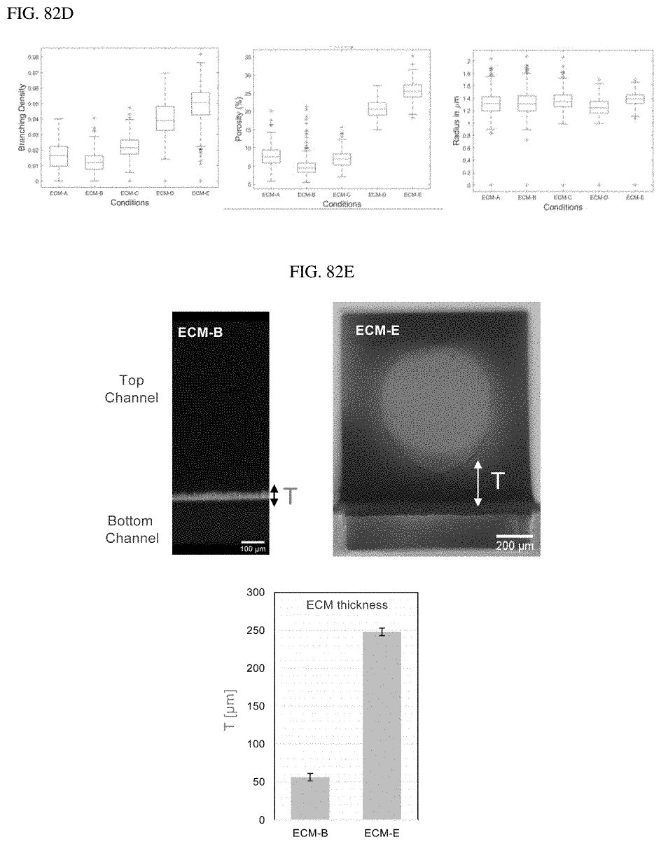

A Tri-culture configuration includes primary hepatocytes cultured in the upper channel and primary liver sinusoidal endothelial cells (LSECs) cultured on the opposite side of the membrane, i.e. in the lower channel, along with Kupffer cells. As this design recapitulates elements of the hepatic sinusoid microenvironment ref¹⁷, we were interested in recreating the bile canaliculi (BC) network that is established between neighboring hepatocytes. In mice, changes to the BC network structure and associated biliary clearance function have been shown to be an early and sensitive indicator of drug toxicity in the liver microenvironment ref²⁰. However, in contrast to rodent hepatocytes, human hepatocytes do not readily form biomimetic bile canaliculi networks in vitro refs²²⁻²⁶. The latter together with the lack of a reproducible, quantitative manner to assess the integrity of the BC network have precluded the use of this clinical endpoint in preclinical studies. Notably, BC integrity is strongly dependent on the presence of a stable extracellular matrix that affords the symmetric mechanical anchoring needed for hepatocyte polarization ref²⁷. Based on these findings, we assessed the impact of various ECM scaffold compositions and patterning methods on BC integrity by using quantitative metrics to describe the MRP-2 stained BC network topology (FIG. 82A-E: Table 8.

TABLE 8
Details of ECM scaffold composition and preparation.

| | ECM-A | ECM-B | ECM-C | ECM-D | ECM-E |
|---|---|---|---|---|---|
| Bottom | — | 0.5 mg/mL Col-I + 0.1 | 0.5 mg/mL | 0.5 mg/mL | 0.5 mg/mL |
| ECM | | mg/mL Fibronectin | Col-I + 0.1 | Col-I | Col-I |
| | | | mg/mL | | |
| | | | Fibronectin | | |
| Top | 0.2 | 0.5 mg/mL Col-I + 0.2 | 0.5 mg/mL | 0.5 mg/mL | 5 mg/mL Col-I |
| ECM | mg/mL | mg/mL Col-IV | Col-I + 4 | Col-I | Viscous |
| | Matrigel ® | | mg/mL MTG | | fingering |

In experiments for this study we used the matrix protocols “ECM-D” and “ECM-E” which promoted biomimetic BC network formation and resulted in the best and reproducible BC metrics (FIG. 76B). In this ECM condition, the primary hepatocytes were covered in a 150-300 μm thick 3D gel of Col-I (FIG. 82E).

Thus, by optimizing the Liver-Chip ECM scaffolding, we significantly improved formation of robust bile canaliculi networks in the Liver Chip. Further, we developed a digital pathology method for the quantitative assessment of BC integrity using structural metrics of the network geometry (see Methods), a sensitive method for the characterization of the Liver-Chip responses to ethanol. In one further embodiment, a liver chip further comprises hematopoietic stem cells (HSCs).

### Modeling ALD in the Liver-Chip

Hepatic intracellular lipid accumulation, or steatosis, is a histological finding of FLD caused either by ethanol abuse, or high-fat diet refs^{9,12.} Therefore, to confirm that the Liver-Chip is suitable for modeling ALD and its progression to ASH, we first probed the capability of the Liver Chip to demonstrate lipid accumulation in hepatocytes on chip upon treatment with ethanol or fatty acids, the latter serving as a positive control as it is a reproducible experimental method for induction of a steatotic phenotype ref²⁹. We monitored the hepatic lipid accumulation using digital pathology to automatically segment images of AdipoRed-stained tissues and quantify the lipid droplets frequency and size. Ethanol concentrations ranging from 0.04% to 0.16% (40 mg/ml to 160 mg/ml) were chosen to mimic the alcohol concentrations found in the blood (BAC) of human patients after alcohol consumption ref³⁰. For comparison, a BAC of 0.08% is the upper limit to for legal driving in the United States and the UK.

In order to demonstrate the sensitivity of the Liver Chip to ethanol induced-steatosis in a reproducible manner, we had to adjust the medium the hepatocytes were exposed during the treatment, for glucose, insulin and cortisol concentration, as described in Methods. Following 7 days of culture treatment with either ethanol, at BACs of 0.08% or 0.16%, or with oleic acid (1 μg/ml), for up to 48 hrs, steatosis was induced (FIG. 76A-Ci).

The average droplet size reflected the ethanol concentration the cells were exposed to refs³¹′³² (FIG. 76Cii), indicating the sensitivity of the Liver Chip to operate within a range of clinically relevant BACs, as desired for disease modeling. Further, readouts may also include monitoring for alterations such as: increase in de novo lipogenesis; decrease in Fatty Acid b-oxidation.

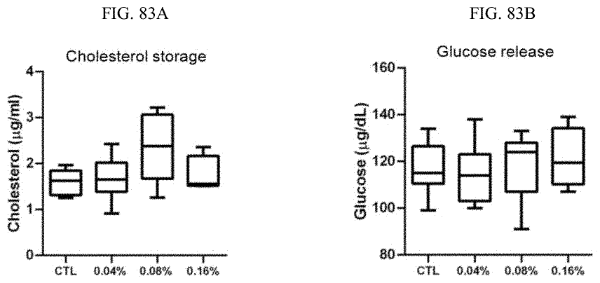

We also assessed whether the lipid accumulation observed in the Liver-Chip in response to ethanol treatment was associated with hepatocellular damage and metabolic dysregulation ref³³. To this purpose we used fluorometric assays for biochemical markers of liver function, including albumin, cholesterol, glucose, and glycogen (FIG. 77A-B). We found that the 48 hrs exposure to ethanol at human-relevant BACs resulted in a significant increase in cholesterol levels, which was more pronounced in the effluent (FIG. 77A) compared to the cell lysates (FIG. 83A).

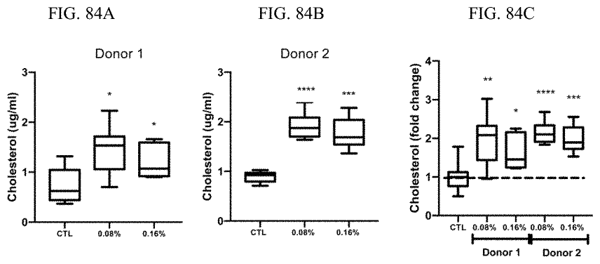

We confirmed the reproducibility of this finding in Liver-Chips populated with hepatocytes from at least 2 different donors. Whereas baseline levels of cholesterol release exhibited donor-to-donor variability (FIG. 84A), the increase in response to ethanol was similar between the two donors (FIG. 84C). Glycogen storage, as determined in the cell lysates, showed a trend for increase upon exposure to 0.08% ethanol (FIG. 77B), while no changes were detected in glucose release (FIG. 83B), except when we used toxic ethanol concentrations above 0.32% (data not shown). Similarly, albumin release was not affected by this limited length exposure to ethanol (FIG. 77C), in line with clinical data in patients where such changes would usually indicate more severe deterioration of liver function, refs^{34,35}.

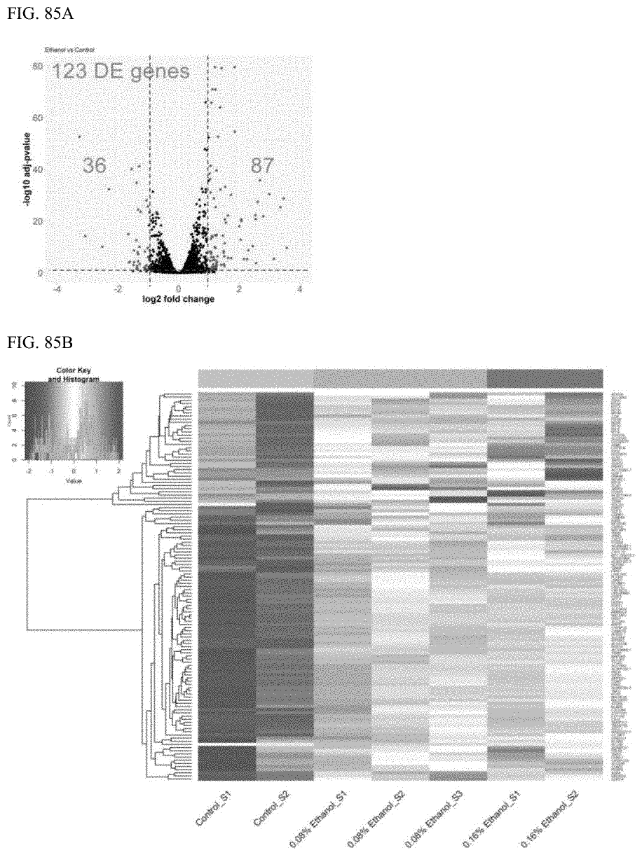

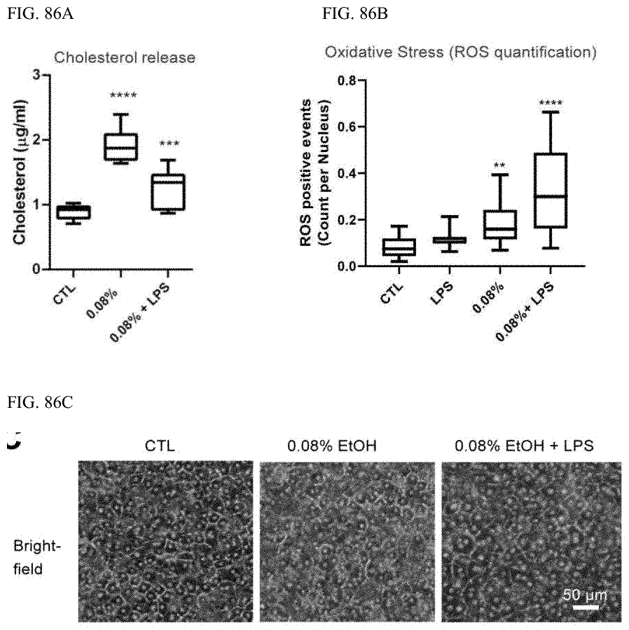

To assess for changes in gene expression underpinning the above phenotypic responses to ethanol exposure, we performed RNA-seq analysis as outlined in the Methods section. The differentially expressed (DE) genes and the associated stratification, based on the magnitude of differences between ethanol-exposed and control chips, were plotted as shown in FIG. 85A. In the group of the ethanol exposed chips, we identified 123 DE genes (red dots, adj. p-value<0.05 and |log₂ FoldChange|>1), of which 87 were upregulated (red in heatmap) and 36 were downregulated (blue in heatmap) (FIG. 85B). Pathway analysis of the DE genes using the KEGG bioinformatics database revealed changes in the expression of genes implicated in functions consistent with the phenotypic and other readouts discussed herein (Table 9).

TABLE 9
Pathway analysis based on the 123 DE genes using the KEGG database.

| ID | Description | GeneRa | BgRatio | pvalue | p.adjust | qvalue | geneID |
|---|---|---|---|---|---|---|---|
| hsa04978 | Mineral | 0.123077 | 0.007327 | 1.84E−08 | 3.00E−06 | 2.87E−06 | 4502/4493/4499/4489/ |
| | absorption | | | | | | 4494/4495/4496/4501 |
| hsa00982 | Drug | 0.076923 | 0.009096 | 2.92E−04 | 2.38E−02 | 2.28E−02 | 2330/2326/7365/124/ |
| | metabolism - | | | | | | 1558 |
| | cytochrome | | | | | | |
| | P450 | | | | | | |
| hsa00830 | Retinol | 0.061538 | 0.008464 | 2.15E−03 | 1.17E−01 | 1.12E−01 | 7365/124/1558/5959 |
| | metabolism | | | | | | |
| hsa00250 | Alanine, | 0.046154 | 0.004548 | 3.11E−03 | 1.27E−01 | 1.21E−01 | 27165/122622/443 |
| | aspartate and | | | | | | |
| | glutamate | | | | | | |
| | metabolism | | | | | | |
| hsa04930 | Type II | 0.04615 | 0.005811 | 6.24E−03 | 2.03E−01 | 1.94E−01 | 5313/6517/90216517/ |
| | diabetes | | | | | | 9021/5105/10 |
| | mellitus | | | | | | |
| hsa04931 | Insulin | 0.061538 | 0.013643 | 1.17E−02 | 3.02E−01 | 2.89E−01 | 6517/9021/5105/10998 |
| | resistance | | | | | | |
| hsa00140 | Steroid | 0.046154 | 0.00758 | 1.30E−02 | 3.02E−01 | 2.89E−01 | 7365/1586/1312 |
| | hormone | | | | | | |
| | biosynthesis | | | | | | |
| hsa04964 | Proximal | 0.030769 | 0.00290 | 1.50E−02 | 3.06E−01 | 2.93E−01 | 27165/5105 |
| | tubule | | | | | | |
| | bicarbonate | | | | | | |
| | reclamation | | | | | | |
| hsa00010 | Glycolysis/ | 0.046154 | 0.00859 | 1.81E−02 | 3.07E−01 | 2.94E−01 | 5313/124/5105 |
| | Gluconeogenesis | | | | | | |

[symbol]

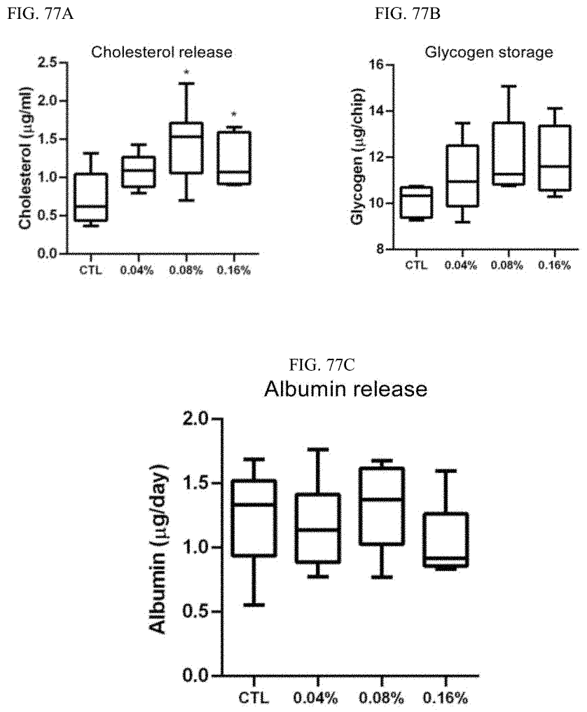

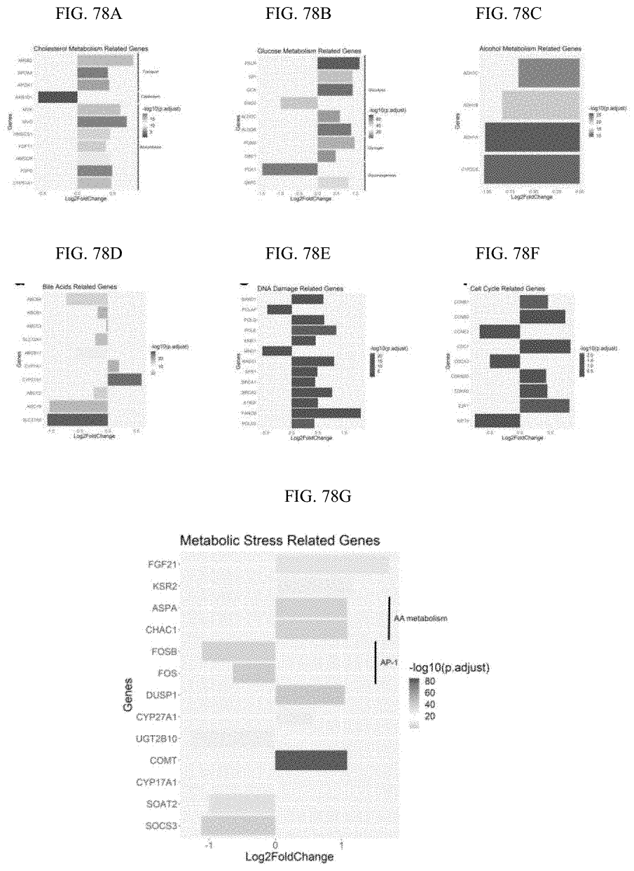

Related to lipid metabolism, we found upregulation of three genes related to cholesterol transport (NR0B2³⁶, APOA4³⁷, and APOA1³⁸) and seven to cholesterol biosynthesis (MVK, MVD, HMGCS1³⁹, FDFT1⁴⁰, HMGCR⁴¹, FDPS⁴², CYP51A1⁴³), along with downregulation of AKR1D1⁴⁴, involved in cholesterol catabolism (FIG. 78A).

Specifically, glycolysis/gluconeogenesis, steroid hormone biosynthesis, and insulin resistance/type II diabetes were among the pathways affected by exposure of the Liver-Chip to ethanol. Further, the expected ethanol-induced dysregulation of glucose metabolism and storage was recapitulated by the ALD Liver-Chip, as depicted in the altered expression of genes involved in glycolysis, gluconeogenesis, and glycogen metabolism (FIG. 78B). Ethanol-treated Liver-Chips also exhibited significant changes in the expression of members of the alcohol dehydrogenase (ADH) gene family, including ADH1C⁴⁵,ADH1B⁴⁶, and ADH1A, all involved in alcohol metabolism⁴⁶ and CYP2E1, one of the major risk factors for development of ALD which high levels is one of the clinical endpoints in chronic ALD (Caro A A, 2004 Ann Rev Pharm Toxicol) (FIG. 78C). We detected similar effects in genes associated with induction of oxidative stress, a major pathogenic mechanism of ALD. FIG. 78C.

Further, exposure to ethanol affected the expression of clinically-relevant genes involved in bile acid production and processing, hallmarks of cholestasis. Here, we identified changes in the expression of a number of specific genes such as the major BC transporters ABCB4 (MDR3) ref⁴⁷, ABCB1 (MDR1) refs^{4,8}, ABCC3 (MRP-3) ref⁴⁹, SLC10A1 (NTCP) ref⁵⁰, ABCB11 (BSEP) ref⁵¹, ABCC2 (MRP-2) ref⁵¹, and ADCY8 ref⁵² or genes associated with bile acid processing such as SLC27A5, involved in fatty acid elongation⁵³, CYP7A1^{54,55} which regulates the overall bile acid production rate and CYP27A1 ref⁵⁶, associated with the bile acids synthesis alternative pathway (FIG. 78D). Our data also show that the ALD Liver-Chip picked up the impact of ethanol treatment on genes involved in the metabolism of alanine, aspartate, and glutamate. In line with previous data shown the link between FLD and DNA damage in hepatocytes ref¹⁶, our analysis revealed upregulation of POLE and POLD2 ref⁵⁷, both involved in DNA replication and repair, as well as of RAD51 and FANCB, which are expressed at DNA damage sites and implicated in homologous recombination refs^{58,59} (FIG. 78E). Further, we saw upregulation of E2F1 ref⁶⁰ and CCNB1 ref⁶¹ and downregulation of KIF14 ref⁶² and CCNE2 ref⁶³, all participating in cell cycle regulation (FIG. 78F). Notably, several of the DNA damage-associated genes identified in our data have not yet been implicated in the pathogenesis of FLD pathogenesis, suggestive of the potential of the ALD Liver-Chip platform to be applied for detailed characterization of the alcohol-induced DNA damage. Lastly, ethanol exposure led to the dysregulation of several markers of oxidative stress in the Liver-Chip, such as genes of the metallothionin family (MD) refs⁶⁴⁻⁶⁶ (FIG. 84C) and DUSP1 ref⁶⁷ (FIG. 78G). Ethanol exposure altered the expression of FOSB, a gene of the stress responsive Fos gene family (FIG. 78G) that has been implicated in the pathogenesis of metabolic liver disease ref⁶⁸, in further support of stress responses induced by ethanol in the liver chip.

### Modeling Two-Hit Mechanisms in ALD/ASH.

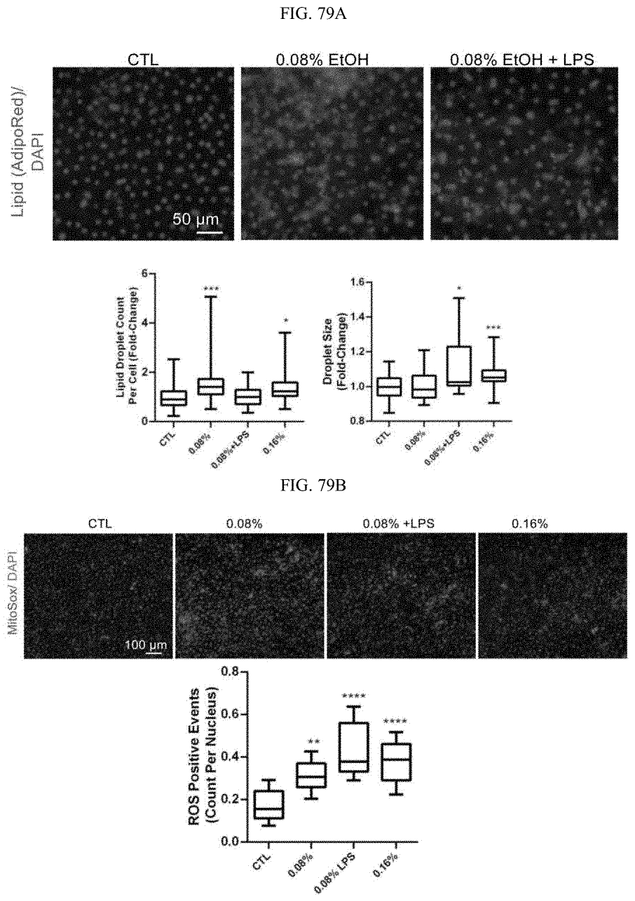

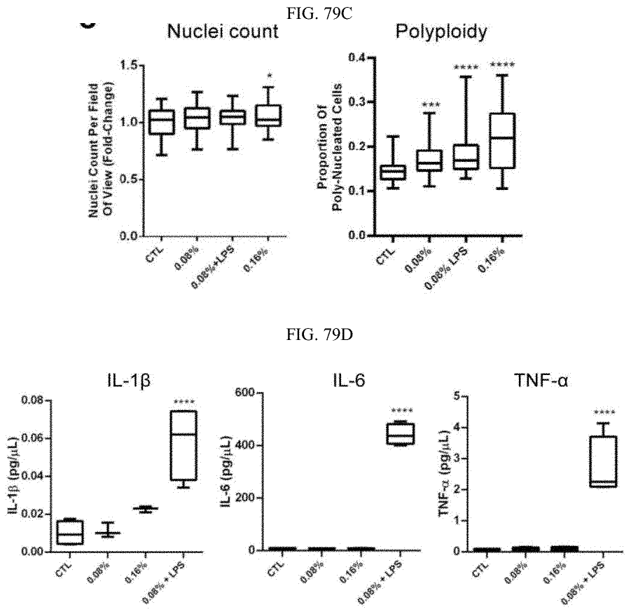

Having shown that at least one embodiment of a Liver-Chip responds to alcohol, we explored the possibility of modeling the progression of ALD, as per the two- or one-hit and second-hit hypothesis, such that simple steatosis induced by alcohol requires a second hit (insult) for progression of ALD to ASH ref⁶⁹. Ethanol consumption in humans compromises the intestinal barrier function resulting in increased permeability to intestinal endotoxins, e.g. bacterial lipopolysaccharide (LPS), from the gut to the liver via the portal vein ref¹⁸. Such a permeabalized leaky barrier is considered one of the “second hits” in the progression of ALD to inflammation (ASH) ref⁷⁰. Therefore, we tested whether exposure to ethanol and LPS together would worsen the Liver-Chip pathology. Indeed, we found increased steatosis and associated cholesterol release (FIG. 79A. Further, we evaluated ethanol and LPS effects on hepatocyte pathology, as related to the development of oxidative stress. To this purpose we used MitoSox staining, a fluorescent marker of reactive oxygen species (ROS) and quantified the number of ROS events per nuclei. Our findings revealed a dose-dependent increase in ROS events in response to ethanol, which, indeed, was further exacerbated upon co-exposure to ethanol and LPS (FIG. 79B). The specificity of the response is highlighted by lack of any effect upon treatment with LPS alone (FIG. 85C).

Use of image processing to automatically segment the cellular boundaries and determine the number of nuclei per cell, revealed changes in polyploidy, a marker for cell stress ref⁷¹, in both the alcohol− or alcohol+ LPS-treated groups (FIG. 79C). Interestingly, exposure to ethanol or ethanol+LPS had a minimal effect on the number of hepatocytes nuclei counted per field of view.

These results are parallel with our RNAseq data showing the effects of alcohol on genes related to DNA damage and cell cycle, and consistent with the reported disease mechanisms. Induction of CYP450 by ethanol and free fatty acids and the consequential oxidative stress rampage were previously implicated as mechanisms involved with increased DNA damage in hepatocytes and associated progress to hepatocellular carcinoma (HCC) ref¹⁶. However, because this is the first, human relevant, in vitro model that demonstrated oxidative stress and DNA damage induction by ethanol or LPS, and ethanol together with LPS, this may be due to the sustained expression of P450, such as the ALD-relevant CYP2E1, in one embodiment of a Liver Chip.

Inflammatory cytokines are significantly elevated in alcoholic patients with advanced liver disease ref⁷² and have been proposed as therapeutic targets, as inhibition of TNF-α action was protective against alcohol-induced liver injury in mouse models ref⁷³. Therefore we assessed the secretion of cytokines in response to ethanol and LPS, as our Liver-Chip model contains Kupffer cells which are a major source of proinflammatory cytokines ref⁷⁴. Whereas as anticipated treatment with LPS led to a robust increase of proinflammatory cytokines release in the Liver Chip co-treatment with 0.08% ethanol+LPS resulted in further increase in the production of IL-6 (FIG. 79D), similar to findings from clinical and experimental studies demonstrating the role of IL6 in the progress of the disease ref⁷² Sheron Clin Exp Immunol 1991 and Pere Gines and Shiv Sarin CMGH May 2018. Since development of steatosis, as assessed by the lipid droplets count and size was not different between the Liver Chips exposed for 48 hrs to ethanol or ethanol+LPS (data not shown), the cytokines rise is most likely linked to the increase in oxidative stress (FIG. 79B) and the associated cell injury or other mechanisms as previously described (Dirk Schmidt-Arras and Stefan Rose-John, J of Hepatology 2016).

Patients with alcoholic liver disease frequently manifest clinical and/or histological evidence of cholestasis ref⁷⁵, which is either decrease in bile flow due to impaired secretion by hepatocytes or obstruction of bile flow through the intra- or extrahepatic bile transport network. Cholestasis is diagnosed based on accumulation of the potentially toxic cholephiles in the liver and the systemic circulation ref⁷⁵. Currently, the mechanism of alcohol-induced cholestasis remains poorly understood. We have recently shown the ability of our human Liver-Chip to develop bile canaliculi and recapitulate in vivo findings on drug-induced destruction of bile canaliculi transporters ref¹⁷. In the current study, one embodiment of a Liver-Chip was optimized to develop more extensive, biomimetic BC networks.

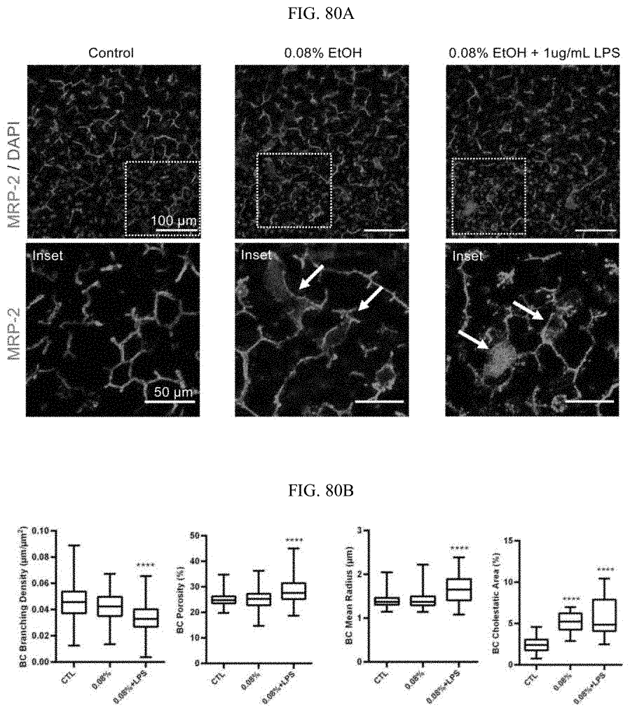

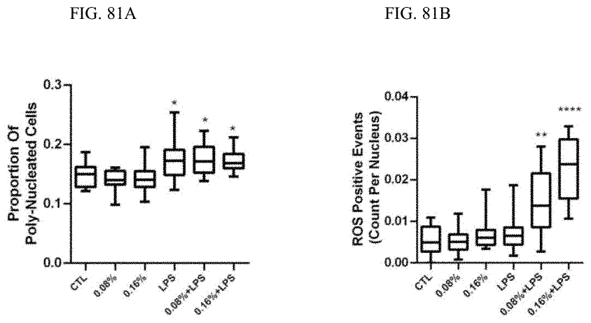

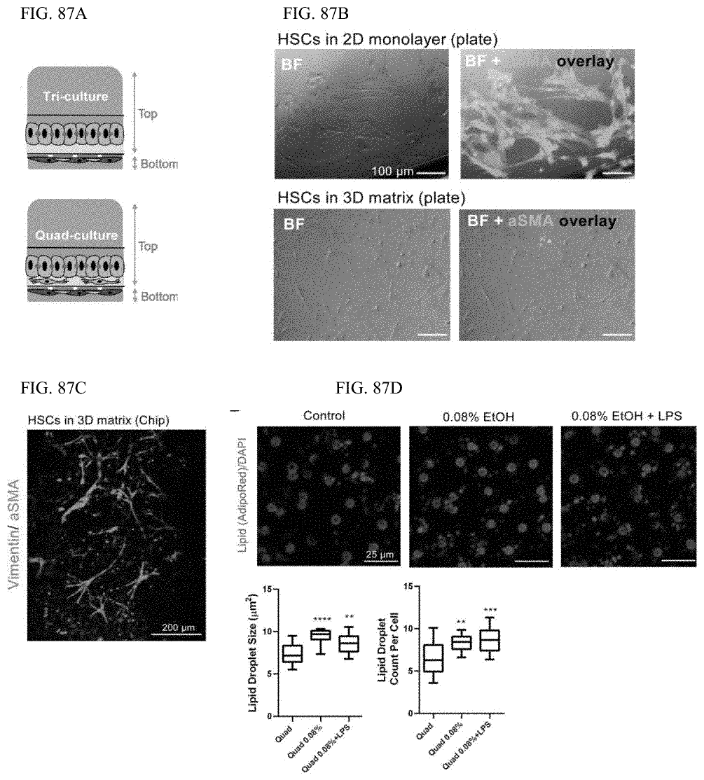

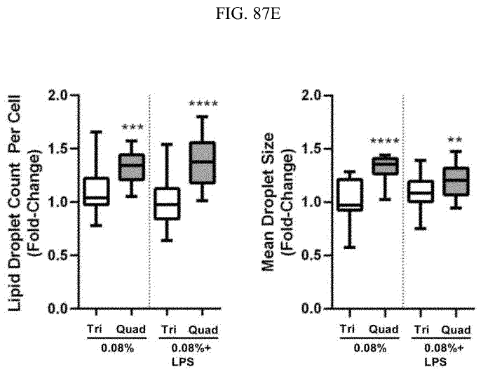

Given that RNA seq analysis of hepatocytes in the alcohol-treated Liver-Chip depicted altered expression of bile canaliculi-related genes, we used quantitative analysis of the MRP-2 stained BC network to assess the sensitivity of the Liver-Chip and demonstrated alcohol-induced cholestatic changes at the level of the canaliculi (FIG. 80A). In the Liver-Chips treated with 0.08% ethanol, we identified areas of markedly dilated BCs, a marker of cholestasis, that were significantly expanded in those with 0.08% ethanol+LPS together, as anticipated by the more severe hepatic injury (FIG. 80B. Further, in the latter the average BC radius increased throughout the BC network, whereas the branching density was decreased, both signs of the worsening of hepatic damage, in line with the two-hit hypothesis for the pathogenesis of ASH/NASH ref²⁰. We would like to also mention here our preliminary study on leveraging the optimized ECM scaffold (FIG. 81A) for embedding of hepatic stellate cells (HSCs). This approach enabled the development of a quad-culture Liver-Chip (FIG. 87A) used for a proof-of-concept experiment to evaluate the contribution of HSCs to the onset and progression of ALD in the Liver-Chip. Our preliminary results suggest that HSCs added to the Liver-Chip robustly embedded in the 3D matrix (FIG. 87B and FIG. 87C) and supported a quad-culture Liver-Chip, that showed more pronounced ethanol concentration-dependent steatosis (FIG. 87D), than the tri-culture Liver-Chip (no HSCs) (FIG. 87C).

Modeling of the Hepatocyte Recovery Following Abstinence from Alcohol with the Liver-Chip.

Clinical data have shown the potential of the steatotic liver to repair the steatotic injury following timely abstinence from alcohol or with diet modifications ref¹⁹. Thus, we assessed the responses of the “alcoholic” Liver-Chip to withdrawal from ethanol or ethanol+LPS. Treatment of the Liver-Chip for 48 h with ethanol or ethanol+LPS as above, was followed by a 5 day-long treatment-free “recovery” period. By the end of the recovery period, ethanol withdrawal resulted in normalization of oxidative and cellular stress, whereas this was not the case for the Liver-Chips exposed to alcohol+LPS together (FIG. 81A-B). Although it is possible that longer recovery period might improve the condition in the latter group, these data are promising as they suggest that the ALD Liver Chip shows an insult-dependent recovery similar to lack of repair of the hepatic injury in patients with more advanced alcoholic disease. Further, they underscore the potential of the Liver-Chip to model recovery from ethanol-induced damage in a clinically relevant way, and to uncover potential new targets to leverage regenerative mechanisms in the human liver for therapeutic or prognostic purposes.

Considering the global rise in FLD-associated co-morbidities, there is a great need to advance human in vitro models to understand disease mechanisms and support the development of safe and efficacious diagnostic and therapeutic approaches for FLD. Here we describe further optimization of a human Liver-Chip platform ref⁷⁴, to model an unmet medical needs, such as for modeling progressive alcohol-induced liver disease (ALD). The human Liver-Chip supports a complex co-culture system containing up to four of the main cell types found in the liver and recapitulates relevant cell-cell interactions, improves nutrients availability in the cells via continuous perfusion and maintains long-term cell viability. The unique properties of this design allow for continuous exposure to ethanol followed by withdrawal in the same sample for modeling recovery. It also enables the study of several ALD endpoints and multi-modal assessment of the injury in the same chip.

We recently showed the use of our human Liver-Chip platform for creating safety profiles and studying toxicity for drugs in development and confirm clinically relevant mechanisms of action ref¹⁷. In this study we describe how we adapted the human Liver-Chip system by new designs, e.g. gels, and methods for analyses, e.g. RNA analysis, to model aspects of alcohol-induced liver disease assessed by clinically relevant endpoints.

We demonstrate how by perfusion of the Liver-Chip with relevant (BAC) to model heavy drinking, we were able to detect pathological changes associated with ALD in a sensitive and reproducible manner. As here our primary goal was to leverage the Liver-Chip optimized for safety and toxicity studies and add new capabilities to advance it as a platform for modeling human liver diseases, we applied a short term experimental design We show that the ALD Liver-Chip model recapitulated disease endpoints like intracellular accumulation of lipids, development of oxidative stress, cellular damage and metabolic dysregulation upon exposure to alcohol up to 48 hrs refs^{19,76}. RNA seq for gene expression profiling supported all the experimental findings and hinted at additional disease-relevant pathways.

Alcohol-induced changes observed or measured were reversed by withdrawal of ethanol for several days, mirroring the effects of alcohol abstinence in human patients.

Furthermore, we introduced innovations, as described herein, to embodiments of a Liver-Chip model that greatly increase its range of applications for translational studies. By optimizing the chemical and mechanical properties of the ECM patterning for the Liver-Chip we induced a stable, biomimetic bile canaliculi network in the hepatic tissue, as assessed by quantitative imaging metrics. Of note, in vitro human hepatocyte cultures have thus far failed to demonstrate sustained formation of BC networks. Our advances hence augmented the human Liver-Chip with new capabilities by enabling visualization and quantification of changes to the biomimetic BC network, a sensitive maker of liver injury refs^{20,21}, which was also confirmed in our study. We also applied the newly optimized ECM protocol in a proof-of concept experiment to include hepatic stellate cells in the Liver-Chip optimized for studying liver diseases. We report here our promising early findings on the potential impact of HSCs on ethanol-induced steatosis, as modeled in the ALD Liver-Chip.

The expression and functionality of enzymes of the CYP450 family, such as the ALD-relevant CYPE1A, are maintained in this platform for prolonged periods, as compared to most of the currently used hepatocyte culture systems ref¹⁷. The ALD Liver-Chip exhibits alcohol treatment-induced effects in the expression of genes related to nutrient metabolism and metabolic stress, which is in agreement with the dysregulation of steatogenic enzymes and transcription factors identified by in vivo studies ref⁸. We also identified ethanol-dependent dysregulation of cell cycle- and DNA damage-related genes. Furthermore, the ALD Liver—Chip exhibited altered gene expression of cholesterol metabolism and BC transporters, a finding corroborated by reduced BC network integrity by the optimized here quantitative imaging. Thus, our Liver-Chip provides a very promising system to model human ALD and study clinically relevant metabolic events, such as ethanol metabolism, lipogenesis, biliary function, and oxidative stress.

Next, we assessed the capability of the Liver-Chip to simulate progression of ALD, for instance when liver injury is combined with leaky gut. Dybiosis is a chronic alcohol-driven intestinal complication that leads to increased permeability of the intestinal lining and systemic release of intestinal endotoxins from the gut into the systemic circulation. The circulating endotoxins drive hepatic inflammation and release of proinflammatory factors which further induce tissue damage and deterioration of liver function refs^{73,77}. Here, we modeled this scenario by perfusing the Liver-Chip with LPS and ethanol combined. We show that this treatment increased oxidative stress (as per ROS quantification) and hepatocyte polyploidy, and it compromised the integrity of the BC network. Moreover, withdrawal from LPS and ethanol for five days did not diminish the oxidative stress, in contrast to the rescuing of the pathology induced by ethanol alone. This suggests that chronic alcohol intake coupled with systemic inflammation worsens the liver damage and may significantly compromise recovery in ALD/ASH, similar to the responses of patients with more advanced disease⁷⁸. In animal experiments administration of antibiotics to reduce endotoxemia or inactivation of Kupffer cells with gadolinium chloride can both prevent liver injury ref⁷⁹, suggesting that the ALD Liver-Chip model could be useful as a platform to determine human relevance of proposed mechanisms for new therapeutic approaches.

Embodiments of ALD Liver-Chip presents multiple advantages over other in vivo and in vitro FLD models. Rodent models of NAFLD/NASH, although they typically develop the disease histopathology and have been instrumental in the elucidation of main pathogenetic mechanisms such as insulin resistance, they were not successful in recapitulating the variability in the patients responses refs⁸⁰⁻⁸². With respect to rodent models of ALD/ASH, unfortunately they were notoriously resistant to the hepatotoxic effects of alcohol alone. They develop significant chronic liver injury when exposed to combinations of alcohol either with a toxin or major dietary modifications refs^{83,84}. Other, non-rodent, animal models such as Caenorhabditis elegans, opossum, Ossabaw pig, and primates were recently introduced, but these studies are still at the early validation stages ref⁸¹.

In vitro models used to further elucidate the molecular mechanisms underlying FLD include standard cultures of primary or immortalized, patient- or rodent-derived, hepatic cells, co-culture and 3D cultures ref⁸¹. The main caveats with these models include a lack of the dynamic environment provided in the engineered microphysiological systems and of the in vivo-relevant tissue-tissue interfaces and corresponding cytoarchitecture. As a result, no fluid flow or associated mechanical forces can be applied, oxygen and nutrient transport are limited, and metabolites may accumulate to levels well beyond, or below, the physiologically relevant, impeding the translation of findings for patients care. In addition, the lack of fluid flow compromises human disease modeling such as the ALD/ASH as it does not support real time perfusion with ethanol to mimic circulating blood alcohol concentrations. Lastly, 3D cultures, arguably the more advanced in vitro model, do not support yet visualization of the dynamic changes in cell morphology and cell-cell interactions.

In summary, we report how we have combined the human Liver-Chip ref¹⁷ with new bioengineering approaches and multimodal profiling to develop an innovative platform for modeling progressive liver injury in response to alcohol (ALD). Our findings demonstrate the potential of the ALD Liver-Chip to model comorbidities and improve translation of preclinical data to the clinic, to uncover novel pathogenic and recovery mechanisms, and to identify the windows for successful intervention in patient cohorts.

### EXEMPLARY REFERENCES (REFS) ASSOCIATED IN PART WITH SECTION 3, HEREIN INCORPORATED BY REFERENCE IN THEIR ENTIRETIES

(ref) 1. Bedogni, G., Nobili, V. & Tiribelli, C. Epidemiology of fatty liver: an update. World J. Gastroenterol. 20, 9050-9054 (2014).
2. Asrani, S. K., Devarbhavi, H., Eaton, J. & Kamath, P. S. Burden of liver diseases in the world. Journal of Hepatology vol. 70 151-171 (2019).
3. Hotta, K. et al. Association of the rs738409 polymorphism in PNPLA3 with liver damage and the development of nonalcoholic fatty liver disease. BMC Med. Genet. 11, 172 (2010).
4. Meroni, M., Longo, M., Rametta, R. & Dongiovanni, P. Genetic and Epigenetic Modifiers of Alcoholic Liver Disease. Int. J. Mol. Sci. 19, (2018).
5. Chacko, K. R. & Reinus, J. Spectrum of Alcoholic Liver Disease. Clin. Liver Dis. 20, 419-427 (2016).
6. Orman, E. S., Odena, G. & Bataller, R. Alcoholic liver disease: pathogenesis, management, and novel targets for therapy. J. Gastroenterol. Hepatol. 28 Suppl 1, 77-84 (2013).
7. Rehm, J. et al. Global burden of disease and injury and economic cost attributable to alcohol use and alcohol-use disorders. Lancet (London, England) 373, 2223-2233 (2009).
8. Magdaleno, F., Blajszczak, C. & Nieto, N. Key Events Participating in the Pathogenesis of Alcoholic Liver Disease. Biomolecules 7, 9 (2017).
9. Scaglioni, F., Ciccia, S., Marino, M., Bedogni, G. & Bellentani, S. ASH and NASH. in Digestive Diseases vol. 29 202-210 (2011).
10. Toshikuni, N., Tsutsumi, M. & Arisawa, T. Clinical differences between alcoholic liver disease and nonalcoholic fatty liver disease. World Journal of Gastroenterology 8393-8406 (2014) doi:10.3748/wjg.v20.i26.8393.
11. Chang, Y. et al. Alcoholic and Nonalcoholic Fatty Liver Disease and Liver-Related Mortality: A Cohort Study. Am. J. Gastroenterol. 114, 620-629 (2019).
12. Toshikuni, N., Tsutsumi, M. & Arisawa, T. Clinical differences between alcoholic liver disease and nonalcoholic fatty liver disease. World J. Gastroenterol. 20, 8393-406 (2014).
13. Stefan, N., Häring, H.-U. & Cusi, K. Non-alcoholic fatty liver disease: causes, diagnosis, cardiometabolic consequences, and treatment strategies. Lancet Diabetes Endocrinol. 7, 313-324 (2019).
14. Bertola, A. Rodent models of fatty liver diseases. Liver Research vol. 2 3-13 (2018).
15. Santhekadur, P. K., Kumar, D. P. & Sanyal, A. J. Preclinical models of non-alcoholic fatty liver disease. Journal of Hepatology vol. 68 230-237 (2018).
16. Seitz, H. K. & Stickel, F. Risk factors and mechanisms of hepatocarcinogenesis with special emphasis on alcohol and oxidative stress. Biol. Chem. 387, 349-60 (2006).
17. Jang, K. J. et al. Reproducing human and cross-species drug toxicities using a Liver-Chip. Sci. Transl. Med. 11, (2019).
18. Bishehsari, F. et al. Alcohol and Gut-Derived Inflammation. Alcohol Res. 38, 163-171 (2017).
19. Menon, V. K., Gores, G. J. & Shah, V. H. Pathogenesis, diagnosis, and treatment of alcoholic liver disease. Mayo Clin. Proc. 76, 1021-1029 (2001).
20. Meyer, K. et al. A Predictive 3D Multi-Scale Model of Biliary Fluid Dynamics in the Liver Lobule. Cell Syst. 4, 277-290.e9 (2017).
21. Segovia-Miranda, F. et al. Three-dimensional spatially resolved geometrical and functional models of human liver tissue reveal new aspects of NAFLD progression. Nat. Med. 25, 1885-1893 (2019).
22. Deharde, D. et al. Bile canaliculi formation and biliary transport in 3D sandwich-cultured hepatocytes in dependence of the extracellular matrix composition. Arch. Toxicol. 90, 2497-2511 (2016).
23. Swift, B., Pfeifer, N. D. & Brouwer, K. L. R. Sandwich-cultured hepatocytes: an in vitro model to evaluate hepatobiliary transporter-based drug interactions and hepatotoxicity. Drug Metab. Rev. 42, 446-471 (2010).
24. Abe, K., Bridges, A. S. & Brouwer, K. L. R. Use of sandwich-cultured human hepatocytes to predict biliary clearance of angiotensin II receptor blockers and HMG-CoA reductase inhibitors. Drug Metab. Dispos. 37, 447-452 (2009).
25. Nakakariya, M. et al. In vivo biliary clearance should be predicted by intrinsic biliary clearance in sandwich-cultured hepatocytes. Drug Metab. Dispos. 40, 602-609 (2012).
26. Reif, R. et al. Bile canalicular dynamics in hepatocyte sandwich cultures. Arch. Toxicol. 89, 1861-1870 (2015).
27. Li, Q. et al. Extracellular matrix scaffolding guides lumen elongation by inducing anisotropic intercellular mechanical tension. Nat. Cell Biol. 18, 311-318 (2016).
28. Bischel, L. L., Lee, S.-H. & Beebe, D. J. A practical method for patterning lumens through ECM hydrogels via viscous finger patterning. J. Lab. Autom. 17, 96-103 (2012).
29. Breher-Esch, S., Sahini, N., Trincone, A., Wallstab, C. & Borlak, J. Genomics of lipid-laden human hepatocyte cultures enables drug target screening for the treatment of non-alcoholic fatty liver disease. BMC Med. Genomics 11, (2018).
30. Elgammal, A. et al. The Blood Alcohol Concentration Testing Emergency Room Investigation Analysis Study: A 1-Year Review of Blood Alcohol Concentration Testing in an Emergency Department. Adv. Emerg. Med. 2015, 1-5 (2015).
31. Takahashi, Y. & Fukusato, T. Histopathology of nonalcoholic fatty liver disease/nonalcoholic steatohepatitis. World J. Gastroenterol. 20, 15539-15548 (2014).
32. Bala, S. et al. The pro-inflammatory effects of miR-155 promote liver fibrosis and alcohol-induced steatohepatitis. J. Hepatol. 64, 1378-1387 (2016).
33. Tsukamoto, H. Metabolic reprogramming and cell fate regulation in alcoholic liver disease. Pancreatology vol. 15 S61-S65 (2015).
34. Petersen, M. C., Vatner, D. F. & Shulman, G. I. Regulation of hepatic glucose metabolism in health and disease. Nature Reviews Endocrinology vol. 13 572-587 (2017).
35. Dyson, J. K., Anstee, Q. M. & McPherson, S. Non-alcoholic fatty liver disease: a practical approach to diagnosis and staging. Frontline Gastroenterol. 5, 211-218 (2014).
36. Smalling, R. L. et al. Genome-wide transcriptome analysis identifies novel gene signatures implicated in human chronic liver disease. Am. J. Physiol. Liver Physiol. 305, G364-G374 (2013).
37. Kang, M., Kim, J., An, H.-T. & Ko, J. Human leucine zipper protein promotes hepatic steatosis via induction of apolipoprotein A-IV. FASEB J. 31, 2548-2561 (2017).
38. Castera, L., Friedrich-Rust, M. & Loomba, R. Noninvasive Assessment of Liver Disease in Patients With Nonalcoholic Fatty Liver Disease. Gastroenterology vol. 156 1264-1281.e4 (2019).
39. Iravani, F., Hosseini, N. & Mojarrad, M. Role of MicroRNAs in Pathophysiology of Non-alcoholic Fatty Liver Disease and Non-alcoholic Steatohepatitis. Middle East J. Dig. Dis. 10, 213-219 (2018).
40. Stättermayer, A. F. et al. Role of FDFT1 polymorphism for fibrosis progression in patients with chronic hepatitis C. Liver Int. 34, 388-395 (2014).
41. Min, H. K. et al. Increased hepatic synthesis and dysregulation of cholesterol metabolism is associated with the severity of nonalcoholic fatty liver disease. Cell Metab. 15, 665-674 (2012).
42. Tommasi, S. & Besaratinia, A. Dna hydroxymethylation at the interface of the environment and nonalcoholic fatty liver disease. International Journal of Environmental Research and Public Health vol. 16 (2019).
43. Kakehashi, A. et al. Proteome characteristics of non-alcoholic steatohepatitis liver tissue and associated hepatocellular carcinomas. Int. J. Mol. Sci. 18, (2017).
44. Nikolaou, N. et al. AKR1D1 is a novel regulator of metabolic phenotype in human hepatocytes and is dysregulated in non-alcoholic fatty liver disease. Metabolism. 99, 67-80 (2019).
45. Aljomah, G. et al. Induction of CYP2E1 in non-alcoholic fatty liver diseases. Exp. Mol. Pathol. 99, 677-681 (2015).
46. Li, H., Toth, E. & Cherrington, N. J. Alcohol Metabolism in the Progression of Human Nonalcoholic Steatohepatitis. Toxicol. Sci. 164, 428-438 (2018).
47. Farrell, G. C., Van Rooyen, D., Gan, L. & Chitturi, S. NASH is an inflammatory disorder: Pathogenic, prognostic and therapeutic implications. Gut and Liver vol. 6 149-171 (2012).
48. Pirola, C. J. & Sookoian, S. Multiomics biomarkers for the prediction of nonalcoholic fatty liver disease severity. World Journal of Gastroenterology vol. 24 1601-1615 (2018).
49. Atilano-Roque, A., Roda, G., Fogueri, U., Kiser, J. J. & Joy, M. S. Effect of Disease Pathologies on Transporter Expression and Function. J. Clin. Pharmacol. S205-S221 (2016) doi:10.1002/jcph.768.
50. Brunt, E. M. Nonalcoholic fatty liver disease and the ongoing role of liver biopsy evaluation. Hepatol. Commun. 1, 370-378 (2017).
51. Arab, J. P., Karpen, S. J., Dawson, P. A., Arrese, M. & Trauner, M. Bile acids and nonalcoholic fatty liver disease: Molecular insights and therapeutic perspectives. Hepatology vol. 65 350-362 (2017).
52. Liang, J. Q. et al. Dietary cholesterol promotes steatohepatitis related hepatocellular carcinoma through dysregulated metabolism and calcium signaling. Nat. Commun. 9, (2018).
53. Benedict, M. & Zhang, X. Non-alcoholic fatty liver disease: An expanded review. World Journal of Hepatology vol. 9 715-732 (2017).
54. Liu, Y. et al. [The role of cytochrome P450 in nonalcoholic fatty liver induced by high-fat diet: a gene expression profile analysis]. Zhonghua Gan Zang Bing Za Zhi 25, 285-290 (2017).
55. Chiang, J. Y. L. The gut's feeling on bile acid signaling in NAFLD. HepatoBiliary Surg. Nutr. 7, 151-153 (2018).
56. Chiang, J. Y. L. Targeting bile acids and lipotoxicity for NASH treatment. Hepatol. Commun. 1, 1002-1004 (2017).
57. Kuttippurathu, L., Juskeviciute, E., Dippold, R. P., Hoek, J. B. & Vadigepalli, R. A novel comparative pattern analysis approach identifies chronic alcohol mediated dysregulation of transcriptomic dynamics during liver regeneration. BMC Genomics 17, (2016).
58. Owada, Y. et al. Novel non-alcoholic steatohepatitis model with histopathological and insulin-resistant features. Pathol. Int. 68, 12-22 (2018).
59. Nomura, Y., Adachi, N. & Koyama, H. Human Mus81 and FANCB independently contribute to repair of DNA damage during replication. Genes to Cells 12, 1111-1122 (2007).
60. Denechaud, P. D. et al. E2F1 mediates sustained lipogenesis and contributes to hepatic steatosis. J. Clin. Invest. 126, 137-150 (2016).
61. Gentric, G. et al. Oxidative stress promotes pathologic polyploidization in nonalcoholic fatty liver disease. J. Clin. Invest. 125, 981-992 (2015).
62. Yang, T., Zhang, X. B. & Zheng, Z. M. Suppression of KIF14 expression inhibits hepatocellular carcinoma progression and predicts favorable outcome. Cancer Sci. 104, 552-557 (2013).
63. Natarajan, S. K. et al. FoxO3 increases miR-34a to cause palmitate-induced cholangiocyte lipoapoptosis. J. Lipid Res. 58, 866-875 (2017).
64. Waller-Evans, H. et al. Nutrigenomics of high fat diet induced obesity in mice suggests relationships between susceptibility to fatty liver disease and the proteasome. PLoS One 8, (2013).
65. Ruttkay-Nedecky, B. et al. The role of metallothionein in oxidative stress. International Journal of Molecular Sciences vol. 14 6044-6066 (2013).
66. Si, M. & Lang, J. The roles of metallothioneins in carcinogenesis. Journal of Hematology and Oncology vol. 11 1-20 (2018).
67. Ye, P. et al. Dual-Specificity Phosphatase 26 Protects Against Nonalcoholic Fatty Liver Disease in Mice Through Transforming Growth Factor Beta-Activated Kinase 1 Suppression. Hepatology 69, 1946-1964 (2019).
68. Schulien, I. et al. The transcription factor c-Jun/AP-1 promotes liver fibrosis during non-alcoholic steatohepatitis by regulating Osteopontin expression. Cell Death Differ. 26, 1688-1699 (2019).
69. Tsukamoto, H., Machida, K., Dynnyk, A. & Mkrtchyan, H. ‘Second hit’ models of alcoholic liver disease. Seminars in Liver Disease vol. 29 178-187 (2009).
70. Szabo, G. & Bala, S. Alcoholic liver disease and the gut-liver axis. World Journal of Gastroenterology vol. 16 1321-1329 (2010).
71. Wang, M.-J., Chen, F., Lau, J. T. Y. & Hu, Y.-P. Hepatocyte polyploidization and its association with pathophysiological processes. Cell Death Dis. 8, e2805-e2805 (2017).
72. Urbaschek, R. et al. Endotoxin, endotoxin-neutralizing-capacity, sCD14, sICAM-1, and cytokines in patients with various degrees of alcoholic liver disease. Alcohol. Clin. Exp. Res. 25, 261-268 (2001).
73. Hines, I. N. & Wheeler, M. D. Recent advances in alcoholic liver disease III. Role of the innate immune response in alcoholic hepatitis. American Journal of Physiology—Gastrointestinal and Liver Physiology vol. 287 (2004).
74. Jang, K.-J. et al. Liver-Chip: Reproducing Human and Cross-Species Toxicities. bioRxiv 631002 (2019) doi:10.1101/631002.
75. Tung, B. Y. & Carithers, R. L. Cholestasis and alcoholic liver disease. Clin. Liver Dis. 3, 585-601 (1999).
76. Lieber, C. S. Alcoholic fatty liver: its pathogenesis and mechanism of progression to inflammation and fibrosis. Alcohol 34, 9-19 (2004).
77. Järveläinen, H. A. Effect of chronic coadministration of endotoxin and ethanol on rat liver pathology and proinflammatory and anti-inflammatory cytokines. Hepatology 29, 1503-1510 (1999).
78. Zhou, Z. & Zhong, W. Targeting the gut barrier for the treatment of alcoholic liver disease. Liver Research vol. 1 197-207 (2017).
79. Thurman, R. G. II. Alcoholic liver injury involves activation of Kupffer cells by endotoxin. Am. J. Physiol. Liver Physiol. 275, G605-G611 (1998).
80. Kucera, O. & Cervinkova, Z. Experimental models of non-alcoholic fatty liver disease in rats. World J. Gastroenterol. 20, 8364-8376 (2014).
81. Kanuri, G. & Bergheim, I. In vitro and in vivo models of non-alcoholic fatty liver disease (NAFLD). Int. J. Mol. Sci. 14, 11963-11980 (2013).
82. Lau, J. K. C., Zhang, X. & Yu, J. Animal models of non-alcoholic fatty liver disease: current perspectives and recent advances. J. Pathol. 241, 36 114 (2017).
83. Bertola, A., Mathews, S., Ki, S. H., Wang, H. & Gao, B. Mouse model of chronic and binge ethanol feeding (the NIAAA model). Nat. Protoc. 8, 627-637 (2013).
84. Dastidar, S. G., Warner, J. B., Warner, D. R., McClain, C. J. & Kirpich, I. A. Rodent models of alcoholic liver disease: Role of binge ethanol administration. Biomolecules vol. 8 (2018).
85. Kasendra, M. et al. Development of a primary human Small Intestine-on-a-Chip using biopsy-derived organoids. Sci. Rep. 8, 2871 (2018).
86. De Chaumont, F. et al. Icy: An open bioimage informatics platform for extended reproducible research. Nature Methods vol. 9 690-696 (2012).
87. Schindelin, J. et al. Fiji: an open-source platform for biological-image analysis. Nat. Methods 9, 676-682 (2012).
88. McQuin, C. et al. CellProfiler 3.0: Next-generation image processing for biology. PLoS Biol. 16, e2005970 (2018).
89. Steger, C. An unbiased detector of curvilinear structures. IEEE Trans. Pattern Anal. Mach. Intell. 20, 113-125 (1998).
90. Love, M. I., Huber, W. & Anders, S. Moderated estimation of fold change and dispersion for RNA-seq data with DESeq2. Genome Biol. 15, 550 (2014).

### IXX. Microfluidic Channels Comprising Intestinal Cell Layers

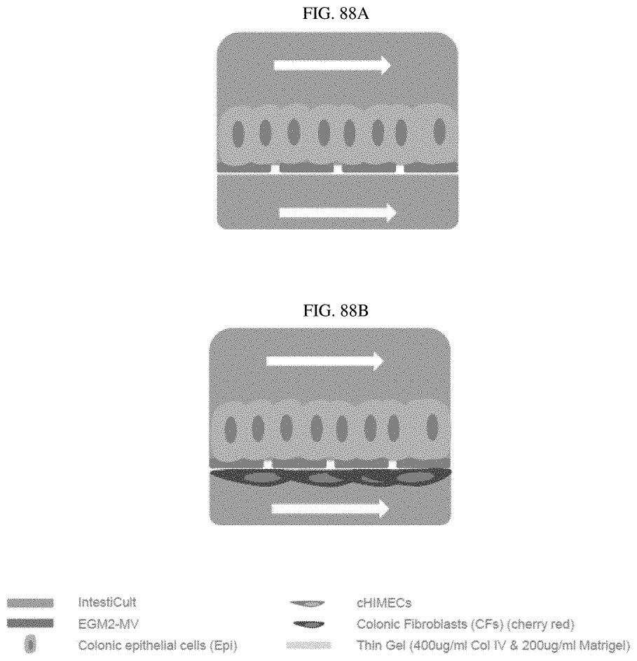

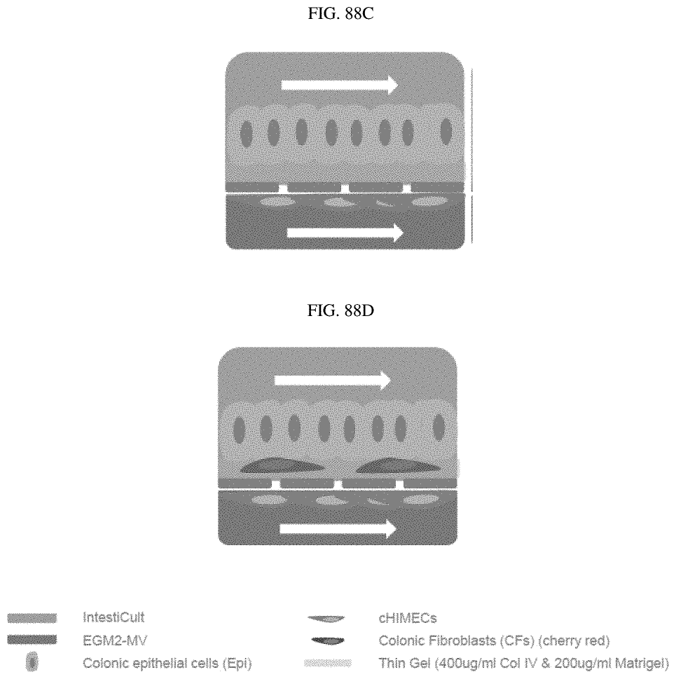

In one embodiment, the present invention contemplates a microfluidic device comprising at least one microchannel and a membrane adhered to at least one layer of an intestinal colonic epithelial cell. See, FIG. 88A-D. In one embodiment, the membrane comprises a thin gel layer. In one embodiment, the thin gel layer is collagen IV (400 μg/ml) and Matrigel® (200 μg/ml). In one embodiment, the membrane is further adhered to a colonic fibroblast cell. See, FIGS. 76B and 76D. In one embodiment, the membrane is further adhered to an endothelial cell (e.g., cHIMEC). See, FIGS. 88C and 88D. In one embodiment, the epithelial cells and the colonic fibroblasts are adhered to opposite surfaces of the membrane. In one embodiment, the fibroblasts are adhered to the membrane underneath the epithelial cells. In some embodiments, the top and bottom membrane surface is in contact with the same culture media (e.g., IntestiCult®). See, FIGS. 88A and 88B. In some embodiments, the top and bottom membrane surface is in contact with different culture media (e.g., EGMS-MV and IntestiCult®). The data presented herein demonstrate intestinal cell growth and migration of colonic fibroblasts across the membrane by Day 8. See, FIG. 89. Further assessment showed that this colonic fibroblast migration was accompanied by intestinal barrier formation as measured by apparent permeability coefficients (Pₐₚₚ). The data show that after four (4) days of culture barrier functions were intact in both the epithelial/endothelial cell layer embodiment (See, FIG. 88C) and the epithelial-colonic fibroblast/endothelial cell layer embodiment (See, FIG. 88D). However, barrier formation was not observed until Day 8 in cultures having either an epithelial layer or an epithelial/fibroblast layer. See, FIG. 89. These data show that the incorporation of colonic fibroblasts together with the colonic epithelial and endothelial cells in a microfluidic channel accelerate the establishment of an epithelial barrier.

### XX. Microfluidic Channels Comprising Kidney Cell Layers

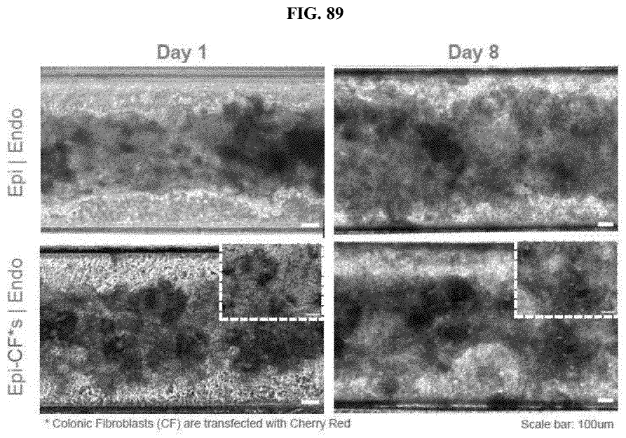

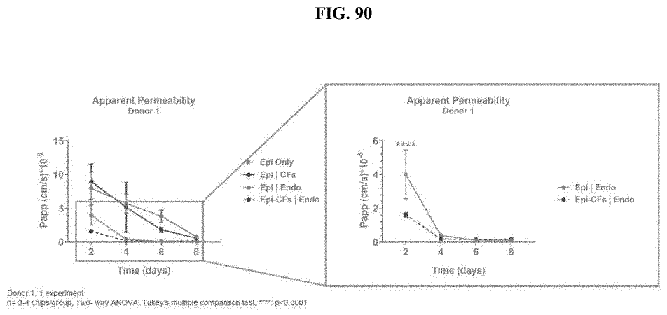

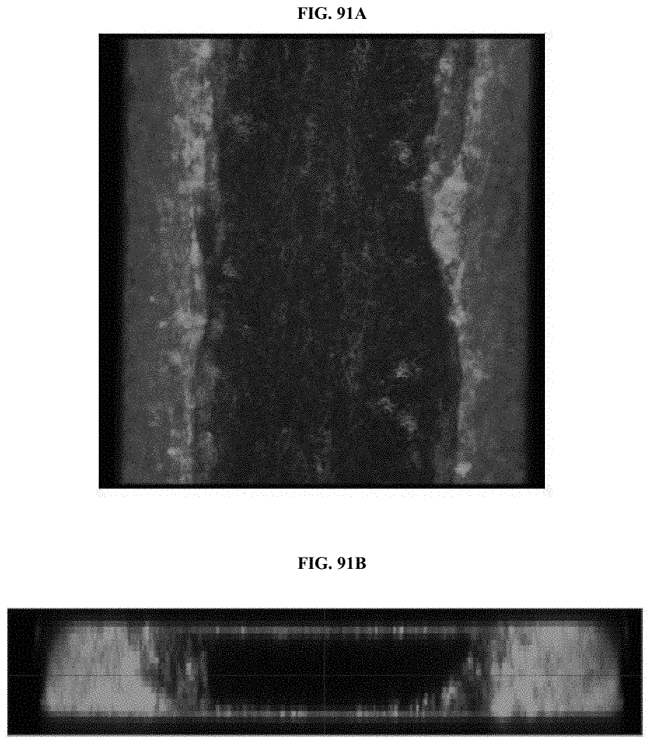

In one embodiment, the present invention contemplates a microfluidic device comprising at least one microchannel and a membrane, wherein a kidney glomerulus endothelial cell layer is adhered to a bottom surface of the membrane and are layered with a gel overlay. In one embodiment, the gel overlay comprises a thick gel layer with a lumen. In one embodiment, the thick gel layer ranging between 50-200 μm In one embodiment, the thick gel layer is collagen I (5 mg/ml)+fibronectin (62.5 μg/ml)+collagen IV (62.5 μg/ml). See, FIGS. 90A & 90B. In one embodiment, the membrane is further adhered to glomerulus mesangial cell. In one embodiment, the membrane is further adhered to a glomerulus podocyte cell. In one embodiment, the endothelial cells and the podocyte cells are adhered to opposite surfaces of the membrane. See, FIG. 90C. In one embodiment, the mesangial cells form a mesangial matrix. See, FIGS. 91A and 91B (section 1). In one embodiment, the mesangial cells and the podocyte cells form a basement membrane. See, FIGS. 91A and 91B (section 2). In one embodiment, the mesangial cells and the endothelial cells form a glycocalyx coat. See, FIGS. 91A and 91B (section 3). In one embodiment, the podocytes and the endothelial cells create a slit diaphragm. See, FIGS. 91A and 91B (section 4).

### EXPERIMENTAL

### Some Exemplary Methods.

Liver-Chip culture. Liver-Chip culture was established according to our previously published protocol⁷⁴. Briefly, primary human hepatocytes (3.5 million cells/mL) from two healthy donors, Donors G and Q, were cultured on one side of a porous membrane (pore size ˜7 microns) in the Emulate microfluidic chip ref⁸⁵. Extracellular matrix sandwiching and composition in the Liver-Chip was optimized to support BC integrity (FIG. 81A-B and method sections below). On the other side of the membrane (the lower chamber of the chip) human primary LSECs (3 million cells/mL) and KCs (0.5 million cells/mL) were seeded to mimic the hepatic sinusoid architecture (FIG. 76A). The two cell compartments were perfused independently, and flow rates were optimized to enable optimal survival and maturation of the different cell types ref⁷⁴. In the Quad Liver-Chip experiments, primary human hepatic stellate cells (50,000 cells/mL) were mixed within the collagen coating (FibriCol at 0.5 mg/ml concentration) and add to activated chips and let incubate overnight before hepatocytes seeding on the top of the collagen and HSC mix. The seeded hepatocytes were overlaid with ECM as described in FIG. 81A-B and below, and maintained in William's E Medium (WEM) containing Glutamax (Gibco), ITS+ (Corning), dexamethasone (Sigma-Aldrich), ascorbic acid (Sigma-Aldrich), fetal bovine serum (Sigma-Aldrich), and penicillin/streptomycin (Sigma-Aldrich), and incubated at 37° C., 5% CO2. The vascular channel of the Liver-Chip was maintained with human endothelial media (Emulate, Inc.). Two days after seeding, the Liver-Chips were connected to the Human Emulation System™ (Emulate, Inc.) and both chip channels were perfused at 30 μL/h to provide a continuous supply of fresh medium for the duration of the experiments. At day 5 in culture, the basal medium was modified to DMEM low glucose (1 g/L) supplemented with non-essential amino acids solution (NEAA, 1:200 dilution), glutaMAX (Thermo Fisher) ascorbic acid (Sigma-Aldrich) and penicillin/streptomycin (Sigma-Aldrich). Moreover, ITS+ premix (Corning) was replaced by ITS (GIBCO, 1:500 dilution) in order to achieve a more physiological relevant concentration of insulin, while the FBS supplement was removed.
Treatment. To model FLD, cell culture medium was supplemented at day 7 in culture either with ethanol (0.04%, 0.08% or 0.16%), with or without lipopolysaccharides (LPS, 1 μg/ml), or fatty acid (oleic acid, 1 μg/ml). The Liver-Chip was maintained for 48h in treatment medium and either assayed or allowed to recover for 5 days in basal medium.
Optimization of Liver-Chip ECM for BC integrity. We iteratively modified the original ECM coating protocol⁷⁴ in order to improve BC formation. Five different combinations of ECM composition and deposition strategy were tested (FIG. 82B), where ECM-A denoted the original protocol. Briefly, for ECM-A, top and bottom channels were coated by incubating with 100 μg/mL rat tail collagen-I (Corning) and 25 μ/mL fibronectin (Gibco) overnight at 37° C. Hepatocytes were seeded as described above. After seeding, Matrigel® prepolymer solution was prepared on ice and injected into the top channel, which was then incubated at 37° C. overnight. The following day, the top channel was gently flushed with warm medium. In conditions ECM-B, —C, -D, and -E, the hepatocytes are sandwiched between two 3D-ECM gel of different compositions, including Collagen-I (FibriCol®), Fibronectin (Gibco), Collagen-IV (Sigma), and the collagen cross-linking agent, microbial transglutaminase (MTG) (Modernist Pantry LLC). The underlying gel was prepared by injecting the gel prepolymer solution after surface activation and incubated overnight at 37° C. The next day, the gel was then flushed twice with 100 μL of warm medium at 187.5 μL/s flow (Eppendorf Xplorer automatic pipet), generating a 3D-ECM on the membrane with a thickness of 30-50 μm (data not shown). Afterwards, hepatocytes were seeded as described above. One day after the hepatocyte seeding, the same method was used to prepare the overlaying gel in conditions ECM-B, -C, and -D. In condition ECM-E, the overlay was prepared via viscous fingering method²⁸. Briefly, 5 mg/mL of bovine collagen-I (FibriCol®) was injected into the top channel and a pipette tip filled with 200 μL of warm medium was immediately injected into the channel inlet to apply hydrostatic pressure. This will cause an interface instability between medium and prepolymerized collagen-I, enabling the medium to flow through the middle part of the prepolymerized collagen-I, thereby creating a lumen (FIG. 82F). The chips were then immediately moved into humidified incubator to promote gelation of the lumen formed thick collagen-I ECM on top of the hepatocyte monolayer.

To assess and confirm ECM scaffold formation during the optimization procedure, we stained the gels with a fluorescent dye: Briefly, 1 mg/mL of N-hydroxysuccinimide (NHS) ester dye (Atto 488 NHS Ester, Sigma Aldrich) was mixed with 50 mM borate buffer (pH 9) in 1:500 ratio. Directly after ECM formation, the prepared staining solution was injected into the top channel and the chips were incubated for 25 min at room temperature in the dark. The top channel was then rinsed three times with PBS prior to fluorescence imaging.

To assess the effect of each ECM protocol on BC network formation, we quantified the MRP-2 stained BC network topology (see method sections on IF staining and Image analysis for details). Optimal conditions thus identified were ECM-D and ECM, and these protocols were then used for engineering ALD/ASH Liver-Chips.

HSC 2D and 3D culture on plate. To assess the a-smooth muscle actin (αSMA) expression in 2D versus 3D culturing conditions, HSCs were seeded into wells of a 48-well plate either in a 2D-monolayer or in a 3D ECM scaffold (type ECM-D, see FIG. 82B) that is also used on the Liver-Chip. Briefly, for the 2D culture, HSC suspension (0.05 million cells/mL) was added directly into the wells. For the 3D culture, the HSCs were first suspended in a 0.5 mg/mL Collagen-I (FibriCol®) matrix (0.05 million cells/mL) that was then added into the wells. HSC were cultured for 3 days in either conditions, and medium was refreshed every day. After 3 days in culture, the samples were fixed and immunofluorescence staining was performed as described in the separate method section to assess the αSMA activation.

Immunofluorescence staining. Cells on the Liver-Chips were washed 3× in 1×PBS then fixed with 4% paraformaldehyde for 20 minutes at room temperature (RT). Chips were then washed 3× with cold 1×PBS and blocked using 1% BSA in 1×PBS for 30 min to 2 h at RT. Cells were permeabilized using a 1×PBS solution containing 1% saponin and 10% serum matching the species of the secondary antibody for 30 min at RT. Cells were then washed 3× in 1×PBS and blocked again in a 1×PBS solution containing 1% BSA for 2 h to overnight at 4° C. All incubations with primary antibodies were carried out in this blocking buffer overnight at 4° C. This was followed by a two-hour incubation with secondary antibodies (Cell Signaling, Danvers, Mass., USA) in the blocking buffer at RT. Immunostaining was performed with the following specific primary antibodies: anti-MRP2 (1:50, Abcam), anti-Vimentin (1:50, Abcam), and anti-smooth muscle actin (anti-αSMA, 1:100, Thermo Fisher). DAPI was used to identify cell nuclei. Images were acquired with either an Olympus fluorescence microscope (IX83) or a Zeiss confocal microscope (LSM880).

Live cell staining. Liver-Chips were stained in the upper channel with AdipoRed (1:40 dilution in PBS, Lonza) to visualize lipid droplet accumulation, Tetramethylrhodamine, methyl ester (TMRM) (0.1 μM in hepatocyte medium, Thermo Fisher) to visualize active mitochondria, and MitoSox® (5 uM in hepatocyte medium, Thermo Fisher) and MitoSOX® (5 μM in hepatocyte medium, Thermo Fisher) to visualize cellular oxidative stress, and cholyl-lysyl-fluorescein (CLF, Corning) to visualize bile canaliculi. Each staining solution was prepared and added to the upper channel, incubated for 15-30 min at 37° C., and washed three times before imaging. NucBlue (Thermo Fisher) staining was used to identify cell nuclei during live imaging. The stained chips were imaged using either an Olympus fluorescence microscope (IX83) or Zeiss confocal microscope (LSM880), and were de-blurred with Olympus cellSens software.
Image analysis. Analyses of lipid droplet accumulation, ROS events, and nuclei were conducted using ICY ref⁸⁶, ImageJ-Fiji ref⁸⁷, CellProfiler ref⁸⁸, and Matlab (MATLAB, MathWorks Inc., Natick, Mass.). For ROS measurements, the histogram of the fluorescent images was adjusted to remove the background signal, followed by quantification of ROS events in the region of interest (ROI) based on minimum and maximum size and fluorescent intensity using the batch processing tool in ICY. ImageJ-Fiji was used to preprocess the AdipoRed images for analysis of lipid droplet accumulation. Here, the AdipoRed channel was median filtered, corrected for illumination, and then filtered with a Laplacian filter to emphasize droplet edges and remove larger background structures. The DAPI channel was filtered with an adaptive contrast enhancement algorithm (CLAHE) and then thresholded, followed by multiple dilation and erosion steps to yield a binary image in which nuclei in close proximity, which likely belong to a single poly-nucleated cells, are merged. These preprocessed images were further processed in CellProfiler where a pipeline first automatically segmented the fields of view into estimated cell boundaries using the nuclei as reference points, followed by thresholding and detection of lipid droplets in the AdipoRed channel within each estimated cell boundary. Using CellProfiler modules as well as Matlab scripts, we then computed mean droplet size (i.e., the projected area of the droplet in μm²) and the number of droplets per cell. Values of treatment groups were normalized to the median values of the associated control group in order to express fold-change values and thereby mitigate baseline variability due to donor-to-donor and cell batch variability. Furthermore, we computed the proportion of poly-nucleated cells by binning the detected nuclei according to their perimeter, which revealed two distinct populations, i.e. single, well separated nuclei, indicating mononucleated cells, and closely neighboring nuclei fused during thresholding, indicating poly-nucleated cells.

For measuring bile canaliculi network properties, MRP-2 stained chips were imaged at 40× using a confocal point-scanning microscope (Zeiss LSM880, Airyscan). Fields of views were randomly chosen along the entire length of the channel in order to catch heterogeneity caused by ECM deposition or erosion in flow. In each field of view, a z-stack was recorded and combined using maximal intensity projection in order to fully capture the bile canaliculi network. In subsequent image analysis, each field of view was first segmented into 16 sub-windows, and analyzed for three quantitative metrics: Porosity, branching density, and average radius (FIG. 82C-E). The area fraction taken up by the BC network, which is a measure of porosity, was determined using an ImageJ-Fiji macro that filters and thresholds the signal, followed by the ImageJ particle analysis which detects the area occupied by BC elements (Area_BC). BC porosity was then computed as the ratio of Area_BC to the total area of the field of view. Average radius and branching density were determined and applying the ImageJ-Fiji plugin “ridge detection” ref⁸⁹, to detect and measure the radius and length of all BC segments in each window. Then, we computed the average radius measured in each window as well as the branching density, defined as the summed length of the BC branches in the sub-window divided by the area of the sub-window.

The proportion of activated HSCs was determined by counting the proportion of α-SMA positive cells among the vimentin-positive cells found in two chips. For the control group, we found a total of 1 out of 33 vimentin positive cells that were also positive for α-SMA, and for the 0.08% ethanol+LPS treated group that number was 1 out of 47.

### Biochemical Assays

Cell Lysing. Cells in the Liver-Chip were lysed according to the Protocol for Emulate Organ-Chips (Cell Lysis for Protein Extraction (EP135 v1.0)). In brief, we used Tris lysis buffer (MSD, #R60TX-3]) to directly lyse the cells while still adhering to the chip, collected the lysate, and performed downstream assays.
Albumin. Albumin secretion was quantified in Liver-Chip effluent collected from the top channel using the Human Albumin SimpleStep ELISA® Kit (Abcam, #ab179887) according to the manufacturer's protocol.
Cholesterol assessment. Cholesterol was quantified in Liver-Chip effluent according to manufacturer's protocol for fluorometric detection (Thermo Fisher). Medium in the top channel was changed to standard hepatocyte medium without FBS prior to the experiment. The following sample quantification was used to determine amount of cholesterol in the hepatocytes channel effluent: Net effluent cholesterol=[cholesterol from effluent] μg/ml MINUS [Cholesterol from dosing medium] μg/ml. The same quantification method was used to determine the cholesterol concentration of the hepatocytes cell lysate as described above.
Glycogen quantification assay. Hepatocytes in the top channel of the Liver-Chip were lysed as described above, then diluted at a range of 1:500 to 1:1000. Glycogen levels were determined using a standard assay according to manufacturer's instructions for fluorometric detection (Abeam, #ab65620).
Glucose quantification assay. Glucose was quantified in Liver-Chip effluent collected from the top channel. Sample concentration were adjusted be within a 50 mg/dl to 200 mg/dl range. Glucose was quantified using a standard kit according to manufacturer's instruction (Abcam, #ab65333).
Gene expression analysis. The cells from both top and bottom channel were separately lysed from the Liver-Chip according to the Protocol for Emulate Organ-Chips: Cell Lysis for RNA Isolation (EP161 v1.0). In brief, we used PureLink RNA Mini Kit lyse buffer (Thermo #12183018) to directly lyse the cells while still adhering to the chip, collected the lysate, and immediately frozen in dry ice. Gene expression levels were analyzed by RNA sequencing performed by GENEWIZ.
RNAseq/Pathway analysis. The RNAseq dataset consisted of 2 vehicle samples, 3 samples treated with 0.08% alcohol, and 2 samples treated with 0.16% alcohol. Since differential gene expression analysis between the 0.08% and 0.16% groups yielded no significant differentially expressed genes, we pooled the samples of the two ethanol treatment groups together and constructed a single larger “ethanol-treated” group (consisting of 5 samples) which we compared to the vehicle group.

To remove poor quality adapter sequences and nucleotides, the sequence reads were trimmed using Trimmomatic v.0.36. Next, using the STAR (Spliced Transcripts Alignment to a Reference) aligner v.2.5.2b, we mapped the trimmed reads to the Homo sapiens reference genome GRCh38 (available on ENSEMBL). Using the generated BAM files and the feature Counts from the v.1.5.2 subread package, we calculated the unique gene hit counts. Of note, only unique reads that fell within exon regions were counted. We prepared a strand-specific library; therefore, the reads were strand-specifically counted. Using the gene hit counts table, we filtered out genes with very low expression across the samples. The remainder were used for differential gene expression (DGE) analysis. For the DGE analysis, we used the “DESeq2” R package⁹⁰ (Bioconductor) and in order to select the differentially expressed genes, we applied the following thresholds: adjusted p-value<0.05 and |log₂ FoldChange|>1. Of the 57,500 genes annotated in the genome, 123 were found to have significant differential expression between the vehicle (n=2) and the ethanol exposed chips (n=5). More specifically, 87 (36) genes were found to be significantly upregulated (downregulated) in the ethanol exposed chips (0.08% and 0.16%). These 123 differentially expressed genes were used for the KEGG pathway analysis.

Cytokine analysis. Liver-Chip bottom channel (containing Kupffer cells) effluent cytokine levels were measured using the U-PLEX® Biomarker Group 1 Human Assays (MSD® Cat No. K15067L) according to the manufacturer's instructions.
Statistical analysis. As indicated in legends of the drawings, one-way ANOVA, Sidak's and Dunnett's multiple comparisons tests were used for parametric data, and the Maim-Whitney U test or Kruskal-Wallis tests followed by Dunnett's multiple comparisons test was used for nonparametric data. Statistical analyses were performed using Prism v6, 7 or 8 (GraphPad). Data was collected from at least 2 independent experiments with at least 3 chips per condition and imaged at 5-10 fields of view per chip (where applicable), unless stated otherwise in the figure legends.
The following Examples I-III relate to FIGS. 93A-H-105A-B.

### Example I

### A Mucociliary Bronchiolar Epithelial Model

A microfluidic device comprising a channel separated by a membrane with increased pore size (3.0 μm vs 0.4 μm) was fabricated by previously published methods. This device allows immune cells to transmigrate from a vascular microchannel to a ciliated epithelial lumen and thus replicate inflammatory infiltrates. Human primary airway epithelial cells (hAECs) were cultured and differentiated at an air liquid interface (ALI) for 21 days on top of the collagen-coated 3 μm pore membrane while differentiation medium was continuously perfused at 60 μL/h through the bottom channel. Human primary endothelial cells were then seeded on the opposite side of the membrane from the epithelial cells, and cultured under similar flow rate until they become confluent to create a tissue-tissue interface. FIG. 93A.

Establishment of a well-differentiated mucociliary bronchiolar epithelium on one side of a 3 μm pore membrane and a confluent pulmonary microvascular endothelium on the opposite side was confirmed by immunofluorescence (hereinafter referred to as “Airway Chips”). FIG. 93B-F.

Using high-speed, real-time microscopy, cilia were observed to be actively beating in a synchronized fashion at a frequency of 16.35 (±2.6) Hz. FIG. 93F, 93G. This ciliary beating pattern generated a regional unidirectional mucociliary transport visualized by recorded trajectories of rapidly moving fluorescent microbeads diluted in PBS and introduced in the top channel. FIG. 93H. Supporting these observations were observations of tumbling small plugs of cell debris trapped in mucus. The measured bead velocity was typically ˜100 μm/sec, which is strikingly close to that reported in human airways. See, Table 3.

### Example II

### Effect of IL-13 on Ciliated Cell Beating Frequency

A microfluidic device comprising a mucociliary bronchiolar epithelium layer, prepared in accordance with Example I, was contacted with IL-13 (100 ng/mL) for 7 days. The data showed a significant increase in the number of goblet cells (16.5% total area vs 50.3%; p<0.01). FIG. 94A, 94B. The data also showed a decrease in cilia beating frequency (14.4% reduction; p<0.001). FIG. 94C, 94D.

This response was accompanied by a reduction in neutrophil velocity. FIG. 95A, 95B. Also observed was a significant twofold increase in neutrophil recruited between HRV infected chips and infected chips in presence of IL-13 (953 vs 1999; p<0.001). FIG. 95A. Real time fluorescence imaging of circulating human neutrophils revealed that many endothelium bound neutrophils, transmigrated from the vascular channel through the large 3 μm pores of the membrane, into the epithelium chamber where they adhered to the epithelial surface. FIG. 95C.

### Example III

### Ciliated Biological Cell Differentiation On-Chip

Cells were cultured and differentiated as previously described. Benam et al., “Small airway-on-a-chip enables analysis of human lung inflammation and drug responses in vitro”, Nat. Methods 13:151-7 (2016). Briefly, hAECs were seeded in the Airway Chips (prepared in accordance with Example I) on a human placenta collagen IV-coated 3 μm pore polyester membrane at a density of 3×10⁶ cells/mL and left to attach for 2 hours (hrs). Five days post seeding, air liquid interface (ALI) was introduced and Airway chips were perfused basally at 60 μL/h for 3 weeks until full differentiation. Epithelium integrity, extensive cilia beating coverage and apical mucus secretion were used for quality control.

When epithelial differentiation was fully reached, hMVECs or HUVECs were seeded onto the opposite side of the membrane, in the vascular channel at a density of 1×10⁷ cells/mL and cultured under flow for 3-4 days in endothelial growth media (EGM2-MV, Lonza, USA). The epithelial channel of each chip was gently rinsed 5× with DMEM. After the final wash, and every 24h thereafter, the epithelial surface was washed with 50 μL of DMEM.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1-151. (canceled)

152. A microfluidic device comprising at least one microchannel comprises a membrane coated with a mixed collagen 1/collagen IV partial gel layer and a cystic fibrosis cell layer.

153. The device of claim 152, wherein said cystic fibrosis cell layer is fully ciliated.

154. A method, comprising:
a) providing;
i) a microfluidic device comprising at least one microchannel;
ii) a membrane disposed within said at least one microchannel, said membrane having a first surface and a second surface; and
iii) a partial gel layer contacting said first surface of said membrane, said partial gel comprising a collagen I/collagen IV matrix;
b) seeding a plurality of living diseased cells on said partial gel layer, wherein said living disease cells are cystic fibrosis lung cells; and
c) culturing said plurality of living diseased cells.

155. The method of claim 154, wherein said cystic fibrosis lung cells differentiate after step c).

156. The method of claim 155, wherein said differentiated cystic fibrosis lung cells are ciliated.

157. The method of claim 155, wherein said cystic fibrosis lung cells exhibit at least one differentiation biomarker.

158. The method of claim 156, wherein said ciliated differentiated cystic fibrosis cells exhibit synchronous ciliary beat frequencies.

159. The method of claim 154, wherein said microchannel further comprises a cell culture media.

160. The method of claim 159, wherein said cell culture media comprises a retinoic acid-related compound, EC-23.

161. The method of claim 159, wherein said method further comprises flowing said cell culture media approximately six (6) hours after said diseased cell seeding.

162. The method of claim 154, wherein said culturing comprises exposing said seeded cells to an air-liquid interface.

163. The method of claim 155, wherein said differentiated cystic fibrosis lung cells comprise mucus-secreting goblet cells and basal cells.

164. A method, comprising:
a) providing:
i) a solution comprising gel monomers; and
ii) a microfluidic device comprising at least one microfluidic channel;
b) introducing said solution into said at least one microfluidic channel;
c) polymerizing said gel monomers to create at least a partially polymerized gel layer; and
d) removing a portion of said partially polymerized gel layer to create a partial gel layer comprising a surface that is adjacent to at least one wall of said at least one microfluidic channel, wherein said surface does not contact said at least one wall of said at least one microfluidic channel.

165. The method of claim 164, wherein said solution comprises gel monomers at a concentration of less than 0.5 mg/ml.

166. The method of claim 164, wherein said solution comprises gel monomers at a concentration of between approximately 1-3 mg/ml.

167. The method of claim 164, wherein said partially polymerized gel layer is a semi-solid gel layer.

168. The method of claim 164, wherein said partially polymerized gel layer is a solid gel layer.

169. The method of claim 164, wherein said removing comprises shearing said partially polymerized gel layer with a hydrodynamic fluid device.

170. The method of claim 169, wherein said hydrodynamic fluid device comprises a pipette or a syringe.

171. The method of claim 164, wherein said surface of said partial gel layer is flat.

172. The method of claim 164, wherein said surface of said partial gel layer is concave.
